(19) United States
(12) Patent Application Publication
(10) Pub. No.: US 2024/0182869 A1
(43) Pub. Date: Jun. 6, 2024

(54) BACTERIOPHAGE COMPOSITIONS FOR TREATING CLOSTRIDIUM PERFRINGENS INFECTIONS

(71) Applicant: APARON LTD., Middleton Tamworth (GB)

(72) Inventors: Joseph Charles Samuel BROWN, Moira Swadlincote Derbyshire (GB); Joseph Riziero EDWARDS, Selby Yorkshire (GB); Sammy James KAY, Oldbury West Midlands (GB); Ellen Olivia DENNESS, Kilham (GB); Christopher Jeremy STANFORD, Middleton Tamworth (GB)

(21) Appl. No.: 18/285,729

(22) PCT Filed: Apr. 8, 2022
(86) PCT No.: PCT/GB2022/050887

§ 371 Date: Oct. 5, 2023

Related U.S. Application Data
(60) Provisional application No. 63/173,041, filed on Apr. 9, 2021.

(51) Int. Cl. C12N 7/00; A23K 10/16; A61K 35/76; A61P 31/04; C12Q 1/04

(52) U.S. Cl. CPC C12N 7/00; A23K 10/16; A61K 35/76; A61P 31/04; C12Q 1/04; C12N 2795/10121; C12N 2795/10132; C12N 2795/10221; C12N 2795/10232; C12N 2795/10321; C12N 2795/10332

(57) ABSTRACT

The invention relates to the field of phage therapy. It particularly relates to providing phages, phage-based compositions and methods for treating or preventing bacterial infections, particularly C. perfringens, in animals, including humans, aquaculture and livestock. The invention also relates to uses of the compositions as a feedstuff and as a biological decontaminator in feed and food products for human and animal consumption.

The invention relates to the field of bacteriophage therapy. It particularly relates to providing bacteriophages, bacteriophage-based compositions and methods for treating or preventing bacterial infections, particularly C. perfringens, in animals, including humans, aquaculture and livestock. The invention also relates to uses of the compositions as a feedstuff and as a biological decontaminator in feed and food products for human and animal consumption.

Clostridium perfringens is a pathogenic Gram-positive rod-shaped spore-forming anaerobic bacterium that is omnipresent in the natural environment. The bacteria can routinely be isolated from the site of infection from hosts. The bacteria is responsible for a plethora of human diseases.

Clostridium perfringens produces toxins, which can be divided into two categories: major and minor. There are 6 major toxins: alpha (CPA), beta (CPB), iota (ITX), epsilon (ETX), enterotoxin (CPE) and the necrotic enteritis B-like toxin (NETB); these have lethal and cytotoxic attributes (Nagahama et al., 2015). There are number of minor toxins; many have yet to be defined.

The chromosomal- and plasmid-borne toxin genes are utilised as a classical toxino-type system which are used to type C. perfringens isolates (Table 1).

TABLE 1
Toxin typing of C. perfringens isolates

| | |
|---|---|
| Toxin | Gene(s) Present |

| Type | cpa | cpb | itx | etx | cpe | netB |
|---|---|---|---|---|---|---|
| A | + | − | − | − | − | − |
| B | + | + | − | + | − | − |
| C | + | + | − | − | +/− | − |
| D | + | − | − | + | +/− | − |
| E | + | − | + | − | +/− | − |
| F | + | − | − | − | + | − |
| G | + | − | − | − | − | + |

C. perfringens is regarded as the most common cause of clostridial myonecrosis (Titball et al., 1999). The disease generally occurs when a traumatic wound is infected by C. perfringens thus contaminating muscle tissue (Titball, 2005), although it has been seen in cases of healthy individuals with no underlying conditions (Boenicke et al., 2006). The disease has been reported in humans, cats, dogs, cattle, sheep, goats and horses.

C. perfringens is also the cause of necrotising enterocolitis (NEC), a severe inflammatory enteric disease with symptoms of bloody stools, bilious vomit and abdominal distention (Obladen, 2009).

C. perfringens type C isolates are responsible for “pigbel” (enteritis necroticans) and possibly identical disease darmbrand (Obladen, 2009). Pigbel is a severely debilitating enteric necrosis that is seen primarily in countries of low wealth where trypsin inhibitors are common in their diet, and a lack of protein.

Food poisoning as a result of the digestion of C. perfringens is caused primarily by type F C. perfringens or the newly discovered BEC genes, becA and becB. Food poisoning as a result of C. perfringens induces a 24 hour case of diarrhoea, which often goes unreported, due to its self-regulating nature.

Necrotic enteritis (NE) occurs when C. perfringens proliferates in the intestines of birds and produce toxins that cause necrosis of the tissues. Usually, a single clone of C. perfringens is responsible for disease, with the variant proliferating immensely and out-competing other clones of C. perfringens and other organisms. Almost all bird species have been observed to develop NE with the primarily afflicted species being commercially farmed broiler chickens; additional bird species such as turkeys (Kaldhusdal and Lovland, 2002), crows (Asaoka et al., 2004) and ducks have also been seen to develop necrotic enteritis.

Subclinical NE (SNE) is responsible for significant economic losses, due to an increase in the feed conversion ratio (FCR). The disease is estimated to affect 40% of commercial broiler flocks (Cooper and Songer, 2009). The disease collectively had been estimated to cost the global poultry industry US$2 billion as of 2001 per annum (van der Sluis, 2000). The disease has been able to proliferate due to the abolishment of antibiotic growth promoters (AGPs) and has been re-estimated to cost the global poultry industry US$6 billion per annum (Wade and Keyburn, 2015). The majority of these costs are due to a loss of productively as a result of SNE, making this form of the disease the most financially debilitating (Keyburn et al., 2008).

Novel pore-forming toxins NETE, NETF and NETG were recently isolated in samples obtained from fatal canine and foal Type A specimens (Gohari et al., 2014). Symptoms of the afflicted disease associated with the presence of the toxins are blood-streaked vomit and blood-streaked diarrheic faeces with a foul odour. Much like in acute NE in poultry, acute forms of the haemorrhagic gastroenteritis are fast-acting, with dogs not exhibiting symptoms in the evening and found dead in the morning in a pool of bloody faeces. When exhibited in foals, the disease associated with the NETF toxin carries a high mortality. Most foals are young, less than 6 days old, with disease often occurring within 24 hours of life, and foals dying within 24 hours of contraction of the disease.

Addition of antibiotic growth promoters (AGPs) in a prophylactic manner was found to improve the feed conversion ratio, with feed utilisation up 2-5%, and growth up between 4-8%. The reduction of morbidity in subclinical infections and mortality from clinical cases was also seen to decrease (Butaye et al., 2003). When an antibiotic is added to poultry environments, even in low doses, the antibiotic is able to remove non-specific bacteria. This changes the gut biome in the host, and leaves the bird exposed to risk of infection from bacteria that may be resistant to the antimicrobial used.

Whilst antibiotics have been able to provide effective protection to birds and to reduce their FCRs, this treatment and prophylactic usage is plagued with issues that are becoming more apparent and devastating. Primarily, the use of antibiotics in an ad libitum manner is causal in promoting and inducing antibiotic resistance that can not only lead to ineffective treatment of birds as a feed additive, but leads to human therapeutics being ineffective leading to morbidity and mortality in humans. A global effort is required to reduce agricultural usage of antibiotics to a treatment only usage as opposed to a performance enhancer.

There is a clear need therefore for alternatives to antibiotics in the treatment of infections in birds and other animals, and to the use of antibiotics as AGPs.

Bacteriophages (phages) are natural bacterial viruses that specifically infect and kill bacterial cells. To infect a host bacterium, a phage will initially interact with receptors on the host cell, irreversibly adsorb and inject its genome into the bacterium. The phage genomic content then migrates into the cytoplasm of the bacterial cell. Host resources, including proteins and genomes are repurposed to fuel phage replication. Replication, transcription and translation of bacteriophage genomes and progeny begins, typically by redirecting host metabolism to the production of new phage particles. Once assembly of new phage virions has reached critical mass, lysis of the bacterial cell with phage endolysins allows newly-replicated phage particles to escape the cytoplasm and go on to infect other susceptible bacteria.

A specific bacteriophage strain is known to be able to infect a narrow host range or a specific microbial species or strain. Specificity in interaction of phage with a bacterial cell is determined by the specificity of adsorption. This is dependent on the nature and structure of the receptors on the bacterial cell surface. There is clear evidence that phage can only infect a subset of a bacterial species; most phage are specific to a single bacterial species, and further specific to some strains within that species (Koskella & Meaden, 2013). For example, a bacteriophage may have the ability to infect Clostridium perfringens, but not other Clostridium species such as Clostridium septicum or Clostridium botulinum and not other bacterial species such Escherichia coli, Salmonella enteritis or Listeria monocytogenes.

Phage therapy was first used over a century ago, but has since then gone through a revival driven by the antibiotic resistance crisis. Improved understanding of phage biology, genetics, immunology and pharmacology have enabled a standardised improvement on treatment success (Altamirano & Barr, 2019).

The specificity of phage allows them to act directly against the pathogenic bacteria. In contrast, antibiotic treatment carries collateral damage, disrupting the microbiome. Phage therapy offers no off-target effects, preventing effects from microbiome disturbances such as antibiotic-associated diarrhoea, mucosal candidiasis, pseudomembranous colitis and even long-term metabolic and immunological disorders (De Sordi et al., 2017; Altamirano & Barr, 2019).

A number of novel bacteriophages have now been isolated. These bacteriophages have been shown herein to be particularly efficacious in lysing Clostridium perfringens in a species-specific manner. Combinations (cocktails) of these bacteriophages have also been shown to demonstrate synergy in their actions against Clostridium perfringens.

It is one object of the invention, therefore, to provide lytic bacteriophages which target Clostridium perfringens in a species-specific manner, and combinations (cocktails) thereof. Such bacteriophages and combinations may be used for treating or preventing bacterial infections, particularly C. perfringens, in animals, including humans, aquaculture and livestock, particularly chickens. They may also be used as feedstuffs and as a biological decontaminator in feed and food products for human and animal consumption.

In one embodiment, the invention provides a bacteriophage:
(a) wherein the genome of the bacteriophage has a nucleotide sequence as given in SEQ ID NO: 11; or
(b) wherein the genome of the bacteriophage has a nucleotide sequence which has at least 90% (preferably at least 95% or 99%) sequence identity to SEQ ID NO: 11, and wherein the bacteriophage is capable of lysing one or more strains of Clostridium perfringens.

The invention also provides a bacteriophage:
(a) wherein the genome of the bacteriophage has a nucleotide sequence as given in any one of SEQ ID NOs: 1, 2, 3, 4, 5, 6, 7, 8, 9 or 10; or
(b) wherein the genome of the bacteriophage has a nucleotide sequence which has at least 90% (preferably at least 99%) sequence identity to any one of SEQ ID NOs: 1, 2, 3, 4, 5, 6, 7, 8, 9 or 10, and wherein the bacteriophage is capable of lysing one or more strains of Clostridium perfringens.

The invention also provides a pharmaceutical composition comprising a bacteriophage as claimed in any one of the preceding claims, together with one or more pharmaceutically-acceptable carriers, excipients or diluents. The invention also provides an animal feed comprising a bacteriophage of the invention or a composition of the invention, preferably a chicken feed.

The invention also provides a bacteriophage of the invention, a composition of the invention or an animal feed of the invention, for use as a medicament or for use in therapy. The invention also provides a bacteriophage of the invention, a composition of the invention or an animal feed of the invention, for use in treating, reducing and/or preventing a disease caused by Clostridium perfringens. The invention further provides a method of treating, reducing and/or preventing a disease caused by Clostridium perfringens in a subject, wherein said method comprises administering a therapeutically-effective amount of a bacteriophage of the invention, a composition of the invention or an animal feed of the invention, to a subject in need thereof.

In another embodiment, the invention provides a process for treating a feed product or of preventing or reducing a Clostridium perfringens infection on or in a feed product, the process comprising the step:
(a) applying a bacteriophage of the invention or a composition of the invention to the feed product.

In yet another embodiment, the invention provides a method of identifying a subject who is suffering from or at risk of suffering from a Clostridium perfringens infection, comprising the steps:
(a) culturing bacteria present in a biological sample obtained from a subject;
(b) inoculating the cultured bacteria with a bacteriophage of the invention or a composition of the invention;
(c) determining whether any of the cultured bacteria are lysed by the bacteriophage or the bacteriophages in the composition,
   wherein lysis of cultured bacteria by the bacteriophage(s) is indicative of the subject suffering from or being at risk of suffering from a Clostridium perfringens infection.

The invention provides 11 new lytic bacteriophages which target Clostridium perfringens in a species-specific manner, and combinations (cocktails) thereof. These new bacteriophages are exemplified herein by reference to their genome sequences, as given in SEQ ID NOs: 1-11. The invention also provide bacteriophages having variants of these genome sequences.

The terms “bacteriophage” and “phage” (and their plurals) are synonyms, and are used interchangeably herein. The term “bacteriophage” refers to a virus that is capable of infecting a bacterium. The bacteriophages of the invention have double-stranded DNA genomes.

The bacteriophages of the invention are preferably of the family Podoviridae, Myoviridae, or Siphoviridae. The families of the preferred bacteriophages of the invention are given below:

TABLE 2
Families of the preferred bacteriophages of the invention

| SEQ ID NO: | Phage | Family |
|---|---|---|
| 1 | MN6171 | Podoviridae |
| 2 | RO96 | Siphoviridae |
| 3 | AE3110 | Myoviridae |
| 4 | ACP5 | Podoviridae |
| 5 | ACP8 | Podoviridae |
| 6 | VLR28 | Podoviridae |
| 7 | ACP10 | Podoviridae |
| 8 | ACP11 | Podoviridae |
| 9 | ACP22 | Podoviridae |
| 10 | ACP45 | Myoviridae |
| 11 | SMS460 | Myoviridae |

The bacteriophages of the invention are generally present in isolated or purified form, i.e. a form in which they are separated from their natural environment. In particular, the term “isolated” includes being substantially free of cellular material or contaminating proteins from the host cell or tissue source from which it is obtained, including being free from bacterial (e.g. Clostridium perfringens) cells. The term “purified” does not require absolute purity (such as a homogeneous preparation). Instead, it is an indication that the bacteriophage or bacteriophage preparation is relatively more pure than in the natural environment (compared to the natural level, this level should be at least 2-5 fold greater, e.g. in terms of mg/mL). Purification may be achieved according to any method known to those of ordinary skill in the art that will result in a preparation of bacteriophages substantially free from other nucleic acids, proteins, carbohydrates, lipids, subcellular organelles or bacterial cells.

The bacteriophages of the invention may, however, be combined with one or more other bacteriophages of the invention (e.g. in a composition of the invention) or other known or unknown bacteriophages.

The invention also provides variants of the bacteriophages of SEQ ID NOs: 1-11, wherein the variants have at least 90% sequence identity to one (or more) of SEQ ID NOs: 1-11. Preferably, the variants have at least 95%, 96%, 97%, 98%, 99%, 99.5% or 99.9% sequence identity to one (or more) of SEQ ID NOs: 1-11. More preferably, the variants have at least 99%, 99.5% or 99.9% sequence identity to one (or more) of SEQ ID NOs: 1-11. The variants are capable of lysing one or more strains of Clostridium perfringens.

In some embodiments, the invention provides variants of the bacteriophages of SEQ ID NOs: 1-7 and 9-11, wherein the variants have at least 90% sequence identity to one (or more) of SEQ ID NOs: 1-7 or 9-11. Preferably, the variants have at least 95%, 96%, 97%, 98%, 99%, 99.5% or 99.9% sequence identity to one (or more) of SEQ ID NOs: 1-7 or 9-11. The variants are capable of lysing one or more strains of Clostridium perfringens.

In some embodiments, the invention provides a bacteriophage:
(a) wherein the genome of the bacteriophage has a nucleotide sequence as given in SEQ ID NO: 1; or
(b) wherein the genome of the bacteriophage has a nucleotide sequence which has at least 60%, 70%, 80%, 90%, 95%, 96%, 97%, 98%, 99%, 99.5% or 99.9% sequence identity to SEQ ID NO: 1 and wherein the bacteriophage is capable of lysing one or more strains of Clostridium perfringens.

In some embodiments, the invention provides a bacteriophage:
(a) wherein the genome of the bacteriophage has a nucleotide sequence as given in SEQ ID NO: 2; or
(b) wherein the genome of the bacteriophage has a nucleotide sequence which has at least 60%, 70%, 80%, 90%, 95%, 96%, 97%, 98%, 99%, 99.5% or 99.9% sequence identity to SEQ ID NO: 2 and wherein the bacteriophage is capable of lysing one or more strains of Clostridium perfringens.

In some embodiments, the invention provides a bacteriophage:
(a) wherein the genome of the bacteriophage has a nucleotide sequence as given in SEQ ID NO: 3; or
(b) wherein the genome of the bacteriophage has a nucleotide sequence which has at least 60%, 70%, 80%, 90%, 95%, 96%, 97%, 98%, 99%, 99.5% or 99.9% sequence identity to SEQ ID NO: 3 and wherein the bacteriophage is capable of lysing one or more strains of Clostridium perfringens.

In some embodiments, the invention provides a bacteriophage:
(a) wherein the genome of the bacteriophage has a nucleotide sequence as given in SEQ ID NO: 4; or
(b) wherein the genome of the bacteriophage has a nucleotide sequence which has at least 85%, 90%, 95%, 96%, 97%, 98%, 99%, 99.5% or 99.9% sequence identity to SEQ ID NO: 4 and wherein the bacteriophage is capable of lysing one or more strains of Clostridium perfringens.

In some embodiments, the invention provides a bacteriophage:
(a) wherein the genome of the bacteriophage has a nucleotide sequence as given in SEQ ID NO: 5; or
(b) wherein the genome of the bacteriophage has a nucleotide sequence which has at least 85%, 90%, 95%, 96%, 97%, 98%, 99%, 99.5% or 99.9% sequence identity to SEQ ID NO: 5 and wherein the bacteriophage is capable of lysing one or more strains of Clostridium perfringens.

In some embodiments, the invention provides a bacteriophage:
(a) wherein the genome of the bacteriophage has a nucleotide sequence as given in SEQ ID NO: 6; or
(b) wherein the genome of the bacteriophage has a nucleotide sequence which has at least 90%, 95%, 96%, 97%, 98%, 99%, 99.5% or 99.9% sequence identity to SEQ ID NO: 6 and wherein the bacteriophage is capable of lysing one or more strains of Clostridium perfringens.

In some embodiments, the invention provides a bacteriophage:
(a) wherein the genome of the bacteriophage has a nucleotide sequence as given in SEQ ID NO: 7; or
(b) wherein the genome of the bacteriophage has a nucleotide sequence which has at least 85%, 90%, 95%, 96%, 97%, 98%, 99%, 99.5% or 99.9% sequence identity to SEQ ID NO: 7 and wherein the bacteriophage is capable of lysing one or more strains of Clostridium perfringens.

In some embodiments, the invention provides a bacteriophage:
(a) wherein the genome of the bacteriophage has a nucleotide sequence as given in SEQ ID NO: 8; or
(b) wherein the genome of the bacteriophage has a nucleotide sequence which has at least 99%, 99.5% or 99.9% sequence identity to SEQ ID NO: 8 and wherein the bacteriophage is capable of lysing one or more strains of Clostridium perfringens.

In some embodiments, the invention provides a bacteriophage:
(a) wherein the genome of the bacteriophage has a nucleotide sequence as given in SEQ ID NO: 9; or (
b) wherein the genome of the bacteriophage has a nucleotide sequence which has at least 90%, 95%, 96%, 97%, 98%, 99%, 99.5% or 99.9% sequence identity to SEQ ID NO: 9 and wherein the bacteriophage is capable of lysing one or more strains of Clostridium perfringens.

In some embodiments, the invention provides a bacteriophage:
(a) wherein the genome of the bacteriophage has a nucleotide sequence as given in SEQ ID NO: 10; or
(b) wherein the genome of the bacteriophage has a nucleotide sequence which has at least 90%, 95%, 96%, 97%, 98%, 99%, 99.5% or 99.9% sequence identity to SEQ ID NO: 10 and wherein the bacteriophage is capable of lysing one or more strains of Clostridium perfringens.

In some embodiments, the invention provides a bacteriophage:
(a) wherein the genome of the bacteriophage has a nucleotide sequence as given in SEQ ID NO: 11; or
(b) wherein the genome of the bacteriophage has a nucleotide sequence which has at least 90%, 95%, 96%, 97%, 98%, 99%, 99.5% or 99.9% sequence identity to SEQ ID NO: 11 and wherein the bacteriophage is capable of lysing one or more strains of Clostridium perfringens.

The invention also provides compositions comprising one or more of the above-defined bacteriophages.

Clustal Omega is a package for making multiple sequence alignments of amino acid or nucleotide sequences, quickly and accurately. It is a complete upgrade and rewrite of earlier Clustal programs. Clustal Omega is used to create multiple sequence alignments (MSAs). This is a procedure for aligning more than two homologous nucleotide or amino acid sequences together such that the homologous residues from the different sequences line up as much as possible in columns. This has been one of the most widely used procedures in bioinformatics for decades, as it is an essential prerequisite for most phylogenetic or comparative analyses of homologous genes or proteins. It was designed to be able to align extremely large numbers of sequences very quickly and accurately. The speed comes largely from the use of the mBed algorithm, which calculates guide trees in O(N log N) time and memory rather than O(N2) or O(N3). The accuracy comes from using Hh align, a sophisticated hidden Markov model(HMM) aligner that aligns pairs of HMMs together. The program is as fast as many alternative “fast” programs such as MAFFT or MUSCLE, but is as accurate as many other packages that are considered to be “high accuracy.” This combination makes Clustal Omega a very useful general-purpose program for making very large alignments. One feature of Clustal Omega that is especially useful is the ability to use an external HMM to help guide an alignment. This is referred to as External Profile Alignment (EPA) in this unit. It consists of taking each sequence to be aligned and aligning it against the HMM to help its alignment with other sequences in the dataset. It can be used with publicly available HMMs from PFAM, for example, or from an expert alignment that the user has built up. This procedure can also be used to iteratively generate an HMM from an output MSA and to input this to realign the input sequences. One or two iterations are usually found to have a small beneficial effect on alignment quality. (See, for example, Sievers, F., Higgins, D. G. 2014. Clustal Omega. Curr. Protoc. Bioinform. 48:3.13.1-3.13.16. (doi: 10.1002/0471250953.bi0313s48).

In the context of the current invention, suitable parameters for comparing two DNA sequences are using the CLUSTAL program Clustal Omega v1.2.3 with default parameters.

The bacteriophages of the invention are all capable of lysing one or more strains of Clostridium perfringens. The bacteriophages of the invention are all lytic bacteriophages. As used herein, a “lytic” bacteriophage refers to a virulent bacteriophage that attaches to a bacterial host and inserts its genetic material into the bacterial host cell. Lytic bacteriophages take over the machinery of the cell to make bacteriophage components. They then destroy, or lyse, the cell, releasing new bacteriophage particles. Preferably, more than 99% of the host bacterial cells are lysed and destroyed in a closed system.

Lysis of a strain of Clostridium perfringens by a bacteriophage of the invention may be demonstrated by: (a) growth inhibition; (b) optical density; (c) metabolic output; (d) photometry (e.g. fluorescence, absorption, or transmission assays); and/or (e)plaque formation.

In some embodiments of the invention, the Clostridium perfringens strain is of Type A, B, C, D, E, F or G strain. Preferably, the Clostridium perfringens strain is a Type A, C, F or G strain.

In some embodiments, the Clostridium perfringens genome comprises one or more of the following toxin-encoding genes: cpa, cpb, itx, etx, cpe netB, tpeL, cpb2, becA, becB, netE, netF, netG, cnaA, pfoA, colA.

Preferably, the bacteriophage is one which is able to lyse 5 or more Clostridium perfringens strains, more preferably 5 or more Clostridium perfringens strains associated with poultry, pigs and/or cows; more preferably those strains which are associated with poultry (e.g. chickens, turkeys, geese, ducks), preferably with chickens.

The bacteriophages of the invention have been shown herein to be capable of infecting and lysing a wide variety of strains and types of Clostridium perfringens (see, for example, Example 3 and FIG. 2 herein).

In some preferred embodiments, the bacteriophage is one:
(a) wherein the genome of the bacteriophage has a nucleotide sequence as given in any one of SEQ ID NOs: 1, 2, 3, 4, 5, 6, 7, 8, 9, 10 or 11; or
(b) wherein the genome of the bacteriophage has at least 90% (preferably at least 95% or 99%) sequence identity to a nucleotide sequence as given in any one of SEQ ID NOs: 1, 2, 3, 4, 5, 6, 7, 8, 9, 10 or 11, and the bacteriophage is capable of infecting or lysing one or more or all of the strains of Clostridium perfringens which are identified in FIG. 2 as being capable of being lysed by a bacteriophage whose genome has 100% nucleotide sequence identify to the corresponding SEQ ID NO.

For example, the invention provides a bacteriophage:
(a) wherein the genome of the bacteriophage has a nucleotide sequence as given in SEQ ID NO: 11; or
(b) wherein the genome of the bacteriophage has at least 90% (preferably at least 95% or 99%) sequence identity to nucleotide sequence as given in SEQ ID NO: 11, and wherein the bacteriophage is capable of infecting or lysing one or more or all of the following Clostridium perfringens strains ATCC 13124, Q143.CN7, JBCNJ055, Q100,CN13, Q159.4C, JBFR063, M030.1C, CP_UoA_CA_39, CP_UoA_CA_24, JBCNI056, JBCNC058, JBCNC056 and CP_UoA_CA_132 (i.e. those strains which are capable of lysing a bacteriophage of SEQ ID NO: 11, which is identified in FIG. 2 by the identifier “SMS460”).

The lytic activities of the bacteriophages of the invention are specific to Clostridium perfringens, i.e. the bacteriophages of the invention specifically lyse Clostridium perfringens. In particular, the bacteriophages of the invention are not capable of infecting or lysis other bacterial species such as Escherichia coli, Salmonella enteritis or Listeria monocytogenes). A variety of other closely-related and distant species (e.g. Clostridium septicum or Clostridium botulinum) have been tested to determine the lytic activity of the bacteriophages of the invention; no lysis was observed.

In particular, the bacteriophages of the invention have been shown not to be capable of lysing any of the following bacteria: Companilactobacillus farciminis, Lactiplantibacillus plantarum, Bifidobacterium animalis, Fusobacterium necrophorum s. necrophorum, Fusobacterium necrophrum s. fundiliforme, Enterococcus cecorum, Enterococcus hirae, Campylobacter jejuni, Salmonella enterica, Escherichia coli, Acinetobacter baumannii, Bacillus licheniformis, Bacillus cereus, Klebsiella oxytoca, Enterobacter cloacae s. cloacae, Bacillus subtilis, Staphylococcus aureus, Listeria monocytogenes, Pseudomonas aeruginosa, Enterococcus faecalis, Enterococcus faecium, Enterococcus durans, Enterococcus avium, Bacillus amyloliquefaciens, Clostridium butyricum, Clostridium septicum, Clostridium novyi, Clostridium fallax, Clostridium disporicum and Klebsiella pnuemoniae s. pnuemoniae.

In some embodiments of the invention, the bacteriophage of the invention is not a wild-type bacteriophage or a naturally-occurring bacteriophage, i.e. the bacteriophage has at least one nucleotide difference compared to a wild-type bacteriophage.

In a further embodiment, the invention provides a composition, preferably a pharmaceutical or agricultural composition, comprising one or more bacteriophages of the invention. The composition may also comprise components which are suitable for the storage of the bacteriophage(s), i.e. which maintain the stability of the bacteriophage(s).

The pharmaceutical compositions described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the pharmaceutical compositions are subjected to tableting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The composition may be a liquid or solid composition. The composition may additionally comprise one or more carriers, excipients, therapeutic agents (e.g. non-bacteriophage agents, such as antibiotics). For example, a liquid composition may comprise PBS, optionally together with a salt buffer containing magnesium, calcium and sodium chloride ions.

The agricultural composition may be a feed composition. A feed composition (e.g. a feed composition, to feed to food-producing animals) may be a solid composition or a liquid composition. In one preferred embodiment, the feed composition is a dried composition comprising one or more bacteriophages of the invention, optionally in the form of dried pellets. For example, the solid composition may be a pig feed or a chicken feed. Such dried compositions have been found to be particularly efficacious.

Suitable dosages of the bacteriophages may be in the range of 2.5×10² PFU per gram of feed to 2.5×10⁶ (e.g. 250-500, 500-1000, 1000-2500, 2500-5000, 5000-10,000, 10,000-25,000, 25,000-100,000 or 1×10⁵-1×10⁶ or 1×10⁶-2.5×10⁶). Some bacteriophages of the invention have been tested to be safe up to 5×10⁹ PFU/mL when administered orally to poultry as a liquid preparation (which is the highest usable number of bacteriophage tested).

The composition of the invention may comprise 1 or more (e.g. 1, 2, 3, 4, 5, 6, 7, 8, 9, 10, 11 or more) different bacteriophages of the invention. In some embodiments, the composition comprises 2-5, 3-5 or 3-4 different bacteriophages of the invention.

In some embodiments, the composition of the invention comprises 2 or more different bacteriophages of the invention:
(a) wherein the genome of each bacteriophage has a nucleotide sequence selected from the group consisting of SEQ ID NOs: 1, 2, 3, 4, 5, 6, 7, 8, 9, 10, 11, or a variant thereof having at least 90% (preferably at least 99%) sequence identity thereto; and
(b) which each bacteriophage is capable of lysing one more strains of Clostridium perfringens.

In some embodiments, the composition of the invention comprises 3 or more different bacteriophages of the invention:
(a) wherein the genome of each bacteriophage has a nucleotide sequence selected from the group consisting of SEQ ID NOs: 1, 2, 3, 4, 5, 6, 7, 8, 9, 10, 11, or a variant thereof having at least 90% (preferably at least 99%) sequence identity thereto; and
(b) which each bacteriophage is capable of lysing one more strains of Clostridium perfringens.

In some embodiments, the composition of the invention comprises 4 or more different bacteriophages of the invention:
(a) wherein the genome of each bacteriophage has a nucleotide sequence selected from the group consisting of SEQ ID NOs: 1, 2, 3, 4, 5, 6, 7, 8, 9, 10, 11, or a variant thereof having at least 90% (preferably at least 99%) sequence identity thereto; and
(b) which each bacteriophage is capable of lysing one more strains of Clostridium perfringens.

In some embodiments, the composition of the invention comprises 5 or more different bacteriophages of the invention:
(a) wherein the genome of each bacteriophage has a nucleotide sequence selected from the group consisting of SEQ ID NOs: 1, 2, 3, 4, 5, 6, 7, 8, 9, 10, 11, or a variant thereof having at least 90% (preferably at least 99%) sequence identity thereto; and
(b) which each bacteriophage is capable of lysing one more strains of Clostridium perfringens.

The ratios of the different bacteriophages in the composition may be the same or different. Preferably, the ratios will be the same (e.g. 1:1:1:1).

Preferred combinations (cocktails) of bacteriophages include those in the following Tables 3 and 4.

In these Tables 3-4, references to particular bacteriophages specifically include references to the SEQ ID NOs: of the genomes of those bacteriophages and variants thereof (e.g. at least 90 or at least 99% sequence identity), as defined herein.

TABLE 3
Examples of combinations (cocktails) of bacteriophages

| Cocktail | | | | | | | | | |
|---|---|---|---|---|---|---|---|---|---|
| ID | | | | | | | | | |
| 1 | MN6171 | RO96 | VLR28 | | | | | | |
| 2 | MN6171 | ACP5 | ACP10 | | | | | | |
| 3 | ACP11 | ACP45 | SMS460 | | | | | | |
| 4 | RO96 | ACP5 | ACP11 | | | | | | |
| 5 | MN6171 | RO96 | ACP5 | ACP11 | | | | | |
| 6 | ACP22 | ACP45 | SMS460 | | | | | | |
| 7 | MN6171 | RO96 | ACP5 | | | | | | |
| 8 | MN6171 | ACP5 | VLR28 | ACP11 | | | | | |
| 9 | MN6171 | ACP5 | SMS460 | | | | | | |
| 10 | MN6171 | AE3110 | ACP5 | ACP22 | | | | | |
| 11 | RO96 | ACP8 | ACP45 | SMS460 | | | | | |
| 12 | MN6171 | RO96 | ACP10 | ACP22 | | | | | |
| 13 | MN6171 | RO96 | AE3110 | ACP5 | ACP10 | ACP22 | | | |
| 14 | ACP8 | VLR28 | ACP45 | SMS460 | | | | | |
| 15 | MN6171 | ACP5 | ACP10 | ACP11 | | | | | |
| 16 | MN6171 | AE3110 | ACP5 | ACP8 | ACP11 | ACP22 | | | |
| 17 | MN6171 | RO96 | ACP5 | VLR28 | ACP10 | SMS460 | | | |
| 18 | MN6171 | AE3110 | ACP5 | ACP11 | ACP45 | | | | |
| 19 | MN6171 | AE3110 | ACP5 | ACP11 | SMS460 | | | | |
| 20 | RO96 | AE3110 | ACP5 | VLR28 | ACP10 | ACP45 | | | |
| 21 | MN6171 | ACP5 | ACP8 | VLR28 | ACP22 | SMS460 | | | |
| 22 | MN6171 | AE3110 | ACP8 | VLR28 | ACP10 | ACP11 | ACP45 | | |
| 23 | MN6171 | RO96 | AE3110 | ACP22 | | | | | |
| 24 | MN6171 | AE3110 | ACP5 | ACP8 | ACP10 | | | | |
| 25 | MN6171 | AE3110 | ACP5 | VLR28 | ACP11 | ACP22 | ACP45 | SMS460 | |
| 26 | MN6171 | AE3110 | ACP5 | ACP10 | ACP11 | SMS460 | | | |
| 27 | MN6171 | RO96 | AE3110 | ACP5 | ACP8 | VLR28 | ACP10 | ACP11 | ACP22 |
| 28 | MN6171 | ACP8 | VLR28 | ACP10 | ACP11 | SMS460 | | | |
| 29 | MN6171 | AE3110 | ACP5 | ACP8 | ACP10 | ACP11 | ACP22 | ACP45 | SMS460 |
| 30 | MN6171 | RO96 | AE3110 | ACP5 | ACP8 | VLR28 | ACP10 | ACP11 | ACP45 |
| | | | | | | | | ACP22 | SMS460 |

TABLE 4
Examples of preferred combinations (cocktails) of bacteriophages

| | Bacteriophages |
|---|---|
| A | ACP22, ACP45 and SMS460 |
| B | AE3110, VLR28 and SMS460 |
| C | MN6171, AE3110, VLR28, ACP22, ACP45 and SMS460 |
| D | RO96, AE3110, ACP45 and SMS460 |
| E | MN6171, RO96, AE3110, ACP5, ACP8, VLR28, |
| | ACP10, ACP11, ACP22, ACP45 and SMS460 |

In particular, the invention provides pharmaceutical compositions comprising bacteriophages of the invention, wherein:
(i) the genomes of the bacteriophages have nucleotide sequences as given in SEQ ID NOs: 9, 10 and 11, or variants thereof having at least 90% (preferably at least 99%) sequence identity thereto;
(ii) the genomes of the bacteriophages have nucleotide sequences as given in SEQ ID NOs: 3, 6 and 11, or variants thereof having at least 90% (preferably at least 99%) sequence identity thereto;
(iii) the genomes of the bacteriophages have nucleotide sequences as given in SEQ ID NOs: 1, 3, 6, 9, 10 and 11, or variants thereof having at least 90% (preferably at least 99%) sequence identity thereto;
(iv) the genomes of the bacteriophages have nucleotide sequences as given in SEQ ID NOs: 2, 3, 10 and 11, or variants thereof having at least 90% (preferably at least 99%) sequence identity thereto; or
(v) the genomes of the bacteriophages have nucleotide sequences as given in each of SEQ ID NOs: 1-11, or variants thereof having at least 90% (preferably at least 99%) sequence identity thereto;
   wherein the bacteriophages are each capable of lysing at least one strain of Clostridium perfringens.

The invention also provides pharmaceutical compositions comprising bacteriophages of the invention, wherein:
(vi) the genomes of the bacteriophages have nucleotide sequences as given in SEQ ID NOs: 1, 4 and 11, or variants thereof having at least 90% (preferably at least 99%) sequence identity thereto;
(vii) the genomes of the bacteriophages have nucleotide sequences as given in SEQ ID NOs: 1, 3, 4, 8 and 10, or variants thereof having at least 90% (preferably at least 99%) sequence identity thereto;
   wherein the bacteriophages are each capable of lysing at least one strain of Clostridium perfringens.

The Virulence Index is a measure of the killing or damaging ability of a phage at a given MOI. (See “The Virulence Index: A Metric for Quantitative Analysis of Phage Virulence”, Storms et al., PHAGE: Therapy, Applications, and Research, vol. 1, no. 1, 2020). The more virulent a phage, the faster it kills a large number of bacteria (or inhibits growth), and the greater the index number. The local virulence is measured on a scale from 0 to 1, where 0 represents the absence of any virulence and 1 represents maximum theoretical virulence. Such results can be used to compare the killing ability of the phage individually or in combinations (cocktails) to assess more favourable combinations, against different bacterial hosts. When applied to a composition comprising a combination of phages, the virulence index may be used to demonstrate synergy between the phages in the composition, when compared to the individual phages.

The invention also provides a kit for the treatment of a Clostridium perfringens infection, the kit comprising two or more bacteriophages of the invention.

Also provided is a combined preparation comprising 2 or more bacteriophages of the invention, or a composition of the invention comprising 2 or more bacteriophages of the invention, for separate, sequential or simultaneous administration to a subject, preferably in a form suitable for or for the treatment of a Clostridium perfringens infection.

In a further embodiment, the invention provides a method of treating, reducing and/or preventing a disease caused by Clostridium perfringens in a subject, wherein said method comprises administering a therapeutically-effective amount of a bacteriophage or composition of the invention to a subject in need thereof.

The term “therapeutically effective amount” is used to refer to an amount of bacteriophages or composition that results in prevention, delay of onset of symptoms, or amelioration of symptoms of Clostridium perfringens infection compared to an untreated control. A therapeutically-effective amount may, for example, be sufficient to treat, prevent, reduce the severity, delay the onset, and/or reduce the risk of occurrence of one or more symptoms of Clostridium perfringens infection compared to an untreated control. In a further embodiment, the invention provides a bacteriophage or composition of the invention for use in treating, reducing and/or preventing a disease caused by Clostridium perfringens. In a further embodiment, the invention provides the Use of a bacteriophage or composition of the invention in the manufacture of a medicament for use in treating, reducing and/or preventing a disease caused by Clostridium perfringens in an animal.

Preferably, the disease caused by Clostridium perfringens is food poisoning, gastroenteritis, cholecystitis, peritonitis, appendicitis, bowel perforation, muscle necrosis, soft-tissue infections, gas gangrene, necrotic enteritis or necrotising enterocolitis.

The bacteriophages and/or compositions of the invention may be given to subjects before they are infected with Clostridium perfringens. For example, young pigs during weaning are very prone to infection. Therefore, prophylactic treatment using bacteriophages and/or compositions of the invention could be administered.

The subject to be treated is an animal, preferably a bird, fish or mammal. Examples of birds to be treated include poultry, and chickens, turkeys, geese, ducks, pheasants, quail, pigeon, crows and partridges. Examples of fish to be treated include trevally, sardines, mackerel, cod, salmon, basa, haddock croaker, bream, tilapia, mullet, lizardfish, whiting, shad, catfish and shellfish. Additional examples of subjects include prawns, oysters, shrimp and lobsters.

Examples of mammals to be treated include pigs, cows, sheep, horses, cats, goats, dogs and humans. Preferably, the subject is a chicken, turkey, shrimp or a human.

The bacteriophages and compositions of the invention may be administered to the subject by any suitable route. Liquid compositions may be administered to the subject orally, intramuscularly, intra-dermally, subcutaneously or by intravenous injection. Solid compositions (e.g. feeds) may be administered orally.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation.

Preferred embodiments include about 5×10⁶ PFU/g in 500 g (USA 453.592 g/1 lb), 250 g (USA 226.796 g/0.5 lb) or 125 g (USA 113.398 g/0.25 lb) in a single dose as a dried bacteriophage composition, included as a feed additive pre-mix.

In some preferred embodiments, the bacteriophages of the invention are administered to the subject (e.g. a chicken) at a dose of 10⁴-10⁹ or 10⁴-10⁶ PFU/subject, e.g. 10⁴-10⁵, 10⁵-10⁶, 10⁶-10⁷, 10⁷-10⁸ or 10⁸-10⁹ PFU/subject. For example, the above doses may be administered (e.g. to a chicken) in a 1 ml dose.

In yet a further embodiment, the invention provides a process for treating a feed product, the process comprising the step: (a) applying a bacteriophage or a composition of the invention to the feed product. In yet a further embodiment, the invention provides a process for preventing or reducing a Clostridium perfringens infection on or in a feed product, the process comprising the step: (a) applying a bacteriophage or composition of the invention to the feed product.

The feed product may be one which is susceptible to Clostridium perfringens infection, e.g. raw or partially-cooked meat. The meat may, for example be poultry (e.g. chicken, turkey, goose, pheasant, quail or pigeon), pork or beef.

Bacteriophage detection methods enable rapid and specific identification of viable bacterial cells. Bacteriophage can be used as detection tools in numerous ways to modify, lyse, isolate and extract their bacterial hosts to enable detection.

Bacteriophage amplification assays use the production of phage progeny or host bacteria death as the detection signal. The growth of phage is measured by formation of plaques on a Petri dish. Plaques form when infected hosts lyse releasing phage progeny, which allows reinfection of bacteria. This assays require infection with bacteriophage followed by chemical inactivation of extracellular phage (i.e. virucide), and the subsequent detection of plaques using a fast-growing “reporter” organism. Each plaque is considered to be representative of one bacterium originally infected. Plaques can be extracted and tested for added specificity using PCR.

Phage capture utilises certain attributes of bacteriophage such as endolysins or tail spike proteins to selectively bind bacteria. Endolysins are enzymes, produced by phage, to break down bacterial cell walls during lysis. They comprise two domains, one to catalyse cell wall break down and one is a cell wall binding domain which specifically recognises areas of their host cell wall. Phage tail spikes selectively attach and bind to host cells to allow infection. This allows capture and isolation of the target organism via downstream separations and detection using culture, ELISA or qPCR methods (Jones et al., 2020).

In yet a further embodiment, the invention provides a method of identifying a subject who is suffering from or at risk of suffering from a Clostridium perfringens infection, comprising the steps:
(a) obtaining a biological sample derived from the subject;
(b) culturing bacteria present in the biological sample;
(c) inoculating the cultured bacteria with a bacteriophage or composition of the invention;
(d) determining whether any of the cultured bacteria are lysed by the bacteriophage or the bacteriophage in the composition,
   wherein lysis of cultured bacteria by bacteriophage is indicative of the subject suffering from or being at risk of suffering from a Clostridium perfringens infection.

The method may also be performed using a biological sample which has previously been obtained or derived from the subject. In such a case, the subject will be eligible for treatment by a bacteriophage or a composition of the invention for said Clostridium perfringens infection. Hence, above method of identifying may be followed by the step of administering to the subject an effective amount of a bacteriophage or composition of the invention.

The following embodiments are also preferred:
1. A composition comprising at least one phage, wherein said composition comprises:
a. at least one phage having the genomic sequence of phage vB_CpeM_SMS460 (SEQ ID NO: 11) or phage vB_CpeP_ABCP22 (SEQ ID NO: 9);
b. a phage variant comprising a genomic sequence at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 11 or SEQ ID NO: 9, wherein said phage variant is capable of infecting or causing lysis in a bacterium;
c. a phage variant comprising a genomic sequence that encodes a least one polypeptide variant encoded by either phage vB_CpeM_SMS460 or vB_CpeP_ABCP22, wherein said polypeptide variant has at least 70%, at least 80%, at least 85%, at least 90%, at least 91%, at least 92%, at least 93%, at least 94%, at least 95%, at least 96%, at least 97%, at least 98%, or at least 99% sequence identity to the encoded polypeptides identified as CDS for either phage vB_CpeM_SMS460 or vB_CpeP_ABCP22, wherein said phage variant is capable of infecting or lysis of a bacterium; and/or
d. a mixture comprising any one of (a)-(c).
2. The embodiment of claim 1, wherein said composition comprises at least two, at least three, at least four, at least five, at least six, at least seven, at least eight, at least nine, or at least ten phage, wherein each phage is selected from 1(a)-(c).
3. The embodiment of either claim 1 or claim 2, wherein said composition does not include phage that are found in nature.
4. The embodiment of any one of the preceding claims, wherein said composition provides a dose of each phage in the range of 10⁵ to 10¹³ pfu, preferably, wherein said dose of each phage is in the range of 10⁶ to 10¹² pfu; 10⁷ to 10¹¹ pfu; 10⁸ to 10¹¹ pfu; 10⁹ to 10¹¹ pfu; 10⁹ to 10¹⁰ pfu; or at a dose of approximately 10⁶ pfu, 10⁷ pfu, 10⁸ pfu, 10⁹ pfu, 10¹⁰ pfu, 10¹¹ pfu, 10¹² pfu, or 10¹³ pfu.
5. A method of treating, reducing and/or preventing an infection caused by one or more bacterium in an animal, wherein said method comprises administering to said animal a therapeutically effective amount of the composition of any one of the preceding embodiments.
6. A method of identifying an animal suffering or at risk of suffering from a bacterial infection, comprising the steps:
a. obtaining a biological sample derived from the animal;
b. culturing a bacterium present in the biological sample;
c. inoculating the cultured bacterium with a composition of any one of embodiments 1-3,
d. determining whether the cultured bacterium are lysed by the phage, wherein when any of the cultured bacterium are lysed by the phage, the chicken is determined (1) to be eligible for treatment by bacteriophage for said bacterial infection (2) to be suffering from a bacterial infection; and/or (3) at risk of suffering from a bacterial infection.
7. The method of either embodiments 5-6, wherein said bacterium is selected from at least one of Clostridium spp.
8. The method of embodiment 7, wherein said Clostridium spp. is C. perfringens.
9. The method of any one of embodiments 5-8, wherein said animal is a human, livestock or aquaculture.
10. The method of embodiment 9, wherein said livestock is selected from chicken, pigs, or cattle.
11. The method of any one of embodiments 5-10, wherein said animal is suffering from food poisoning, gastroenteritis, cholecystitis, peritonitis, appendicitis, bowel perforation, muscle necrosis, soft-tissue infections, gas gangrene, necrotic enteritis or necrotising enterocolitis.
12. The method of any one of embodiments 5-11, wherein the composition is administered to said animal as a food additive, orally, intramuscularly, intradermally, subcutaneously, or by IV injection.
13. The method of any one of embodiments 6-12, wherein said lysis is measured by a change in:
(a) growth inhibition;
(b) optical density;
(c) metabolic output;
(d) photometry (e.g., fluorescence, absorption, and transmission assays); and/or
(e) plaque formation.

The disclosure of each reference set forth herein is specifically incorporated herein by reference in its entirety.

### BRIEF DESCRIPTION OF THE FIGURES

FIG. 1. Toxin gene typing of C. perfringens isolates carried out polymerase chain reaction.

FIGS. 2A-2B. Bacteriophage host range of the bacteriophage of the invention. Black blocks indicate hosts.

FIGS. 3-4: Virulence index plots of individual bacteriophage and cocktails of bacteriophage on C. perfringens strain JBCNJ055 “Cocktail 9” is a combination of the bacteriophages MN6171, ACP5 and SMS460.

FIGS. 5-6: Graphs relating to the pH and temperature stability of the bacteriophages of the invention.

FIG. 7: 92 hour Kaplan-Meier survival distributions for 10 day old Cobb 500 chicken embryos inoculated with C. perfringens CP4 (2×10⁶ CFU) and subsequently inoculated with either a high dose (10⁸ PFU), low dose (10⁶ PFU) bacteriophage cocktail (ACP22, ACP45 and SMS460) or PBS.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Purification of the Phage and Sequence Characterisation

Samples of poultry faeces and intestinal contents and waste water (all in the UK) were screened for the presence of bacteriophages. Samples were hydrated and filtered through a 0.22 μm filter before screening. 100 μL of filtered sample was added to 500 μL target host bacteria (e.g. ATCC 13124) in BHI (brain heart infusion) broth. This was briefly mixed and added to 3 mL molten media (0.5% w/v BHI agar). This was then homogenised and poured over a set agar plate (2% w/v BHI agar). After overnight incubation under anaerobic gas conditions, plates were checked for visible plaques.

Each individual plaque was picked into SM buffer (100 mM NaCl, 50 mM Tris-HCl (pH 7.5), 8 mM MgSO₄, 0.1% w/v gelatin). 100 μL of this was propagated as described above. After overnight incubation under anaerobic gas condition, the plaques were picked. Each phage underwent at least 3 rounds of purification. Phage were collected by placing 5 mL SM buffer over a plate with a high plaque count. After overnight incubation at 4° C., the SM buffer was collected and filtered through a 0.22 μm filter. DNA was extracted using classic alkaline lysis methods.

The DNA from each of the bacteriophages was sequenced using Illumina sequencing methods. Genomes were assembled using Unicycler algorithms. The open reading frames (ORFs) were predicted using a combination of Geneious and GLIMMER algorithms.

### Determining Open Reading Frames and Coding Sequences

Open Reading Frames (ORFs) were identified with the minimum size of >150 genetic code and start codon parameters remaining default. Each ORF were extracted and a Basic Line Alignment Search Tool (BLAST) was conducted. The non-redundant nucleotide (nr/nt) database was selected with program BLASTn. The maximum hits was altered to 500, with all other parameters remaining constant. All sequences deemed irrelevant (not bacteriophage or viral genome sequences) were removed from the Hit Table. Sequences from the Hit were analysed opting for the consensus gene (e.g. should more than half of the genomes be labelled as ‘major capsid protein’, this label was transferred over as the coding sequence). Where no Hits were obtained, the resulting ORF was labelled as a ‘hypothetical protein’. This was repeated for all ORFs.

TABLE 5
Bacteriophages of the invention and their closest genome relatives

| | | | | GenBank |
|---|---|---|---|---|
| SEQ ID | Bacteriophage | % Sequence | Closest Genome | Accession |
| NO: | designation | Identity | Relative | Number |
| 1 | MN6171 | 57 | CPD2 | NC_048738 |
| 2 | RO96 | 51.7 | phi8074-B1 | NC_019924 |
| 3 | AE3110 | 53.3 | phiMMP03 | NC_028959 |
| 4 | ACP5 | 83.9 | CPD2 | NC_048738 |
| 5 | ACP8 | 83.3 | CPD2 | NC_048738 |
| 6 | VLR28 | 86.3 | CPS1 | NC_048661 |
| 7 | ACP10 | 84.7 | phi24R | NC_019523 |
| 8 | ACP11 | 98.3 | phiCP7R | NC_017980 |
| 9 | ACP22 | 86.6 | CPD2 | NC_048738 |
| 10 | ACP45 | 88.7 | Clo-PEP-1 | KY206887 |
| 11 | SMS460 | 88.4 | Clo-PEP-1 | KY206887 |

The nucleotide sequences of SEQ ID NOs: 1-11 are given in the accompanying Sequence Listing which forms part of the description of this patent application.

### Creating a Phylogenetic Tree

Relevant sequences were extracted from the National Center for Biotechnology Information (NCBI) viral genome database. Sequences were aligned with the built-in Clustal Omega program (version 1.2.3) ran under default parameters. The Geneious Tree Builder tool was selected. The Tamura-Nei genetic distance model was selected with all variables remaining at default except Bootstrap replicates which was changed to 10,000 to increase statistical analysis of the nodes.

### Example 2: Characterisation of C. perfringens Isolates

Bacterial colonies from various isolates were putatively identified as C. perfringens based on round black colonies, usually with lecithanase present. Single colonies were moved to BHI agar in triplicate and incubated anaerobically at 37° C. overnight. PCR confirmation of C. perfringens was carried out by screening for the C. perfringens cpa gene.

### Toxin Gene Identification

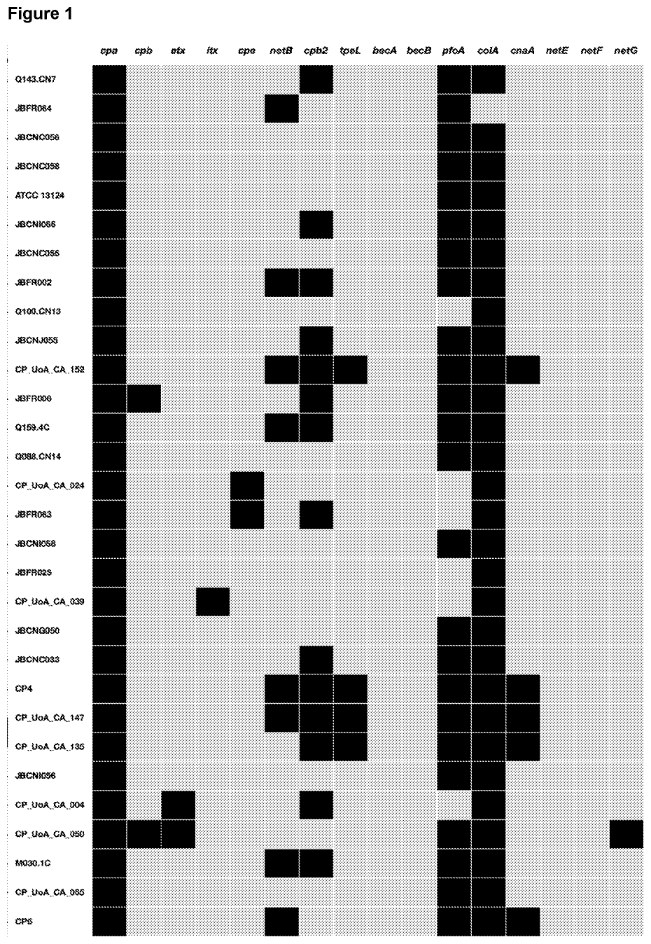

C. perfringens isolates were grown overnight on BHI agar under anaerobic conditions and then several colonies were collected aseptically. PCR reactions were performed on each colony using primers for a number of C. perfringens toxin genes (FIG. 1).

### Example 3: Bacteriophage Host Range

The host range of a bacteriophage is defined by what bacterial genera, species and strains it can lyse; it is one of the defining biological characteristics of a particular bacterial virus.

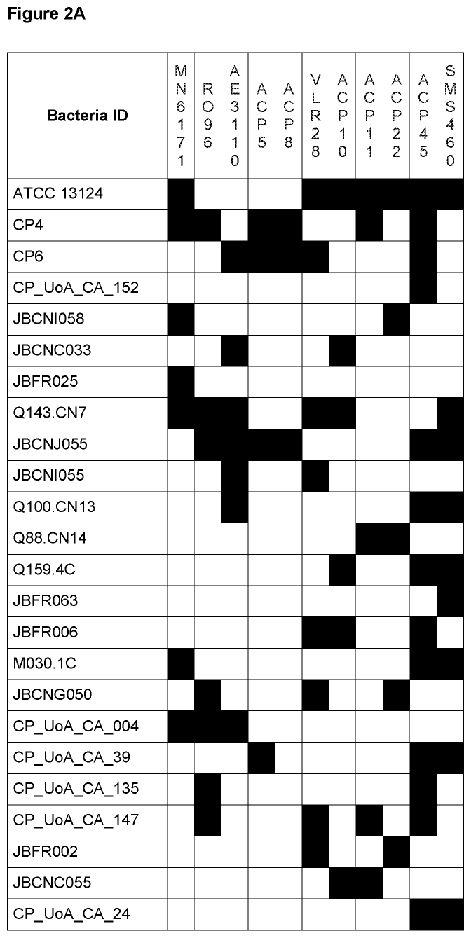

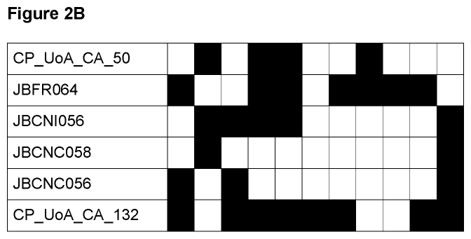

Each of the eleven selected bacteriophages (of SEQ ID NOs: 1-11) were assessed on their lytic ability/host range against numerous C. perfringens isolates; this was done by a spot assay method. A bacteriophage was presumptive to be lytic if zones of lysis could be clearly observed in all three triplicate repeats. All bacteriophage lysates were screened against thirty isolates as a first round of host range analysis. Further into the study, more isolates of C. perfringens were isolated from broiler chickens, some with putative NE. Some of these isolates were then included in the analysis of host range of the bacteriophage; these results are given in FIG. 2.

### Host Range Results

The results of this host range analysis show each bacteriophage has a broad host range capable of lysing C. perfringens of different types and source origins. The results also show that when multiple bacteriophage are used in combination, this broadens the range of the phage cocktail to successfully lyse a larger cohort of C. perfringens.

### Example 4: Calculating Multiplicity of Infection (MOI)

In a 96-well plate, 100 μL C. perfringens adjusted to 0.1 OD (at 600 nm) known to contain ×10⁸ bacterial cells was added to 100 μL bacteriophage at ×10⁸ PFU/mL. This represents an MOI of 1. Bacteriophage were diluted to varying PFU/mL titrations and added to 100 μL bacterial culture to represent MOIs to 0.00001. A positive control utilised 100 μL bacterial culture and 100 μL bacteriophage diluent; a negative control utilised 100 μL bacterial culture media and 100 μL bacteriophage diluent. The plate was incubated at 37° C. overnight. MOI was recorded as the lowest dilution of bacteriophage that prevented growth of C. perfringens. The results are shown in the table below.

TABLE 6
Multiplicity of Infection (MOI)

| Phage | Individual MOI | Cocktail MOI |
|---|---|---|
| MN6171 | 0.01 | 0.01 |
| RO96 | 0.01 | 0.01 |
| AE3110 | 0.1 | 0.01 |
| ACP5 | 0.01 | 0.001 |
| ACP8 | 0.01 | 0.001 |
| VLR28 | 0.01 | 0.001 |
| ACP10 | 0.1 | 0.001 |
| ACP11 | 0.1 | 0.01 |
| ACP22 | 0.1 | 0.01 |
| ACP45 | 0.01 | 0.01 |
| SMS460 | 0.01 | 0.001 |

### Example 5: Planktonic Killing Assay (PKA) & Virulence Index Method

Experiments were carried out according to Storms et al., 2020. In a 96-well plate, 100 μL C. perfringens adjusted to 0.1 OD (at 600 nm) known to contain ×10⁸ bacterial cells was added to 100 μL bacteriophage or bacteriophage cocktail at ×10⁸ PFU/mL to reflect an MOI of 1. A positive control utilised 100 μL bacterial culture and 100 μL bacteriophage diluent, a negative control utilised 100 μL bacterial culture media and 100 μL bacteriophage diluent. Optical density readings (at 600 nm) were recorded every 5 minutes for a total of 24 hours with 10 seconds of shaking prior to each reading. Results were recorded in triplicate and data was recorded as a single curve.

To establish a virulence index, curves were generated for each phage. Utilising the trapezoid rule, the area underneath the curves to 180 minutes or start of bacterial stationary phase. Using the two areas calculated for the free-phage control (Ao) and the culture infected at an MOI of 1 (Ai), a local virulence (vi) index score could be calculated.

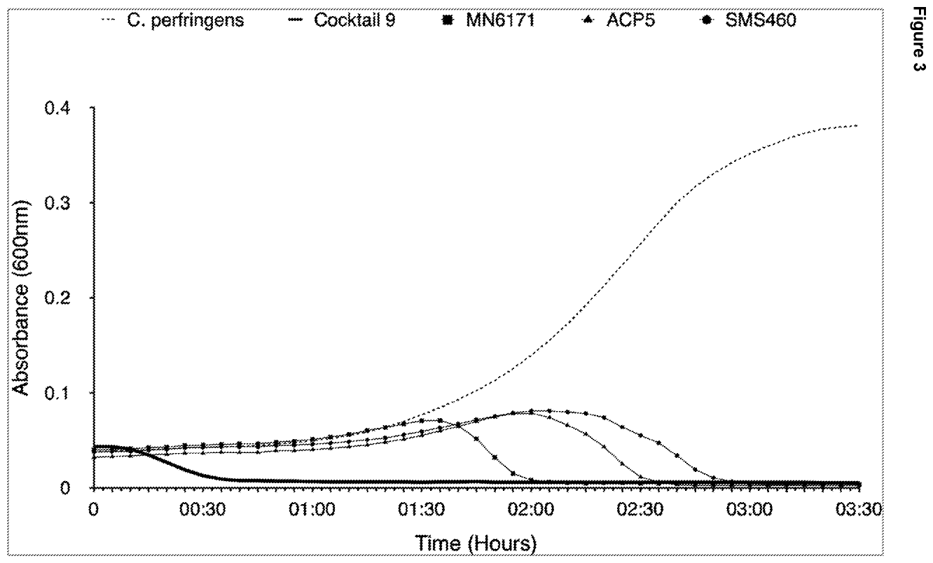

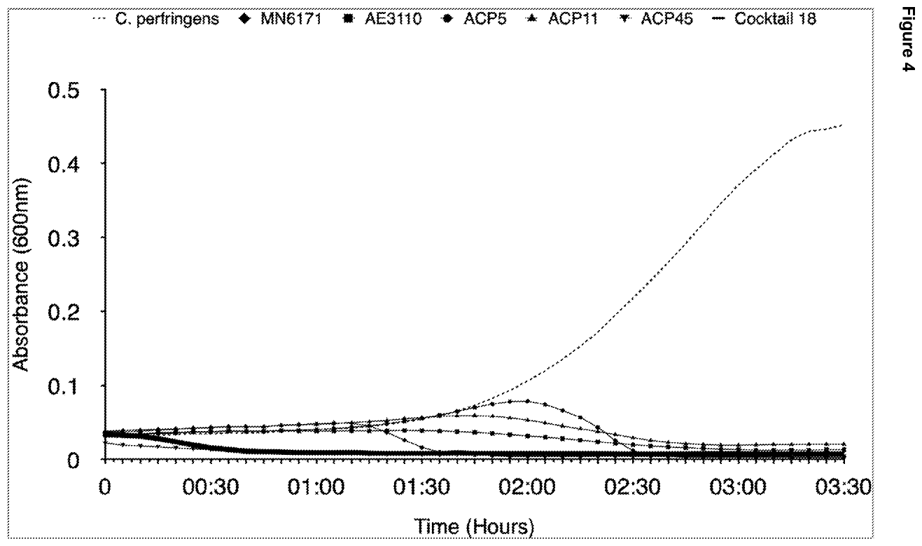

Representative results are shown in FIGS. 3-4. Tabulated result for 30 cocktails of the selected bacteriophages are given below. Details of the bacteriophages which were in the cocktails are given above in Table 3.

TABLE 7
Virulence Index - Single bacteriophage vs. Cocktails

| | Virulence | Cocktail | Virulence | Cocktail | Virulence | Cocktail | Virulence |
|---|---|---|---|---|---|---|---|
| Bacteriophage | Index | ID | Index | ID | Index | ID | Index |
| MN1671 | 0.19 | 1 | 0.79 | 11 | 0.99 | 21 | 0.97 |
| RO96 | 0.67 | 2 | 0.85 | 12 | 0.99 | 22 | 0.92 |
| AE3110 | 0.24 | 3 | 0.86 | 13 | 0.34 | 23 | 0.95 |
| ACP5 | 0.86 | 4 | 0.77 | 14 | 0.99 | 24 | 0.98 |
| ACP8 | 0.92 | 5 | 0.96 | 15 | 0.98 | 25 | 0.99 |
| VLR28 | 0.18 | 6 | 0.99 | 16 | 0.92 | 26 | 0.99 |
| ACP10 | 0.19 | 7 | 0.91 | 17 | 0.95 | 27 | 0.99 |
| ACP11 | 0.03 | 8 | 0.86 | 18 | 0.95 | 28 | 0.99 |
| ACP22 | 0.14 | 9 | 0.99 | 19 | 0.98 | 29 | 0.99 |
| ACP45 | 0.91 | 10 | 0.99 | 20 | 0.97 | 30 | 0.99 |
| SMS460 | 0.95 | | | | | | |

### Example 6: pH & Temperature Stability

500 μL phage cocktail (ACP22, ACP45 and SMS460) was diluted into 49.5 mL buffered SM solutions at different pH (2.5, 3.5, 4.5, 5.7 and 7). They were then incubated at 37° C. at 50 rpm for 180 minutes. At set time points, the titre as PFU/mL was calculated via standard double agar layer propagation methods. For dried and pelleted bacteriophage cocktail (ACP22, ACP45 and SMS460), 0.5 g pellets were used in 49.5 mL buffered SM solutions.

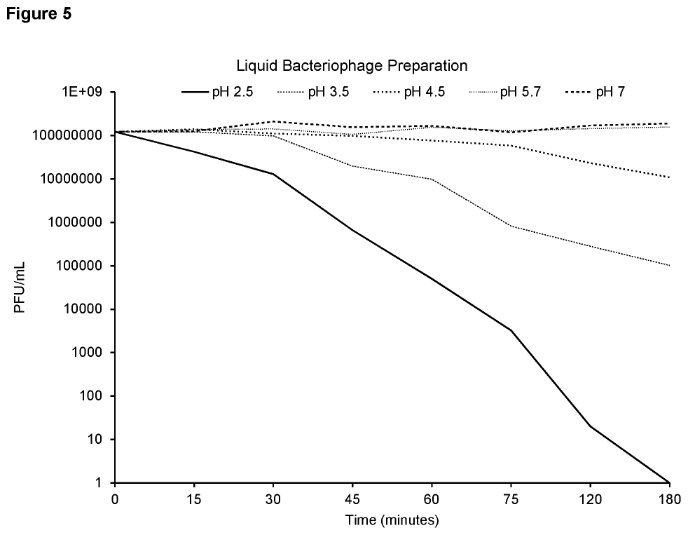

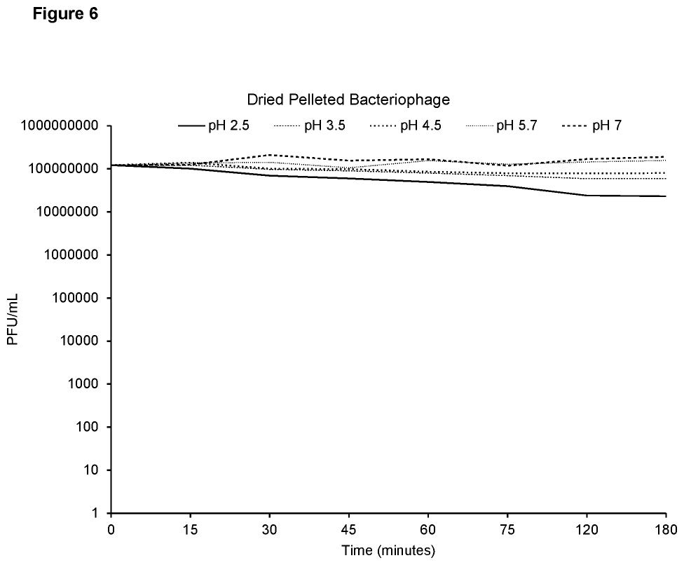

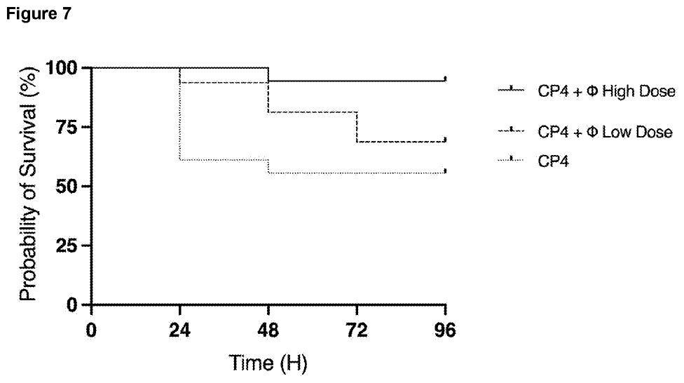

The results are shown in FIGS. 5-6. These results show that when the liquid bacteriophage cocktail (ACP22, ACP45 and SMS460) is exposed to acidic pH, like that of the gastrointestinal tract, the bacteriophage (ACP22, ACP45 and SMS460) are slowly inactivated. In comparison, when the bacteriophage cocktail (ACP22, ACP45 and SMS460) is dried and pelleted into a feed formula, the bacteriophage cocktail (ACP22, ACP45, and SMS460) can withstand the acidic environment of a gastrointestinal tract.

### Example 7: Liquid Gavage Live Bird Trials

Live bird trials were carried out by Southern Poultry Research Group in Georgia, US. The trials were carried out under the auspices of Dr. Charles L Hofacre DVM, MAM, PhD at 529 Sanford Nicholson Road Nicholson, GA 30565, USA.

440 day-of-hatch male chicks were obtained from the Aviagen Hatchery in Blairsville, GA. The bird strain used was Ross x Ross. Birds were sexed at the hatchery. Only apparent healthy chicks were used in the study.

TABLE 8
Experimental Design

| | | | Dose of Test | |
|---|---|---|---|---|
| Treatment | | C. perfringens | Article | # |
| Group | Treatment | Challenge¹ | in Feed or Water | Replicates |
| T1 | Non-Challenge Control | No | — | 4 |
| T2 | Challenge Control | Yes | — | 9 |
| T3 | Bacitracin methylene | Yes | 55 gm/metric ton | 9 |
| | disalicylate (BMD) | | | |
| T4 | Arden Phage Low Dose | Yes | 10⁶ PFU/ml* | 9 |
| T5 | Arden Phage High Dose | Yes | 10⁹ PFU/ml* | 9 |
| T6 | Phage Control | No | 10⁹ PFU/ml* | 4 |

¹DOT 14: Gavage approximately 1,500 oocysts of E. maxima as 1 dose/bird DOT 19, 20, & 21: 1.0 ml of a 1 × 10⁸ CFU/gavage/bird C. perfringens
*To be dosed by gavage of 1 ml/chick.

### Bacteriophage Administration

On days 18, 20, and 21, 1 mL of bacteriophage cocktail (ACP22, ACP45 and SMS460) was orally gavaged prior to C. perfringens gavage on days 20 and 21.

### Challenge Administration, Sample Collection and Analysis

The challenge model consisted of coccidia from approximately 1,500 oocysts (provided by Dr. Fuller) E. maxima on DOT 14 gavage to each bird and C. perfringens strain CP6 in order to target a flock mortality of 5-10%. Gavage on DOT 19, 20, and 21 using 1.0 mL of a 1.0×108 CFU/mL of C. perfringens combination previously published by Hofacre, et al. (1998).

Necrotic Enteritis lesion scoring. On DOT 22, one bird per cage was humanely euthanised, weighed necropsied and lesion scored (Hofacre, 1998).
Lesion score 0=Normal
Lesion score 1=Slight mucus covering small intestine
Lesion score 2=Necrotic small intestine mucosa
Lesion score 3=Sloughed and blood small intestine mucosa and contents

### Results

### Performance Data Analysis

Means for pen weight gain, feed consumption, feed conversion (adjusted for mortality: feed consumed/final live weight+mortality weight), lesion scores, and cause of mortality was calculated. The mortality was assessed by gross lesions on necropsy. Statistical evaluation of the data was performed using STATISTIX for Windows program (Analytical Software, Tallassee, FL). The procedures used were general linear procedures using ANOVA with a comparison of means using least significant difference (t-test) (LSD) (T)) at a significant level of 0.05.

### Study Interpretation

This study was designed to evaluate an Arden Biotechnology bacteriophage cocktail (ACP22, ACP45 and SMS460) at two dose levels for prevention of clinical necrotic enteritis (N.E.) and the subclinical effects of C. perfringens on broiler performance. In addition, the bacteriophage alone (ACP22, ACP45 and SMS460 without challenge) was evaluated to determine if the phage also had any negative effect on broiler health or performance.

### Clinical N.E. Results

Broiler chickens in T2-T5 were challenged with E. maxima on DOT 14 and C. perfringens on DOT 19, 20, and 21. The challenge control had necrotic enteritis mortality of 5.56%^{A}. The low dose (T4) had numerically lower mortality at 2.22^{AB} and the high dose (T5) was significantly lower with 0%^{B} necrotic enteritis mortality (Table 9). There were no significant differences in necrotic enteritis lesion scores, but all treatments were numerically lower. There was no effect by the phage control (T6) on mortality or necrotic enteritis lesions.

### Subclinical N.E. Results

Prior to initiation of the challenge, there were only small differences between treatments (Table 10). As the C. perfringens challenge was reaching its peak on DOT 22, both doses of phage had numerically lower FCR (Table 3). It should be noted that often the treatment with the greatest mortality may have greater feed intake; this is due to less competition from cage mates. As the birds were beginning to recover from the C. perfringens, there was no difference in weight gain to DOT 28. However, both doses of Arden Bacteriophage (T4 & T5) had FCR similar to the antibiotic, BMD (T3) (Table 12). These treatments were significantly better than the challenge control (T2). There was no negative effect of the bacteriophage alone on body weight, FCR, or feed intake at any time point measured in the study.

### Overall Conclusion

The higher dose (T5) significantly prevented necrotic enteritis death and loss. Both bacteriophage doses (T4 & T5) had a significant effect preventing negative effects of C. perfringens on the birds feed efficiency (FCR). There was no effect of the bacteriophage on broiler performance or liveability when compared to the non-challenged control.

TABLE 9
Clinical NE Results

| | | Lesion Scores | | |
|---|---|---|---|---|
| | | | (Non parametric analysis) | |
| | | | Kruskal - | |
| | | | Wallis | |
| | | Mean | One-Way | Dunn's All- |
| | NE | Score | AOV | Pairwise |
| | Percent | (Hofacre, | (Mean | Comparisons |
| Treatment | Mortality | 1998) | Rank) | Test |
| T1: Non-Challenge | 0.00A | 0.00A | 4.50 | 4.50A |
| Control | | | | |
| T6: Phage Control | 0.00A | 0.00A | 4.50 | 4.50A |
| T2: Challenge | 5.56A | 0.78A | 20.83 | 20.83A |
| Control | | | | |
| T3: Bacitracin | 2.22AB | 0.33A | 13.83 | 13.83A |
| Methylene | | | | |
| Disalicylate (BMD) | | | | |
| T4: Phage | 2.22AB | 0.67A | 19.67 | 19.67A |
| Low Dose | | | | |
| T5: Phage | 0.00B | 0.67A | 19.67 | 19.67A |
| High Dose | | | | |

**DUE TO UNEVEN REPLICATE BLOCKS, TREATMENTS 1 AND 6 ARE COMPARED TO EACH OTHER. TREATMENTS 2, 3, 4, & 5 ARE COMPARED TO EACH OTHER. **

TABLE 10
Day 0 to 14 Performance Results

| | Feed | | Non- | Weight |
|---|---|---|---|---|
| | Intake | Adjusted | Adjusted | Gain |
| Treatment | (Kg/Cage) | FCR* | FCR | (kg) |
| T1: Non-Challenge | 4.46A | 1.291A | 1.303A | 0.353A |
| Control | | | | |
| T6: Phage Control | 4.43A | 1.385A | 1.385A | 0.321A |
| T2: Challenge Control | 4.57A | 1.330B | 1.330B | 0.344A |
| T3: Bacitracin | 4.55A | 1.353AB | 1.353AB | 0.338A |
| Methylene Disalicylate | | | | |
| (BMD) | | | | |
| T4: Phage Low Dose | 4.54A | 1.324B | 1.324B | 0.345A |
| T5: Phage High Dose | 4.50A | 1.388A | 1.388A | 0.326A |

*Adjusted FCR is adjusted for mortality
**DUE TO UNEVEN REPLICATE BLOCKS, TREATMENTS 1 AND 6 ARE COMPARED TO EACH OTHER. TREATMENTS 2, 3, 4, & 5 ARE COMPARED TO EACH OTHER. **

TABLE 11
Day 0 to 22 Performance Results

| | Feed | | Non- | Weight |
|---|---|---|---|---|
| | Intake | Adjusted | Adjusted | Gain |
| Treatment | (Kg/Cage) | FCR* | FCR | (kg) |
| T1: Non-Challenge | 10.61A | 1.346A | 1.352A | 0.812A |
| Control | | | | |
| T6: Phage Control | 10.61A | 1.402A | 1.402A | 0.762A |
| T2: Challenge Control | 10.63A | 1.368AB | 1.431A | 0.781A |
| T3: Bacitracin | 10.47A | 1.361AB | 1.403AB | 0.776A |
| Methylene Disalicylate | | | | |
| (BMD) | | | | |
| T4: Phage Low Dose | 10.45A | 1.349B | 1.349B | 0.772A |
| T5: Phage High Dose | 10.51A | 1.388A | 1.388AB | 0.760A |

*Adjusted FCR is adjusted for mortality
**DUE TO UNEVEN REPLICATE BLOCKS, TREATMENTS 1 AND 6 ARE COMPARED TO EACH OTHER. TREATMENTS 2, 3, 4, & 5 ARE COMPARED TO EACH OTHER. **

TABLE 12
Day 0 to 28 Performance Results

| | | | | | Overall |
|---|---|---|---|---|---|
| | Feed Intake | Adjusted | Non-Adjusted | Weight | Mortality |
| Treatment | (Kg/Cage) | FCR* | FCR | Gain (kg) | (Percent) |
| T1: Non-Challenge Control | 16.46A | 1.461A | 1.465A | 1.201A | 2.50A |
| T6: Phage Control | 16.54A | 1.495A | 1.495A | 1.151A | 0.00A |
| T2: Challenge Control | 16.07A | 1.460A | 1.541A | 1.185A | 7.78A |
| T3: Bacitracin Methylene | 15.93A | 1.453A | 1.483B | 1.154A | 3.33AB |
| Disalicylate (BMD) | | | | | |
| T4: Phage Low Dose | 16.17A | 1.452A | 1.470B | 1.166A | 2.22AB |
| T5: Phage High Dose | 16.31A | 1.473A | 1.473B | 1.149A | 0.00B |

*Adjusted FCR is adjusted for mortality
**DUE TO UNEVEN REPLICATE BLOCKS, TREATMENTS 1 AND 6 ARE COMPARED TO EACH OTHER. TREATMENTS 2, 3, 4, & 5 ARE COMPARED TO EACH OTHER. **

### Example 8: Live bird trials using bacteriophage in feed

Live bird trials were carried out by Southern Poultry Research Group in Georgia, US. The trial was carried out under the auspices of Dr. Charles L Hofacre DVM, MAM, PhD at 529 Sanford Nicholson Road Nicholson, GA 30565.

500 day-of-hatch male chicks were obtained from the Aviagen Hatchery in Blairsville, GA. The bird strain used was Ross x Ross. Birds were sexed at the hatchery. Only apparent healthy chicks were used in the study.

TABLE 13
Experimental Design

| | | | Dose of Test | |
|---|---|---|---|---|
| Treatment | | C. perfringens | Article | # |
| Group | Treatment | Challenge¹ | in Feed or Water | Replicates |
| T1 | Non-Challenge Control | No | | 10 |
| T2 | Challenge Control | Yes | | 10 |
| T3 | Bacitracin methylene | Yes | 55 gm/metric ton | 10 |
| | disalicylate (BMD) | | | |
| T4 | Phage Safety Control (10⁹ | No | 50 gm/metric ton | 10 |
| | PFU/g) | | | |
| T5 | Bacteriophage in Feed (10⁶ | Yes | 50 gm/metric ton | 10 |
| | PFU/g) | | | |

¹DOT 14: Gavage approximately 1,500 oocysts of E. maxima as 1 dose/bird DOT 19, 20, & 21: 1.0 ml of a 1 × 10⁸ CFU/gavage/bird C. perfringens.

### Bacteriophage Administration

Bacteriophage (ACP22, ACP45 and SMS460) were applied via the feed for groups T4 and T5 at 50 gm/metric ton. The feed was be made available ad libitum throughout the trial. The bacteriophage were in all the feed administered to these groups regardless of the ration.

### Challenge Administration, Sample Collection and Analysis

The challenge model consisted of coccidia from approximately 1,500 oocysts (provided by Dr. Fuller) E. maxima on DOT 14 gavage to each bird and C. perfringens strain CP6 in order to target a flock mortality of 5-10%. Gavage on DOT 19, 20, and 21 using 1.0 mL of a 1.0×10⁸ CFU/mL of C. perfringens combination previously published by Hofacre, et al. (1998).

Necrotic Enteritis lesion scoring. On DOT 22, one bird per cage was humanely euthanised, weighed necropsied and lesion scored (Hofacre, 1998).
Lesion score 0=Normal
Lesion score 1=Slight mucus covering small intestine
Lesion score 2=Necrotic small intestine mucosa
Lesion score 3=Sloughed and blood small intestine mucosa and contents

### Results

### Performance Data Analysis

Means for pen weight gain, feed consumption, feed conversion (adjusted for mortality: feed consumed/final live weight+mortality weight), lesion scores, and cause of mortality were calculated. The mortality was assessed by gross lesions on necropsy. Statistical evaluation of the data was performed using STATISTIX for Windows program (Analytical Software, Tallassee, FL). The procedures used were general linear procedures using ANOVA with a comparison of means using least significant difference (t-test) (LSD) (T) at a significant level of 0.05.

### Study Interpretation

In this study, the bacteriophage were administered continuously in the bird's feed. Treatments 2, 3 and 5 were C. perfringens challenged on days 19 and 20. Treatment 4 was for the safety evaluation at the high dose of phage.

### Clinical Necrotic Enteritis Results

The Eimeria maxima given at 1,500 oocysts/bird was a new passage and was highly infectious. Therefore, only two days C. perfringens challenge were administered. The challenge control (T2) had 29%^{A} N.E. mortality, while the antibiotic BMD (T3) had 18%^{A} and the 10⁶ PFU/g dose (T5) of phage had 18% (Table 14). The 10⁶ PFU/g (T5) dose of phage did have a significant reduction in necrotic enteritis lesions (0.3^{BC}) versus the challenge control (0.94).

### Performance Results

Prior to the challenge (on Day 14) there were small differences in body weight and FCR (Table 15). At the peak of the challenge, the non-challenge (T1) had the heaviest body weight (Table 16) and the 10⁶ PFU/g dose (T5) of phage body weight was similar to the antibiotic BMD (T3). The 10⁶ PFU/g (T5) dose of phage had not-adjusted FCR similar to the non-challenge control (T1) and the antibiotic treatments (T3). On Day 28 in treatments that were C. perfringens challenged, the 10⁶ PFU/g (T5) dose phage and the antibiotic, BMD, had very similar body weight gain and feed efficiency (Table 17).

### Safety Results

The phage safety control (T4) with no C. perfringens challenge at 28 days had the numerically lowest overall mortality (5%c) versus not-challenged (T1) (11%^{BC}). This treatment had FCR, body weight gain, and feed intake similar to the not treated-not challenged control (T1).

### Overall

The 10⁶ PFU/g (T5) doses of phage were as effective as the antibiotic, BMD (T3), in preventing N.E. mortality in a very strong necrotic enteritis challenge. Also, the 10⁶ PFU/g (T5) dose of phage successfully prevented the negative effects of the C. perfringens on bird performance. In addition, there does not appear to be any negative effect of the phage on bird performance.

TABLE 14
Clinical NE Results

| | | Lesion Scores | | |
|---|---|---|---|---|
| | | | (Non parametric analysis) | |
| | | | Kruskal - | |
| | | | Wallis | Dunn's |
| | NE | Mean | One-Way | All- |
| | Percent | Score | AOV | Pairwise |
| | Mor- | (Hofacre, | (Mean | Comparisons |
| Treatment | tality | 1998) | Rank) | Test |
| T1: Non-Challenge | 0.00B | 0.00C | 20.00 | 20.00B |
| Control | | | | |
| T2: Challenge | 29.00A | 0.90A | 38.75 | 38.75AB |
| Control | | | | |
| T3: Bacitracin | 18.00A | 0.60AB | 33.00 | 33.00AB |
| Methylene | | | | |
| Disalicylate (BMD) | | | | |
| T4: Phage | 0.00B | 0.00C | 20.00 | 20.00B |
| Safety Control | | | | |
| (10⁹ PFU/g) | | | | |
| T5: Bacteriophage in | 18.00A | 0.30BC | 28.25 | 28.25AB |
| Feed (10⁶ PFU/g) | | | | |

TABLE 15
Day 0 to 14 Performance Results

| | Feed | | | |
|---|---|---|---|---|
| | Intake | | Non- | Weight |
| | (Kg/ | Adjusted | Adjusted | Gain |
| Treatment | Cage) | FCR* | FCR | (kg) |
| T1: Non-Challenge | 4.77A | 1.170B | 1.195ABC | 0.429A |
| Control | | | | |
| T2: Challenge | 4.79A | 1.165B | 1.180C | 0.420AB |
| Control | | | | |
| T3: Bacitracin | 4.87A | 1.186AB | 1.217AB | 0.430A |
| Methylene | | | | |
| Disalicylate | | | | |
| (BMD) | | | | |
| T4: Phage Safety | 4.92A | 1.180AB | 1.184BC | 0.424AB |
| Control (10⁹ | | | | |
| PFU/g) | | | | |
| T5: Bacteriophage | 4.75A | 1.173B | 1.180C | 0.417AB |
| in Feed (10⁶ | | | | |
| PFU/g) | | | | |

*Adjusted FCR is adjusted for mortality

TABLE 16
Day 0 to 22 Performance Results

| | Feed | | Non- | Weight |
|---|---|---|---|---|
| | Intake | Adjusted | Adjusted | Gain |
| Treatment | (Kg/Cage) | FCR* | FCR | (kg) |
| T1: Non-Challenge | 11.52AB | 1.424C | 1.492BC | 0.885A |
| Control | | | | |
| T2: Challenge Control | 11.43AB | 1.488AB | 1.737AB | 0.830BC |
| T3: Bacitracin Methylene | 11.55AB | 1.470AB | 1.662BC | 0.861AB |
| Disalicylate (BMD) | | | | |
| T4: Phage Safety | 11.98A | 1.413C | 1.416C | 0.867AB |
| Control (10⁹ | | | | |
| PFU/g) | | | | |
| T5: Bacteriophage | 11.36AB | 1.454BC | 1.624BC | 0.859AB |
| in Feed (10⁶ | | | | |
| PFU/g) | | | | |

*Adjusted FCR is adjusted for mortality

TABLE 17
Day 0 to 28 Performance Results

| | | | | | Overall |
|---|---|---|---|---|---|
| | Feed Intake | Adjusted | Non-Adjusted | Weight | Mortality |
| Treatment | (Kg/Cage) | FCR* | FCR | Gain (kg) | (Percent) |
| T1: Non-Challenge Control | 17.335AB | 1.499B | 1.563C | 1.327A | 11.00BC |
| T2: Challenge Control | 15.085CD | 1.611A | 2.215AB | 1.153B | 33.00A |
| T3: Bacitracin Methylene | 16.095BC | 1.581A | 1.867BC | 1.236AB | 24.00AB |
| Disalicylate (BMD) | | | | | |
| T4: Phage Safety Control (10⁹ | 18.045A | 1.490B | 1.524C | 1.300A | 5.00C |
| PFU/g) | | | | | |
| T5: Bacteriophage in Feed (10⁶ | 15.955BCD | 1.571A | 1.893ABC | 1.233AB | 24.00AB |
| PFU/g) | | | | | |

*Adjusted FCR is adjusted for mortality

### Example 9: Trial of Phage in Birds in ovo

Day of lay Cobb 500 broiler chicken eggs were obtained from broiler breeder stock. Eggs were incubated at 37° C. and automatically rotated every 45 minutes whilst maintaining a constant humidity of 52.5%. On day 9 of incubation, viability of the developing embryos were screened using a heart rate monitor. Eggs that were 55±10 g and contained an identifiable, viable embryo were used in subsequent experimentation.

For challenge groups, logarithmic phase C. perfringens, cultured in FTG, was adjusted to 2 ×10⁸ CFU/mL in 10 mL volumes. The adjusted cultures were centrifuged at 1600×g and the pellets washed using sterile PBS. This process was repeated in triplicate. The subsequently adjusted and washed culture was then serially diluted, using PBS to 2×10⁷ CFU/mL and drawn into 1 mL sterile syringes and fitted with a 30 g needle. Prepared inoculum and phage treatments were used within 30 min of production.

On Day 10 post incubation, all eggs were screened using a heart rate monitor. The exterior of the egg was sterilized through the application of 100% ethanol and allowed to dry prior to injection. The eggs were punctured into the air sac using a 21 G sterile needle. Eggs were injected with 100 μL of the inoculum into the allantoic cavity for a challenge of 2×10⁶ CFU subsequently followed by a ˜100 μL injection of 10⁸ PFU or 10⁶ PFU of phage cocktail or PBS as a negative control and sealed with paraffin wax. Bacteriophage control groups were first injected with ˜100 μL of PBS followed by a subsequent injection of 10⁹ PFU positive controls. Sham infection groups received two subsequent 100 μL injections of PBS. In all groups, both injections were given within 20 m of one another. Untouched groups were used as viability controls. Post injection the eggs were returned to the egg incubator.

All in vivo bird trials used bacteriophage cocktail consisting of ACP22, ACP45 and SMS460.

TABLE 18
Experimental Design of Eggs

| Treatment | | C. perfringens | Eggs/ | # |
|---|---|---|---|---|
| Group | Treatment | Challenge | Group | Replicates |
| T1 | Non-Challenge | No | 9 | 2 |
| | Control | | | |
| T2 | Challenge | Yes | 9 | 2 |
| | Control (CP4) | | | |
| T3 | Bacteriophage | No | 9 | 2 |
| | Control | | | |
| T4 | Injection | No | 9 | 2 |
| | Control (PBS) | | | |
| T5 | Bacteriophage | Yes | 9 | 2 |
| | (10⁶ PFU/mL) | | | |
| | & CP4 | | | |
| T6 | Bacteriophage | Yes | 9 | 2 |
| | (10⁹ PFU/mL) | | | |
| | & CP4 | | | |

Mortality was assessed at 24 h intervals across a 96 h infection window. Mortality was defined as a lack of observed embryonic activity, the inability to obtain a consistent heart rate and confirmed through gross pathology. 96 h post challenge, all embryos were culled by decapitation and examined for gross pathology.

### Results

No mortalities were observed within the untouched control, bacteriophage control or injection control groups. Embryos challenged with C. perfringens CP4 resulted in an average cumulative mortality of 40%. Both bacteriophage treatment groups of ACP22, ACP45, SMS460 significantly improved embryo survival (P<0.001; Mantel-Cox Log Rank Test). The high dose and low dose resulted in a cumulative survival of 5% and 28% respectively.

### Example 10: Use of the Bacteriophage in Diagnosing Bacterial Infections

Bacteriophage are used to diagnose bacterial diseases caused by C. perfringens through routine microbiological screening. Bacteriophage that target a specific bacterial pathogen are embedded within BHI agar (0.5% w/v to 0.8% w/v) at phage concentrations of ×10² to ×10⁵. Up to 100 μL reconstituted patient sample (e.g. faeces/stool sample) is then spread over the agar and incubated at 37° C. for 12-18 hours anaerobically. After incubation, the presence of plaques or zones of clearance suggest the sample contains the target bacteria. The bacteriophage within the diagnostic assay are then offered as treatment, because lysis is known to be effective.

### DISCLOSURE OF ORIGIN

All bacteriophages were isolated in the UK from either poultry or pig faeces/gastrointestinal tract or waste water. Any animal samples were part of grants and collaborations which were freely donated as part of data collection.

### REFERENCES

Asaoka, Y., Yanai, T., Hirayama, H., Une, Y., Saito, E., Sakai, H., Goryo, M., Fukushi, H. and Masegi, T. (2004) Fatal necrotic enteritis associated with Clostridium perfringens in wild crows (corvus macrorhynchos). Avian Pathology, 33(1) 19-24.
Boenicke, L., Maier, M., Merger, M., Bauer, M., Buchberger, C., Schmidt, C., Thiede, A. and Gassel, H. (2006) Retroperitoneal gas gangrene after colonoscopic polypectomy without bowel perforation in an otherwise healthy individual: Report of a case. Langenbeck's Archives of Surgery, 391(2) 157-160.
Butaye, P., Devriese, L.A. and Haesebrouck, F. (2003) Antimicrobial growth promoters used in animal feed: Effects of less well known antibiotics on gram-positive bacteria. Clinical Microbiology Reviews, 16(2) 175-188.
Cooper, K.K. and Songer, J.G. (2009) Necrotic enteritis in chickens: A paradigm of enteric infection by Clostridium perfringens type A. Anaerobe, 15(1-2) 55-60.
De Sordi, L., Khanna, V., & Debarbieux, L. (2017). The gut microbiota facilitates drifts in the genetic diversity and infectivity of bacterial viruses. Cell host & microbe, 22(6), 801-808.
Gohari, I.M., Arroyo, L., MacInnes, J.I., Timoney, J.F., Parreira, V.R. and Prescott, J.F. (2014) Characterization of Clostridium perfringens in the feces of adult horses and foals with acute enterocolitis. Canadian Journal of Veterinary Research, 78(1) 1-7.
Gordillo Altamirano, F. L., & Barr, J. J. (2019). Phage therapy in the postantibiotic era. Clinical microbiology reviews, 32(2), e00066-18.
Hofacre, C. L., Froyman, R., Gautrias, B., George, B., Goodwin, M. A., & Brown, J. (1998). Use of Aviguard and other intestinal bioproducts in experimental Clostridium perfringens-associated necrotizing enteritis in broiler chickens. Avian Diseases, 579-584.
Jones, H. J., Shield, C. G., & Swift, B. M. (2020). The Application of Bacteriophage Diagnostics for Bacterial Pathogens in the Agricultural Supply Chain: From Farm-to-Fork PHAGE, 1(4), 176-188.
Kaldhusdal, M. and Lovland, A. (2002) Clostridial necrotic enteritis and cholangiohepatitis. In: The Elanco Global Enteritis Symposium. Indianapolis, USA.
Keyburn, A.L., Boyce, J.D., Vaz, P., Bannam, T.L., Ford, M.E., Parker, D., Di Rubbo, A., Rood, J.I. and Moore, R.J. (2008) NetB, a new toxin that is associated with avian necrotic enteritis caused by Clostridium perfringens. PLOS Pathogens, 4(2) e26.
Koskella, B., & Meaden, S. (2013). Understanding bacteriophage specificity in natural microbial communities. Viruses, 5(3), 806-823.
Nagahama, M., Ochi, S., Oda, M., Miyamoto, K., Takehara, M. and Kobayashi, K. (2015) Recent insights into Clostridium perfringens beta-toxin. Toxins, 7(2) 396-406.
Obladen, M. (2009) Necrotizing enterocolitis—150 years of fruitless search for the cause. Neonatology, 96(4) 203-210.
Storms, Z. J., Teel, M. R., Mercurio, K., & Sauvageau, D. (2020). The virulence index: a metric for quantitative analysis of phage virulence. Phage, 1(1), 27-36.
Titball, R.W., Naylor, C.E. and Basak, A.K. (1999) The Clostridium perfringens α-toxin. Anaerobe, 5(2) 51-64.
Titball, R.W. (2005) Gas gangrene: An open and closed case. Microbiology, 151(9) 2821-2828.
Van der Sluis, W. (2000) Clostridial enteritis is an often underestimated problem. World Poultry, 16(7) 42-43.
Wade, B. and Keyburn, A. (2015) The true cost of necrotic enteritis. World Poult, 31(7) 16-17.

### SEQUENCES

The Sequence Listing filed with this patent application is fully incorporated herein as part of the description.

SEQUENCE LISTING FREE TEXT

| | |
|---|---|
| <210> 1 | <223> Clostridium phage vB_CpeP_MN6171, |
| | complete genome |
| <210> 2 | <223> Clostridium phage vB_CpeS_RO96, |
| | complete genome |
| <210> 3 | <223> Clostridium phage vB_CpeM_AE3110, |
| | complete genome |
| <210> 4 | <223> Clostridium phage vB_CpeP_ACP5, |
| | complete genome |
| <210> 5 | <223> Clostridium phage vB_CpeP_ACP8, |
| | complete genome |
| <210> 6 | <223> Clostridium phage vB_CpeP_VLR28, |
| | complete genome |
| <210> 7 | <223> Clostridium phage vB_CpeP_ACP10, |
| | complete genome |
| <210> 8 | <223> Clostridium phage vB_CpeP_ACP11, |
| | complete genome |
| <210> 9 | <223> Clostridium phage vB_CpeP_ACP22, |
| | complete genome |
| <210> 10 | <223> Clostridium phage vB_CpeM_ACP45, |
| | complete genome |
| <210> 11 | <223> Clostridium phage vB_CpeM_SMS460, |
| | complete genome |

1. A bacteriophage:
(a) wherein the genome of the bacteriophage has a nucleotide sequence as given in SEQ ID NO: 11; or
(b) wherein the genome of the bacteriophage has a nucleotide sequence which has at least 90% (preferably at least 95% or 99%) sequence identity to SEQ ID NO: 11
and wherein the bacteriophage is capable of lysing one or more strains of Clostridium perfringens.

2. A bacteriophage:
(a) wherein the genome of the bacteriophage has a nucleotide sequence as given in any one of SEQ ID NOs: 1, 2, 3, 4, 5, 6, 7, 8, 9 or 10; or
(b) wherein the genome of the bacteriophage has a nucleotide sequence which has at least 90% (preferably at least 99%) sequence identity to any one of SEQ ID NOs: 1, 2, 3, 4, 5, 6, 7, 8, 9 or 10, and wherein the bacteriophage is capable of lysing one or more strains of Clostridium perfringens.

3. A bacteriophage as claimed in claim 1 or claim 2, wherein the bacteriophage is capable of lysing at least 5 different Clostridium perfringens strains.

4. A bacteriophage as claimed in claim 1, wherein the different Clostridium perfringens strains comprise one or more or all of the following Clostridium perfringens strains: ATCC 13124, Q143.CN7, JBCNJ055, Q100.CN13, Q159.4C, JBFR063, M030.1C, CP_UoA_CA_39, CP_UoA_CA_24, JBCNI056, JBCNC058, JBCNC056 and CP_UoA_CA_132.

5. A bacteriophage as claimed in claim 1, wherein the genome of the bacteriophage has the nucleotide sequence as given in SEQ ID NO: 10 or 11.

6. A pharmaceutical composition comprising a bacteriophage as claimed in any one of the preceding claims, together with one or more pharmaceutically-acceptable carriers, excipients or diluents.

7. A pharmaceutical composition as claimed in claim 6, wherein the pharmaceutical composition comprises one or more additional bacteriophages, wherein the genomes of the one or more additional bacteriophages have nucleotide sequences as given in any one of SEQ ID NOs: 1, 2, 3, 4, 5, 6, 7, 8, 9, 10 or 11, or variants thereof having at least 90% (preferably at least 99%) sequence identity thereto, and wherein the additional bacteriophages are each capable of lysing at least one strain of Clostridium perfringens.

8. A pharmaceutical composition as claimed in claim 7, wherein the pharmaceutical composition comprises 1, 2, 3, 4, 5, 6, 7, 8, 9 or 10, preferably 3-5, of said additional bacteriophages.

9. A pharmaceutical composition as claimed in claim 7, wherein:
(i) the genomes of the additional bacteriophages have nucleotide sequences as given in SEQ ID NOs: 9 and 10, or variants thereof having at least 90% sequence identity thereto;
(ii) the genomes of the additional bacteriophages have nucleotide sequences as given in SEQ ID NOs: 3 and 6, or variants thereof having at least 90% sequence identity thereto;
(iii) the genomes of the additional bacteriophages have nucleotide sequences as given in SEQ ID NOs: 1, 3, 6, 9 and 10, or variants thereof having at least 90% sequence identity thereto;
(iv) the genomes of the additional bacteriophages have nucleotide sequences as given in SEQ ID NOs: 2, 3 and 10, or variants thereof having at least 90% sequence identity thereto; or
(v) the genomes of the additional bacteriophages have nucleotide sequences as given in each of SEQ ID NOs: 1-10, or variants thereof having at least 90% sequence identity thereto.

10. An animal feed comprising a bacteriophage as claimed in any one of claims 1-5 or a composition as claimed in any one of claims 6-9, preferably a chicken feed.

11. An animal feed as claimed in claim 10, wherein the animal feed is in dried form, preferably in the form of dried pellets.

12. A kit comprising:
(i) a bacteriophage as claimed in claim 1 or claim 2; and
(ii) one or more other bacteriophages.

13. A bacteriophage as claimed in any one of claims 1-5, a composition as claimed in any one of claims 6-9 or an animal feed as claimed in claims 10-11, for use as a medicament or for use in therapy.

14. A bacteriophage as claimed in any one of claims 1-5, a composition as claimed in any one of claims 6-9 or an animal feed as claimed in claims 10-11, for use in treating, reducing and/or preventing a disease caused by Clostridium perfringens.

15. A method of treating, reducing and/or preventing a disease caused by Clostridium perfringens in a subject, wherein said method comprises administering a therapeutically-effective amount of a bacteriophage as claimed in any one of claims 1-5, a composition as claimed in any one of claims 6-9 or an animal feed as claimed in claims 10-11, to a subject in need thereof.

16. Use of a bacteriophage as claimed in any one of claims 1-5 or a composition as claimed in any one of claims 6-9 in the manufacture of a medicament for use in treating, reducing and/or preventing a disease caused by Clostridium perfringens in a subject.

17. A bacteriophage or a composition for use, a method or a use as claimed in any one of claims 14-16,
wherein the bacteriophage are administered to the subject at a dose of 10⁴-10⁹ PFU/subject, preferably about 10⁶ or about 10⁹ PFU/subject.

18. A bacteriophage or a composition for use, a method or a use as claimed in any one of claims 14-17, wherein the disease caused by Clostridium perfringens is food poisoning, gastroenteritis, cholecystitis, peritonitis, appendicitis, bowel perforation, muscle necrosis, soft-tissue infections, gas gangrene, necrotic enteritis or necrotising enterocolitis.

19. A bacteriophage or a composition for use, a method or a use as claimed in any one of claims 14-18, wherein the subject is a chicken, turkey, shrimp or a human, preferably a chicken.

20. A process for treating a feed product or of preventing or reducing a Clostridium perfringens infection on or in a feed product, the process comprising the step:
(a) applying a bacteriophage as claimed in any one of claims 1-5 or a composition as claimed in any one of claims 6-9 to the feed product.

21. A method of identifying a subject who is suffering from or at risk of suffering from a Clostridium perfringens infection, comprising the steps:
(a) culturing bacteria present in a biological sample obtained from a subject;
(b) inoculating the cultured bacteria with a bacteriophage as claimed in any one of claims 1-5 or a composition as claimed in any one of claims 6-9;
(c) determining whether any of the cultured bacteria are lysed by the bacteriophage or the bacteriophages in the composition,
wherein lysis of cultured bacteria by the bacteriophage(s) is indicative of the subject suffering from or being at risk of suffering from a Clostridium perfringens infection.


SEQUENCE LISTING (11 sequences)

SEQ ID NO: 1 - DNA, 16770 bp, Unknown
  Clostridium phage vB_CpeP_MN6171, complete genome
gttgtttaaaggagagtatcaaattatgaaaacaataaacagtaactaactacaatattattgtaacatagtatcatatttatgtcaacaaaaattataaaaataaaaaataaaaaattttaaaaaaatactttacaaatgatataaactttagtataataattgtaaggggtagaccatacgaaataaaattaaacatataaacgcttatatcttgtacaaaatgtattaaacaaaattcttaaaactctagtaaaaaagtcttgacaaactcaaaagactagagtataataaaagcataggaagggaaaacacttccaaaagtaaaatacaaaaatatttaataaaagtaatggaggtaatgaaaatgataagagattacattgaaaatgaaagaaaaaaattaaggaagttatcaatatttgaaaaagttggtgaaataagattcaacaataacgatgaaggtgacttaatggaatttgaaggaattttatgttatatagatgttccatatcaaaaagtagaattaaaaagagaaacaatacttttaaaagttaattaataaaagcttagagggaaaacacttccaaagtaaatacaaaaatatttaatgaaagaaagaggtaatgtaaaatgctaaacttaaaagaacaattaaagaaagaccaacaattcggatttgattttatggttaatagagagagtggaaaaatcaaggattggaaagacgaaatcgtaacagttacacacgttgatttaatggaaaatgtaacagacaaaatgacaggcgaagttaaaaatttaatagtattcactataaaagaagacgacaatcacttctactttggcggtaacgctataacaaatatgtttaaagatttctttgagaaatacggaaaggaagcaattcaaaacttagttgagaatgaaggcttacaagttcaagtattaggaaaaatatcaaaaacatttaagacagaatacatcacattagagataaaataaaatttaatatataaagttcacgtagcaccttaaaagattagagatactcaattagggtatctctttttatgtgtataggtatagatgaagggtacgcacggggtagaatccgccttcactattgacgtttgaatgttgcggaacgacctatacatatacaaagggatatcaatctctaaaataaattaaggtggtgataatatggagcttgacgaagctttagtaaaaattacagagcaaaacgatacaatcgtatctctacaagaagagataagaaggaataaggaagccttagaagaaaaggacagagaaactcaaagattaagagaaaataatatgaagttatttttaagagtttcaggtggcgcccctaacgaggaaggagaagccttcaaccaaccggaagtaaaacaaactttcgacgacttcataaatgactgggatatataaaaaggagtgataaataaaatggcgggtaaaaagaacaatgttgaagtaactcaagccctaattaatgacgtagggggagaacttaaagcagaattacaaccaatattggacgaccaaacaatgacggttgaacaaaagagtagaaccgtctctactaagttattcggatatagtgtattaaagaactcatttattagtgcattaataaataaggtatttatgactaggttttggagtaaagcttgggaaaatcctttaaagatgttcgttgacggaaatataacttatggttatacaatagaagacttatacgtagaagcgtcagacgcagttgattattctgcacactttgaaggtggttcaactgaatgtgatgatatattcaagttagttgataataaagtctatgcgaactatttatctattaattttgaaaagaaatttaagacttcaattaaagactttgatttaagaagagcatttactagcgaatacggtctaggggatatgattagtcaattaatgaataagaatataaggggtctttacagattagaatataatctattaaaagatatgcttactaagtatatgagtggtatatcacagaaagatgttggaatgggtgctaatgctacaccaactcaattcatacaggataatcaagttactaaggtaactggagcaactactaaggagcttatagaaaatttattactgaacatcagaatttacggagagcaattcgaatttgaaagtgataaatataatagtgcgggtgtaatgcaatttagccctatgagtgatactgtaatagttactactcctcaagtatatgggtatatagataattacttagcaaaaacttataacgaggatagggtaaaaatattaaataaaataatccttattgatagtttacctgacgttaatgtaatcaagagtggtgttgctggagctacagaatacactaacgacaaaccaatagctctattaatggacgctaagctattacagttaaaaccttcattactagaaatgagagaaatagaaaatcctaacgcattagctaaaaactacttcttacatttccatggtttagttggtattgtaccattcttaaactgtaagatatttacggctactgatgaacaattaccaaaattggaggggtagaacatgaaagtaaaggaactattaggggacggttattccgttcccgttgaaacaaatgaagtaataagagccatgtttgataaggtggctaaagaattattttatgataatcactatgaacaaggttacggagctattacaaaaggtggtacacctttagggaacagagttaatgaagttttctttttagaacaaaaggaacaagcttcaatgtttggagatttagagaataatataaccggaagtttttcagatagttctataaatattcaagcgtcatgtttatttaacgaacatgagataaaaagagctgttaaagatgatttagatttacaagacatttacgatagaatgttaaaaaatcttagaacttctctactattacaaagaagaaaagcatttttaagctcacttacaaagtatatgaatgataatgaccttaaagtattaaaagggtctaaattaagcgatattgcttcaattacaaagttaggttttcaagtaccttcaagtgaatacaacgtatcagataaagagatacaatgttttgagagtgatataatttgtttaactgatttcaaaaaattaggtttgatacttaatgaacatgataatccatttattaactggttacaatttaactcaatcattgagaatacattaccggcaaatgttaatgaagaagtcgtaaatccggatatggtaatgttcgataagagagctttaaaaatcttagttaatgatagtcagtttacttatttctttaatcctaattcattaacacatacaatcacatatactgaaaatataatcatagcaacaaatccaatcgcaaatgtttgcgtattaaaggaagaacaagggggagtatagaacatggctacaatagatttattaagggtagaccaacaaatatttaataatagttatgaacacgtagcagactttaaaagtcctagcactcaaagatactattttgataataaggttatttcatcatttaataaggatattgtagttcaaccttctcgtattagcgttacttgtccatatccttatagatatttcttagacaatagtatcaattatgtaagagcctttgaagacagtaaatattattattactttattaacgacattgtatttacggctcaagctagaacagagcttatactggagcttgacgtatggcaaacttacctatggaattattccgtacttgatagtgttgtcgaaagatgtcacgtaaaaaggtggaatagtgacggtacacctacaaaagaaagtatgtacgcagatgaagcaattccaactggtgaatatataatggataatggaaatacagattataacattgaaactttaacagataccgttgttatagcgagtaccgttccattaggtattttaacagagagctcgggtggaggtactaatccaccgagtggctcaactgattgtggagattgggaaaatggtgtaatgagccgtaacggttttagatttatgaaaggatatgaaggttttggagcatatctatataaagatagtgggggagttcctactattggttatggtgtaactaagtcagaacctacaatatttgacgacctagtttcaagacaaccagttcccgaggaatacgcttctcaagttagttacaaattaaaacaaacaaattacggaaaacctatcgtagatttctgtaaggagattggaataactaaacaaaatcaatttgacgctttatgtgatttagcatttaatgctggtgtgggtgcagtagttggcacacctacttatacatcattacctactgcattacgtgaagacccattcaacgaaagttatatccgtcctatatgggaaaattacattgtacgtgacgcttccggtactgtccttgagggtttaaaagctagaagaaaagcagagtgcgatatatactttaaaaatatttatgagtttagaccaatcgtaactatcaatcaaaatggttcatacggcaatccaatcacggctaatgacggaaacggttggctaccggagtgtagcacaaaacccgagggagtttatgtggataataaagccggtaacgattggcttgtaccagttacaggaactataagttctatgtaccctgcctacccttccggagcaccacataatgccgttgacattgcttgtcccgaaggtacaccggtctatgcttcaaaagacggagtagttagagttagaaaagaacttacaacaagttatggaaaatacttgattatagctcatggagatagtgacgtagtttacgcccataatagccaattactggtaaacgttggtgataacgtaaaacagggtcaaatgatagcaaggtcgggaaatagtgggaactcaaccggagcacatttacattgggaaatacgtaacccaaacggagaaataattcaacacggagtaaaaaccgttaaccctatgccagggtataaggtaggacaaaaagtataaaggagtgattacatggcagttgatacatgggaaagtaccattaatgacttaccatatggtctatatctgtatttagttgataatgatagcacggggtcaagtgtaataggagcttcgggggataatgttttctcctgcttctatgctccctatgttagagttgaggatttagaatatagacgtattccatatgatacgtcaaggtttggaaaagtaacggaagtaagacccgaacttagttatgagcctatggtaaacagaataaattcaattaaaaatggtagtagattttttgatgttagtccaaatgtctatgacgccccattatctttagaaggtagaaggtcttggagaaatgaaagtaagttgcatacttacccttacacttatttaactttatatgacgggataaatgaaccattcaatattaaaccccagtattgcaaaggtaataagttagatattggagttaaaatgtcattagatgataaacttacaacttcattatttattcaaaattataaaggtgacacccacgggagatttgagggtctagtaataggagctaacttggagcttccaactacttcaagtaactatgcaaactttatttcgagaactcgaagcacattgcttcaaaactcaaaaaatatacaagaaaatttcattgttaataatgccgtggcagaaaataattatgaaacttcaaaagatacggctttattaagtggtggggtaaatggattagggaatatattaagtggtaacttaggtgggttattaggtacgggagcaaatctcttaggttcggcaattagtgcaaacaatagtttagcgaatactagaatgataaatagtatgaataaaaatatggctttaaagtcaatggttgctacagttgcagacgctaagacagtaccaaatacattaatctcaaaaggttctgatatcatgtatggattacgtaacatgggtaaatctcttaaaatgtttaagttccgtcaacgtgacgaatacatggaagtaataggcgactttttcgctatgtatgggtacgcacaaaatagattaatggaagtcaatactaagagccgtaaatattacaattatattaagactaaggaagttaacattacaaccataagtattccaaatacttacttggataaattaagagccatatataataatggtgtaacaatatggcacgttgatgaccctaacgtaacaccattaaactatagtaatgataatacagaggtggaagaatgataataggtagtagagctggacatagtaaaaattgtttaggggcggtaagcttacgtaatgagtgggagtgtatgaatattctcgagaaagaagtaacaaaaatattaaaagagcatgggcacacggtgatagattgtaatagctctgccaataccgaaaatggtgagttatatgaggggtgtagaaaagcaaacgcccaacacattgatatatttttatcttatcacatgaacgcttccaagaagcataacgcttatggtactgaatgttgggtacaccctaacgctagagcttcatgtaaagaaatggctcaaagaatttgtgacaatatgtcaaaattaggtttctataatagaggtgtaaaaactggtttattatatgaaatgaataacgtaaatgctccaaacataataatcgaaacttgtttttgtgataatgaaaaagatataagtatatggtcacctacaccatatgaggttatggctagacatattgcaaatgctattgacccttcaataccattagaagttaaaccaaaaaggtggcaagtcagagtttacgcatttacaacaaaagaggacgcagaaaaatgttctaaaagaataacaaatgaattaaaagcttataacgtagtagaggagatagattaaatggacgagttagtaaacttaatatcaaatataggttttcctatagtagcatgtttattcatgttcaatcaaaatcaaaaattaactgaaactatcacggatttaaaagtcactttatccggtatcgaaaagaggttggaagcatgggaaagaaaagaaatataaataaagtttgtgcagatagaagagggcttctcttcttactgtacaaaaatttagcaacaaatgtttttaagtgggaggggctaccaaatggaattgagagccgtcatatagagaattttttatttgaacatggacaagtaggtttctacaatgataaagatttaggaatgatttgtttacctatttccaactatggggagctaaatatttatggtgacccaacaaaattttatatgtactctaaaaatggcaactatagtaaaatcattcctagcgataaaataattaggtgtatggataatcccaacttagtaccaactaggcaatatgtaaactactacgtacaggaaatgttagatatagaaactgctatacgtgccaacctaagaaaacaagtaaaaccatactttgctattgctacagataagaataaatataccgtaaaaagtattattgatgactacgaaaacggtgaagacgtagtaattatagataagacattaggtgaagacgggttcgacggattaaagctcctaacggctaacgttgagtatttagttgataagttaagaagcgaagagagaagccgagagagtgctctattaacttacttgggtgtaagtaatgtaaacatggaaaagaaggaaagattgattacagatgaggtcaatggtaatgaagagtttgtaaatcttaaccttagtataagagcgaaaggtagattagacgctatgcaagaacttcaaaaaatatatccggaagtaaaatgcgttcttaatatggaatacttagagcaatttttcaaatcattaagaagtgaggtctttgaaaatggggtattataatatagaacttagaaaaatccaaaatatatttaactttgattatccattcttcgacgaaagtaaaagagcagagtttgaaaggctctttttacgtcgatatgcttttcgtcaaattggttacccaaccattggtgagtttaagtatagattacaaacttaccttgaagaaaacaatgattactttactcaattatgggagatagagttaagatgtaaagccattgattttatgaataacaaagaatacaccgaaactttaagtcgtgatatttcagaaaaggaaaaagaggaaataattaataagagtttacaagaatttttaagcaatgaaatatcatcaaatagtggttctaattctacagtaagtgacactctaaattctaatgtcaatgacggtatggctaacttaggatttgaaagtggattaactggaaatactaaagaaactacgaccgaaactttggtgactgaaaacactacacattcaaatagctctaataattctaatagctccgataattcaaatagggacaaagtatttacagagaatgttgtaactcatggggttggtaacataggagttacaagtgcggcagatttaaaaaaaggttggattgacgttacttactcattaataaataaagtgatagacggagcatatgatttatttttacagagatactaaggaggtttaagaaaattgaagtatttcgagattaacgatttgttaaataaataccctgataagctgatttatattataataagtggtaggggatatggtaagtcatggagcttacaaagaaaaatgattgatgacttcatagaatatggagctcaaagcgttgtactaagaaggttaaaaactcaagccgacgaaatgcaagaaacttacttcgataagctccttgagagtggagcttatgacggttacacatttaaaagagttcataataaaatgtatatcgacggtaaactattttgtaccttcttggctcttaatgggagtggtgttggtcgtggtggttcatatggtaaagtcatgaacatagtcgttgaagagataatgccggaacccggggagcataaagtaaacagagagtatcaaaagcttgagagcttcttggctacagttgatagatttgaggacaggataaaagtattttgcgttggtaacaatactggttactattcacctatatttgacgcattaaaactttatccaactcttaaagaagaaggttataccagcaatgaggttgccgtaattcaaaaagctcaatcaagtaatgaatttaaagaaactgtgagaaaaactaagttagggcgcttaatggaaattacagggacagctaaatataacattgataacgaaaatatttcaaatgacactttcaactgtgtaaataaaaaggagctaaaagaaactaataaactaaaactaaagtttagggtgagagtggataaggagaaagttatcactatatgggaatgttgcggtaaagaagtatcatacttctatgttgataattctactaaaggattaactcaagagtatttctttgattttgaatatcaaaagggtaataataaacatattactcaactaaatccatacaccattaaagagcttgaattaaataataagctaggttacatttattttaacaaccccgaaactaaatacttattcaatcaaattaatttattcttcaagaatacgaaaggtggaaaataaaaatgattaatagactagaaagatatgatttaaattgtagcgtgttcaactcatatgattacgacgattgtcttgacttaaatagtttgctatgtagattttttactaaaataaatgaatgtatagaagcaagtaataaatccttgacatttcttgagtggctttatgaggtgggattaaagcaagaagttgttacattattaactcaatggaaagataacggaactcttgcagagcttatttcagagcaaatacttacagagataaaacaaaacattgagaataataaaaatgctatagagcaattacaacttaaagatacaactcacgacgaaaaaatacaaactttagaaactagagataatgagatactaggggagattgaaactttaaaatctaaagatacagagcttgaaggatcaatatcatctgttagtagtgagattgaaaatgtgaacactaaaatagaagaaactaaaaatgagttaaaaactttaagtgaacagatatttaacattgcaaatgaaatgaaaattaaaaacggtgtagatataacatttttcggtgcaaaagacgatgttaattacgatagtacttcatcaattattaaggcaattaatttcgctaaaactaataaatataatactttattcataccaaatggtaaatattatattactcaaagtttaaatactaaaggattatatgttgtaggtgtaggtacacctgaaataccttttatgacatgggattatacaagaccttcgtcagatttaaatgattttaagaaatatttttcattatgtcaaggttcaataataggttctgactatgacggaaatattttttctaacggtttaaatgcaactaatataggtataataggtaataggagagcaaccaaccaaaatggtgtatcacaaatgaacggaggcgacttaataaatttgagtaattgtagaattcatggttgtggtaaagacggtgtaaatgcactttatggtttaattgcaactatagtaaataatagaacttggttatatcaaaacggtcgtcatggtatttatattggtaaggaaagtggagggtatactggtgaaaccaattttataactataaaagattcatttatcaataggaatgaaaaggacggtattaaaataaattctctaggtagaagtattcatatagaaaacgtagatttagagcaaaatggtgagccgagcgattcccaaagacaaaaaggtgatgatgtagatagtattatattcggttgtagaattaatatggataatgacggctcaaacttcacaggtgggtcaattgtttttaataataattattctgaagaaacttttggtttactttatttagaaaccccaactaatagaatcacgaatggtgtaacaattaataataattattggagaccattaaaccaaaattcatatagttgtggcatagcacttaaaggttggatagagaacattgaaataatgaataataatttatatggtaaagatgaggttaaacttcttgataataacgtttatgctattaaaacaagtaatagagtaactggtggtcaacgtaatgcttgtatatggaaacaaaaagctgacttttacggttctatagttgatataactgctacaggtaaatctgaaatatattctttcgataatctcattacagggtcttcatacgaagattccttcacaagtattttcataaataaatctgttataccttatgaacttcaatatgacggaaacagaagccctctttatggattagtattgtgtaacgcaaatgatgaaatggttggtgttataaaagatgtgtcatatactaatggtgtattaaaagtgaggggagacgttacccgtattatggtaaatggaaaaggtagtttttttaaaacgagtggcattacatttatgagtgggaacggaactgtaaaaactatggttatagattgggacggaagtgttataactatatagaaagagagggaatatccctctcttttacatatattcattaactaaaatcttacatacttcatatacatatacacttctaaatctatttaacattagatttagtaattgagttccttcgtattgatgaataatgtcttcttcttgtttttcattatctttcattaatgaaagttttctaaactctacaattttaaactcttcatatggtaagttctttttaaatagttctgtaagttcgtttaactcatttaaccaatactcatattcctcacgatattctaaagcgatttgttcatcaagcttattcacgtcatttaaatgtaacttttcaaaagcttcattagaactttcataagagtttaataaaaattcctttgtaagtgttaaatcatttttcatttcttcaagctccttttcttcaattttatgttgttctatatttatattattatctttaggtagagccataaaagacccaacacctaaagatattgataacaatataccgtaaattaaaaccttcataatatttcacctatctttcgtaactcattttatagattattgtaacatctcccttaaaaaagaaatgaggaaatataccattaactttaattataaagtagtcctcgtgttctgacacaatagtaccatgatatatttcaccttcaaatatgaatttacaatattctctcattagaattttactcccctctcttagttattaaaatatagttttcctttaatccgatagtgatttctaacatatctttaatttcttgaaagttcctaaattctgtaccgaagtaagaaaatcttacacccttagttgattttaatagctcctttaattgctccttagtgatatggtcaccttctaaaattacgtgtcttgtttttccagtttttgttgtttttattaccttcattattatacactccttaacatttcagtttctattatatcaatgaatttatttactactctatgaccttgaacagagtttaaagcaccttcgggatatgcttcatatattaactcaaaagccttgtctattctttcactaaaagaaagtagtaactcaaaaggcttgtccgatttttgaattgcttctaaaaccttctttttaaattccttaattggagtgtcattaccttttaatgctcttctaaatgctccattatctttaaataatcctatgttactaactatattaaaaaattctgtttggatagccttattcacgctagaacgtacattcttagtatttattttcttatataatctatcattatttacaattttcttaatatcgttttggttaacgcttccgtcttgtaaaagactgtctcttatttttgttttcttttgatttacaattttctttttattttcactttgataagtagcttttgcaatattaccaactatagttaccttttcatctgaaagaccaaaatattttttggcttttttcatattgtccttgtcttgtaagaaaatgtaactattgtttttaactgccctgttccattttctcatgaagtcttgtttgcttgtcctttttaccattttatttactcccctttcatgtctttattataccattatataaggggtattacaaccaccttttttaaaaattttttaatgaggaacgtaagtcgtattttgaattacaaaatctatattagttaaaagtgtaccacctttaacttgcttactttgtaatttcccttctattgctttacctaaataaaattcattaaaaccttgttttgctaatatagattgagtttttttaggtacacccgcacacgcaacacataatttatttgaattagtgtcatgaagcatatactttttagctcctagagatttaaatttatcaaattcatgttctaagtcccatttacctaattcgtatccgtctaactcataatcaatctttttaagagcttcaattaattgcaccttagttaaagagcaataaatgctatctgtatcagtgtataagaagttttcaactcctattgcttcaattatcatagtttgaatttcaactctagcgttagccgttacgaatgaagcaaaaggaacgtaatattcttcgctatcatattctagtaaatcaatttcgtttccattttctctcaattcttttttacaacctatatcccatgaaaaaataccgtcatcatttaaaaataaattatcaagtgtttttctaacctttgacccaaacttgccatacaatgaatttaatagtacttttattgatgaataaagagcgtcaagccctttttctcctttatttattcttcttttaacttcgtctttcattaagtataactcttctatatactctcttaataaacccttttcagctttatataataagaccttaccagtttttaattctgaaaatgtgatatcctcgtctataaaatcaatctctgaatggtaaccgaaatcataatatttatttaagaagtcatgctctacgttagttattgtaaagttatagttagcaatttcttgcacacctttcttatagatttgtattttattttcatcattccaatagtttctattaatgaaagtattcccgttttgaattactttaccttccatttcaaaactcattttcttaaattccatagttgcatttacagaacctaactggataacttccaaatcgtattcttttttctttggttttacataatcaaagcctgtattaataatatatacatggtcttcaaaatttaaattcatttcttccatgtaatcattcatttcttcaatactattaaattccattggtgctccatatggtaataatccgtagcacatctgtgcgggatagcttgagtttttatctattgataccccttgtttctctactattttatcaatgttttttaagtttgcgtgtgtccaccctccacggtatgagtaacgttccatatctttaatttcttgttttagttcaattcttttatcaatttcaaagtgttcattaaagttctctctagcttctttgatatccattatttcagtttccccgttactgaaagtataagcgatagccgtattaaaagctattgaagcagaagtttttaagttatctgataaataaatagttttctctttaccttgaaaagttaaaagagtatcataataaaagtcttcaactaatttcttagttgcgtataagtcattatataaatattcaagctctaaggcatctaaaatatgaccattctctcttactctgtcatagtcatattcattgttaagcttgtaatacatttcttccatttcaacgaacttgtacgctttttcaaggctagtttgtgcaacttttagcgtgtcccaaaattctaaataaatttttacttcttgcactactaacttccctttattttttcccttagtgattatcttttgatttgtgatagtgtcttttaaacaaaccttagctccataatgtacaccattagcttgagttatattgtagcaaccagtatctaacattaccttattaaaagagcaacctttcttatcgttgtatgctattacaccagctgaatatacataacctaaattatttaaaacatattgcatgaagctattttcatattttacgttatgcactccgactttaatagttacgtctaaattctttttatctctcttttggttcttctttaaatagaagcttaaattattttgaactgtatcatatacgttttggaaaaacatttcccacgtttcacatacaaacatgttattatctacgtcaccaattgccatagctccgaaagcgtatgtttttacttcttgccccatgttagtatcaagtaacatttcttctctagtacggcacgtttcagtatcaattacgtaatggtttgataacggtatctcttcaagtgttgtaaagcacttatcttttattatattgtagtgttcattgtttattatatttaactctgatacttgataaatcatttttcaaaatctcccttgcttgtccttctatatttattatataataaaaaaaaggcactttcaagtaccttttttaaaaattatatccatatcatctatcattatcactaagtagtctttctcaactttaacacctagtacttttaagtcttctattaaactaaatgcttccgtttcacttactatatcatattcgtacccttgaaatatcatgtatggcatagaattactatttgttgatatccctcggattttaatatctgtatctttgtaacaaaattcttcaataaactctttaagcttcatttttatccctcccaattctcgtttaaataatttattctctctatatctgttgttgagttgagtaataagtctatcatatcgtcttcaagttcattaggtaactttttgttagtctttgcaataataaaatcacaatcaccccaatgtccatttaagtttttatcaaaaatgcattcatcacatggtgtaaaagcacccctttcttttatgaacattatatgtctttctctacaaacttctttcatgtttttacacatttcaatccctcccttaatataatatattacttaataaaattattataactagtacaattattatgaaaaataatgtagtagtaaaagtattgttatataaatataaaaatgaaattacacttaaaataaatgcaccacataataatacgcattgaaatatttctctcattaagttatttttagtaaacattttttcattctctcctttaccagtaaatcatttattaactttattatacatttataaccaccccttgtcaataccttttttaaaaattatttttattttttcattgacataattataatgttatgctataattatattgtagttagttactgtttattgttttcataatttgacactccctttaaacgac

SEQ ID NO: 2 - DNA, 58428 bp, Unknown
  Clostridium phage vB_CpeS_RO96, complete genome
gtatagtaaggtatagtaatataacctaggataacctataggaaagctatctataaagcatagggaatagactatagtaaaggcatatgagtagcattatgtaacagcatccatcaataatgtttagtagatggcatggcaaatcatgtgcaacagcatatataaggcgccacacaacaaatatgtggatagttgtgcagaaagcaatgcgaaatgcgctcagaatacgcctgtgcgacacgctatttcaataaatatgtaaaataagcgctaataatggaatcctgtagcgcttttaggaaaaacttttaagaaaaatttttttaaatttttggaaaaatctttttaaaaattctcagtttagaaaaaattttttaaaaatttcgcccctgcctttttgaaaactataaaacagcccctccgtgaaactcttcggaaaggctgttggcagtgtttatttaattggaacgaagcaatccgagattcttacatgaggtgttgaggaaagtcttaccatatttattgtaaagccttcaggaatttcaatccaagaggtttcatccatctcttcaaaaatacttaatcctaaaggcgtgttgtatttattaatacgccattcatatttgcaatcttcaaagcgtaaggaaatttcttttgacattcctgtaacctttcgtaaggaagctacccctgctcggttgacaattaggaataaggtaccgtcatggttacgtctataataaactttatctaattccattaaaacggcctcctataagtcttcgcttttcacaaaaactccgtcaatcatttttcctgtgcgccccgaaataactccataagctactcctaaagcttctttaggcactactccaagttgttgacaaaggataattaatgttacaaaagtatctccaaggccatctacaagctcttcacggtcgttacgagccatagcagcagctacttcccctacctcttcaataacttttaagaattgtttctcaggagctgctttgtccaaacctttgttacgtgaccattcttctacctgtgtaattagttcttcaaaattcatattaaattccctccacataattagttttaaatgtttttaaagcctgctccacagtgctgtactcaattgccgaaagtgtttcaaatacttttttatggtgttctggaagctcgtttaaatcagtgatttcctttaagtgataagctgttaaaacttcttttgacaaaggctcgtcatagcccacaatgttatggaaaaagcttgtccatccttttggatagtttacagccatgtctgggctagtggctgcatagaaatgtttagacatcttgtgaccaactccattctttatacttttcgttaaactcttcaatttctttcttagacattttaacatcgttcataagagactgttcgtgaaagttttgttcatacattttgtccaattccttatcccaaacaactgtctgatagcctcttacgcctgttttaacgcctatgatatgtccctcaataaggtaatgattaagccctgaagaatctttcaggtaacttccctctggaataatgataattacctccttaacctttgataaatctatcttaacaccttttggcggatttgtcaatactaaatatcaaactttttaacagagaactccttacgctttttataatgcttaggataatgctttcgccaaatcttgtagaaacgttctatagccactgaatcggagtaagcatcgacgcctatcacaaagggaaatgtccgcagtccgtacacagtgaataagtaagctttcttttcctttttaattagcattcgcaaagtcctcctgaatcttttctaattgtagtgaaagagttgatttaatctttaagttaagcgcctgatactctgcaattccttcaagtacgctgtcagaggttcctactgctgagtaggtatcaaccatttccctgagctcttcaaaatcttttttaagaagtgctaacaaatagtataattccgcctcgttaaattccattaataaagtcctccgttcaaaagtctttcgtaagtgtctgcaacgtctagttgaaagtccacaaggtctaagtcaagcttatccataagctgcaaagtctttttgattgatggattagccgtcccattgattgccctagtggtatctccgtgctgtaaataagaccaagtgactcctgccctatttctcgctctatcaatacatatccaaaaagctttcaaataaatgtcctgagtgtcttggtaaactgtatccattagtattctccacctttcaacaatactctgtaattagtgtaaacaagccatcctaaaacaattacaaatgcccatgccggcaaaaaggttcctgctactgctataataactaattgaatagccacataccctgagattagaacagtcattaacacaagcgtacacgcaaatttaaataagtcttccataatgtcctcctaagtcttttctatagtcttactataacatgaaaaagaggccttgtcaagccccctttttgaaagtttattataagttttcaagcgcccattctgaaggtgtactccacttaggctcatagaaattgattcgctcctgtgagataactacctgagcgtgtgggttatgcttatttaataggattttagaaatagcttctcccaactcgttaattgaataggactctaaaggcttatcggaaacctttttctcacgctcttcgatagacgtgttggcgtactcttccacaagtgggataagctttaataaacttcccacaaggatgtctgttccttcataccaagtaaaagtttcagaattgcctttgtcaatagttactacaaccccatcgtaactttttacataaagcacttcagcataactattatcaacctctagcccaatcttttctaccgccttaataaaatctgtcgttaacatgtgtaaactcctcctaaatgcctgtaataaggctactttggttcttatccattggaatgatatttaaaactgtaatatcagcaggtgataagtcttcgctaagctgttctgaaagctcttttttaaggctttccacaaactccattgtcatggcttctgtagaagtagcaaagttaagcatcttattaccaatcataacgactactagaaaatctttcataagactgcctcctaaatatatttctcaatgagtgccttagcctcttctaaacggcgtgctttgctgcctagttgaatagcttgttcattgattcggtcacacttttggaaaagctctaactgatggttgttaagctctctaattttgctatctaaagcttttaatgcagctttttctgaaggctcttccaaaatctcaaatacaaattggtcttcattatagattgcagtatcccaaataaatccttcaccttcgtcgtcctcaatgcctaagtcaccacagttatccattacaacttcatattcttttccaacagtaaagtattgatgtggttcataagtgtttttaacacatactactttcatgccttctttaagattgactctagttaagcgttccccagtgcttttcatgatgcgttcctccactacttccatttttacatgggcttccccattcaaatagtttaaaatatcctgttcctctcttgaagttcttccaagagtatcgtcaccttctattaaaagtactccattgcgtttaattaattcatgcactgaaccagtttcccagatactgtagcaacaatcgtcaataacacgcaatttcatgccttctttaaggtctttcttagtgtatttcatttaaactacctcccaagtatgtttaatttttgcagcatcaagcattttagaagcttgttctttattctttctgttaaacaagtgtggacaacctccgttgaaagttacttcaataggttcttcaggcttacagtcagctgataacaacgagtgtgtgaaagtaattccactaacatagttttctccaataagtaaacggtagttttttggaaagcgttcatcaaaaggagttttagcaaacttttctacaacttctgttaaaacgtgaatgtcacatgccttaatacggcctctccaattgtaatcaacataacctgtatctaaaatagaaattactgtaagcacattttctcctaaagaggaatagatgtatacctcattgccttcggtattcttaatgcttaggtagctaatcctttcaacctctttcttaagctctgcataattcatttaaaatctcctccttaatgtcttctacaaaactactataacacactttaattaattagtcaacaagatacttcaaaaattctccattaaggtctacccccacaatctctaagcctactcaaaagcttacttctagtcttgaaggtgtagtctaaatcccatatttcatgaagtagtaagccacggtctgggtcattcgttgtggtaagcgtaaatccatactccactggtgaatgcttcatgatgacatggagaacctttttccgaagctctttggactcgatgtgttgaatgtcttctggaataattccaacaattgtcgaggcttgtcggataacgtcttctggaagcatgtaaacaatttcgtggctctgtgtattgatgtcctcttcagggtcttggatacggactcttaaagcgtcttttcggaactcttttctagcttcataatatagggctgtacataaagctatcaggccagctgtggagatgaaaagaattgttccaacagtgtccccaaaccttccgtagacgaatgtacctacccatacaaaagcttttactaacaggaaggttccgccgataaccacggctgctatgaaagcatataatatatacattgaaatggccttcaggtaatatttcttaggcattttcgacggcctccctcacaaagcagatgtcaacgccttctacactgtcgtaattcttcgcatattcttccaaaaactgttcgcaggcattttcatatgcgtaatcattgttgataactgtctgggaggatgcctgaacaatagtgtctgttaggtagcggtaacctacctctaaagtgattttagaagggttttcctcaaacatttcaggagcccaattccacacgcttccgactatatataaggtgttatcggattcacatatactcttaacttcaacctcctcacccgctaatacaagcatttctggaacaactagctcatctccgtaccaacctgtttccaaatctttcttaacagttaatgtatcgcctactttaatttccattgctaaactccccttccaaaagaatacttaatccatctaccacagtgtctggagaataccattttacctctctgtcaaaataatcctttagttcgtctttgcagtagttttccgaaacaattttggtagccttatcctcaaagctaatctcaaggcttacgctagcatcttcgttaagcttgcttaaaagcattcccaaagttagttcggaaggtgtttctttaggaattgagtttggggcaggtaaggctgtgtccgtcgatacttctgtatactggtcactatttaaaaagtaacgcttatgcctttcatcaacgtatacgtgagcccctccgtcaaattcattgacaatctcgtaagccttccctaattctaagatgtcttcatcaggcgtgtccaattctgttttagtaataatcgcaaatcttttcataaagcatttcctccctcttctttatggattcataatagcatacaaaaagaggcttgtcaagcccctttttaaaagtttatttaaatttctttttaatagctttccactcgtcgtcgttacatcctaatacagctattccattagcatataccatacctttttctttagtcatttcatgggcatatgagacaatgtccccacacttgaagtagtattcccaatcttttgaagcttcgaaagagtctgtcgcccaaggcattccttcacgatattcgaagccattcatagctccccaacgtctcccaatttccatgtcagtaactgatggaatagacaatttaatagtgttctccatgcagtctctaatagcttctagctcagcctctgtaacatctttaggaaccttgaacagggcttcatcgtggatgaaagcaaggaagttaaagcagaattttttaccacgcttttcagataactcagctaaaactttacgcatagctacgagagttgccttagtctgttgcgctgctgagccttgtacaatggcattagtagcttggcgtaaagctcttgaagctgtggaacgttttactttatcttcttcagtaaggctattccaactcttattaaggattccgaagtcttctttcttaaagcgtctacgagttccgaaaatggtttccacataacgttgtttgatggctgtcttcttattaccttcaatccatttctgaacctgtggataactgtttaagaaagtatccatgaagctttgagcttcgtctttagaaatctttagaatgtctgctagagagcctgctcccattccgtatagaatcgctaatacaatcatcttagtttgagtacgttctggagttccatctcgaacgtcttcgtaaggcttattaaagattgtagaagctactgaagaatagaaatccacaccacttgcgtaaacagctttaatgtcagggtcatttgaaatatgtcccaataaacgcaattcttgaccgctgaaatctgctcctacaataagggagtcttcgtccacttggtacattgctcttgcaggcttactgacttgctgtaaattaggattagaagcgctataacgaagtgttacagtaccagtctgcttgaagtctccgtgaagctttccatcaggttggataaaggtgtcaattggctctaggaaagaagacatatatttaactaagtcctttaactccaataaatctttaacttctggggcgtgtgttgcaatcttcttaagagcttttgagtctgctgaaagtttgttgcggctattagaagcctctacctcacctttatcgtttatgattgggtctaagataattgcacccgaagacttcatggcaaagcgtttattattacttataccatgctcatcaaagccgatgtgaccgtctaaaatggatttccgattgcttgtaacatgcttactccagtcattgtcgaagtaaagagctttctgaagctgtacaggagagcctaagttaatatcgccaaaatgtttagctaggttatcctcaagctctttaatacgattagagactctttcacgttcgctctctacaatatccatgtcaattaggaagccttctgcttcagcttcaaaagtagtcataatacaaggacgttcaatgcggtcgtaaacctttttaatcttagcaaagttaggcttattaagaatgttcatttgccattcaaaaagcaagaagcctacataagtgtcttttgcagcataccaacgagcaatcataacagctacatcagcaaacttagctttcttgccgaacatttcatcatacgtttcggatggaatatctaaatactttgtgacaagcactttcaactggtaactaggaaggtcttcgtcaagcaggtgcataataattaaagtatcatgtaccccagtctttggaatgattcctaacgaagcttttgccaagcctaagtcaaacgttgcattatgccatacagtagctttatccgaagcgaatacctttttgatacagtcttctgcaatctgtaaattagagatatttaaagcactaggtgagccgtcttcctcacggtgtcgcataggcacataaccattaattaattcaccattgtaacggaatgttaaagagtatcctgcaatagcttcctgatacttatttgtaccaccatggtcaccaactgtttcaaagtccattgctaagtattcattatcacagttaatagctttctcagtccactggataagctgaaattcattatttattagagggaatttttctagctcagcattcattaaggcgttcaacttagcttctcgttccgcttcctgtatctttgcatgaaggcgtaatgcctcagcctttgagaagttctttgtgagtttgtcctcctcacggcttaccttaccttcctccatgtattgcttaacttgcttaaggcgttctaaatcctttgcggagttcttcatggatagaatctttgcccacgcctcttccatagttaacaggggtttcttccccttaaccttcttaattgcttccttagcttttttatcatcaggtaagttaactgaaatcttcatataaacttcctccttgtttattcgataccttatgataacacaaaaagagacctttgtcaaggcctctcgaacaactttctaaattagcataccagtgttaacaagcctgttcatatcgaattggccagccacttggtaagtctttttaaaggtgctcatttctttagcatatttaatagctttatctaagcttttgtaacatttgacttgatggagtttgccagagccatcgtcaagtctttctaacacgtaataattagccattaatcatcgtctccccaatcgtcgtcgtcaaagcttccatcatcatcccacatgcagtcgtcacaagtacatccacagtaggagcaacagccacatggataggtgtcgtcagcccagtccatatcgtcttcttcatcgtaggcttcatcatcagtaataaggcctcgtgcagccgcctctatctcaagcgctattaaggcattaaggaaatcccattctccaatcttttcattagggaatgaatcgcctttctcataggcattttccatgtaagctgtgtttaagatttctgagtggcgctctttgatttcagggtcttctacaacacatcgtaaagctgctagagctgctaaaagctctttgtcaaacattcctaaagacatttagatttcccctttcttaaagctctgcaatgctttgataagcttgtcaacagtttcttcatccatgtaaaaatcgcttgttggaaaggcattgtcttcctcaacatcctctacaaaaagctctatgttaatctttcctccgtaggcctgtgaaagctcaatatattcgataaaggaagtgtttccctcagcttcatttactctaattaacatttctaaatcgctccaatcaattctccaagtgttacgaacgctccatacataattttaatggagtccatgttataaccattttcctcaagtatatcataagcttctccaacttccccattttcccagccatctagaatgccttcattattccatccataccacatctctgacatttcgtcagttgttaaattctcttcaaatagtttacgcattactttttacctcctttgacaacgactcccaaagttgtttcaatgctatcttctgcattataccaagctgtgtgaacatctacaacttccatctccacagccatcggacgaagcttttgaatatccttagcataaataccttctggagccgttaattcagcaatctccaaagtgatgtcctgcattgggtctactacctttaataagtctttcaatttcattctaaaattcctccttaatcttctactataaagaaaattcctaaagctactgagacgaatacgatagcacctaatgtgattaataccccaagctctcccaaaggtttagttatcgctagtaaccagtaaaatcctgcaaagaaaatccccattaacaaaactactaataaagctactagtaagctgtaaatgattcttcgtaaaagttccattctaaaatccctccaatttgactactttaacattcccatcatcgtggaaatcctcttcaaaagtctccgcaaagttaatatacgcgaatgtggtgcttattgctgtaactaagaaaacatcaaagcagcctgtgttagtgttgaggatttgcacctcatatacgcttagtgaattatccatgttccacaatcctttctacaacgcccattttagtaaaacaaaaggcaatataaactcgattaaccatacagctacttcaataagcaaaatcaaaacggctgctcctatctttatatattgcttcattaagcccactcacctttcttagcttcggcaatacgtcgcttcttgccttcgtacatacgtttcttaaattgtcgcatatcataaacaccaatccctaaaagctgttggataacttgttctgataactcgtagtcttttagaaggttgatggaattgtctaatccaattttttcaacaagcatttcaatatgattattaatatctttcatagaaacttttaatagcttaggcatatcagctttagcaacatcgtttgcagccaacttcccgaagtgtcctaacgtaagctcttcaaaaggcttagctaaagtcttacctaaagagtaagcagggattcgttcagcgtgttctctaatctgtttacaaagcttgcatcgaggattcttacaagtgtcgtatggattgcttgtacgctctttataaaaggttgctgactccatgtcgaaaccaactgtccaagtatgctcttcaattagctctgagatagccgctaatggattactcccttctaaatacacttctaacgctttcttacggctgattttattatttgttctaggcatttatatattcctccaagtagttttattagttatctttgtctatgaatataatatagcacaaaaaagaggcttcgtcaagcccctttttgaaagtattttaaatttgttgcataatctttttgcgagcggaggtaaggtgtctactcaaggtgcttcttgaaggcatatctggaagcacttcaatctcacgcatagtgtaatcgtagataagatttagctcaaggactttcttctccatctcagttaaatcggcttttgccattgcctttaacaagtcagttctaagagctgctcgctccatttccaatgatgaggcaatattatttcgtgaataaccatttccttccaacacttctaagttgattggagattctttatataagtcctcagtacggattcgcttctcctttttatcagaagcaaagctatactccattttagaacagcgagcgaacttgttagtgccttccaaatcgcctctagtatcgtcttgcatataatatgttgagaggatgtttgctaggctatctggcaatgctacgtcgccaaagcgttcgtaatacatctctaaaacctcttcacaaaaaagctctctaacctcaagtggtactttaactttcataacaactctattaatcaatcttattaaaaccatacggaactcatttgctgtttccgcttctactaacttttcgtagtatgatttctcagagattaatagattagccattagcaacctcctgagccatattaataattatttcagcatatttgtccatttgcattaagtcgctctccccattgtttatgaaggtcaagtgaggctcaatcatccctacctcaatgtctcccttattgtttaaatcaagtgtttcacctaaagcttcagcacggcctaatctaataagagggtcggcatccaccttaaccaagataaatccataatcgtttataagtgttttggactcgttctcgaaacgaacgtccgtaactagtgtggagaagccttcgtcccagtctttggcagcctgtattaaaaggttattaatccaaacgttttcgtcaacctctctcatagcatttccaagggtaacataggcgtctcttggcttctctttgtcctccatgtagaagaagtatcttttatatagctgtttcaaagggtctgcaaaggctatcggcatagcttctgcaaaacccatttcaactaactttttctggatagcttgagcaaatgtatctttacctgctctggaggttcctgttatggcaattctcaactgctgtttcattcggtagcctcctcaagatatttccatacataacctttatgttgagtttcttttccgttacacacacttgatacgtgtgcttgtgaaaatccatttctgcctgcatcagacgctgaagggaattttaatatgctaccatctttaatatttactcctacaacaggcttaccaaaagcttttgctcttttcttcaaacaattaccgtggttagtattttctttaactgtagcccattcaagattgcttgcttcattattatacacgacttcgtctaaatggtttactactggcttatcttcatagttgggaataaatgccgtggctactaatctgtgtactctaaactgtctaggaactttattgctgtataaagtaactaccttgtatccatctacattgttagactgtttcaaaacttttccagtagagattcttctcactctacctaaatcagaaacctcgtaggcggggtatccatcaattactttccaattctccatcatacttattggcctcctcctcaactaataaagcctcttccttagctttttcaacttctcctaaaggaacaagtacgaaattcttttcaatatgagttaagcatatagctacggcgtcagatatgtcatctataactaaatcaatgggctcattccagttaatatatttagtagtgcccattagcacggcctctttcgtggagcgcccgtggcctccaacaacctttttaactgtggtaggtgaatactcataaattttaggaaacccctcgttggcagcaatcatgtccaacaccccagcaaacttaacaaggactagttgagttcctagacgagcgattgtctgctccttaacaacatagtctgggcggaatgtttcaagaagaatctgaacagcttcagcaaagtctttcaatcggcgccctacgttatgcttagggttcgtgctaatgatgtctgtatggattagcgtaaggtatccagaaggctctctgtcaattactgcaacgcctgttgaaacgctgcttacatcaaaactaataaagcgtattttaatcctctcctaacattatatcagctgcatattcaataaatctatagaatgcttctgtatcccctagttcaatctccttagcggagtaggctgctatatcaactaaatcaaaggctaactcaagtccagtaataacgtcttttaaaatgatgttggattcaccatcttcacgttgtaagaaggttgattcaagcatttccttggatagttcactaatttgcagtgcagcaggcttaataaaatcgtgtccacggaatttaataaacactgcataagccttctcctgtaagttgatttctggaatgccttcataaatgctttcattcattacttaacatctcctctaatcttcttgatttcttctaaagccttctggtcaaacttagatacggctttcttctcaagctcttccaattccttataagtaaggctgtttgccaaggcgtatttatagtcgctgaaagtaaatccgctaagctctaatggaggaagcgttcctgcatgggcatgttcaatcgctgtgaagaaacggttgcgaacgtctttcttgtcttgttcggtaatatttacaccgaatacttgtaagtctggattcttcgcatagtcttcttcagtcatttcccaagacttgtgagcgcagtttacatagactattagatacatgtctaagtcttttagcatagcgtagttcttaacctgccacacgtgttttgagttaggctgtctcatggagaatgcactagtagccgctggagtagtttgtttagacttaacctctaggcctactcgaaactcttctccactacttgggtcagtccaaagcataatgccgtccatagagcctcctgtaatgaactccatacggcctgatttatactttttaaaggtggttgagaactcctcgaagtgcgggaagccttcttcagtacgttcaaaacggaagtggcactcctcttttgtgaagcgttggtaatgcttctccatcattaagacttgttcctgaatcatgtctccaacagcagtccctatcatttgccaacgactttggtgagcttggcacttaggagggtcttttctatcacttccctcaatcttgtcaatagcaaatagagtctgctctcttaaacacttaccagttccacttgaacggaatactggaacgttttctggaaagtcctcgaaaggatttctgtcaactattttaagatagtctctcataagctggtggtggacttcgctatcgtatgtatagcctgtttctccccacttatttaattgtcgtttaaagtcctctgcgatagtatctaccagagtctttcctgctgctttattgattgctgttagccctttagccattctacatattcctccatgaaattatattttgcttcaggtgatggaattagtttttccagaagcatgtccattttctctatcattgagaggtgtttagattcctcttcacgcaactttattgagtggataacattgtctctttgggtaatccattcaagattagatgctcgattatccttcttattgccgttgatatgattgaccacattacagccagcaggcctttcacaaaaggtttctgctacaattcgatggattcttgcattataatatttgccttcaagagggattctaacatgtaagtagccagagtcttggggctttaataacctatcagtcaacttgttttttactaaaccttcagaactaacctcgtaaatatctgagtggattcccttaaacactaaaggcttccaaagttccttagttgctgtcattgtttagctccttcctacccaattagtataacacaaaaaagggcttttgtcaagcccttctgaacatctttgttatttattgcgctctttatcaagcttcaataactcttcctgagcgtcttctaacatcttctggtggattagtaattccatcttcaaggcgtcagtgtagcctagagaatattctcctgtaggctttccgatacgctcaatgctttctctgataagcttaacagaagtcttctcatatcgaatcttctctttaaggttgttcatttgtgataggtaatagctcattggattcctccctttgaaagctcctcaataagagttttgttaatttcccagtcacagctaggcacaaagctttctgtttggctttcagacatttctgcatagtaacactgactgtcaaaactatcaaaatagactacctgatacttccctccaacagttcccattataagaaagtgtaataagttgtcaggatacttccagaaagtccctacttctctggttaattccatttaaattcctcctcaacaaattcccattgataccctttatggctttttcttaacttgttacagcacctagatacatgacctctatcaaatccttttgaagaagcttctactatacttttaaaaaaccgtattgcaccagttttaatattagtggatttaacaggctttgactgagcaaccaccattctctctgtccttgttccgtggttgacattctctttatgcgtaacccactccaagttttctacacagttgttttctttattctcgtctatatggtttacacaaggcttgttatcagggttatctataaaagcaagtgctaccaatctgtgtacttccctgttcttaccttttagaactaccttaggataaccacctctagttaaatgttgctttataattcttatttgaagtcgatttacaagactcattaccctgcctttgttggatacgaaataatcggctccgaactcctcaataggtctccatacctcgttaaaattcgattccattaaatacactccctacttcttctaataatgattcttctacatatccaattactggtaagaatattccttcaacgctttctccttcatgacctgaacggccttttacaatctctaaagcgaatcttccgtcacagctgtcgaaagccaatactacagaagcatcctcaagtaaggccattgtctttttaacgttagagcgttttggaatatttaactcacgctcgtctccacctttttcgtttgagtcttcttcagcctgtgttacagcgataattactgacttagtacgtcctgccaatctacgtagtgctttagaagtctcagcagcatctccgcctgctgttttagaagtgttcttttcatagtgcatatagtacattgggtctactacaacaaagtctgcgcctgtctccactatatcacgttcaagttgagcaaccgaacgattaacaaagtcgtcgtcgtctactgctctcaatgtaaatgagccttcaataaagtcatttaagttagtaacaaagtcaacgaaatcttgttcttcgtttaagtcttcaaatgatgcttttgtaatatctgaaatattgaatccagccagataatctgagccattgattcggcttttcttaacctctttacgagctgataggaagctgactgctcttgaggtgaactcatatttaggcatttctaaacagtagtaaagaacattagccccattcatagcagcttctagtgcttctactaacattacggtagactttcctcgaccacttctaccaaagatggtgtaaaggtttcctgactgataacctcctccaataagctctgttaaagacttgaagtgactgttccagaacttaacagacttaccttccttgcggtcgtagaactcttgtagataccactcagaggccttctcaaggcggtgtccattagctccattaactaagttctctgctttgattgtttccaaggcatggatagtctgattaatgaagtctgttgggtcgtccttttttactgagttttcccagaactctcctaaatcagcattgataaacgccgctgtagccacttgtagacgagagttctttaagcttttagtaagacttgcataagactcttccgcagagctttggaagtcgaactcaggaacctttcttaaaagtgtttggtaagcaggagcattaccatcgttttccctactgtaattaataatgtagttgtatgcctctttatgaataggcgactgaaataaatcctcgcttactccatatctatttaaagcctgtacatcgttttcaattataacttttgataataacatctcagctgaaactgaagccatcctaatccctcctatattcctctatgtgaatcgccctcgaaatgatacacgatagttcgttcacgaacacggtctgctaaacgttcatcaaacacttgttccaagtagtcaattggatggttacttgtaaagattgatggaagcttgtttaccattcgtttgttaatgatgtcgtgaaggtctgctcggaaagcttcagtagcgcttcgattaccaatttcgtcaaatactactaaaggagctgactcagctagttccatcatttcgtaatactctcgtgaagtcttctctgcaatgtcctttgcgattccatttctagtaaacttgttgtaaagcgtttggaaactgtttacgtcaaggaagtaagcaggcggttgtttcatgtttgttttaaggattaccgaagcttgccaagacatgaacatatactcgttcagtaaagcggccgctgtggaagtctttccattacctttattttcactccatagatagatattttttataggccgttcatcttggttaatgttgccgtccttctggaagtctctcttgaagctttctacataaacttctaactgtttataaacatccttctgagcatctctcgcaggagagttgaataagaagttgtctgaatagtcgtaaggaaccttggcagctccaaatttaccacctgttcctgagcctccacataattggatatgtatatgggagagtgttggattaagctctttttctgtctttcctgtaacttttttaagcttttccaaaagggtttgttttctcattgggaatcctcctaaaaatttctacctcactattgtaacacacttgtaactatttgtcaaggccttcattgcctttatttatttgtttgctactactcttataaaatagcataaaaaagaggccttgtcaagcccctttctaatggttattactggatataccaaagctttcttctaagcaatttaggttcacttattttctttagtggaggtgccttgtggaactcttgccaatactggtttaaatcatatccagaaggcatgtcgtaactgaatagcttgtgagttttccagaaagcatcgtcgatggatttagcagcaaggtttcctttttcgtcgttgtcagtgcctataataatattctttacgtcgacttttttgataaggtctacttgacgctcaatcaagttagctgctcctagagatattccaacaaagcccatctcataagctgtcatggcatctatctccgcctcacatattacaagcgtgtttggaagcttttcgtaaatcacatggtacccaaacaacatattcttcaagtggttgttacctgcctcgtaaaagaagtctttgttatccgttcgacggtattttaaagctgttgctaatcccattccattaatgtacggaaggcatactttactctcgtcctcagaggttctgtagagctgctgtgtctcctctgagattcctcttcctaataggtatggagatggattatggacaggtatgtcaaagattgacttctctttcttaacgcctagcttaacagatattgcaggagcttcgtaaggtctaatccaaaactcttccttcagcatttcaaaagctgtttcgtaatctgtgtcgtgaagctctgccacaagttgtatgaagtttcctgaatcgcctgtacctgagtctccccaagtgcctgcccagtcgttattatagtttacgaagaacgatggatgattgtcgtcacgaaacggtgaagcagctattaatctgtcttccgtccactttgcccgctcccactcaaagcgctctaaatattcttgtatatcaatgtgtattttaatatcctttgtcaagtggactatgtcaagcatttacaaacctcctagcgaatacgtgtagcataaatcttcttctctgctttgtcaataaactcaatctcaaatactcttgtaccaaacgtcataagctttaatccgtcagtcttaggagcgtccgttaagaagcttcctccctcaagtgctggctcgctttctacagcgttcttataaataactgtgtacatggtttccattaatcttcctcctttaacttgaagcccatacaaaaatagctaagcatgctatgaaaaataggaatatgaaaacatctatcataaagattgctaaccacgttaaaataatagaagctactattgccatgaatattagaaaaggcacgaaatacttactttgttccattaatcttcctccttgacaattgataaaatttccctatcctgacctaaatgtcggttgaattgctctaaagcatcttccaatgagtaggcataaacattatactctttcctatcttgttcaattttaccttgtgtagtccatgcaactacttgcgatacaagtctgtatttgttttggttatacattagtcttcctccccataccctctaacttcatcgatagcagctattacttcctctgctgtccccactgtaaagtattctgaaaaatagtcggaaggttttgatacagctactgaataaggtttattttcagcatacatatgcccgaatccgtttagagaatgtactacatgaacttcccaaccatttttaaactcttcactaaatactactttgcaatcgtttaatccaactttccaactcactgttaatcctcctcttcaaataatgccttagcataccctgaccaattgtctactcctgctgcttccaaagcgtttagaaatgcctctgcctttactagtctttcatgctcgctataagaaataattactgtatcctgcataaaatctctcctttacaataatgaataaagccaacttattaaaacgcatatccaaatgaatacaacaaaacatgtaaacaaagcttctggaactatattccacaggcaaatataaagtaagcttaccccacatattaagattacggctacttttaagtcctcaattacctctctcaagaatagttggcctcccatacctctttgatgaatcaatcataacataactttaaaactttgtcaagcttttcttaacaaaaaaaagaacagccgttaagctgctctaatcgaaaggaggtgattctttatgaaagaggtttttggaatgagctgataaacagccaatgccttatggacggaatcgaaccgcctcacagaacttatccaaaggggacaacattctgtatcccgaatataaggccgctggtagccaactccagcttcagaagacttcactagggagctactctggacttcttggctaatgcgctgttggtgacctccctacaggccttggttaacagcgtataggtaatgttcatccttttggctgaggcagttggacgcctcccatcacgagtgtagcaactttggccgaggactgctactgccctcctataaatcacatgaagctccgctcctagtcgttttagcacgctacggaataagctttacagcaagggattattgagcgttcccctagccaaccctccatggaacacttctggcatctatccgaagtgcttattgggaatatgtaaggcgccagtagaggattcgaacccctttctcaatcagcctccgacaagcgctataggaagactgttgcgcattaacctaaactatgctaactgactggttagggatgtcgggcgaggctttggtgagtagctagctctcctttgcagtcggggacttcccttatgtaatagcagataagaaatccatcttacctacttaatgtaattatttacgtttacttttggcacgcattttagcaaggcgctccatcttcttcttagcagcttcctgctcagctttcacaatggcacggttccaagctttctcacttttcatactttcattagtcattccacaaacctccataagacttatttgaacacgctgtgaaggagtcgaaccctctctaatggttttggagaccattgtgcagccgttacacttacaacgcatgaacgaggcagtcgtcaccgcctctgctacgccctctcataccctcaagggtgggtactcctgctattatttttactgccttttacagcagcccacaggcgggagcaatctgtagccctagaccgctatagccgagaagcatctcgaactatgttacgacaaaccgtagtagtgccgccttacatataatactatatcatattttaaagcctttgtcaagcgttttttaacactttttaagcttcttttattttagcgaagttgaaacccatttggattatcttcttgttgccgtgttcatctatgataaaggcgtggcgtggctttgtggtaagctgtacaacagtgtaggtattgccgttgataacagtacctgctccacagtaattgccttggaaaaggactgtgtcacctatcttaaatctactgtaatccattttatgcctccaagtatataaaggtgtagcctaggtgttgacgataaatccccctacagcatcgtgacactgcttcaggcctaaatccatccttcttagattctgaagtacttttataaactacttcttcgccagtttctaggtttactcctttgacaggtttagatgttttagatacaattctagcaattgcagttccgtgattaacattctctgaaggagttgcccattcaaggttgtctacatgattattaaaaggattttcgtcaatatggttaacttgttctttatctgaagggttaggtataaatgtagttgccactaagcgatggactaaataacttttccattggccgtctaaattcttcaatctaactctcatatacttttcgtgatgattgtttcttgaaggttttaatattctctcttttactaaaaggctttttatgtagccttctttacttataaggtaatttcccccaaaacctttaaggggaaaccattctaaattattcatttaagcatccccttactggttttagtttaaatcatgccaacccttgaagaagtagcgcaagtcgtgttggaataccgactcacgagattcccactcaacgtcttctacgactcctgtgtcaatagctacttgcaagtcttctggaatagcttcatataccaaagtaccttccattaagatgtctgggttaaatacagttataaaagtcttcactgacttagcctttgccatgccattatgaataatttcgtaagcagcattccataatgtttctgatactgcttcctcatagatattgctctcaggaaagtcgtctcggtaaaacgtatctgagaaggtgattgatacaggatgtgggtacagcccttgcttatctacgggttccgcaattactttcgtaaagaactctaatttacgcatggctacttgcctcgcttcttgttcttcttagccttttcaaagtcgattgcttcggcttgtgctaccttttcttcgtaagcttctttacgagctttctcagccgactccgcaaagttcttaatagagttattgatagattctaattgagcttctgataacaagtgtttatacttagttacaagaacttctgagtcagagtatagagagtctaataacatctcgtctccccagtcttctccattgctttcagcagattccatcaagcgattaagaacgaacatgttctcagcgattttagtaatcttatctaagcgttctttaccttggaagccagctgataaagagccgatgaaagtgttcgtcataacaactgtttgtaacaggcggaagtaatcctcttcggtaaacttgattggattaacttgtggattgtctttcttcattcttttgtcctccctataataggagagaggccgaagcccctcgtatagtttttagaaatcgattcctgaagggtctactgtaccagcttcgtcagcgcctttgtcagtggcagctggagtagctgtaggtaagtttggtaaacgagttacatcgaaaccaattttcttcaagtcttcaatttggcgttcaacagtgctttcgaagattgcgttctcgaaatcttcttcaggtactgtagcgccttctgtagctttgaatacttctaattcttcaggcgttaagtcttcatcatctaacggtgtgaaagtgtatcgattagccgcctcacaagtaatctcgaaggctttcttagggaatttcttagcgttagtttcttttgataagaagttcgttaaggcgtcgatattagcgttgtcacgtcctaagttagtttccaataagattggctcaccagctgggtaagtattagttttcttagtgccttgtgtgaagtctgccaaaggaattacgccgaataagataagcggttttggagttaagatacgagctgctttatatgagtctttgtcagcttgagaaccacgttcaatttcccctgattcaactttagcacggtggtctgctaacatgatttcagtagcttcatgatacaagtcacgaacgtctgtacgtccttctgcatggtatgataatgttggtaacacttgcggttcaaaaactgaaacgacttggtttacgtgtaagttttctaagatgtcttctggaatgcgcgcacgaatgctttgtccttttttaaggtaagtctttttggattgctcaccagaaccttttgattgcgtttgtgctgcctgtgcgattgcttctaaacctttagccataattaatttcctcctaataagttctttattcatctttactacttcattagtatagcatattataaagtataagtcaagcctttttgaacatttgttaaagtttattattacagttttgttacaattgtttcaacaaccttacaattaacgactccacaaaagttcttgtgcaaaatcacttgacacatttttaggaatatgttatacttgtctttagagagagagaaagagaagcttattaaatagtttcacaaccgactttaggaggttgtagctgagtgttaacgaagctgctcttgaacttaaagaccgtaaggtcttatggtttaagcatagctatagcatatagtatagtggaaaggcgttcggaaagttccctcacagcctaaagactcctttcagtcgcctttgaaactttttcgtgagagctgataaaaaaggtattgacattccatcgaatacgtgatatgattggttcatcaaatagttaagggagaggttattatgaaaactttgtataaacgacagggaagagctgttgtaggttttaatgaaagcacaggggaaatggtcaagtatgaaaagtcctctgacgcagtgttagatggattccatcaatcagcaattagtgactgctgcttaaaaagaagaggtaagcataagggattcgtttggaagtatttagacgagcacgaggagggcatttaagatggcatggattagaactaaagagaataagctttataaagacgctgagggaaacaaggttgacacaaaggattggcagaacgctccagtagagctttggaatacaactacctttatggagtatgtcggacaccttaaccaacagaagtttgggaaggttcctgtcaacgcttccctacagactttacgagcaatcatgaagaaggatattgaacgcttcggtgcagaggccttgaagatattcttagagatggctgttgcagactatcgaggcaattcacgatatccaacgctttcttacagccaagctagactgctttatatggaacgcttcatgagtgtagctttagagaaagctgagcagaaggctactgacgaagccactccacaaactgaatataccttagaggaggttgttgacctattttaaactaccgtaatgataacttcataaagctagatgtaccacatgtctatttaagagacgctgacaactgctataagataaccattgaaagcagcttgccgaaagaccctttcaatatcaatgtgaatgctattaaagaagcttcccataagcgtgctgtggaatccattaacaaggagaaggtggccgaggttgtcttagacttggataggagattaaattaccttgctgacgaggagtcagatatgggtaaaggcatgaggatagcttatagagtagctttagaaaagcttaaagaatgctttgaactgtaataggaggagattttatgaaagaggtttggaagcctgtagtaggttatgaaggtttatatgaagtgagttcggaaggccgtgtcaaaagcttggctagggttgttaaggcttctcgtggaaatggaatgaggggagttccagagaagctgttaaatctcgtagtaggtactcatgggtacgttgttgtaggcctttggaagggtaataaagttaagcagaagcgtgttcatatattggtagcagaagcttttatagaaaacactttcgataagccatatgtaaatcatgttaacgaggataaggctgataatagagtaagtaatttagaatgggttactgccaaagagaatacaaaccatggtacatgtatcgaaagaagtcgatatactaactctactccagtaaaaggcactcatgaagacagtgggaaagaagttatttttatgtcagcaatggaggctgagaggaatggatttaaaagcagattaatttatggttgttgcaatggtaaaagaaatagccatttaaaatatcgatgggaaaagataactaaagaagaatataaaaaagcccttgagaagtaacctttctcttgggcttttatttttacttaaactttaacttgaggatatgctaattccatcatttttaatccaaagttatcatagttgtaagtcgttacattagtatttccaccataacgcttgattaaatcttgcgcctctttaacatacgtcattgttggtaagtagatacgtttacctccgattacgaaccactgttcggtagtattagatttagtcttcttgtaatagatgaacataatatcatcgtctcctttgtattgtcctagatttggtttatcaggttttggtgcaggtgctggagcaggcttttgcgctgcttcataaggtggtcggataactccccagacaccatccatattgtggtgaataggtgctgctggactgcctacttcaaggcttgggttattccagttctggtcgacactgacaaatgttccatcagctcttccgtcctctgttgcgatagctgtgtgaccgtattgggcatagtatcctagaccccatatcataacgtcaccagccttaatttgtgtagagcttgtcgtacggattcgttggaaacctgctggaagaggttggtcaactaaggcaatcgcattgccatacggacgccatccgaagaagcgttgcataacgtctacagtcaagtccatacactgagtgccgtaccaaccgtctgcgtctacgccttttcctacaaggccgtttacatagcttactacatcgtttactttaaccatgtgtgtaactcctttctaaatcttttcccacaacccacccgaagcttttgaagagtgcttttaactcctgttgaatgttaagttgggaacatttcctcaagtgatacgtttactaaccttcccgcaaacaagttgttacggcttgcccgaagctctaccacaactccaaagtactgacctttcttaacgttgataacaactgtcgcagtacctacattttgaagccttacaggtttatcccaaccagaagaaacagctgacattaacattactccaccttgagtatttgtagattggtcagcaagctctttccaagtgttcttgatttgcattttagcgtaggcgtagtcagactcattagcgccgtctccaccttgaagaagaactgttgcttggaacttaattctagcgtcacgttgcgcttggaaagcctcatagccagtagttcttagtacaagagagtctacgctggctacattagtgttaaaacctacagattctagcgctccaaatctaagcagcgtgccactactaaagttattcttattagaaccttgaccccatccagaaatattaggacttcttccatcaactttgataagtccgccgctaagctctgtcgtaccaacagttgaagctccttgtatagttttgaatgaaagcttttcagagcttaggctagcagcggtttgaactcctgtttgattatttactccaatgatgtttaaagcttcacctgaaaagtctcccgaagaagttttgataaggtttttacctgtttcaatgtcatatattcttgaggaattaactagcacacctctttgaatgaaggtagactctttacttctaagtctttctgcttccccagctggaattatatacgttgtattatcctgtaggatagacccttcagtaatagtagtagttccttggttacgtgactgtcctccgataaccgtttcagtcttattccattcgttaatgaatttagaaccatagatgtcgataccttggatagtacctactttaatctggcttgcgtcaaggtttgttacattgattttagaagcgtcgataactcctgctgtaagcttacttgccgaaaggtcagctaccatagcgtctgtgataacagcgtcctcgatgtaagtcttacctgtaatctgaacctttgcaccatcgatgataatgccctcgctagaaacgttaatctggttgattacgtcgtctttctcaactcgtaggttaatctggtcttgaagctgtgtgatttgactcttatcagctttgtcaccaactgtcaactggatttcatcaaccgtttgtgagatttgactatagcttgtttccaatccatctgtcttagttactagggaagtgatttctccattgatgtttgtaatgcttgcaatagcgtcttctggagctggttgccaatcacctatcttatcaccttccacaaggtagacttctgtaaagtcaaagttatctccgttcttaacggcacttctaactccttgatagataagtaggacatcaccttcagtagcatttgtagtaagctctgcgaacaactctccctcagctttcccatcggtagggatagtaactatattagaagcccatgcaagcttaggaacattgtagatacctactgtgattccttctggcaaagtaccttcagtaagcttgtacttaaatttaagcttatagtctgtacctaacttagttggaatagataatggaatggtctttctacgagccgttgtgttagggcttgtgtcaggcactgtaatcttttgagagttaagagctaggttacggccactaatcataagcttgttgtatctatccaaagcttcttgagcagctgtcttagcagtgtcagcgtcttccacagcttggttagccttgtcaagagattcctcagcgtctttctgagcagccttagcagtgtcctctgcttcaaatgctttagaagtatccattgaaagcttccatactttctcaacgccatcccaaactttaatctcatggattgtttggccagcttcgttaacacgtgtagagaaccatgtgtcgccatcgatagggtttggcttaggtgtatcaccaatacttatagagttctttccaataaggtcagtgatgattcctgcttggccttcctcaagcttactaatctgttctctagtgctttcgataataacagtgttagagcctacttggtctttaaggtcgtttaaagcgtctcggatacctgcaatgtcagaggtgtcgtaagtgtctatttcacggaagttagtaaaggttacctcatcaaggcttgggtcgtcttcgtgataaacgtattcgataacacgtgcttccaatccaatttcacgagagcctacacgctctttcatccatactgtgtcgccaagggctagggtttctccttcccatccttgaacacgatttagaaggattacgttaacagtatacgtatatactggctcaatgtgttttagaagctcttccacaccttgtcggtaaacttgctcagggtttgaagcagcagccttgaatggcattgccttgaagaagaacggctcgtcatagtctgagtgagcttggttatgaacaatcagggggctttctttatcctttgtgaaaccttcaatagggtttactggagcccagttagcaagtgtccatacttttccttcaacacctgtatagtaaggtaggatagctgtcttaattgtatcgtactgcacgtcccgagtgattccaataacgtctctgttataggtaaagtaacgacctgtctgaacgcctctgttcttatagactttgacaatccgtttagtctggttaaaggctgttctctcagctgtaaacttaaagtctacatcataggttttctgaatgtttccgaacacttctcgcatgtttgtgtactcagatagaacgaaaggcacttcgccttcttgccatgtgtcagcagcgtattccactccccatccagtaccttttacagctttcgttaaggtgtctccaaggttgtggccatcgaaaggatagattacagtgtcattcatttcgctaatcgagaagtcctcacactccactaaaatacgagagccaacctcttggaaaacttcttctgtatggatgatattaaactggcgtaagtcaccatccttatcctttgttaataccttgttaaggcctgtcaggtattgggtctcttcacactccaaaggtacagagaaagagaacgttgaaaccaacgtctctatattctccgtcatggtaggctcaaagtatggtaccccttcgctaacgttattgacaaaatgcgctactgtttgtccgattgcatttaatactgtaatcatacttttcctccttatttaaatcgttcgttatatactatgtcaatcttacctttaaagttattaactccaatgtagttatcacctttggttattgggaaccagtcggtcatagggtctactaaagctggcgttaattctccgttaaggtaggtttgagctttgcttgagtcaattacaaccgtgtcgccttcatggaacatgattggagttccttcaataggattctctctgtagttctctaagttttcccacacgattaatctacgtagtgacaagtggcctagcttaggtctgttatgatactggctgaagaacactccaactctggcaaacttcttgccccaagactcggatggaaggtcgaaccagcctgtgtccattgtgtctttaagcattccttgaccataattgaaaactgattcaggagctgggttagtctttctgttagagtattgtccaacaactaatctccagcggttatctctgcgttgtacagaaaccttgccccaaaagtctttccacttggttccgttataaagcgttctacgtttagtccaatcgcttgcataagccacgtaacaatctaggttaccttcttgaggtcttgagccaactttcatgtatgctatagggctgttgttctcatcataaaatactacgaagatagcattcagagagtttttaacaggcttacctgtagctaccacagaagcttctaaatagaagtcgtcaacgttcttttgaaggttagcaataatgcctgctccatggtgtcccttatggttcgtgtcaccgtagttccacttgtcctgattacctattccgttggctctttgaacgcttccttgaagcttagccgaaaccccttcaaagttaggaagctgcgtgtcgttcatgttcatccagcctgctccatcggtggtggtaaacttactatagaacttacgtggacgtaggtttactttagtttcatcgttagtgtctttaggacggcggccaattgatacgttagctgaagtgtctgtaccaacaaggtcgacataaccagctggctcgtcaaaggttaactgataggttggataagcgtctgccgtacccatgttgttaagctcgataaacggtacagagccttccgtactgtcaatctcaacagtttctacaacctgtttaccaatagaggtattctcaagcattacgaaagtcagtgttaacgttcctacgtgaagcgtttgagaaaggtctgttgaaccatctagcataactttgtagtagcggtctttgttatcagagaattggattaccgtttctccaacgtctaggaagtcagcaagctcttccatcaacttaggcatatcctcttcggcaggagctttgataaagcagtccattgagatataccgagcgtcttctgaagctccaaagttatagcgtctcgtaccaccgttgataggtgttagagagtttactttaggggcaagaacgcttctgttaatgtttcttacaaacaagaaccaaggctgcttctggtagttgtatcggaagactcttggaaggctgtcgtactgtctgtccatcaagaagctaaatgataagcttgagtcaagagccattgaagattctcggtcactaggtgtcttacgagcgaatcccacgtatatataaccattagagtgaatgaagtgtcccttagagttattgattgtcatttcaacagtttggtctaactccgtcttaatgagatagtctctatcagggtttctagcctcagcgcttgcaagaacctctctactacccatgggaacctcgtcagcgtcaattggagtggccactaaattaaactctttgatacgttcattaatcttcctaatacggtcgtcgaacgtatagcagtcaccgaagaactctgggtaattgctttccacatagccgtatacatcgtaagcgtgagtgacttgaaccattgagccaaacatgtactcttcagttgttgagataggtctctctaaagattccatacggtagtcaggaaggttcggattaaatccccactcgtcaagctgtgattgagtaactgtaatagaggccgtcataactacgtggtcaacttcaatgaaaggcagttggttatactcttctacatcggatggcttgtttgcccagaacagcttagcgaagtattgcttgctggcttcttttgagtagtacccaagaagtcttacgttgtcgctttctttaagctttccagcaagtcctttaaacacgaatgagctagcgtttcgattaacctccgctgtgatgtctgtaataactccgttaacctcaagcttgacagtatccatgatggaatccacatatcctgaaagtgtatcctcgccattgtaaaggttgtctgcaacaagcttattactcttaacaaacgatactacctcacctttaggattaatccactcttggtaagtcgatgggtgaacaactgttgttaatccttttccgtaaagcttctgtaaaacttgctgttgcttagttccatcaggtaaccatctccagaagcataggtcaagtcctccgactccttgtgtgatagagcgtgctgtaactgacgttgtgaagatgtcagctaaacctcttagacgttgttgtttctcttcgtccgtagaaaggtcagcgaacactttaggaagaactttctcaacagcactcttgaagtcgatattgaattgcattgccacaggctctttacggtaaactgtctcagctcttgctacgtttccatcaagcttgttagcgatagcgttgtaccactcttgacgaaactctgtccagttgtcattagactgttgtacgaataccctagcagaagcgtctcccttaacagctgagataccaatacggtaataagctgctccagtaggaatgtcagcgattgatagatagcctccgtacttacctgtttcatttcccagtaacttagctgacacaaaagactcttcgttagcagctgagtagaaggcgatgtttccatatacgttatccttgttaggcggaacagtttgtgtcgtaagtttatacacagatagtttactagctccgtctttaattggaataaactctttcatagcccagtcttctttttgagctccaccagtgtcttttgttttccatattccttgatagttaatgtaagcccattgagtagcttttttgaaatcccatttatttgctgagtagaagtcttctccagcttcgatgtaattagaagcacctactacagataccataggccgtgcaaactctaactgaatgctatcttctgcttgaataaatccagctaacttattcttatcatacttgaaagaagggtagtcagtaactggtggattctctgctccccagaacattgcgaaagcttcgtcccattcccagttctcagaaaggaatctagtagagtaagtaatccgtgtgaactcttttgtcacaggctgttcaataacgtggtagtagtcctcgaagttagaagttcctttaaaggctacagcgtttgttggaatgtcttctccagtgcttcgtacatccactgagaaagtaaccaagtcgccttctttaattctcttgataggattaatgaactcttcccagtaaaacattagtccttggttagcacgttgagtagtagcgaccgctgctccgtttgaaagtgttgattcgttatactcagctccattgaaaagtaaagtaggcttagataagaatttacttcctcgcaagatgttttcgggatacatttggtcattgtgtccttcagcgatagaaggattcggtgttaagcttcctttaacctttcctctaaagtctactgggaaagtaatcttaaaggtgtcctctaaggcgtggaactcctctgggttaaatctttcatagccagcagaacggcctaagccccactgtttagtaggggctagaccagttaattcaaatggtgtataattaggtttattagaacatgtcatttaaacgtctcctttactttttgccgacagctcggttagcgatacgtttgacacgttgctgctcctctgtcatagctttgttagtggctcttgcaaattctctatcgttgatgtataagggaacgataacttcttgaacgcctccgccaccaaggccacctcccattgcttgaagttcattggccacagctgctgcgaaaggtctcatgtaagtagcgttttggataggcattgtaacttctcttcctcgctcgttatggattacgccctgattgataccgccctgtcctaactgtgttccgacacctactgcggtagccattggagagtattgtgcattgcctccaccaattccgtaaccaacgcctgaagtaagtcctccacgagccttgaagattccgccaacctttcctgcaacccatttatccgcttgtctaagcttgctttcaaagccatcaataccgccttgaatcttacccgcagtttctccaaaggctctaaacacgtcgttgattctagcggctaattggccaagctcttccatggcactagaagtatctgcgtcagcgtcgatttgaatatgggttccgtcaacttcttcagacttgtcggcaatcttttggatttctcctaccgtctgctctttctgagtcttagcatggtctactgtaccaaccatttgttgacgtgccttttcgataagctcatccctagttgttccagcggcagctatagattcatctgacatgtgattaattgaactgacagtttcaacatactgtttagaggctgctgctacagtttcagaatactgctggttagctgccgcgatagagtcattggccatttgttgagtgattgtcccagtgctactagcctgcgctctaagtgtttcaactatactgcttgactcagttgtagcaatattttgcaggatttcagagttctgttgttgcagggcatttatagcggcgccagtttcttgacgctgtgcataatcctgttgacccatgctttgatacaattctaaaatcttgttgttattagtttgaatggtatcggaaagctgttggccatgagttagctcaccttgcatcataatagatttctgctcgtcagttaaagtcttctcattagtgaagaatgtctgcaaggcatctaaacggctttggttagctgtcccgaaaactgctacttgctcatttaaagcttcttgcaaggcaggtagattctcttggatagtctggttcataatacctgaaatatcggcttctccaccatgcttacctttagccgctgctgaagcaagtttcaactccatttgacctactgaatctttaaggattgttgaagcctgtccccacttaccttcccaatcatctggtaggaagctgaactcttgaatgcttttgttaagctgatacataacctcattggtttcgtaagcacgtttagagattccaattccccacacttgagactgtgctaaagactcttggaaaggaatgttagtaaggttagtacctgcatctcgaacagcttcaacaaacttgttggcatccttagtaccagagattaatccattagatgtttccaataagtcgcttgtacgcttgaaacctttaccaaagtttgataatccttcatctactccatcaagagccttgttgaatcctttaacagcgtctagtgcaaacatgtcggtaagcgctccaccaatttttaatttgatggctttcacagagtctcctagttggtctaattgacctgccatagtctcacgcatagcagcagcctgtttagcagctgtaccagtagagtctactaatgcttttgataagtcttgtacttgctctttggtagatttaaggacgaacgacataccagccgccccgggctttccgaacaggattgttgaaagctcttggatttgaatatcatccatacctttaacagcatcttgtaagtcgtagataacatccatcgtaggacgagcattaccctcagcatctttaaactgtactcctaagctttccatagcagcggccgcaccgtctgttggattgataaggttagtgaaggctgtacgtaaagaggtacctgctttagaacctttaataccgttgtcggccatgatacctgccatagctgaaaggtcttccagagaagctcctgctagggaagctgatgaaccagcgtattgtagaacgtatcccatgtcctgcatgtcagcagccgtttcgttagcggtcttagctaaaacatcggcaacgtgttctgccttagaggcttccagattccatacgttaagggcggttgctacgattccagcagtttcttgtacacctgagccagaagcttcggcagcgtatacgattcctttcatagcctctgaagtttccttagctgtaaaacctagcttagcaaagtcaaccatagcttcggcaacctcttgagcacctttagaagtcttaccgccgatttcaatagccgtgtctccaagcatatccatgtcagcttgtgtaccgcctgcaataatctgtacttgtttaagggttccttcaaagttagcatagtccttgatggaatccatcgcaaagcgtcctgcaactttagtagcttcagcagcgcccattacagcagctgccataggagtagcagcggccgttagggtaccgaacccaacgcttgttgataggaagcttctaccaagacctaacatagagctgttggctaagcctgctccaccagttaacttgttgaagaacttaccgaagattcctgtagtctgctcagtgcttttacctaagcgtgattgagttccttccatatctttcattttacttgtgtagtcagagttgtctaccctaaccttaattcgcatttccttatctgccatttaaatttcctcctagtcaaagtagtcgaacaggttagatagctcttcctctttaaaggctgctctatcaagttcatccattgtttccgccatgtctctagtagtcttaggtttgtttgtttcgttaactaattcaaagccttggtacttctctaatttgacaggcttcttagattgcactagagaaaggaataacgaagcttctcttgattgtctttggtaagctcctcgtaacattccattaaactcatatggtgtacttgtccaaaagtcgctggaaggtattcctaagtctgtacacaataccataaagtatcttacgtctagcttcggaagctcgtcctcgtcttccgcctcctctgggtaatgagcttgtgttgtttttgaacaactgtttaactagaatacttgaagcatacgagctgttaactttctcgaaatattcttctaaggtaaactcatagctatccagagagtcttggagaagttctgccacatctagtacggatactccttcgtaatcagactcattctgaagcattgcccagaagatatataaggactggttgaaaggcgtttgctcagcaatgaaagaagcgtagtcttggtcaaaggctaactccagaaagtggatagacttgtatgttaagcgtaggttaaggtctttgccttggaatctaataatcatgttgtaatctcctttatatttgttatgtaaaaagagggcaagggaatttatcccaagccccctctgtgcagtctatttccaagttaatgttactgagtcttcgctaacgctacttgccgaaagctcggtcggggcttctactgccccgcgggctttacctcaatatcaatcaatgctgaaacagctgtgttgacctttgagcgaacgttgaatgatgcagtaccttctttaacacctgtgattaaaccagattcattaattgttgctaaagctggagtcaaggcagtgtaagttactgatttgtcagaaacattttcaggagtgaatttaattgtagcttgaagagtttctccaactttaacttccttagtttcgttcgtgaactcaactttcttaggtaagattgtaggagctacttgttgctcttctaaagcgcctttaccttggaatgttaatgagtaagtcacggcatcttcgtaaccaaactcgtaagggaagtcagttaagattgcttgacctacataagttaatccactagggaatttaacacgaacacgtacacaatctccgttaacccaagctgcgttcaatgcttggaaaccagcgttatcctctacgaacaatccgtcacagtcaattgaccaagacttgttacctgcttctgcatcgtcccatccgaatccttctttgttagatacgtctaaggttgctgctgtacggtttaatgtaccgcctcgttgtgctgctactttaatccattcgccaaggtcattctgaacctcaattaatactgctgtacctcgaatgtagctagatttacatgcttccatttgtcagtctcctttatgtgtgttattatggctctatcgtagagtcgattacctcgaagtctaagaatagatatgcacgctcgtttttataagttccgtttgcagcttctgtgtaacgaacgtggtcgagggatttctctccaatacagaagccgtcctctaagttaagaggagagttgataagtaagtctacaagaaagtcggttagactcttaacttcaaaagagctgtcgtagtcactccatatgtgaagggttaggttgtgcatcttcccgatagcatccttagagattgttctacctgaagttagctcctcaccaatcttaatgaatggatattctggaaacttattagttccgtcccagacattgattccttgcgctctaatttcctttacgatagccctttgcaaagctacagaggaacttcttgctcggaaaggtggtctattgctcattggtaattcctcctacactttgattctgctaagctgctcgacaagaatatcagaatactcttctaaagcgtctgtaaagttagggaatgcttgatgtcggtaagttccttcctctaggtaaacagcgtaacctctgtgtccataagggtttcctgaaacgtctgactctacaattcctgtaacgcttttacccttcccttctacttccgtcttagactttattccatcgtagtaagttcctgtgtctatgttacctctacgaacgatattaaatctaacagctgtggccatctgtgcggagttcttttcgacaacgcctttaacctcattaacaaactctctctctaatcctgcgaaatcgatttgtttagttatttctaagaaccctgccatataactccgtcctccttgtctcttcggatgtctctgtactcaaccttataagaaactgtctggtctgttccagtagtgttatcccaatagacatttagaatctctccagtggagtggcgtcggacagcgtctacccgaaggttctttaactcgaatactttatgctgtatcaagtagcgtttctcccactgttggtcttggttttgcaatagctttccttgaactcgtagggagcctacgcttacaggaaggtctttaacaacgattgttccacgagtcttataagagattgttccgtagtcgtcgtcagggtcttcccaatcattaaattggattaaatccacaatgtcgttataacgcatttgtaagctcctttcttaaagcatgaagaatgccttgttgcgatactctccttgctgttttaaccagcggttaagaatgccataccagtttgcgtagatgtcgtctttatagtcgtaacgagtgatgtcagtagactctgtcttcaagccttctgagttaagctgtgacatacggcttgctgtaagctcgtcagcaatgtaatccaactccatcggaaagtctgtaacttccttgttaatgaataagcagatatgctgtctagcacgcttcttcaagaccttcagaacgttgtctcggatagtgctgttaccgtagcctagcataagctttaagtcggtgatttcaatagcttctgtttcaggcggtagggtaacgtcttcaaacgacttgaacagctccgtgttctcaattagttccatttagcttcctccttaaaagaagaccaaggcgattcgcctcagcctccttgggatagtttattaagctggtgcctgaacagttacaacgcagtttgcagtagcgcctgaaccatctttagcagttgctgtgatagttgtagtaccttctgcaacagccgttactgtaccgtctgcataagtggcaaactcagggttgctgttagtgatttctaactccttgttagaagcgtcttcaggtgccactgtaaaggttaatgctttgcttccatcagaagctagtgacatagtcttttgagaagctgttactgtagctactaaacgtggattagtcttagctgtaacggcagttgacttagcactctcaacgccttcctcgacagctgtcacctcaaacttgtatgaagtgtttggtgtaaggcctgtaatagttttcgtaagctctgctacgccttcctgaacctttactccatcttggtaaatattataagtctttgccattgcttttcctcctgttgtgtgataaagtaaaagaggctacggaacatttccgcaacctctcgtttagcttactagaggcttgcccctccgtcttcccaagtcagagtaactgagtcctcagtaaccccgcttgcttgaagattttcgggggctgttactttcccgcagctaccttgttgatgaagattgcacgtttagcatctttaacgaatgctacgtagtgttggtcagcagtgatgttagtttgacgtttcataccttcacgcacttcttcaaccatgtagtcacgtttcataattaatgataaagctccacgttttaatacgaacgcttcgttttcaccaaccttgttagaaactacaatgttcattccgtagatttcacctaagtggcctttgattacagacacgccttgttgaatgtgaatgaagtctttagatttcaaaattgttgcacggcgtttaggtgagatgaataagtaagtttcttcatcaaactcttcaccaaatgctacttgagcatcaataacccactcgtagctatcagtcatttgaacatctgacggagttaatgcacgcatagctgtaaacaagtcgttgtcaattttacctgcgatagccattgctaattgacgaactgtttcatcaacaggacgaacataagcatttaaaacagcttcatcagttaatgtaactgatttagcaacttttttaacagtcgctgtacgttcagcagcagttaatttaactgattgtaattctgcaccttcagctaagtcttccgctaatccaatgaagttccattgtggtactgttaaagtgtcaccaggacgtccttgtaatgtacggtcaatttctgcaaatggagccaatactaagttgtctaacattttctggtgaaggaacgcaccaattacctcgggattaattaagtctgaaattttagttacttcgtgtgccatatttaatagtctcctctaatagtttatagtttatatgattatctgttgttatccacgttgaaccaactggcgatatttagcagggttagttcgagcaagttctgtcgcttcttcaacagttaaatctgttaaccgttctacagttttagaaccgccaatgtttgacatgtcgtctgagtttgttggagctgctttagctgcgattccagcaaccttctggtcaactagtcctgaaacgatattctggatttcaatatctaattctgaaaccttgtgcttaacggcttcgggattgtctgataaaacgtacccagctaatgaggcaggcaatccagcttttgctaaagcttcttgagctactccacgcatttccaacatttgcttatctttttgaatagctgccatttgagcttgcatttgttttaattgcttttgctcaggtgtttcagcagggtttaactcagccaatcgctcttgaacgatattctctaagttcttttctttccaagcgttaatgcctttagaaacgctttggtcaatcataggggctactacgccaagaccttcgtctgtctttaagaaactagatactgcgtctttcgttaagacgtgaggttggatagcttgtgctaattctggattagattcaatagctccaacgataacctctggtgtcaaatcaacttgttgtccttgtccttgttcttctggattcataatataagattctccttctgccctccacagttcaattaagccctgtgcgtgcgtttagattagtttgtgtaaagttcttttcctcttgagcgccctttaacattaccgctctataagacgtatggaattaggatgcaaacacagtgtggatgtctaggtagcaaaggcatggtggctagggtatatagaccctcattaccaatacctccttcagcaagctctcggcaatctgcacaagtaccgctttcagtaagctctggacggatgcaccatttccataattcaatctcatgctgtttagaagcttcgataacagcgaggttaattactgccgacatttgagcttcaatgtgtcgacgtagtgtataggacaatctatcccgaagcttttggatagtggttacgatgtcctctgtagatggaactgttccaacagcgatggcctcaatctctactaataattgcaagaagatttcttcaaagtagtcgtaagcctctgcaataacttgttcgatgtaggcattatctgtcgctgaaggtatagccttatttggaaaaagctgtcccaaagtctcttctaaggtgtctgacatgctctctaaaagcatctccctgaactcttcttgatagtctatcaatgctgcttgaagctttctgatagcctcatactgactcacctgaaggattgcaaaagcatccaacttagggtcagctaggtttactataagctcattaacaaagccatcaagaaggacagtgaagcggtcagtgaatctatggtacttgtggttctgagcggtcatgagagcaactaaggcgtcttcaatttcctgttgacgtttctctttatccttcttgcgtgccattgttattcctcctagtcagtcttaggtttcttttggatagctttctcttgttccttcgcagccactgggtcagtagttgtgatggtagatgtgttagcagggttcttatctaacttgttagcagctgccgttgaggctccttgtacagcgctagtctgtttgaagttctgcaaggcgatgtctgctcgtttctgacgttgtgcgtctgctttctcaacctcttggcgagcgtcagtaatccatgggaattgattaatgatagtttcgtcaggaagcatgtcacgaagtttaacagccatgtcagctaactctgcgtaagattgtggtaagttacgtacaaagacaggtgttacttcattcggtttcaaatctttctctttgctcattaactcaaggtagctacagataagttcataacgcttagctaatactttgcggaacttagattctttaacagctgatttattctcaagcggttgcgtagctgctttcaaagcttgtcctgaagctgcggtgaagtctttagaaaccaagtcaggtgtttgtgataggctgaagatgtctagcttagcacggttcttaatgttctcgatatgcttgtcgttaacatctttcgtaatgaacttgacattaccagtatcgtctgttacaattactctgtcattcttcatgttgctaattgaatcgctgtctgcgctaaggtcaaatccttgtagccacaagtaagcgtcattccaataagcaatatcattaacgctgtcagaaacagctaggttgtaagcatcgattaaagatagttgagcttcaaagtcacctaagcgttcttcgttagcaatgatttctaatacagggaactttttaagtaggttaggatgttctacataacctctaatgtccaacacttctggaatctctttgtagacgtctctctcgtcgtcggtagagaatttaagaatcttatcctcagtgtaaatctcataagttctaattacgtgtcctgtaatatcactgataacagtattgtaatagatggctgctaatggctcttcgtcaaggtctgctgagtaagcaattaaacagttcattggagaaacagctttgaagcgatgcttgttgttacggtcaatccaatggatttcgaaacagtgtccgaagattcctgagagcttaatttcctctgagttaacgtctgttacatagttctctttcataactggttgcatggctttgatagttttctcatcgcctgattccgtaattggttcaccagccaagtaagctgtggaagtgtctaccaatagtttagggaagttatgaacgattttagagttaggtttgtttgcgtcgtcgaaagtacgtgattggatagcaatgtgattgcctttgtaatagtcgtacaatactccataggcgcttgaagaacgagagtgcatttcagcaagggttactaattctcgaatatcgtagttgcgtctaatgaaatgtttacgtcggatgttagtagtgattatatcaccagcgttaatttgtccgtttgggattgccatatagaatcctccttatatttttcgctgatacttgtacccaagacagatactagacgtcttataggcctagtaccgacttgttgatggttcttattctgttacgtggaatgaggtcgttgacagcatatcgtaaggcatccatacagtggtcaaagccgttttgcttaggcttgttctcgtagtcgtcagagcctttagcaggtttccactcgtagttctcaagctctttaatcgtgtgaacacagcgaggatgaacgtagattgtatagccttgtaaatactggattccatgaagtactgtgttacgtcctttcttaacaggcttaatcttgcgtgcccctaagcgtttaatctgctcgatggtttcaaggttagcagagtcagcatagataagttggttggtaagcttcttacgcttaatcacatcgaatatctcaggagctatcataccagttttaacaaactcgtcgaatatataaattacacggtctgcttcagataccagtgtagaaaggattacggtagggtcgttagtgaacccgaagtccaacccgaagcgagcttcatagcccatgttaagtagctgtttgggattgaatgttgaatctatcttccagttggtataaatccgtttacctaatgaagcaaacttaccaagagcgtagatttcataatagactgggttagtgttcttcaagtcttgtaaatggtctaggtaagctttaggaaggaatctattatccttgtatgttgtacagattaccttagagcctttaggacgcttagtggcagggttgtggaagtattggtaaacccagttgtctttagaggcagggttatacattacaaacacttgttgatgccgtgctgtaggatgccgtaaacgtgtttgaagttggtcgtaatcctctcgtgtaaactctgttgcttcctccataatgatgtctgtaagaccttggatagacttgattcgctcagggtcttctaatcctttgaagatgtattcagttttgttaggcagcttgattgtcatgtatgaagcttttacctcgcagaagtctagcagattccattcctctaagcagactagaaactcttggaagatggagtcacgaatagtgttgtcaaccttcctaagaacgaggaacttacgtctttctgataggccttttagaatagcgttttgtacaacgaacttagacttgcctgacccagctcctccgtaaaataccattgttctatgttgtccttcaggctggttgtacatgtatggtaaataggcttcgttaaatgttcttttggttatggttaattttattggttcagccattatgtgtcctccttaaaattatgtaaaggcctcaaggaaactccctcaaggccgtgtgaaggttattctgctggagtctttgtcaactcgtagaatcccattgcggaaagccctgctacaagtccgctaatagcacctacgtataatgtgattccaagaagtggtgacatagctaacccaaacaccattccgatagctagagaaactagtggtaagtatttaggagagactacatccatagacttaactaagttaactaagcctgtggtaacgccacctaaaacaactgcgatagaaattactacgtccattaatgcacctcctttcaattagctcatgattagcttaacaatggtcactaaagctgaccctactcctgtgacaagagcagtggccacagcccacatctgcttttgactaaactctttattcttgcggtctgaagcttcgtcagcaaggtagctttccgtttgtctacggcgctcctcagtaagcagtttaagcatttcgtcagactggcctttgatgtggccgattagaacgtccgaacgctcagtgtttttgataagcaagtcacgaacgcttgaaatgtctgttgtcattctgtttaagtctgactgaatagaagcaatctggttggcatgagtgttaacgtctttaataaggtcgttgacgataacctctaagttactctcttcaaatggcattggtatctccttccttaaaaaccagaaaggaagtcataacttacgtcactatccccttcttcaacttcggtaaagtctccttctacaaactcttcttcctcaacctcttcagcttcatttggaacgtcgtcagcaatgtctaccacgatattaagagactgttctaccttgacatcaggggctaataagtcatggaatttagccaagctatttagtgcgcttaattgaacgcttgtgtcaataggtttctctacgacatctcctttcacagtaaccgtgtaatcagagctgtttcgtaaagcgatattagttagctcttctaatacctgtgttggactcataatagctgcgtcagcattctctgaaacaagccattgtaaataagctttcacatcctcacgttttaacacacgataacctgagctttcattatagcctacggaacggcagctttcagcgatattcatagtttgcatataatgtctcgcaaagcgtttcattttaggttttaatgtagttaagtcaactccctcgtattcgtctggctccttagaagtagttaaatacttgcgttgacgttcttccaactctctgtttccaattcttaccataagtgcttcctcctttctggagtatttattcaagctcatttaagagcctcttagattagaccttatctggctctttttgtcacattttttaatttattgtgaaaaagtgcttgacattttaaggtagccgtgttatagttatttatgtaaaagacaaacaggcgctaaagcataccttaaaagccacaggaagctctctgagagcttttgagtagcttctagggtaattatccctgaacacattagaacgccttacacaggcatttaaggaggaatacagatgagagcaattaagatgtactccatcaagagttatcagcagtatttagagaagcttggtacagtacgaaaagcggccgcagacttcgagcttaataatagcttttcaaagaaggctcatgaattaatcgctaaccagtatgcagaagatattgagaagctttcccaaaaggttcaagagtatcacgagcttattgagaatatcgtttctgacgaagcagaagaagacgaaaagcagcttatggaatacttaggaaccattagaaagcagcagaagattactgaagagttagagtctggaggtaaggaataatggaatgcattgaattggataactactatagctttttaaatggaggttacttggaagaagaccaagttgatatagaaaactcaattaacgttttgcttaatgttataaatagcaaaattgaagacattactgaagttgagttcgagactgctgtagaagaagttactgcagccattgagaatcttactaattctttaagtgagctttcagaagtgtttactgaagctaagcaatacttggctaaggaagataagaagtgggaggagaaagtctaatgaggattgcgagtattgatagaggagcgtatactccagtgcatgtgagcactggtaataagcttgtgacattagaaagttatgccaatattattacattcagtaaatcagaagcagaagaactcatccaagttctgcaacaagctattaaagagcttccagaagaacctaagtacgttgtgatactacctcagaaagcatctgaggtgttttgggagtatttctatttagctgacaatggctctatttgtaatagcaataaaccatatgaagtcttagaagacggtgttatgacactaagtgaaattagagcttttagtgagcattatgaaccttttgcaattcctgttgaagagtttaaacaagaagagattggagagttttaatatgagcaaactaccttttgagaaagaattgaaagaggcttgtgcaaagcctacattaacgcagcgtgaaacagctttaattgagcttgtaggaaagcttcaaaatgccttggtggattccaattgggataagctgattgctgaacaggaattagagtatgcagaagagcgccttactagtgttgtatgggaaaaagaagacttgaacgaggagatttatagccttgaaagagaaattgaagacttagaaggtcaggtacacgacttggaggacgagcttgaaagctctaaggaaactgtagaagagcttacagaagaagttgatacgctttcagaacgtgttgaagagcttgaagagattgtagaaagccttgaggactagtttatggtaggagatatttggattgccttattaaggaaactaaaacgacgcttttgccaacatgagtatgggtcacatgttcaaataggtatatcaggtttgccataccgtgcatgtaataaatgcgggaaagctgattacactaactattaggaggaatttttaatgaaccgtattaaagtatatactaaagacatgtgccaagcctgcaagatggctaaacgagtgcttacagaagctggtgtaagctttgatgaagagtttgtggacgttcgtaccgatacagagactgtggagatgcttcgactagcaggtttccgaagctttccagtagtgatgttggacgaagacttcgacacagctattagtggattccacccacctcgtttgaaggcctttattgaaactgttaaggaggaagcttaatgacttacgttaataaagaggatttattacagtaccttagaaacacacttgaaacggaagctagagcgttgccagagagttgggtgagtggttttgacgacgcatttagaggactcattgaaagtgttgaagaaggacgctttgatgtagaggctgctccagaaagcattccagagcctattaaattagctgttgacggaaacattgtagcagaatcaaacacaggaagctctttaacgcctattaacaccttgcatcacaattataccaaagacgttttagaagctctcctagaggcttctgaggacttctatgatgcctctgagattcaagccttgttgaaaggcgctatccaagctattccaaaaggtaaaggagtgattatatgaagcttatcgatgccacagtagtattaaatcaaattgacaaggcggaggaagagtttggaaacaccttcgcctctggcacagacttcgaaaaaggttgggatgaaggctatcttgacggattcagagatatcaaaggccttgtgaaagacattatcatagaggatgaaattcctttggctactccttcagacatgattccaaaaagtgagtttatccgccttttagaagctatccaagaacgttctgaagactgggacgaatgtaacttcattgatggaattatggaagcctataaataaagcataaataaggccttcgggaagctaacctgaaggcttttcttattacatattaaaacgagtctctaggaagcttactaaaggtcgctgctggcactttgcgttaggtgaggccatacgatttaataagctttctacaaactcgtccttaaagtaattaacaactgactccatccccgacctgtaacctagacgtctcaaagctttaggaaggcttaggggaacgtttcaggaagcttttaggaaaggtcaagtaattactaactcgctggagaccgctcgttagtagcttcgctaactaactcttaactaatacgacagtatttcccacatgctttaaattagtctcactatttgatggaatattttataactgtaaagtaaatttaatgtttgtaacacaaatgtaacataaaggatattgacaaggcgcctgaaaggtgctacaatatgcctatagaagaggacttaggagggtttttagtgaagcttataaaagaggttaaatcagtagacgttgagacaggtttagagacagtttacagaagcgctcatgcagctgctaaggatttaggaaaggattcaacaagcgttcgcaacgcctgtgctaaaaataaggtgtatgcagggcatgtgtggagttatactggaagagagtttgagaaaggttctccactagcttctaaaaagcctatcaaaaagtatttggagggagattggaaggttgttaatgggtttcctaactactccatcacaaagactggtaaggtcgttaataacctaactggtaagattctaaagccttttataaatgataggggatatgcggaaattggactaggcgatgggaatagtaaaagactccatcgggtattgctttccacttacaatccaatagaaggttgggaagagcttactgtaaaccatatggatggcaataagcttaataacaacctagataacttggaatggttaacacatgaggataaccttaaacactactacgacaacaataagcgtatcctagtctttgacgatgctactggagaggttttagaagaggttacacgcatggagaaagctgccactaagtatggagtacctaaaggggttatcaaagagctttgtgacctctatcatgagggcatttacgctcaggagaacggttatggattctgtcgtatggaagattttgggttagatggttaaaaagtgttaggagaggcttgacaacaggcctcttttatgctatactaatagagtatcaggaaaggattgataagatgttattcaagaaggacgaaaacaagcttattaaagagcttcgagaagagattgacgaactgatttatggtaaagaggtcaacaacgtaatcaactatcagttagaaagacgtgttgaagagcttcaaaaagaggtagacttcttaaagcttagagaagcaggttatgaagaacgcttaaaagcagcttctaataaggaagtagcagctattgaaatggctacctctgaagttgaagagaacttgcgtaaacaagaagcccacaatcgcctattcaagaaatttgaagaggtttgtcaaaagctttcagtagaagaggagcaaaacaaacgctgggaacaggaaatgggacgccttcaagaggagtacaacggccttaaacgtgagtatatggagcttcaaaaagctattcctagcaagcctcaactaacagccactattaaagttgatactaaagagattcaggaaatgcttgagaatgccctgcaagagattaaggaagagtattccccacgccttaaagaggagcctaagtatgtttattggattgaagaagaaggggattatggtagacactatgagagcgactcccctcagcagctattaaaaaaggtaaacgtcttttattcacctagtgatgcaggtattttaacgctttctgaaagtcttattgaaatggttactcgtaaaaagcctaaagacgtgattattgaaacttactcagaaacgcctgtggagttccattcaacacgcttcgctaaaacagttaaaattacgctttgcagaaagcttgctaaaggagttggaaactatattgtctgagaaatttactaaacgctatatcttagcagcctttatcatagcctatatggtaactgttggagttatcattggagtactttggttagtatccttagtaactggttgggaacagcttttcttctatcttggaattctattcgtgatagacctcctttgggaaactactaagtacgcctataaccgctataacgaagataaacataagacttgggaggaactttaatatgaattggaaagcatttagcaacactttactaattttacttatttcagtggtagcaggattcggtatctacacacttattacagcgcttttcggactaagcgtcactattgtggttggaagacttattgtaatctacttagttattcgatatgccttctacgatacactaaaagacatttacgaaggctttaaggatactccatataagtaaatattatttgaaggggcttgacatagcctcttttttatggtatactataataaacaacaggaaaggtgtgttataagctatgttagaacgcattaaagggatgttgagaaaaattcgtttagcaaacggcttggttacttcctaacaaaaggagtcctaatccatcctacaacttaataaacgcttgtgtgaatactccactagaaagaggcaaagcctctagggctgccgacaagcggcgccctctctatttgtggaatctattcttaaaggatactcaggaagaggtattaggaaaggtagctttaaagctttcggacgtctattgatggaatgcttatcggatagcttgtcggaacagctttcggaggcctgtcgggaagtgtgtcggaacgccttataggggattttcgggaaattataaatcgggaagctataatgccgcttttcccaaaaactgtgtgagatggacagtcgcgcgccccgcccgccctcggaaaagctaccccctgcccttcccaaagacccccgcccgccgttgccacgcctgcgccctgcgccttccttcacgcttcgcgacaccttgcgagtgcttcgcgacgctctcacgtcacgccgtccgacacgctcacacacgcgactctacatagactctgtcactttgctgttaacattgcattgagatagagtctttctactttggataatgtgtattatgtaaactagccgtcgtgacagccttgcggacagtgttcggacacctttcgggtgcctctataatgcctgtcgtgtgcctgtgagtgtggtctttagtgttaacctgtggtgaaagttatcggacaccctcgcacatgcctcctcgcaaagcccaccacaccagcattccgcaaagccttcttatttcctttaataaaaacaaatccaccgcaaagctttcggttaggcttctcagattcgccttgagataacgcatgctaacgccctttagataccctttagaaaaacccccataacaacgccctagaaaaagctttggaataatattgttaggcgccctctcactcctcttaaaaatcccctatgtttaataggcttttaccatttctttcttacaattcctttcttttcttatcctttttgatggattaataacagcatttaagaatagtctttgcatattcctttgcccactgctttattatgtgtgtgagagagggcttctcgtaacgtttcgagaaagaagttgggtagttaaccattagaaagctttataaacgttgtaagaaatgcctaggttagatggattagaaaagcattccacaaattaagataggcagccttgaatattacttttaagaaagcaaaaagaaaagccttcgagaagttatctcaaaagctttcctgattcctgtttattaagtttgaaaaaaatgttttaaattatttgtgtgcttctctgtagtttttcaataaggttggcttatgtgctttataagcttcggtatattccttttgtgcttcttcctcatgattgattagataggcgccgattacattagactttactgcaatttcatatgcttgcttttcggaaagtttttctaaagcttccacacgttccgtaagaatagctttatcttttgcgttttctactgccttaaatgatgtgattcctgtggctgttgccaaagcgattactaggattgttaagcctgtaataatgtttttaaattgttttgacatggttgaaaacctctttcatataatttttttatttgtggtttacaatgcttttattcttcgattaaatattctaattccaaaatagctgttgcgataccttggtaaacttcttccgaatagtcgtcaatcatcattaaatctttttgagtttcctcaagttgtttcaaaatttcttcataattcattttttaattccctcctatttgtttgttgttatcttacaaatatagaataccaaagttattgttttttgtcaatagttttatttaaactataaccatttatcagaaaagactgcttttgttatctcattatctaataaattattgtaatcggttagcaactctattaaatcacttgttccattaatactcaagtccctaagtaaccactcactttctaagacataacctaacctagagtaaaattcatctattgattcgtaacttaaaatagaatagctttcttgttcttgaaataagcaaaatagtaaatgaataacgtttaatttcatgttcataacttcctctctttatcttatgaattaatattaccatagaaaggggctttttacaacccctttttataaactttttcaatattctagtatataactttcttgttgccgtgtggactgattccagtagcttaattctaacgctatgctatcccactcaaaaacttcgatagttttcttgtcagttcccttgagctttctcaaaacatagttttcagcttcttctaaggtgtcaaaagacttgataaaagtgtccgagttgccttgtgtaatgttaacaataaacattcttaaattcctccttaaggcgttttacaatggctttattataatcttcttcggcttgctcgttccataggtcaataacttcaaaaaagtcttcggaaacttcttctaatacgcggttaaaattaagcgtttcaaatagaaactcttcaccttttatataataaagcatattagcaacttgactagggctacttaaatccgtgtaaaattcaccaaagttattactttcatattcttggatttctccaatggcatcaaaaacgccatattcttccaagtctttaatttcagcatttacgccatagttaaaaatttcaccgtgtaagtcacagtagtaaccatcgaaaccattttccaaagtttctataatttctttaaaaaatcctttttcatttaaaaatacattgctttcttttgtcatgtctataacttcctctctttatcttataagtctataatacactatgttttgttttttgtcaatagctttttgtaaacttttttaactactctattttaaattttgctgtaaagtcgtcgctgtcgctgtctgcataaacatagcagtatgtttcttttgttaccttgttacgttccacccttataataatgctaacattatctaacacaagataacctgtatactctgcattattaatttcttctaaagtaatttcttttgtaatttttgtcatgttaaaaaccgccttttctgttattttattttaggctttttaaagccactttctaactatattcattgtagcatattaattggattattgcaatacttttttacattttttgttctatacttttcaaaatgtcaattagttcagccttgctcatgtcttcgatgcgttgtttttcagattctttattaaaagcctttactagcgcatataaccaacctataaaagtccatccgaaaaagatattaataagaataacgcttttcttattttcagcctttctaattaatattatgatagatggtaaaagatataatagtacagccattttatgaaacctcttttcaagttttattttaggcttttttaaaggattgccataaccttatcgaatgagtatttgttacttgttatcttatacgtttatcttactacttttttaaatacttgtcaataactttttacaagtttatttctttatattctattacatgtaaatgccttttgtagaatgtttgaccgtttgcataaaaatataaatcgccgtcacggtctgccttaatttttttagtttcttctctctccctaattttattatagtaagatacttccatatttttcttgtcccaatcaaaaacgataatggatggattgtaaagaatgctgtcaacgtagattttttgtgtcattttagaacttcctttcttaattttctatattatagcatttttgaaaaccattgtcaacgcttttgactaatctttttcataaattgtttggatggcaattgtcaacgccattacacaagccataaaataagctactgtgctatgccctgtgaaaaagcttgtcaaaaatccgcctagtgccatataaaaagctttactcattttcttgttacctcctttcttgatggcttaataataacactttcaaaaaccattgtcaacacttttggtaaacttttttaaaaaggttgcacgtccgcttcttcgtcgctttctatttcgtctaatggtatgccgttttttacatcgggcaatgaagaaaacttttcccgagctttctcataagaatgtccgcccgcttcctcgtagccgtattctagcaaagctttatttgtggcttcgcaaaccgcttcgtcatcttttgaaatggcttttaataatcgcaaagtataagcttgttcctccgtcactcgtgcaataataaagcgttttttagctttctttcttgaaaaattgatagacttatcaagtctttttgaaagctttttatatgctatttctagcgcctttaaaataaccaccgttgaaattactgttacaataatattaagcattgtctttttcctccgctttcattgtaccatgttttaagaagtggtgaatattccgaaaatcattttctaggctcataatctcccatgcgcttttttctgcttctcttattgctgtcttggaacaatctttataagtttttgagcgaccacttcccgaatattccacactagccaccatggcggatttacttaatacaaggcttcttttacctctatttaaggttttgaaaatcctttggcaactatttaatgccttttgtaattctttttcgtcaagtcctccgcctagtttttggctagcctttaaagtggctagctctcttttaagtcgtgcattttctttttgtaaactctctttttcaaacattgttaacgccatttttagtattctcctttcacatattgagtaaatgttaccatttctcgaaactctttacaagtactcatataacttttatataatttttcaatagttttattatacatatgccgaaacctcccgatattctttcaataaagattggtcaaactctggaaaagtgttttttgcgtttacttcgctggctatgttctgaatatctgctaaaatgttatagctttggataaggtagtaagattcttcccaagcgcctttatcgtagtttctgtatacgctttttaaacgtttaaaagaaaagtttatcggatacactcgatataatggattgccatatacatcgctgccacgtttttcgtactgcattaaaacattgttgtaaaaaacttgcttccgtccgttgtctaattcgattaatttcatcattttataaaacctctttcttaaatttatttttgtagttcgttgctacacctttaatatatcactttcaaaaatgcctgtcaacaataaaatacaacttttttaaattctttttggcagcttttacaaaaaacggcgctttatatagtagaaacttttaaaaacaacctgaaaaagctgttataaaagtgttttgatggattttaagaaagaacgtgcaaaaaggcttgtaaaaagagcaagaaaatcctttaaaaaggcgtaaaaatgcttaaaaagccccttatagggcacttatcgtggaaactgggggggggggata

SEQ ID NO: 3 - DNA, 17738 bp, Unknown
  Clostridium phage vB_CpeM_AE3110, complete genome
acatattccccagttattaccagtagataaagcatcactatttattaacaaattgtttacaaacatattttataaaaaccattgaataacttaacataatttgttataattaaagtatcaattaagagaggtgatattatgagtgatgacagctttatacaatttatatttataaatattttatgtgttctaatagaactcataatattttatttattaggtttaatgtgagaggtgatattatgaattattcgtacaaagttgttgtatcagaagattcagaagaagagttcaatagtaaatttaaatatgaattaaaatgtaatgatgatttaaaggttattgatattgattttgaatttagagaagaaaatccgttattcaataaatttacagcaacaatttattttatataatttaagaggtgttatagtgttaaaaataatatgttactggttactatttaatttattcgttctaggttatttatattatgtaaagaggtgattaaattgattaaaaaaataattcaattaatactactcgttatcatgggaatattagttggtatatcatttatgtttctatataacaatacatttttaacaagttcaattttcattatatcaagcttattatttgtaatgtttttaagctgtatattatattaagaggtgaaaaaaaaatgaaattcaaatcattattaaatttaataaaaatagaatctaaaagatgcgttttattagaagactatgacagaaaaatcaaattagatatgatttatacattactaagaaggtcacgctacacacaaaaatttattgataactttataaattatgaatgtaaagataaagaaggtttagaaattgtattaaatgatttactaaatacaagaatacaagaacttaatacaagaatatcatatgagaaaatgttttttgaaaactatagtgatacttatgataacataaatcaactagaaaaacaaatcgatttttatgagggtataaaagaaagaataaaagagggtaaataaaccctctttttcttattcacttcctttatgttcttgtaaccatgaaccataacaaccattattcataagagtatttgaaccaagagaagtaaatccattgaatggactaacatcataatgtccattattgaatattatgttattcataaagacattattattccaacttgttgatgctgaaacgtctattgctttaggcttaatatggtaattgtcataactagcaaatgtattattagaaattatatttgattctaatacaccagttaatttaatatcatataagccaccttccacatttttatcactaagagttaaatttttagtcccgttatcttcaaaggcattattacttataacacacgctttagggtcaactaggattaaaccacaatctttttgtcgccaaaatgtatttccagtaattacagaacttcttaatttttcagcgtatatgccactataagtatcaatattagcgtaactaccatcaaaataacaatttgtgatttttaatatttcaacatcataatttgattcattcttgatgtatataccaccaccccatgaaccaccaacaaattgacaattatctatttgttgagaacttgaagctatccataaagcatatttacgattcttaccccaaatattagaatttgttaggtaacagtctgttattccaattttcacggaacaacttctaaaatccaacttatccatacataatgaataagctccaccttcaccaacatattcaccattataatacacgtacaaaccacccacaatatctactggatattcatctcccatttcacaacgaatacgctcaactttgctcatatttaggttacataaatctaaatgccaatgattggtagctttattttttattaagtgtaaatcatgtaaataacagttgcttacaaattcattgtaaataccacttgtttttataattggagtatcattgttatttataattattgtgtttttatctccataaatctcaacatcatcttttaaaataatgggctcgtcaagttcataagtttgtccatatatttttatagttttaaaaccgttagctattgcttgatttacttctctaccaataccttctttagatgtacataaataacttaatttatctatcatagattttaacatttctttatatgctgttacatcatcatttatattattttctaactctgtaaaatcacttttgacttcttccttaaatgataatatttcatctttcataccattaattttattgtttaaatctctgaatacagtttcgtttattattgttgctaaagtaccatcaattaaccattgatttaattgtttagcgacttcttcttttaatccttgcccaactaaccattttgttaaatctataacttgattttgtgagtcaattaattcatttattttactaaagaattgacataaaatttcttgcaaggataaccctgtgtaatcataagctgtaaatatactacaatttaaatctataggttttaatttatttatcatttttaacactcctttaacttgatttaaatttaacactatctaaataaaatctattaatgttagttatatcacccataataataatattaccactttttgttatatttacaaatctaataacatcaccttctatattgtctttagcatatattggtattatcgtatctgataatggtctatattttaaaggtaatctataaattggtttacccgtttcacctccttttaacacacccattaatgaaacctcattattaccagttttgtaatatgaaaaatttgaatattcactatcaataacgttactgaaattttgaaaattatttaataaattttcatttgttattattattggttcttcaaaaaaacatatatccctaacatttccctttgagtccttataagaaaataaataattatcattatacgctgaaattaatgtattttcccttatatcatcacttaatattaagcctttgtaacatagttttccttctaaagcaattaattcattacaacatctgttatcattaaaccttatatcaccgtaacaaatagaaccagctatacctgtagtatttaatgaaatttgttctaggtttattaaaaaattactggtgttataaatgttcataacagcacctttaccctcttttattattaattgttccttaggattaccgccaccttcaaacctagggtttaaaacaatattacttcttccttgattcattaatatagcagtaccaccccttatttgttcaaatgcaatatttaaaaacttattattattaaaatttgttgttgtttgtgattcattttggttatttccaacacataaaccgttaatacaatctaaacttccatcactaattatattttcattaacccaaccagaattatctgattttaaaactattccatttttacattttcttaaatctttaaatgatatttcattattaaatgaacctcttaataaattgtcgttgtagtctttaccttctattatgattccatcaccaaaaccactaacagtatttattttaattttattgtatgaacaactagaaaatttaattgcaaaactttgtatattagttaaatctacataagaaggaattgaataattttgaattttattaattgatagttctgaattacttaatctaataaaatcaaaaccgttgcaatttttaaagcttaataaaccattaaatataattattttgttattccaatttttaatagtaatatttttatctattgttaaagattcttgtatttcaatttttaaacaatcatttattttattctctgtaatatcatttattaaattgtaaaatttttcttcatttgtttcccccatataatcagataaactaatactttgtttagcatttattttttttatttcttcaacttgtaccatatcaccatctttacctttaatattaacgggtaaaggatttggtaaattaccattattgctccaatacaaaataccatcaactgtaacatgaggtttataggtgtagcccgtatcaccttttaatacttctttataattaactcttcccgttatcttactcattatatcacctcacatatactattaataatttctgttctaataacacctttttgtaatattgttacagaataattaaaagaacctttttcataagttgggaatattaaatcagtatcattaaattctttttcttcttttatagaattaaatacaaattctattttatcaccttcttgtaatttataaccacatacatttaaatctaaaatacaagtatcaaattgattaacagttattttatttctgtcaattataatcattatttcaccacctaataaacatacataaaaagataccttgcacctattataattttttcgtctaagtttactgttacatcaacccatttttgctttaattctgctgatgaagttacgccaatgttaccctgtgaaattaattctgttttttctttttgtttgttgtttttatcgccagtattatttatattcacattatttttattatttgttttacttgatgtactactattactatcataagcatctgtagattgacttgaatttgtcaaataaccatcttctaattttacactagctataccatcaccgattgcactctctttagtgttattggttgagttattactttcattattatttatatttgtagaagtattaccaacttcattttgtgatgttaataaattatttatttcattctcttctagttccctaataaaagtttcttttaaatctttattcaacataaagtcaatatctttacatcttaattctatttcataaagttgttgataatatggcataactcttaaaagatatgctcttagttgttgcttaaaccttgctatagtttcaaatcctatttcatgaaagtaataatgatttagaaatttttcttcaaaatagcacttagcctcttcttcatagtaaggataatcaaagtcaaataagttattctctaatgtccttaactctagtgtgtacttcgccattttcaccacctccaaagctatccacaaccttagttacttttatgttagttccgaatttttcatttatcattttacaagctaataacctagttttatattccatgtctaagttagtttcaatgtattcattattagcgttaacttcatcaactaaaagtctttctcttttttctgtatttgcattatctaaacccaaaaatgaaagtagttcactctcaacagcctttctttcatcttctaatttatctaccaaatataaaacacctgtttgcaacagcttaaaaccatcaaaaccttgttcggctaactctttatctaatataacaacttcctctccattatcaacatcatttacaatgttttttactgataataaattattatttgttgctgtaacgaagaaaggtttcatttgttgttttaaatttctttttattactgtttctatatcatccattttttgagcgtaatgtctgataaaatgttctgttggtaagcataaatcattcgctcttatttttattccatctttaccatcttctaatattttttgatattcaccatttcttgaaactatacttaatttgatattatcaccataaatatttaaatcacctaaaccatgacatggtaaacacattaaacctaatttatcatcgttaaagaaaaaacattgtccattttcaaataaaaaatcctcaatatgtctactttctaaaccagtcggtaagttctcccatttaaacctattacaagccataagtttataatgtaaaaatagccttgtttggttatcacatattgtattttcatttagtaaaatgttaggattttgagatattatagattttctttttcccatctaatcaccacctttttaaaaggggaaatttattccccttgtttttgattattttttaagctgtcaatatctgattgtatctttgttaatgttatgttaagttctgaaattgcatttgatagtttactattctgattaaacattatcatacacgcaactatgggaaacccaacgctatttattatatttattatttcttgcattaattacactcctccacaaaacaataccatcttttagcaatttcttgttgacagttttcagcatcttttttgctgttaaaatattgaacaacaactctataaagttcattttctttaatagttgggtcgatagcatttgcaattagtaaagccattttttcgtatggtgtaggtgaccaaacttcatttatgtcatgagggttatcacaaaacatagtttcaaatattatgttaggtgcattaacctctctcatttctctataattactttcccttacacctctattttgtaaacctaaagtttcaaagttatcaactaatctttgtgcaatatctcttattgatttccttgcaccttttgctacaaatgaacacgttccattacctttatgttcttttgaaacgtccatatgaagtgagataaatatatcaacgttattggaattagctttatttatcccctcacttagctccccattttctgttctagcattagaaccacaataaactacctcatgcccgtgtttttctagaacttcaacgacctctgtacataaaattctcatacaagcttgttcatcccttaatgcaattgctcccttgcaatttgggctatgcccgtctctaatacctattttcattaattatacctccctgttatccatagaataattaccaatttcaactccttcattatcaatatgccatattgtagtaccattgtcaaaaatacctttgagtatatttaagtaattattaggaattttattagtttttatattaattcctattgtttttatatagttataataataccttgaattaatattaattttcatcattttattttgtttatatccaaattgggcaaaataatcacctattttttcatagtatctttctgttaagccataacggtaaagtcttaactcttgtccaccattacgtaaaccataaattatatttgacccttgactcaataaagtattaggtgttgacctcatgtcgttcgctgtagctaaagctgaactgatagccgattccctagttaattgactctgcatattcgtatgttttttatccatgtatgaaccaacaccaccaaagacactatctatcacaccaccaacgttaccagtaaatgcacttgcaacaccaccaacaacactatttgcgatacctaaattcatgttattttttgctattttatcatttaaaatcgtttggtttacggtattttgaattgattgtgctgtctgattttttgaagttgcaacccaatttgcatatgctgaactagtgcaaggcaattcaagtgcatcatttgacaccaaagcctccgtcattccgtctgtatcacctttataggtttgtacaaatatcccatatgaacaacggtctgaaactgacagctttataccaactgaacaattaggaattgaacaaaattgaggttttaaaatcatagggtcatttataccatcacttagcattaaataagtgtacggataattatataatttactttcatttctccagtttcttttaccacccacactcttttttactttgtaagtttcaaactcacctacaaatttagaaccttttaataattttttgattctgaaaacgtgaggatttgctttaatctcgggtctaatttcagaaatattaccaaaccttttattatcatatggtatctcttctaattctaaatctgcaatatcaagaaatggcgtgtaaacaacactaaaaacattgtcaccacttgcaccaatgacagctgtattttcaccagccacggtagagtcaatataatagctatatacggcatatggtaacccgtacattttggattcccacgtatcataatccttattagttattgtcatactttgtcacctaccttatagtttggcatagggttaacagttttaacaccatttgcaacaactgttcccttttcgtttctaatctcaaaatgtaaatggtcaccacttgagtttccagtatttccacttagtgcaattaattgcccttgttttacggtgtcaccttcatttacttttaactctgaattatgagcgtaaacaacttgtgagtcatcatgctgtataattaaaaatttaccatagctagttgttaattctcttcttttaataacagttccgtctttacttgcatatactggtgtaccagttggggttgcaatatcaacaccattatgtggattcccactaggataagttgggtaatatgcacttatctgccctgttgttggaattaaccaatttgaccctgcatcattatcaacatattttccatcttttttaccactgggcaaccaaccgttaccatctgtataaccaactatttgaccattttgtccatattttaaaatttgtctttgaggataaacgccactttcaaataatgctccctctctctgccttctatttggatttgcttttaaattttttattgctgttgctatatttttcgtatcttttggattagctaaaaacatttgccacgcatttaatccacttaatccacccataccgtaattgaatgctatagatacccaaacatcaaattggtatataggaatatcacttaaatttatattagcctttagcatagcatttttaacgggtataccaaattcattatttaataaatcataggtaacttgactagctttttcctctgatactggaaaaggttcaagttgtctataatatttagtttgataattttctgttacaccatacccaccagttttaaaggattcaccattaaaatatgctccatattctgcaaatccttcttcttgttttacaaataaaagtccatttgctgatattattccatttgctatatcgccatttgccgaaccagttccgtcagaaccacccccaacgtttgtttcgactttccctaaaggtactgtagaagtaacaacaacaccacgacccatatcagctattttttcatattctagcatgatattttcaccgtaagaaatatcttcagtttcatattcatttgttggattatcgccattccatctattaacgtgacatctatcaacaaaactttctaaaatttcaaagtcaaattgataagtttgaaatacatcaacggatattactaataatgttgagttttctgttttataaacaaagtcgtctatgaaataataaatatttttcatattagtgtcataagctgaaatatagttaatattattctccatgaaccatccgtaaggcttgtctaccactaaacttacacgggaaggctctacctttatgttatattgacccacccatactactttagaagaaaaataattattttgattacttatacttttaaagttaaaaaggttctgatatgaggggtcaacataaggtaaagaataaaaatatacgtttttattcacttgttagaattgcaaaatttgaaaaagtatttactggtagactataattttcatagtaataataatttgttgttaaattattagggtcatcaattctatctaagccattgtaattcaacattataggcattaatcttttatccataattaaagctataggtttgattgtgtcatttgtcaccaaattagttaaatcagttggcaaattactaattgacttaatattatattcacctcttaaaagtatgctatctgtacttactaactttgcataaatctcgggtttcacaaatattattatgtcactttttaaacattgtgtatcaaatttactagtattgtattttgtacttgggagtgtgaactcttctacaattttaacaatactctcaattaagttagaatttgcaacttctttatatggtgcttgtttaaaattaagattttcaccgttggagtctaaacccttagcaaattcctttaaagctttttctatttctttgtatttatctttactcatagaagtttttaaattagatatcattttagaaattaaatcatatagcccaaactcttttaaaaatgctgactttaatctttcaacactaagactcattttaacttttttatcaaatctactatttgcaactaatccgtttagtcttggtaaagaagagttgaaaacttctttttcctcaccttctagtaaagattgaacagtcgagtcctctaagaacaactcttcaattgaatatccgaatggcatttcaccataaaaaagttcattgaaagggtttgaaaaatgctgctctatatataacatttttcctattttattgcatatagtttcgataaaactattcatatcaatatttattaaattcaaccttagtgcctcttcataagaaggaaaagagttgcccaaatccttgggcatggttgaaattaatttatcgtaaatttccttattagttaacttttccattttaaatcacctcattatgatgctgatttagtgtatattatagcatttagaaatggcactataccacataatccttgtttgtgtaaaaagtagttagtagataagttgtttgggttattaaaatatcttgtttcaaatacaaaatctttaaatcttagaaggtctttgtcaactagcataccaatacaagtttcgtcactatgttcattagaccctgtatcaccactcttaatagttgttggtagttcatccattagtataattctttgttgtacctctgcttttgaaaggttgaaaacatctgctaaaaattcaatatctatttcagcctcgatttcgggtgttgttataaatacacaatcatttaaagatgaaaattgcattactttagcactattatatttatcactttcaaacattaatctcttattcattgcttttaatgatttacaaagattttttacaagactagcatcatcatttaatttaataacttgagagtcttgaatatattgagtttttgtagctgatgcactcatgccaacatcttgattagttaatcccctacaatatttaaaaaacatatctttcatatatttatattcatcacgataagcacctcttaagttagcctctaataatttattaactaagtttgctaaaccatattcatttctaaacgctgttcttaactgaatatctgatattgttattttgtatttatcagcaaaatttcttgaaagataatttgtatataccttaggcactaaagtagctactatgtcttttacttcatcaccactattaaaatgttctttaaacccaactttttctgatgcctctacgtacatatcttctatagtataaccgtattcttccattccaccatgaaatatttttaaaggatttttccaaacttttgaatgtatttgagtcataaatattttatttataagggaacttataaactcattttttacaacttggtaattgaataaagcatccgaaacagctctactatcagataaatctacttgactcaaaacttttttagtttcttcggttgctccttctaaaaaagcctccatgatttctttgtttgccttaacagccattttatcactcccattcttttaatagcttgtccatttttgactcactatcttctttaatttcaatattattttcttcctttggtttgttagtaactttaagaaataattccatgttacttttttgaagtttttcaatttcttttgaattagtttccttttcagaatttaatgtgctgattacattattctgattttctatagtttcttctaattcaacaattcttaaaagggcatcatcaagagtcatttctttactcattttaaaacctccttacaaaatattatacccccttacttcactatacaacgcccacggtcataggtggaaaaatgttaggcacgttctaccgtgtgacttcctaatcatttacgttatatagctaagtaagggggtatactttacgttatgctaattgaaatattattctctttttagtttcagtcactttaaactcaatacctaaagaagttaaaagtgtattaaacattacagcatcatgttctttagattttggaaactctttttcttcttgatttataaaagaaactactctattaagttgtaatctcttttcattaactttttctaaagctaattcctttctgatttctaattctgttaactctctattttcttccattttattcacctctatattaattttagtaggtgggaattatcccacctttaaattatttatttctttcaaatattttttcatccccagtaattaagaagttttctaactctgtaacaccaacttcatagtatactgtttcgttctttttgattaactctaaaccttcaaatactttcttatctgatgcacctaaatactcaaattcatatgatgtattagtatcacccttagcccctaatctagttacctcataagtatggttgataaggtctggtcttttttcagctaagaaaattaatttttctattaaagtcttacctagttgccaaatttgtacactcttagtagtatggttataaacattaacagcaaccttttctgacatcttgaaaccttcaccacatagagggcaagtatctttaattactttcaagtcattgtctaattcagccacacactttacatttatttctctaccatctgctaattttactggatgaactaagtctaattctacttcatcaatgttgttgattaagaaagttataacagccttttctctgttatcctttaatctaaaatataccccacttgaaacgttgtttcctaaattgaaaattgataattttttcatgttaaatctctcctttaattttaaatatttttgttgttttggtttgtttttccttatgagttaattatacattacatttatatattatgtcaacacttttttgcattattttttaaaaaataatccaatttgattgaagtaaaattttgtttcagcattattaaagaaaacagccccaaccttattattgaaattgattctatttataagccctctattacataaagatatatgcttattttcatctgtttggtaatctaaatccaaaaagtattcgtcacattttcctttattgtgattatcaacccagtaataaaacattgtatcactttggcatttccatattttaattaccttgtccacatctacacgaaccttaaaaactggttcaagtttatcagtatcccataattcttttttactacaaacgttaaatctatcgttactgatattttcattatctatattatagcgtgatgatccactcaatacagttaatttaccaaccttagaatttaaggcgtactctttaaactctttgggtgtttcaagtgttttaattgccataagttcatttttgacaactctattaggtttagttgagggatataactttaaagtatcaaaaatggggttgtaataacttgtattattcccaacacatattacttctattctatcctcaaatctgtcaactgttacaattatagattctaatttcttatattcatcttttataattttctcaccaggttcgggcataacttcttcataaataatataataaacgttagggaatgatgaacccttagctaagctattaccatttaaccccgtaaataaacaaaatggttcaccatttataaatcccatatcaccctttaactcatattcaacagttggaaattccttttgtaatacatctgtaaagtatgtcgttgccatactttcagcttgatttttatatctccttaatatcaacgcttgtttaccatattctaaaaactcttctattattaatctttttgttgaatatgatttaccgacaccccttgcccctataacacaatgtattaaatggtttgggtattcttctataaactcttttatttcaaaaaatttactcatgttacctccttaaacttaaatcttttattatatattgaacttctactaaatcaagaccaccgtcacattttatttgtgctaatttacccttgacttctttacctagtttaaactcttcaaaaccttcattacataatatcttttgtgcatcttttggtacaccagcacatttacaagttattttaccgtcattagttctaagcatatatttttttgctcctatagttttaaactctttaaagtttttctcattatccatagcacctaaattgttagggtgaacttctacacccattttctttaaaccttcaattactttttctttaggtatagaacaataaaagctgtcagtatcgctgtataaaaaattttcaactccgcaaccttctattatcattgattgaaggtatactctagcgtatgctgtaacaaaactagcaaagggtttataaaaaggttcgctttcataagtttcaatatattgtccacttgctaaaatttggtttcttcttttttcattttttaactttttaaccctttctaaatttttttctttaccccctctatcgaatgtaaatattttatctttttttactaaatagtcaattgtacgtgttgttcttgtcccaaacttgccataaaaagagttgaggatagtttttatcgctgaatattgagcatctaatttttcacccttatctattctatgctttatttctgattttaaattataaaagtggtttacaaagtttgagaaaatacctttttcagctttatatactaagctagtaccaaactttaaaccattaaatttaatcttatctttttctttaaataaaaaaccatcaacctcaacattttcataataaccaaaatcataatattttgttaaatgttcaagttctacacttgtaatactcatattataattacataaattttcacaaccgttacgataaatttcaataactctgccatctattatattagtgtgtataaaagagttaccattcataagaaccatattcaatttatctctataaagtaaaggtaaatcagtatcagaaaagttctttgcccctaatctaatcatttgtaattcatactctttactcttaggtttaaaataatcaaagcctatattataaattgctacttcttcaccctcaactggtttaacatatcctttgtattcagttggcataccataaggcaataaagcactttcaacttgaaatggatagcttgaatttatatcaaagcttgacccattacattttacaactttattaactattttggggttataccatgtccacccaccttcataacttttgttctccatttccctagccattttgcttaaaagcatctgtctttcttcaatctcaaattgatttataaaagcttttttcttgtttctatcttcaaaatatgttatctctaaagctgatttaaatgatattgaagaacttgttttacaatttgaacttatgtaaatctcttcatcattatattcaaattttaaaagtgagttgtaaaaataatcatttataacttcttttaatatcctaatatcattataaagataccttctttcaatatctgttaaaatatgattatcttctcttattttatcataattaaactcttctttcaatttataaaacatttcatctaagttattgcaaaatttataagccttatctaaagaacatggaactatttttaaagtatcccaaaacttcacctttacactaaaagtattaacatgaccgtctttgtcagtaagttctaaagtgttaggtaaatttacttctaacccataataaacattatcagtctgtactatagaaaaagtcattggtttaaagttctcaatagttctactaattcctaattcgttagtctgtaagctgggattaacataagcgaatccattatcagttaaccagtattgcatatagctaatttcataagcaacgttgtgaacagcaacacttaaagttattactctcttatttttattcttctttatattttcttttttagcttgttcaaatcctatcatttgaaaggtataaaaagcctcgtctaaatcacaaccgaatattacgttattcgtatttgtagcacctaatccaaacgcccaaacttttctcttcttgtctttttgtggataatctttatttaacattttttcatcaacgtaacaagtttcaatgtcaaatattaaatctttactatctattttactatattcagttacttctaaactatgttcgttgacatatgttaaatgtttattcattaattccacttccttatatttttattgtttcaatcccgttttaaatgttgctaataagtcctctaatatataacccatttcaacatcatgcacaccatgagtgtaataacctttttcaaccatattttcttcataaataaactcaaaatatttatctatattctcataaaaatctgatacttttgatacagcatcatttgatgacataataaatttatttacctcacttacaaaactatctattttgtcttttggtaaattcctttgaaatgctccttttaatattcccttaccgctaccatcaccaaaaaataaatcggtaactttattaactaattcctttctgatgttttcttctgcatcttctttaccaacaaactttaataaccctaaatgtgtgtcacttataatcttctgtttactttcatgtgttaagtctaatttgtttaaattttcgactatctttttgcgttcttttgttttttctctctcttttcttttttcttctttttcttttcttttctgttcgttcttaacaaacctgtcagcgtttcttttataatctgtatctctcgccatatcaactattatgttaagtgtagatttgtcataacctaataatttaatagctttcttttggatatccttgtccattaaatagatataagccccattttttacagctttgttaaattgtttttcaaagttctttttctcaatttgctttttcttactttttaatgttattctttttcttgccatgttttctcaccttcctaacgtttttgcgtggtatgatatctataacatcactctctaaaaaatacttatcacctagaatatcaattacaaatatatcgtcacctatattataatttttagaatttaattcataataaaaaggttcaactactacagcgtgaaaagtttctaatcccatgttatgtttacctctcactatccaccacaccccttatcttgttcgctaggtcataaagtttttcaagttcatcaatactcttagcttgtaccattctagtatctaagcataaatcaatagtgcttaattcatctaatgaaagctttacaatatattctttcaaatttgacacctctcttttattttggtataattccccttaacataattctagcattttatacctataatttcaaggtttttattgatttagttgtaaacaatttgttaacagatagtgctgttttatctactgataataactggggaatatg

SEQ ID NO: 4 - DNA, 18147 bp, Unknown
  Clostridium phage vB_CpeP_ACP5, complete genome
accagtagagaaagcagcactatatattaacaaattgtttacaaatatatttcataaaaagcattgaatagcttaacataatttgttataattaaagtatcaattaagagaggtgatacagtgttaaaaataatatgttactggttactatttaatttattcgttctaggttatttatattatgtaaagaggtgattagaatgttcgtttacgttttactttattttactggttgctgtggtgatgaagttcattacataggtgataaagaaggattaagaaaatattgtaaagatgaaaatataaatatatcaaagttagaaccaaatttaactttaataaatggtgattataaagcttatacagtttcaaataatacggattatacaatattaattaaatattataagaatttaaaatatgctgatttatttaatcagtttaaaaagaggtgataatattgattaaaaaactaattcaattaatactagtcattatcatgggaatattagttgggttatcatttatgtttttatataacaatacatttttaacaagttcaattttcattatatcaagcttattatttgtaatgtttttaagttgtatattatattaaggagtgattaaaatgtgcaaaactatgaaagaagtttgtaaagaaagatatttaatgtggaaaaagaaaggttatacttttccaatatgtggcgagtgtgtttttgataagaacttagatggacattggggtgattgtgattttgtttttgaaaaggctaaccatatgttacctagagaacttaaagatgatacaatagatttattacttaactcaacaacgaatatagagagaataaattatttaaaagagattaataggagtgaataaaaatgaaattcaaaacattattaaatttaataaaaatagattctaaaagatgcattttattagaagactatgacagaaaaatcaaattagatatgatttatacattactaagaaggtcacgctacacacaaaaatttattgataactttaaaaattatgaatgtaaggataaagaaggtttagaaattgtattaaatgatttattaaatacaagaatacaagaacttaatacaagaatatcatatgagaaaatgttttttgaaaattatagtgacacttatgataatataaaccaactagaaaaacaaattgatttttacgaagatataaaagaaaggttaaaaggtaagaagaatgattaaactttataaagattatatagaaattacggatatgataataataaataaagatttaaaagaagttgattgtatagagttttcaagtgactatacatttactaacacatttattataaataatgattttattatattaaatggtaaaaattttatagatagatttaagataaaattaaaattaatattacaaataataaaattataaaagagggtaaataaaccctcttttttcttattcactccctttatgttcttgtaaccatgagccatagcaaccattattcataagagtatttgaaccaagagaagtaaatccattaaatggactagcatcatagtgtccattattgaatattatgttattcataaatacattattgtcccatttgcttgatgctgaaacgtctattgctttaggtttaatatgataattatcataactagcaaatgtattattagaaattatatttgattctaatacaccagttaatttaatatcatataacccaccctctacatttttgtcactaagtgttagatttttagttccgttatcctcaaaggcattattacttataatacaagctttagggtcaaccagtattaacccacaatctttttgcctccaaaatgtatttccagtaattacagaacttcttaatttttcagcatatataccactataagtatcaatattagcgtaactaccgtcaaaataacaatttgtgatttttaatatttcaacatcataatttgattcattcttgatgtatattccaccaccccatgaaccaccaacaaattgacaattatctatttgttgtgaacttgatgctatccatagagcatacttacgattcttaccccatatattagaatttgttaggtaacagtctgttattccaattttcacggaacaacttctaaagtctaacttatccatacataaagaatacgctccaccttcaccaacatattcaccattataatatacatacaaaccacctacgatatctatcggatattcatctcccatttcacaacgaatacgctcaactttgctcatatttaggttacataaatctaaatgccaatgattggtagctttattttttattaagtgtaaatcatgtaaataacagttgcttacaaattcattgtaaataccacttgtttttataattggagtatcattgttatttataattattgtgtttttatctccataaatctcaacatcatcttttaaaatgatgggttcgtcaagttcataagtttgtccatatatttttatagttttaaaaccattcgctattgcttgatttacttcacttccaataccttctttagatgtacataaataacttaatttatctatcatagattttaacatttctttatatgctgttacatcatcatttatattattttctaattctgaaaaatcacttttaacctcttctttaaatgatgatatttcatctttcataccattaattttattgtttaaatctctgaatacagtttcgtttattattgttgctaaagtaccatcaattaaccattgatttaattgtttagctacttcttcttttaatccttggccaactaaccattttgataaatctataacttgattttgtgagtcaatcaattcatttattttactaaagaattgacataaaatttcttgcaaggataaccctgtgtaatcataagctgtaaatatactacaatttaaatctataggttttaatttatttatcatttttaacactcctttaacttgatttaaatttaacactatctaaataaaatctattaatgttagttatatcacccataataataatattaccactttttgttatatttacaaatctaataacatcaccttctatattgtctttagcatatattggtattatcgtatctgataatggtctatattttaaaggtaatctataaattggtttacccgtttcacctccttttaacacacccattaatgaaacctcattattaccagttttgtaatatgaaaaatttgaatattcactatcaataacgttactgaaattttgaaaattatttaataaattttcatttgttattattattggttcttcaaaaaaacatatatccctaacatttccctttgagtccttataagaaaataaataattatcattatacgctgaaattaatgtattttcccttatatcatcacttaatattaagcctttgtaacatagttttccttctaaagcaattaattcattacaacatctgttatcattaaaccttatatcaccgtaacaaatagaaccagctatacctgtagtatttaatgaaatttgttctaggtttattaaaaaattactggtgttataaatgttcataacagcacctttaccctcttttattattaattgttccttaggattaccgccaccttcaaacctagggtttaaaacaatattacttcttccttgattcattaatatagcagtaccaccccttatttgttcaaatgcaatatttaaaaacttattattattaaaatttgttgttgtttgtgattcattttggttatttccaacacataaaccgttaatacaatctaaacttccatcactaattatattttcattaacccaaccagaattatctgattttaaaactattccatttttacattttcttaaatctttaaatgatatctcattattaaatgaacctcttaataaattgtcgttgtagtctttaccttctattatgattccatcaccaaaaccactaacagtatttattttaattttattgtatgaacaactagaaaatttaattgcaaaactttgtatattagttaaatctacataagaaggaattgaataattttgaattttattaattgatagttctgaattacttaatctaataaaatcaaaaccgttgcaatttttaaagcttaataaaccattaaatataattattttgttattccaatttttaatagtaatatttttatctattgttaaagattcttgtatttcaatttttaaacaatcatttattttattctctgtaatatcatttattaaattgtaaaatttttcttcatttgttccccccatataatcagataaactaatactttgtttagcatttattttttttatttcttcaacttgtaccatatcaccatctttacctttaatattaactggtaaaggattaggtaaatcaccattattactccaatataaaataccgtctgtagtaacatgtggtttataagtatatcctgtatcaccttttaatacttctttataattaatttttcctgttattttactcaattatatcacctcacatatgctgttaataatttctgttctaataacacctttttgtaatattgttacagaataattaaaagaacctttttcatcagttgggaatattaaatcagtatcattaaattctttttcttcttttatggaattaaatacaaattctattttatcaccttcttgtaatttatatccacacacatttaaatctaaaatacaagtatcaaattgattaactcttattttatttctgtcaattataatcattatttcaccacctaataaacatacataaataaacttctagcacctagtataatttgttcatctatgttatttaagcaactttgccatttttcttttaattctgatgatgaagtcacaccaatattaccctgtgaaattaattctgttttttctctttgtttgttgtttttatcaccagtattatttatattcacattatttttattatttgttttacttactgtattgctattactatcataagcatctgtagattgactcgaatttgttaaataaccatcttctaattttacactagctataccatcaccgattgcactctctttagtgttattagttgagttattattttcattattattaatatttgtagaagtgttgccagtttcattttgtgaagttaataaattatttatttcattctcttcgagatcccttatgaaagtttcttttaaatctttattcaacataaagtcaatatctttacatcttaattctatttcataaagttgctgataatatggcataactcttaaaagatatgctcttagttgttgcttaaaccttgctatagtttcaaatcctatttcatgaaagtaataatgatttaagaatttttcttcaaagtaacattttgtttcttcttcatagtaaggataatcaaagtcaaataaattattctccaatatcctcaactctagtgtgtacttcgacattttcatcacccccaaagctatccacaaccttagttacttttatgttagttccgaatttttcatttatcattttacaagctaataacctagttttatattccatgtctaaatttgtttcaatatattcattattagcgttaacttcatcaactaaaagtctttctcttttttctgtattagcattatctaatcctaaaaatgaaagtagttcactttctactgattttctttcgtcttctaatttatcaactaaatataaaacacctgtttgtaacagtttaaatccatcaaaaccctgttcggctaattctttgtctaagataacaacttcctccccattgtcaacatcattcacaatattttttactgacaataaattgttatttgtagctgtaacaaagaaaggtttcatttgttgctttaaatttctttttattactgtttctatatcgtccattttttgagcataatgggaaataaaatgcgatgtcggtaggcataaatcattcgctcttatttttataccatcttttccatcttctaagatattttgatatttgccatttcttgaaactatgcttaatttgatattatcaccatagatatttaaatcacccaaaccgtgacacggtaaacacattaaacccaatttatcatctttaaagaaaaaacattgcccattttcaaataaaaaatcctcaatatgtctactttctaatccagtaggcaagttatcccatttaaacctattacaagccataagtttatagtgtaaaaatagtcttgtttgattatcgcatattacattttcatttagtaaaatattaggattttgagatattatagattttcttttccccatctaatcaccacctttttaaaaggggaaatttattccccttgtttttgattattttttaagctgtcaatatctgattgtatttttgttaatgttatgttaagttctgaaattgcatttgatagtttactattttgattaaacattatcatacacgcaactatgggaaacccaacactatttattatatttattatttcttgcattaattacactcctccacaaaacaataccatcttttagcaatttcttgttgacagttttcagcatcttctttgctgttaaaatattgaacaacaaccctataaagttcattttctttgatagttgggtctatagcatttgcaattagtaaagccattttttcatatggtgtaggtgaccaaacttcgtttatgtcatgagggttatcacaaaacatagtttcaaatattatgttaggcgcttttacgttcttcatttcatataaattaacttcttttacacccctatttactaaacctaaactagcaaagtttttacataaccttgatgcaatttcttttatagagtttcttgcgccaactgtaacaagtgcctctgttccttgggcttgaccattaaagctattcatgtgtagtgaaataaatatatcaacatttgaattattagcttttcttacaccctcggaaagttcaccattttgtgtacttgcatcactaccacaataaactacttcatgaccatgtttttctaatatttctataacttctttacataaaactctcatacatgattgttcatcacgtaaacctatagcacccttacaattcggactatgaccgtctctaatacctattttcattttaaacctccctgttatccatagaataattaccaatttcgactccgtcattatcaatatgccatattgtagtaccattgtcaaaaatacctttgaggatatttaagtaattattaggaattttattagtttttatattaattcctattgtttttatatagttataataataccttgaattaatattaattttcatcattttattttgcttatatccaaactgggcaaaataatcgcctattttttcataatatctttctgttaatccatatctgtaaagtcttaactcttgaccaccattacgcaacccataaattatatttgacccttgactaagtaaagtattaggtgttgacctcatgtcgttcgctgttgctaaagctgaactgatagctgattccctagttaattgactctgcatattggtatgttttttatccatatatgacccaactccaccaaacacactatctattacacccccaacattaccagtaaatgcacttgcaacaccaccaacaacactatttgctatacctaaattcatattattttttgctattttatcatttaaaatagtttgatttacagtattttgaattgattgtgctgtctgatttttagaagttgcgacccaatttgcatatgctgaactagtgcaaggcaattcgagtgcatcatttgataccaaagcttcagtcattccgtctgtatcacccttatatgtttgaacaaatatcccataggagcatctgtctgaaactgataattttataccaactgaacaattaggaattgaacaaaattgaggctttaaaatcatagggtcatttataccatcacttaacattaaataagtgtatggataattatataatttactttcatttctccagtttcttttcccaccaacactcttttttactttgtaagtttcaaattcacctacaaatttagaaccttttaataattttttgattctgaaaacgtgaggatttgcttttatttcgggtctaatttcagaaatattaccaaaccttttattatcatatggtatctcttctaattctaaatctgcaatatcaagaaatggtgtataaacaacactaaaaacattgtcaccacttgcccctataatagccgtgttttcacctgccacggtagagtcaatataataactatatacggcatatggtaaaccatacatttttgattcccacgtatcataatctttattagttattgtcatactttatcaccccatttatgattcggcatagggttaacagttttaacaccatttgcaacaactgttcccttttcgtttctaatctcccaatgtaaatggtcaccacttgagttcccagtattcccacttagtgcaattaattgcccttgttttactgtgtcaccttcatttacttttaactctgaattatgagcgtaaactacttgagagtcaccatgttgtataattaaaaattttccatagctagtagtcaattctcttcttttgataacagtcccgtctttacttgcatagactggtgtaccagttggtgtagcaatatcaacgccattatgagggttgccacttggataagttgggtaatatgcacttatttgtcctgttgttgggattaaccagtttgaccctgcgtcattatcaacatattttccatcttttttaccactaggcaaccaaccgttaccatctgtataaccaactatttgaccattttgaccatactttaaaatttgcctttgaggataaacgccactttcaaataatgctccctctctttgtctacgctgtggattagcttttaaattttttattgctgttgctatatttttagtgtcttttgggttagctaaaaacatttgccacgcatttaaaccacttaaaccacccataccataattaaatgctatagatacccaaacatcaaattgataaatgggaatatcacttaaatttatattagcttttagcatagcatttttaacgggtataccaaattcattatttaaaaggtcataggtaacttgactagctttttcctcacttactggaaaaggctcaagttgtctataatatttagtttggtaattttctgttacaccatagccaccagttttaaaagattctccattaaaatatgctccatattctgcgaaaccctcttcttgttttacaaataaaagtccatttgctgatataataccatttgctatatcgccacttgctgaaccagttccgtcagaaccgccaccaacatttgtttcgactttccctaaaggtaccgtagaagtaacaacaacaccacgccccatatcagctattttttcatattctagcatgatattttcaccgtaagaaatatcttccgtttcgtattcattcgttgggttatctccgtcccatctattaacgtggcatctatcaacaaaactttctaaaatttcaaagtcaaattgataagtttggaatacatcaacggatattactaataatgttgagttttctgttttataaacaaaatcgtctatgaaataataaatatttttcatgttagtatcataagctgaaatatagttaatattattctccatgaaccacccgtaaggcttgtctaccactaaacttacacgggaaggctctacctttatgttatattgacccacccacaccactttagaagaaaaataattattttgattacttatacttttaaagttaaaaaggttttgatatgaggggtcaacataaggcaaagaataaaaatatacgtttttattcacttgttagaattgcaaaatttgaataagtatttactggtagactataattttcataataatagtaatttgttgttaaactattagggtcgtcaattctatctaaaccattataatttaacattataggcattaatcttttatccataattaacgctactggattaattgtatcattcgttgataaattagtaatatcagttggcaaattactaattgacttaatattatattcacctcttaaaagtacgctatctgtacttactaactttgcgtaaatttcaggtttcacaaatattattatgtcactttttaagcattgtgtatcaaacttactaggattatatttatcactcggcaaggtaaactcttccacaatttttacaatactctcaattaagtttgaacttgcaacctctttatatggggcttgtttgaaattaagattttctccgttagagtctaatcccttagcaaattcctttaaagctttttctatttctttgtatttatctttactcatagaagtttttaaattagataacattttacttattaagtcatataaaccaaactcttttaaaaatgctgactttaatctttcaacactaagagtcattttaacttttttgtcaaatctactatttgcaactaatccgtttagtcttggtaaagaagagttgaaaacttcattttcatcaccatctaataaagattgaacagttgagtcctccaaaaataattcttctattgaataaccgaatggcatttcaccataaaaaaggtcattgaaagggtttgaaaaatgttgctcaatatataacattttcccaattttattgcatatagtttcgataaaactattcatatcaatatttattaaattcaatcttaaagcctcttcataagaaggaaaagagttgcccaaatcctttggcatggttgaaattaatttatcgtaaatttccttattagttaacttttccatttatcacacttcctattctgttgatttagtgtatattatggcatttaaaaatggtactataccacataatccctgtttgtgtaagaagtagttagtagataagttgtttgggttattaaaatatcttgtttcaaacacaaaatccttaaatcttaaaaggtctttatcaactaacattccaatacaggtttctccagtatgaacattagtcccagtatcaccacttttaatagttgttggtagttcgtccattaatataattctttgttgtacctctgattttgaaaggttaaatacatctgctaaataatcaacatctatttcagctttcatttctggcgttgttataaatacacaatcatttaaagatgaaaattgcattactttagcgctattatatttatcactttcaaattgtagtctgtcattcatagcttttaatgatttacaaagattttttacaagactagcatcatcatttaatttaataacttgggagtcttgaatatattgtgtttgtgtaggtgtaccacttaatccaacatcttgattacttaatccactacaatatttgaaaaacatatttttcatatatttatattcatcacgataagcacctcttaaatttgcctctaataatttattaactaaattagataatccgtattcatttctgaaagctgtttttaattgaacatctgaaatagttattttgtatttgtcagcaaagtttcttgaaaggtaattagtgtaaactttaggaactaaagagccaactatatccttcacatcatcgccaccgttaaaatgttgattaaaacccactttttgtgatgcctctacatacatatcctctatagtataaccgtattcttccataccaccatgaaatattttaaggggatttttccacaccttagagtgtatttgagtcataaatattttatttattaaagtatttataaactcattttttacaacttgataattgaataaagcgtttgatacagctctactatcagctaaatctacttgacttaaaacctttttagtttcctctgttgcaccttctaaaaaagcctccataatttctttgtttgccttaacagccattttatcactcccattcttttaataacttgtccatttttgactcattattttctttaatttcagtattattttcttcctttggtttgctagtaactttaaggaataattccatgttacttttttgaagtttctcaatttcttttgaattagtttctttttcagaatttaatgtgctgattacattattctgattttctacagtttcttctaattcaacaattcttaaaagggcgtcatcaagagtcatttctttactcattttaaaacctccttacaaaatattatacccccttacttcactatacaacgcccacggtcataggtggaaaaatgttaggcacgttctaccgtgtgacttcctaatcatttacgttatatagctaagtaagggggtatattttacgttatgctaattgaaatattattctctttttagtttctgttactttaaattctatttctaaagaagttaaaagcgtatcaaacattacagcatcatgctctttagattttggaaactctttttcttcttgatttataaaagaaactactctattaagttgtaatctcttttcattaaccttttctaaggctaattcctttctgatttctaattctgttaactctctattttcttccatttttactcacctctatattaattttagtaggtgggaattaacccacccttaaattatttatttctttcaaatattttttcatctccagtaattaaaaagttctctaagtctttatttgatacttcataatatactgtttcatttttcttgattaactctaaaccttcaaatactttcttatctgacgcacctaaatactcaaattcatatgaagtattagtgtcacccttagcccctaatcttgttacctcataagtgtgattaataaggtcaggtcttttttcagctaagaaaattaatttttctattaaagtcttgcctagttgccaaatttgtacactcttagtagtatggttataaacattaacagcaaccttttctgacatcttgaaaccttcaccacatagaggacaagtatctttgattacttttaagtcattgtctaattcagccacacactttacatttatttctctaccatctgctaattttactgggtgaactaaatctaattcaacctcatcaatgttattgattaagaaagttataacagccttttctctgttatcctttaatctaaaatataccccacttgaaacgttgtttcctaaattaaaaattgataattttttcatgttaaatctctcctttaattttaaatatttttgttgttttggtttgtttttccttatgagttaattatacattacatttatatattatgtcaacacttttttacattattttttaaagaataatccaatttgattgaagtaaaattttgtttcaccattattaaagaaaacagccccaactttattattaaaattgattctatttataagtcctttattacataaagatatatgcttattttcgtctgtttgataatctaaatctaaaaagtattcatcgcatttacccttattatgattatctacccagtaataaaacattgtatcgctttgacatttccatattttaattactttgtcaacatcaacacgaaccttaaaaactggttctaacttatccgtatcccataattcttttttactacaaacgttaaacctatcattactaatattttcattatctatattatagcgtgatgaaccactcattaaggttaatttaccaaccttagaatttaaagcgtactctttaaactctttaggcgtttcaagtgttttaattgccataagttcatttttgacaactctgttcggttttgtactaggatatagctttaaagtatcaaaaatgggattgtaataacttgtattattcccaacacatattacttctattctatcttcaaatctatcaactgttacaattatagactctaatttcttatattcatcttttataattttctcgccagcctctggcataacttcttcataaataatataataaacgttgggaaaagatgaacctttagctaaactatttccgtttaaacctgtgaataaacaaaatggttcaccatttataaatcccatatccccctttaactcatactcaacacttggaaattctttttgtaatacgtccgtaaagtatgtcgttgccatgctttcagcttggttcttatatcttcttaatatcaacgcttgtttaccatattctaaaaactcttctattattaatctttttgttgaatatgatttaccgacaccccttgcccctataacacaatgtattaaatgatttggatattcttctataaactctcttatttcaaaaaacttacccatgttacctccttaaacttaaatcttttattatatattgtacttctactaaatcaagaccaccgtcacattttatttgtgctaatttacccttgacttctttacctagtttaaactcttcaaaaccttcattgcataatatcttttgtgcatcttttggtacacccgcacatttacaagatattttaccgtcattagttctaagcatatactttttagcccctatggttttaaactctttaaagtttttctcattatccatagcacctaaattgttagggtgaacttctacacccattttctttaaaccttcaattactttttctttaggtatagaacagtaaaagctgtcagtatcgctgtataaaaagttttcaactccacaaccttctattatcattgattgaagatatactctagcatacgctgtaacaaaactagcaaagggtttataaaagggttcactttcataagtttcaatatattgtccacttgctaaaatttggtttcttcttttttcattttttaactttttaaccctttctaaatttttctctttacctcctctatcgaatgtaaatattttatccttttttactaaatagtctatagtacgtgtagttcttgtcccaaacttgccataaaaagaattgagtatagtttttatcgctgaatattgagcgtctaatttttcacccttatctattctatgctttatctctgattttaaattataaaagtgatttacaaagtttgaaaaaatacctttttcagccttatatactaagctagtaccaaactttaaaccattaaatttaatattatctttttctttaaataaaaaaccatcaacctcaacattttcataatgaccaaagtcataatattttgttaaatgttcaagttctacacttgtaatactcatattataattacataaattttcacaaccattacgataaatttcaataactcttccatctattatattagtatgtataaaagagttaccattcataagagtcatatttaatttatctctataaagtattggtaaatcagtatcagaaaagttctttgctcctaatctaatcatctgtaattcatactctttactcttaggtttaaaataatcaaaacctatattataaattgctacttcttcaccatcaactggtttaacataacctttatattcagttgacataccatagggtaataatgcactttcaacttgaaatggatagcttgaatttatatcaaagcttgacccattgcattttacaactttattaactatcctagggttgtaccaagtccaccctccttcataacttttgttctccatttcccttgcaagtttacttaaaagcatctgtctttcttcaatttcgaactgatttataaaagcctttttcttattacgctcattaaaatatgttatctctaaagctgatttaaacgatattgaagaacttgttttacaatttgaacttatgtaaatctcttcatcattatattcaaattttaaaagtgagttgtaaaaataatcgtttataacttcttttaatattctaatatcattataaagataccttctttcaatatctgttaaaatatgattatcttctcttattttatcataatcaaactcttctttcaatttataaaacatttcatctaaattattacaaaacttataagctttatctaaagaacatggaactatttttaaagtatcccaaaactttacctttacactaaatgtattaacatgaccgtctttgtcagaaagttctaaagtgttaggtaagtttacttctaaaccataataaacattgtcagtttgtactatagaaaaagtcattggtttaaaattctcaatagttctactaatacctaattcgttagtctgtaaagaaggattaacataagtaaatccattatcagttaaccaatattgcatataactaatttcataagctacattatgaacagcaacacttaaagttattactctcttatttttattcttctttatattttcttttttagcctgttcaaaccctatcatctgtaaggtataaaaagcttcgtctaaatcacaaccgaatattacgttattcgtatttgtagcacctaatccaaacgcccatacttttctcttcttgtctttttgtggataatctttatttaacattttttcatcaacgtaacaagtttcaatgtcaaatattaaatctttactatctattttactatattcagttacttctaaactatgttcgttgacatatgttaaatgtttattcattaattccacttccttatatttttattgtttcaatcccgttttaaatgtcgctaataagtcttctaatatataccccatttcaacatcatgcaccccgtgagtgtaataacctttttcaaccatattttcctcataaataaactcaaagtatttatctatattttcgtaaaaatcagatacttttgatacagcatcatttgatgtcataataaatttatttacttcacttacaaaactatctattttgtcttttggtaaattcctttgaaatgctccttttaatattcccttaccgctaccatcaccaaaaaataaatcggtaactttattaactaattcctttctgatgttttcttctgcatcttctttaccaacaaactttaataaccctaaatgtgtgtcacttataatcttctgtttactttcatgtgttaagtctaatttgtttaaattttcgactatctttttgcgttcttttgttttttctctctcttttcttttttcttctttttcttttcttttctgttcgttcttaacaaacctgtcagcgtttcttttataatctgtatctctcgccatatcaactattatgttaagtgtagatttgtcataacctaataatttaatagctttcttttggatatccttgtccattaaatagatataagcaccattttttacagctttgttaaattgtttttcaaagttctttttctcaatttgctttttcttactttttaatgttattctttttcttgccatgttttctcaccttcctaacgtttttgcgtggtatgatatctataacatcactctctaaaaaatacttatcacctagaatatcaattacaaatatatcgtcacctatattataatttttagaatttaattcataataaaaaggttcaactactacagcgtgaaaagtttctaatcccatgttatgtttacctctcactatccaccacaccccttatcttgttcgctaggtcataaagtttttcaagttcatcaatactcttagcttgtaccattctagtatctaagcataaatcaatagtgcttaattcatctaatgaaagctttacaatatattctttcaaatttgacacctctcttttattttggtataattccccttaacataattctagcattttatacctataatttcaaggtttttattgatttagttgtaaacaatttgttaatatatagtgctgctttctctactgg

SEQ ID NO: 5 - DNA, 17990 bp, Unknown
  Clostridium phage vB_CpeP_ACP8, complete genome
acgattcgacatattccccagttattaccagtagataaagcagcactatctgttaacaaattgtttacaatcatatcttataaaaactattgaatagattaacataatttgttataattaaagtatcaattaggagggtgatattatgaaagttttaagtttatttgatgggataagttgcggtagagttgcattagaaagattaaatattaaagttgataaatatgtggcttatgagatagacagtaaagcaattgaagtgagtaaaaataattttaatgacattattcatatgggagatgtatttaatgctgattttaaaggtgaatattatgatattctattaggtggtagcccttgcactcattggtctatagcaagaggtgataacattggttaagaaactaattcaattaatactagtcattatcatgggaatattagttggaatatcatttatgtttttatacaacaatacatttttaacaagttcaacattcattatatcaatcttattatttgtaatgtttttaagctgtatattatattaagaggtgaataaaaatgaaattcaaaacattattaaatttaataaaaatagattctaaaagatgcattttattagaagactatgacagaaaaatcaaattagatatgatttatacattattaagaaggtcacgctacacacaaaaatttattgataactttataaattatgaatgtaaggataaagaagggttagaaattgtattaaatgatttattaaatacaagaatacaagaacttaatacaagaatagcatacgaaaaaatgtttttcgaaaactatagtgatacatacgataacataaatcaactagagaaacaaatcgatttctatgaagatataaaagaaagaataaaagagggtaaataaaccctctttttcttattcacttcctttatgttcttgtaaccatgaaccataacaaccattattcataagagtatttgaacctagagaagtaaatccattgaatggactagcatcatagtgaccgttattaaatattatgttattcataaatacattattgtcccatttgcttgatgctgaaacatctattgccttaggtttgatatgataattatcataactagcgaatgtattattagaaattatatttgattctagtacaccagttaacttaatatcatataaaccaccttctacatttttatcactaagagttagatttttagttccattatcctcaaaggcattattacttataatacacgctttagggtcaaccagtattaatccgcaatctttttgcctccaaaatgtatttccagtaattacagaacttcttaatttttcagcatatataccactataagtatcaatattagcataacttccatcaaaataacaatttgtgatttttaatatttcaacatcataatttgattcatttttaatgtatattccaccaccccatgaaccaccaacaaattgacaattatctatttgttgggaacttgaggctatccataaagcatatttacgattcttaccccaaatattagaatttgttaggtaacaatcagttataccaatttttactgagcaacttctaaaatctaacttatccatacataaggaatacgctccaccttcaccaacatattcaccattataatacacatacaaaccacctacaatatctattggatattcagctcccatttcacaacgaatacgctcaactttactcatatttaagttacataaatctaaatgccaatgattagtggctttattttttattaagtgtaaatcatgtaaataacagttgcttacaaattcattgtaaataccacttgtttttataattggcgtatcattgttatttataattattgtgtttttatcgccatatatttcaacatcatcctttaatattataggctcgtcaagttcatatgtttgaccatatatttttatagttttaaaacctttagctattgcttgatttacttctctacctatgccctctttagatgtgcataaataacttaatttatctatcatagattttaacatttccttatatgctgttacatcatcatttatattattttctaattctgtaaaatcacttttaacttcttctttaaatgatgatatttcatctttcataccattaattttattatttaaatctctgaatatagtttcatttattattgttgctaaagtaccatcaattaaccattgattaagttgtttagcaacttcttcctttaacccttgtcctactaaccattttgttaaatctattacttgattttgtgagtcaattaattcatttattttactaaagaactgacataaaatttcttgcatggataaccctgtgtaatcataaactgtaaagatgctacagttaaggtcaataggatttaatttatttatgtttatcattattatacctccttatatggtaatgtctgtatcattaaagtcttaaagttttcaaattcatttgttaaattattaaacttttcttttaaatcttctttttcaaatttcaaatcatttattaacttattagtatttactggcactttaaatgaacttgtaccgcttacgctgttttcaaagctaacataagttctttcgttaaaagattttacgttaatgtttttagttaaagggatttcaatttgttctgcaagttcataataaactattactgggtttccactgtcgtgtaaatgtttaaaatattttttaattccatcggtgttaggtgtttctaacttacttataggtagctgaaacgataatatatttattccgctcaaatcaaaaccctctttattttgatttttagggtcatctttattccacaaaccatctacatagataaattgattacatatagcttcatttccagaacttgacttacttccttcaagtttaaaactacaatataaataatcaccccttaaatccacggttagctgttcatttcctgtcattgtgtacttttcaattctctttacagccacgttttttatactatttaactcatcatatacggttgggtttaaacctctaagaccttcatcaaaccctaactcttgcaataaaatatcttttttatcacatttgaaaggactataagaagttgtttcagttccatactcaagttgagtccatttaggtttgtagctatttattaaattatctgcatatatttttagtgttgcttttctttctaataagtttctaatttcatcttttgtgaaacttaatgcactagacttaacaatattaccacttttcgttacaacccctttttgatattgaccccctaaagtcggtgtgtaataaacagtattcctaagtaaatctcccatcccttcagcatcaaatgaaatattgcaattttcattttcctttataatagagattaattcaaatactatgttactattattagaacttttatattttaatcctaaattctttcctgtacttgaaatattaattttattttcatcttgcccaaaggattttatttcaccattgtcaagttttatagtttttccttttataatcatatcactactcataccatcaatagtattagaaatatcatggttttctttgtcctcctgttctaacattgtaacttcaattttctctcttaatctcataacttcatggtctattctatcatctaaagtcataaaattttcgcctgtagtagctgtacgtgcttgtactacttcatcttttgtatcattattttttatggattcacttaattttttaaagtcactatctaacctttcttttaatttggaaaatgttttaccagttgtatcaactctactttcttttacttcatttaatattaagtcagaaatattaaaaggctgtggattttctaaattaccattattactccatgttaaaataccatcttctgaaatatgaggggtataaaaataacctttatcacctttaatatttacagttggggggttttcaagttctcctgtatttgtccaagataaattaccttcatcatcaacgtgaggaataaacataaaaccttgtttaccatgtaaatttactggtaatggattctctaaaccaccgttatttgtccaatacaaaataccgtcaactgtaacatggggttcgaacgtataaccagtatcaccttttaatacttctttataattaacttttcctgttattttacacaattatatcacctcacatatactgttaataatttctgttctaataacacctttttgtaatattgttacagaataattaaaagaacctttttcatcagttgggaatattaaatcagtgtcattaaattctttttcttcttttatagaattaaacacaaattctattttatcaccttcttgtaatttatatccacaaacatttaaatctaaaatacaagtatcaaattgattaactcttattttatttctgtcaattataatcattattacaccacctaataaacatacataaataaacttctagcacctagtataatttgttcatctatgttatttaagcaactttgccatttctcttttaattctgctgatgaagttacccctatattaccttgtgaaattaattctgttttttctttttgtttgttgtttttatcaccagtattatttatatttaaattacttttattatctgttttacttactgtattactattactatcataaatatctattgattgactcgaatttgttaaataaccatcttctaattttacactagctataccatcaccgattgcactctctttagtgttattagttgagttattactttcattattattaatatttgtagaagtgttgccagtttcattttgtgaagttaataaattatttatttcattctcttcgagatcccttatgaaagtttcttttaaatctttattcaacataaagtcaatatctttacatcttaattctatttcataaagttgctgataatatggcataactcttaaaagatatgctcttagttgttgcttaaaccttgctatagtttcaaatcctatttcatgaaagtaataatgatttaagaatttttcttcaaaataacacttagcctcttcttcatagtaaggatagtcaaagtcaaataaattattctctaatgtccttaactctagtgtgtacttcgccatcttcatcacctccaaaactatcaacaactttagaaacttttatattagtcccaaatttttcatttatcatttgacaagctaataacctagttttatattccatatctaaatttgtttcaatatattcgttattagcgttaacttcatcaactaaaagtctttctcttttttctgtattagcattatctaatcctaaaaatgaaagtagttcactttctactgattttctttcatcttctaatttatcaactaagtaaataaccccagtttgtaacatcttaaaaccgtcaaatccctgttcacctaagtctttatctaatataacaacctctttcccattgtcaacatcatttacaatgttttttactgataataaattattatctgttgctgtaacaaagaaaggtttcatttgttgctttaaatttctttttattactgtttctatatcatccattttttgggcgtaatgagaaataaaatgagatgttggtaaacataagtcattcgctcttatttttatgccatctttcccatcttctaagatgttttgatatttaccatttcttgaaactatacttaatttgatattatcaccatagatatttaaatcacctaaaccatgacacggtaaacacattaaacctaacttatcatctttgaagaaaaaacattgaccattttcaaataaaaagtcctcaatatgtctactttctaatccagtaggcaagttatcccatttaaacctattacaagccataagcttataatgtaaaaataatcttgtttggttatcacatattgtattttcattaagtaaaatattaggattttgagatattatagattttctttttcccatctaatcaccaccttttttaaaaggggaaatttattccccttgtttttgattatgctttaagctgtctatatctgattgtatctttgttaatgttatgttaagctctgaaattgcatttgatagtttactattttgattaaacattatcatacacgcaactatgggaaacccaacgctatttattatatttattatttcctgcattaattacacccctccacaaaacaataccatcttttagcgatttcttgctgacaattttcggcatcttccttactattaaaatattgaacaacaactctataaagttcattttctttaatagttgggtcaatagcatttgcaattagtaaagccattttttcgtatggtgtaggtgaccaaatttcatttatgtcatgttcattatcacaaaacatggtttcaaatattatgttagggcaattagtgtctgtcatttcccagtaatttttttcttttactcctctatttgctaatcctaaagtagcaaaatttttacataattttgatgcaatttcctttatagaatttcttgcacctctagcaacccatgcctctgtaccttgtccatttccattactagcattcatgtgtaatgatataaaaatatcacaaccgtatccttttattgccccttgtcttacatcttctctttcatcttttaaagttgtttcacatcttacaactgtatgaccttttttaactaaaattctttcaacttcatcagctaaaaccttcatacacgcctgttcatctcttaaacgaattgctcctttacagttaggactatgtccaccccttaaacctattttcattatttatacctccctgttatccatagaataattaccaatttcgactccgtcattatcaatatgccatattgtagtaccattgtcaaaaatacctttaaggatatttaagtaattattaggaattttattagtttttatattaattcctattgtttttatatagttataataataccttgaattaatattaattttcatcattttattttgcttatatccaaactgtgcaaaataatcacctattttttcataatatctttctgttaaaccataacggtaaagtcttaactcttgccccccattacgcaacccataaattatatttgacccttgactaagtaaagtattaggtgttgacctcatgtcgttcgctgtagctaaagctgagcttatagctgattccctagttaattgactctgcatatttgtatgttttttatccatatatgacccaactccgccaaatacactatctatcactccaccaacgttaccagtaaaagcactagcaacaccaccaacaacactatttgcgatacctaaattcatgttatttttagctattttatcatttaaaatcgtttggtttacggtattttgaattgattgtgctgtctgattttttgaagttgcaacccaatttgcatatgctgaacttgtgcaaggcaattcaagtgcatcatttgacaccaaagcttctgtcattccgtctgtatcacccttataagtttgtacaaatataccataggaacatctgtctgaaacagataattttataccaactgaacaactaggaattgaacaaaattggggctttaaaatcatagggtcatttataccatcacttagcattaaataagtgtacggataattatataatttactttcatttctccaatttcttttacccccaacactcttttttactttgtaagtttcaaactcaccgacaaattttgaaccttttaataattttttgattctgaaaacgtgggggtttgcttttatttcgggtctaatttcagaaatattaccaaatcttttattgtcatatggtatctcttctaattctaaatcagcaatatcaagaaatggtgtgtaaacaacactaaaaacattgtcaccacttgcccctataacagctgtgttttcaccagccacggtagagtcaatatagtaactatatacagcatatggcaaaccatacatttttgattcccatgtatcataatctttattagttattgtcatactttgtcacctaccttataatttggcataggattaatagttttaacaccatttgcaacaactgttcccttttcatttctaatctcccaatgcaagtggtcaccactactattaccagtatttccacttaatgcaattaattgaccttgctttactgtgtcaccctcatttacttttaactctgaattatgagcataaacaacttgtgagtcaccatgttgtataattaaaaatttcccatagctagtagttaattctcttcttttaataacagttccgtctttacttgcatatactggtgtaccagttggggttgcaatatcaacaccattgtgtgggtttccacttggataagttgggtaatatgcacttatctgtcctgttgttgggattaaccagtttgaccctgcatcattatcaacatattttccatcttttttaccactaggcaaccaaccgttaccatctgtataaccaactatttgaccattttgaccatactttaaaatttgtctttggggataaacaccactttcaaataatgcaccttccctttgtctacgttgtggattagcttttaaattttttatcgctgctgcaatattttttgtatcttttgggttagctaaaaacatttgccacgcatttaatccgcttaatccacccataccataattaaatgctatagatacccaaacatcaaattgatatataggaatatcacttaaatttatattagcttttaacatagcatttttaactggtattccaaattcattatttaacaaatcataggtaacttgactagctttttcttctgatactggaaatggttcaagttgtctatagtatttagtttggtaattttctgttacaccataaccgccagttttaaaagattcaccattaaaatatgccccatactctgcaaaaccttcttcctgttttacaaataaaagtccatttgctgatattattccatttgctacatcaccatttgctgaaccagttccatcagaaccacctccaacgtttgtttcaactttaccaagtggtactgtagaagtgacaacgacaccacgccccatatcagctattttctcatattctagcataatattttcaccatatgagatatcttctgtttcatattcatttgatgggttatcaccattccatctattaacgtgacatctatcaacaaaactttctaaaatttcaaagtcaaattgataagtttggaatacatcaacagatattactaataatgttgaattttctgttttataaacaaagtcgtctataaaataataaatatttttcatgttagtatcataagctgaaatataattaatattattctccatgaaccaaccataaggtttgtctaccactaaacttacacgagaaggctctacctttatattatattgacccacccatactactttagaagaaaaataattattttgattacttatacttttaaagttaaaaaggttctgatatgaggggtcgacataaggtaaagaataaaaatatacgtttttattcacttgttagaattgcaaaattagaataagtatttactggtagactataattttcatagtagtaataatttgttgttaaactattagggtcgtcaattctatctaatccattgtaattcaacattataggcattaatcttttatccataattaaagctataggcttgatcgtgtcatttgataccaaattagtaacatctgttggtaaatcactaattgacctaatattatattcacctcttaaaagtatactatctgtacttactaactttgcataaatttcgggtttcacaaatattattatgtcactttttaaacattgtgtattaaatttgctagggttatatttatcacttggcaaagtgaattcttctacaatttttacaatactctcaattaagtttgaacttgcaacttgtttatatggtacttgtttaaaattaagattttctccatttgagtctaaacccttagcaaattcttttaaagcttttactatttctttgtatttatctttactcatagaagtttttaaattagataacattttagatattaaatcatacaacccaaactcttttaaaaatgctgactttaatctttcaacactaagactcattttaacttttttgtcaaatctactatttgcaactaatccgtttagtcttggtaaagaagagttgaaaacttctttttcatcaccttctagtaaagattgaacagttgagtcctctaaaaataattcttctattgaataaccgaatggcatttcaccataaaaaagctcattgaaagggtttgaaaaatgttgctctatatataacatttttcctattttattacatattgtttcaataaaactattcatatcaatatttattaaattcaaccttagtgcctcttcataagaaggaaaagagttgcccaaatccttgggcatggttgaaattaatttatcataaatttctttattagttaacttttccattttaaatcacctcattatgatgctgatttagtgtatattatagcatttaaaaatggtactataccacataatccctgtttgtgtaagaagtagttagtagataagttgtttgggttattaaaatatcttgtttcaaatacaaaatccttaaatcttagaaggtctttgtcaactagcataccaatacaagattcaccctcatgtgtattacttccactatcaccactttttatttgcgttggtagttcatccattagtataattctttgttgaacctctgacttggaaaggttgaaaacatctgctaaataatcaacatctatttcagccttcatttcgggtgttgttataaaaacacaatcatttaaagatgaaaattgcattactttagcactattatatttatcactttcaaattgtagtctgtcattcatagcttttaatgatttacaaagattttttacaagactagcatcatcttttaatttaataacttgagagtcttgaatatattgcgtttgtgtaggtgaaccacttaatccaacatcttgattgcttaatccactacaatatttgaaaaacatatttttcatatatttatattcgtcacgataagcccctcttaaatttgcctctaataatttattaactaaattagctaatccgtattcatttctgaaagctgtttttaattgtacatctgatattgttattttatatttgtcggcaaagtttcttgaaagataattagtgtaaactttaggaactaaagacccaactatatcctttacatcatcaccaccgttgaaatgttctttaaagccaactttttgggatgcctctacatacatatcttctatagtataaccgtattcttccataccaccatggaatatttttaatggatttttccatactttagaatgaatttgagtcatgaatattttatttattaaagtgtttataaactcatttttcacaacttgataattaaataaagcgtttgatacagctctactgtcagataaatctacttgacttaaaacctttttagtttcctctgttgcaccctctaaaaaagcctccataatttctttgtttgccttaacagtcattttatcactcccattcttttaatagcttgtccatttttgactcactatcttctttaatttcattattattttcttttggtttgctagtaactttaaggaataattccatgttacttctttgtaatttctcaatttcttttgaattagtttccttttcagaatttaatgtgctgattacattattctgattttctatagtttcctctaattcaacaattcttaaaagggcatcatcaagagtcatttttaaaacctccttacaaaatattatacccccttacttcactatacaacgcccacggtcataggtggaaaaatattaggcacgttctaccgtgtgacttcctaatcatttacgttatatagctaagtaagggggtatactttacattatgctaattgaaatattattctctttttagtttctgttactttaaactctattcctaaagaagttaaaagcgtatcaaatattacagcctcatgttctttagattttggaaactctttttcttcttgatttataaaagaaactactctattaagttgtaatctcttttcattaattttttctaaagctaactcttttcttatttctaagttatctccaactgtttgctttatttcattgttatcattttgatgtactgtgttttccatttttattcacctctatattaattttagtaggtggggattatcccacctttaaattatttatttctttcaaatattttttcatccccagtaattaagaagttttctaagtctttgtctgatacttcatagtatactgtttcatttttcttgattaactctaaaccttcaaatactttcttatctgatgcacctaaatactcaaattcatatgatgtattagtatcacccttagcacctaatcttgttacctcataagtgtggttaataaggtcgggtcttttttcagctaagaaaattaatttttctattaaagtcttacctagttgccaaatttgtacgctcttagtagtatgattataaacattaacagcaaccttttctgacatcttgaaaccttctccacataatggacaagtatctttaattacttttaagtcattgtctaattcagccacacactttacgtttacttctctaccatctgataattttactggatgaactaagtctaattctacttcatcaatgttgttgattaagaaagttataacagccttttctctgttatcctttaatctaaaatataccccacttgaaacgttgtttcctaaattgaaaattgataattttttcatgttaaatctctcctttaattttaaatatttttgttgttttggtttgtttttccttatgagttaattatacattacatttatatattatgtcaacacttttttgcattattttttaaaaaataatccaatttgattgaagtaaaattttgtttcagcattattaaagaaaacagcaccgaccttattattgaaattgattctatttataagtcctttattacataaagatatatgtttattttcatctgtttggtaatctaaatctaaaaaatattcgtcacattttcctttattatggttatctacccagtaataaaacattgtatcactttgacatttccatattttaattaccttgtccacatccacacgaaccttaaaaactggttctagtttatcagtatcccataattcttttttactacaaacattaaatctatcattactaatattttcattgtctatattataacgtgatgaaccactcattacagttaatttaccgacctttgaacttaaagcatattccttaaactcttttggtgtttcaagtgttttaattgccataagctcatttttgacaactcgatttggtttggttgaagggtataactttaaagtatcaaaaatagggttgtagtagcttgtattattaccaacacatattacttctattctatcctcaaatctatcaactgttacaattatggattctaatttcttatattcatcttttataattttctccccaggctcgggcataacttcttcataaataatataataaacgttaggaaatgatgaacccttagctaaactatttccgtttaaccctgtaaataggcaaaaaggctctccatttataaatcccatatcaccttttaactcatattccacagttggaaattccttttgtaatacatctgtaaagtatgtcgttgccatactttcagcttgatttttatatctccttaatatcaatgcttgtttgccatattctaaaaactcctctattattaatctttttgttgaatatgattttccgacaccccttgcacctataacacaatgtattaaatggtttgggtattcttctataaactcttttatttcaaaaaacttactcatattatctcctttacactaattatatcaccattaatatacatactagcaaattgttctgcctcttttttatttttagcctttattttcatatgatattctcgttcataaattctgtaggttattataaaacctttcataatattacctccttaaacttaaatcttttattatatattgaacttctactaagtcaagtccaccatcacattttatttgtgctagtttacccttgacttctttacctagtttaaactcttcaaaaccttcctcacataatatcttttgtgcatcttttggcacacccgcacatttacaagatattttaccgtcattagttctaagcatatactttttagcccctatggttttaaactctttaaagtttttctcattatccatagcccctaaattgtttggatgcacttctatacccattttctttaaaccttcaattactttttctttaggaatagagcaataaaagctgtcagtatcgctgtataaaaaattttcaaccccacaaccttctattatcattgattgaaggtatactctagcatatgctgtaacaaaactggcaaagggtttataaaaaggttcactttcataagtttcaatatattgtccacttgctaaaatttgatttcttcttttttcattttttaactttttaaccctttctaaatttttctctttaccacctctatcgaatgtaaatattttatccttttttactaaatagtctatagtacgagtagttcttgtcccaaacttgccataaaaagaattgagaatagtttttatcgctgaatattgagcgtctaatttctcacccttatctattctatgctttatctctgattttaaattataaaagtgatttacaaagtttgaaaaaatacctttttcagccttatatactaagctagtgccaaactttaaaccattaaatttaatcttatctttttctttaaataaaaaaccatcaacctcaacattttcataatatccaaattcatagtattttgttaaatgttcaagttctacacttgtaatactcatattataattgcataaattttcacaaccattacgataaatttcgataactctgtcatctattatattagtgtgaataaaagagttaccattcataagaaccatatttaatttatctctataaagtaaaggtaaatcagtatcagaaaagttctttgctcctaatctaatcatttgtaattcatactctttactcttaggtttaaaataatcaaaacctatattataaattgctacttcttcaccctcaactggtttaacatatcctttgtattcagttggcataccataaggtaataaagcactttcaacttgaaatggatagcttgaatttatatcaaagcttgaaccattacattttacaactttattaactattttagggttgtaccatgtccacccgccttcataacttttgttctccatttctctagccattttgcttaaaaacatctgtctttcttcaatctcaaattgatttataaaagcttttttcttgtttctatcttcaaaatatgttatctctaaagctgatttaaatgatattgaagaacttgttttgcaatttgaacttatgtaaatctcttcatcattatattcaaattttaaaagtgagttgtaaaaataatcatttataacttcttttagtattcttatatcattatatagataccttctttcaatatctgttaaaatatgattatcttctcttatcttatcataattaaactcttctttcaatttataaaacatttcatctaagttattgcaaaatttataagctttatctagagaacatgggactatttttaaagtgtcccaaaacttcacttttacgctaaaagtattaacatgaccgtctttgtcagtaagttctaaagtgttgggtaaatttacttctaaaccataataaacattatcagtttgtactatagaaaaagtcatgggtttaaagttctcaatggttctgctaattcctaattcattagtctgtaaactaggattaacataagtgaatccattatcagttaaccaatattgcatatagctaatttcataagcaacgttatgaaccgcaacacttaaagttattactctcttatttttattcttctttatattttcttttttagcctgttcaaaccctatcatctgtaaggtataaaaagcctcgtctaaatcacacccaaatattacatcatttgtatttgtagcacctaaaccaaacgcccaaacttttctcttcttgtatttttgtggataatctttatttaacattttttcgtcaacgtagcaagtttcaatgtcaaatattaaatctttactatctattttgctatattcagttacttgtaaactatgttcgttgacataagttaaatgtttattcattaattccacatccttatattttattgtttcaatcccgttttaaatgttgctaataagtcctctaatatatatcccatttcaacatcatgcactccatgagtgtaataacctttttcaaccatattttcctcataaataaactcaaagtatttatctatattttcataaaaatcagatacttttgatacagcatcatttgatgacatgataaatttattaacttcatttacaaagttgtctattttgtcctttggtaaattcctttgaaatgccccttttaatatacccttaccactaccatcaccaaaaaataaatctgtaactttattaactaattcctttctgatattttcctcggcatcttctttgcctacaaattttaataatcctaagtgtgtgtcacttataatcttttgtttactttcatgtgttaagtctaatttgtttaaattttcaactatctttttgcgttcttttgttttctccctctcttttcttttttcttctttttcttttctcttctgttcattcttaacaaacctgtcagcatttcttttatagtctgtatctcttgccatatcaactattatgttaagtgtagacttatcgtaacctaataatttaatagccttcttttgaatatccttgtccattaaatagatataagctccattttttacagctttgttaaattgtttttcaaagttctttttctcaatttgctttttcttactttttaatgttaatctttttcttgccatgttttctcaccttcctaacgtttttgcgtggtatgatgtctataacatcactctctaaaaaatacttatcacctagaatatcaattacaaatatgtcatcaccaatattataatttttagaatttaattcataataaaaaggttcaactactacagcgtgaaaagtttctaatcccatgttatgtttacgcctcactatctaccacaccccttatcttgttagctaagtcataaagtttctcaagttcatcaatgctcttagcttgtaccattctagtatctaagcataaatcaatagtacttaattcatctaatgaaagctttacaatatattctttcaaatttgacacctctcttttattttggtataatttccccttaacataattctagcattttatacctataatttcaaggtttttattgatttagttgtaaacaatttgttaacggatagtgctgctttctttaatggtaataactggggaatatgtcgaatcgt

SEQ ID NO: 6 - DNA, 17647 bp, Unknown
  Clostridium phage vB_CpeP_VLR28, complete genome
accagtagataaaacagcactatctgttaacaaattgtttacaactaaaacaataaaaaccttgaaattataggtataaaatgctagaattatgttaaggggaaattataccaaaataaaagagaggtgtcaaatttgaaagaatatattgtaaagctttcactagatgaattatgtactattgatttatgcttagatactagaatggtacaagctaagagtattgatgaacttgagcaactttacgacttagcaaataagataaggggagtggtggatagtgaggaataaacataacatgggattagaaacttttcacgctgtagtagttgaacctttttattatgaattaaattctaaaaattataatattggtgatgatatatttgtaattgatattctaggtgataagtattttttagagagtgatgttatagatatcataccacgcaaaaacgttaggaaggtgagaaaacatggcaagaaaaagaataacattaaaaagtaagaaaaagcaaattgagaaaaagaactttgaaaaacaatttaacaaagctgtaaaaaatggtgcttatatctatttaatggacaaggatatccaaaagaaagctattaaattattaggttatgacaaatctacacttaacataatagttgatatggcgagagatacagattataaaagaaacgctgacaggtttgttaagaacgaacagaaaagaaaagaaaaagaagaaaaaagaaaagagagagaaaaaacaaaagaacgcaaaaagatagtcgaaaatttaaacaaattagacttaacacatgaaagtaaacagaagattataagtgacacacatttagggttattaaagtttgttggtaaagaagatgcagaagaaaacatcagaaaggaattagttaataaagttaccgatttattttttggtgatggtagcggtaagggaatattaaaaggagcatttcaaaggaatttaccaaaagacaaaatagatagttttgtaaatgaagtaaataaatttattatgtcatcaaatgatgctgtatcaaaagtatctgatttttacgaaaatatagataaatactttgagtttatttatgaggaaaatatggttgaaaaaggttattacactcatggggtgcatgatgttgaaatgggttatatattagaggacttattagcaacatttaaaacgggattgaaacaataaaaatataaggaagtggaattaatgaataaacatttaacatatgtcaacgaacatagtttagaagtaactgaatatagtaaaatagatagtaaagatttaatatttgacattgaaacttgttacgttgatgaaaaaatgttaaataaagattatccacaaaaagacaagaagagaaaagtttgggcgtttggattaggtgctacaaatacgaataacgtaatattcggttgtgatttagatgaggctttttataccttacagatgatagggtttgaacaagctaaaaaagaaaatataaagaagaataaaaataagagagtaataactttaagtgttgctgtccataatgtagcttatgaaattagctatatgcaatattggttaactgataatggtttcacttatgttaaccccagtttacagactaacgaattaggtattagtagaactattgagaactttaaaccaatgaccttttctatagtacaaactgataatgtttattatggtttagaggtaaatttacctaacactttagaacttactgataaagatggtcatgttaacacatttagtgtaaaggtgaagttttgggacactttaaaaatagttccttgttctttagataaagcttataaattttgcaataacttagacgaaatgttttataaattgaaagaagagtttgattatgataaaataagagaagacaatcatattttaacagatattgaaagaaggtatctctataatgatattagaatactaaaagaagttataaatgattatttttacaactcacttttaaaatttgaatataatgatgaagagatttacataagttcaaattgtaaaacaagttcatcaatttcttttaaatcagctttagagataacatattttgaggatagaaacaagaaaaaagcttttataaatcaatttgagattgaagaaagacagatgcttttaagcaaaatggctagggaaatggagaataaaagttatgaaggtgggtggacttggtacaaccctaaattagttaataaagttgtaaaatgtaatggttcaagctttgatataaattcaagctatccatttcaagttgaaagagcattattaccttatggtatgccaactgaacataaaggttatgttaaaccagttgagggtgaagaagtagcaatttataatataggttttgattattttaaacctaaatgtaaagattatgaattacaaatgattagattgggagcaaagaacttttctgatactgatttacctttactttatagagataaattaaatatggttcttatgaatggtaattcttttattcatactaatataatagatgacaaagttattgaaatttatcgtaacggttgtgaaaatttatgtaattataatatgagtattacaagtgtagaacttgaacatttaacaaaatattatgattttggttattatgaaaatgttgaggttgatggttttttatttaaagaaaaagatgagattaaatttaatggtttaaagtttggtactagtttagtatataaagctgaaaaaggtattttctcaaactttgtaaatcacttttataatttaaaatcagaaataaagcatagaatagataagggcgaaaagttagacgctcaatattcagcaataaaaactatactcaactctttttatggcaagtttggaacaagaactactcgtactatagactatttagtaaaaaaggataaaatatttacattcgatagaggtggtaaagagaaaaatttagaaagagttaaaaagttaaaaaatgaaaaaagaagaaaccaaattttagcaagtggacaatatattgaaacttatgaaagtgaacctttttataagccatttgccagttttgttacggcatatgctagagtatatcttcaatcaatgataatagaaggttgtggagttgaaaactttttatacagcgatactgacagcttttattgttctatacctaaggaaaaagtaattgaaggtttaaagaaaatgggtgtagaagttcaccctaacaacttaggagctatggataatgagaaaaactttaaagagtttaaaaccataggggctaaaaagtatatgcttagaactaatgatggaaaaatatcttgtaaatgtgcaggtgtaccaaaagatgcacaaaagatattatgtaatgaaggttttgaagagtttaaactaggtaaagaagtaaagggtaaattagcacaaataaaatgtgatggtggtcttgatttagtagaagttcaatatataataaaagatttaagtttaaggaggtaacatgggtaagttttttgaaataaaagagtttatagaagaatacccaaaccatttaatacattgtgttataggtgcaaggggtgttggaaaatcatactcaactaaaagattgataatagaagagtttttggaatatggaaaacaatctttaatattaagaagatataaaaatcaagctgaaagtatggcaacgacatactttacagatgtattacaaaaagaatttccaactgttgaatatgagttaaagggtgatatgggatttataaatggagaaccattttgtttattcacaggtctaaatggaaatagtttagctaagggttcatcattccctaacgtttattatattatttatgaagaagttatgcccgagcctggggagaaaattataaaagatgaatataagaaattagaatctataattgtaacagttgatagatttgaggatagaatagaagtaatatgtgttggtaataatacaagttattataaccctatttttgacactttaaagttatatccttcaaccaaaccgaatagggttgttaaaaatgaacttatggcaatcaaaacgcttgaaacacctaaagagtttaaagagtatgccttaaattctaaggttggtaaattaactgtaatgagtgggtcatcacgttataatatagataatgaaaatattagtaatgatagatttaatgtttgtagtaaaaaagaattatgggatactgataaactagaaccagtttttaaggttcgtgtagatgtggacaaggtaattaaaatatggaaatgtcaaagtgatacaatgttttactattgggttgataatcacaataagggaaaatgtgacgaatactttttagatttagattatcagacagacgaaaataaacatatatctttatgtaataaaggtcttataaatagaatcaattttaataataaagttggtgctgttttctttaataatgctgaaacaaaattctattttaatcaaattggattattctttaaaaaataatacaaaaaagtgttgacataatatataaatgtaatgtataattaactcataaggaaaaacaaaccaaaacaacaaaaatatttaaaattaaaggagagatttaacatgaaaaaattatcaattttcaatttaggaaacaacgtttcaagtggggtatattttagattaagggataacagagaaaaggctgttataactttcttaatcaataacattgatgaggttgaattagatttagttcacccagtaaaattagcagatggtagagaaataaatgtaaagtgtgtggctgaattagacaatgacttaaaagtaatcaaagatacttgtcctctatgtggtgaaggtttcaagatgtcagaaaaggttgctgttaatgtttataaccatactactaagagtgtacaaatttggcaactaggcaagactttaatagaaaaattaattttcttagctgaaaaaaggcccgaccttattaatcacacttatgaggtaacaagattaggggctaagggtgacactaatacatcatatgaatttgagtatttaggtgcaacagataagaaagtatttgaaggattagagttaatcaaaaagaacgaaacagtatattatgaggttggtgttacagagttagaaaacttcttaattactggagatgaaaaaatatttgaaagaaataaataatttaaaggtgggttaattcccacctactaaaattaatatagaggtgaataaaaatggaagaaaatagagagttaacagaattagaaatcagaaaggaattagctttagaaaaagttaatgaaaagagattacaacttaatagagtagtttcttttataaatcaagaagaaaaagagtttacaaaatctaaagaacatgaggctgtaatgtttaatacccttttaacttctttaggaatagattttaaagtaactgaaactaaaaagagaataatatttcaattagcatagtgtaaagtatacccccttacttagctatataacgtaaatgattaggaagtcacacggtagaacgtgcctaacatttttccacctatgaccgtgggcgttgtatagtgaagtaagggggtataatattttgtaaggaggttttaaaatgagtaaagaaatgactcttgatgatgcccttttaagaattgttgaattagaggaaactatagaaaatcagaataatgcaatcagcaaattaaattctgaaaaggaaaccaattcaaaagaaattgaaaaacttcaaaaaagtaacatggaattatttcttaaagttactaacaaaccaaaggaagaaaataatattgaaattaaagaagatagtgagtcaaaaatggacaagctattaaaagaatgggagtgataaaatggctgttaaggcaaacaaagaaatcatggaggcttttttagaaggtgcaacagaagaaactaaaaaggttttaagtcaagtagatttatctgatagtagagctgtttcagatgctttattcaattaccaagttgtaaaaaatgagtttataagttcccttataaataaaatatttatgactcaaatacattcaaaagtttggaaaaatcctttaaaaatattccatggtggaatggaagaatacggttatactatagaggatatgtatgtagaggcatcagaaaaagttgggtttaaagaacattttaatagtggtgatgaagtaaaagacatagtagctaccttagtacctaaggtatatacaaattatctttcaagaaattttgctgataaatacaaaataacaatatcagatattcagttaagaacagcgttcagaaatgaatatggattagcaaacttagttaataaattattagaggctaacttaagaggtgcttatcgtgatgaatataaatatatgaaagatatgttttttaaatattgcaagggtttaactaatcaagatgttggaatgagtagttcacctacaaaaacccaatatattcaagattctcaagttattaaattaaatgatgatgcaaatcttgttagaaatctttgtaaatcattaaaggctatgaataaaagattacagtttgaaagtgataaatataatagtgctaaggtaatgcaattttcatctttaaatgactgtgtatttataacaactcctgaagttgaggcagagttagatgttgattttttaggagatgttttcaatctttcaaaggtagaggttcaacaaagaattatactaatggatgaactaccaacacaaataaaaagtggtgagagtggtacaaatacacatgatggtgaaacttgtattggtatgttagttgacaaagaccttctaagatttaaagattttgtatttgaaacaagatattttaacaatccaaataatttatctacaaactacttcttacacaagcaaggtttatgtggtatagtgccatttttaaatgctataatatacactaaatcagcatcataatgaggtgatttaaaatggaaaagttaactaataaggaaatttacgataaattaatttcaaccatgcccaaggatttgggcaactcttttccttcttatgaagaggctttaaggttgaatttaataaatattgatatgaatagttttatcgaaactatatgtaataaaatagggaaaatgttatatatagaacaacatttttcaaaccctttcaatgaacttttttatggtgaaatgccattcggttattcaatcgaggagttgttcttagaggactcaactgttcaatcattactagatggtgatgaaaaagaagttttcaactcttctttaccacgcctaaatgggttagttgcaaatagtagatttgacaaaaaagttaaaatgagtcttagtgttgaaagattaaagtcagcatttttaaaagagtttggactatatgatttaatatctaaaatgttatctaatttaaaaacttctatgagtaaagataaatacaaagaaatagaaaaagctttaaaggaatttgctaagggattagattctaacggagaaaaccttaattttaaacaagctccacataaagaagttgcaagttcaaacttaattgagagtattgtaaaaattgtagaagaattcactttgccaagtgataaatataatcctagcaaatttaatacacaatgcttaaaaagtgacataataatatttgtgaaacctgaaagttacgcaaaattaggaagtacagatagcatacttttaagaggagaatataatattaagtcaattagtgatttaccaaataacattactaatttggtatcaaatgaaacaatcaagcctatagctttaattatggataaaagattaatgcctataatgttgaattacaatggattagatagaattgacgacccaaatagtttaacaacaaattattattactatgaaaattatagtctaccagtaaatactttttcaaattttgcaattctaacaagtgaataaaaacgtatatttttattctttaccttatattgacccctcatatcagaacctttttaactttaaaagtataagtaatcaaaataattatttttcttctaaagtagtgtgggtgggtcaatataacataaaggtagagccttcacgtgtaagcttagtggtagacaagccttacggttggtttatggagaataatgttaattatatttcagcttatgatactaacatgaaaaatatttattatttcatagacgactttgtttataaaacagaaaactcaacacttttattaatatcagttgatgtattccaaacctatcaatttgactttgaaattttagaaagttttgttgatagatgtcacgttaatagatgggacggtgataacccaacgaatgaatacgaaacagaagatatttcttatggtgaaaatatcatgctagaatatgaaaaaatagctgatatggggcgtggtgtcgttgtcacttctacagtacctctcggaaaagttgaaacaaacgttggtggtggttctgacgggactggttcagcaagtggtgatatagcaaatggtattatatcagcaaatggacttttatttgtaaaacaggaagaaggttttgcagaatatggggcatattttaatggagaatcttttaaaactggtgggtatggtgtaactgaaaattatcaaactaaatattataaacaactagaaccattccctgtatcagaggaaaaagctagtcaagtcacttatgatttattaaataatgagtttggaataccagttaaaaacgcaatgttaaaagctaatataaatttaagtgatattcctatatatcaatttgatgtttgggtatcaatcgcatttaactatggaatgggtggtttaagtggattaaatgcgtggcaaatgtttttagctaatccaaaagatacgaaaaatattgcaacagagataaaaaatttaaaaccaaacccaaatagaaggcagagagagggagcattatttgaaagtggtgtttatcctcaaaggcaaattttaaagtatggtcaaaatggtcaaatagttggttatacagatggtaatggttggttgcctagtggtaaaaaagatggtaaatatgttgataatgatgtgggtacaaattggttaatcccaacaacgggacaaataagtgcatattacccaacttatccaagtggcaacccacacaatggtgttgatattgcaacaccaactggtacaccagtttatgcaagtaaagacggaacagtcattaaaagaagtgaattaactactagctatggaaaatttttaattatacaacatggtgactcccaagtagtttacgctcataattcagagttaaaagtaaatgaaggtgacacagtaaaacaaggtcaattaattgcattaagtgggaatactggtaatagtagtggcgaccatttacattgggagattagaaacgaaaagggaacagttgttgcaaatggtgttaaaactgttaaccctatgccaaattataaggtgggtgacaaagtatgacaataactaataaagattatgatacgtgggaatctaaaatgtatggtttgccatatgctgtatatagttattatattgactctaccgtggctggtgaaaatacagctgtcattggtgcaagtggtgacaatgtttttagtgttgtttacaccccattccttgatatttctgatttagaactagaagaaatcccatatgataataaacgatttggtaatatttcagagattagacccgagattaaagcaaatcctcacgttttcagaattaaaaaattattaaaaggttctaaatttgtaggtgagtttgaaacttacaaagtaaaaaagagtgtgggtggtaaaagaaactggagaaatgaaagtaaattatacaattacccatacacttatttaatgctaagtgatggtataaatgaccctatgattttaaagcctcaattttgttcaattcctaattgttcggttggtataaaattatcagtttcagaccgttgttcgtatggtatatttgtacaaacttataagggtgatacagacggaatgactgaggctttggtatcaaatgatgcacttgaattgccttgcactagttcagcatatgcaaattgggtcgcaacttctaaaaatcagacagcacaatcaattcaaaatactgtaaatcaaactattttaaatgataaaatagcaaaaaataatatgaatttaggtatagcaaatagtgttgttggtggtgttgcaagtgcatttactggtaatgttgggggtgtaattgatagtgtctttggtggtgttggttcatatatggctaaaaaacacacgaatatgcagagtcaattaactagagaaacggctataagttcagctttagcaacagcgaacgacatgaggtcaactcctaatactttattaagtcaaggttcaaatataatttatgggttgcgcaatggtggtcaagaattaagactctataggtacggattaacagaaagatattatgaaaaaataggcgattattttgcccagtttggatataagcaaaataaaatgatgaaaattaatattaattcaaggtattattataactatataaaaacaataggaattaatataaaaactaataaaattcctaataattacttaaatatccttaaaggtatttttgacaatggtactacaatatggcatattgataatgaaggtgtcgaaattggtgattattctatggataacagggaggtataaaatgaaaataggaattagagatgggcatagtccaaattgcaagggagcgattgctttaagagatgaacaggcgtgtatgagaattttatgtacagaagtaattgaggttctagaaaaacatggccatgaagtagtttattgcggttctaatgctagaacagaaaatggagagctaagtgaggggataaataaagctaattccaataacgttgatatatttatctcacttcatatggacgtttcaaaagaacataaagctaatggtacgtgttcatttgtagcaaaaggagcaaggaaatcaataagagatattgcacaaagattagtcgataactttgaaactttaggtttacaaaatagaggtgtaagggagagtaattatagagaaatgagagaggttaatgcacctaacataatatttgaaaccatgttttgtgataaccctcacgacataaatgaagtgtggtcacctacaccatacgaaaaaatggctttactaattgcaaatgctattgacccaactattaaagaaaatgaactttatagagttgttgttcaatattttaacagcaaaaaagatgctgaaaactgtcaacaagaaattgctaaaagatggtattgttttgtggaggagtgtaattaatgcaagaaatagtaaatataataaatagtgttgggtttccaattgttgcatgtatgataatgtttaatcaaaacagtaaactatcgaatgcaatttcagagcttaacataacattaacaaagatacaatcagatattgacagtttaaaaaataatcaaaaacaaggggaataaatttccccttttaaaaaaggtggtgattagatgggaaaaagaaaatctataatatctcaaaaccctaatattttactaaatgaaaatgcaatatgtgataaccaaacaaggttatttttacattataaacttatggcttgtaataggtttaaatgggagaacttacccattggtttagaaagtagacatattgaggactttttatttgaaaatggacaatgttttttctttaaagatgataaattaggtttaatgtgtttaccttgtcatggtttaggtgatttaaatatctatggtgataatatcaaattaagcatagtttcaagaaatggcaaatatcaaaacatcttagaagatggtaaagatggcataaaaataagagcgaatgatttatgtttaccaacgtctcattttatttcacactatgctcaaaaaatggatgatatagaaacagtaataaaaagaaatttaaagcaacaaatgaaacctttctttgttacagcaacaaataataatctattgtcagtaaaaaacattgtaaatgatgttgacaatggaaaagaggttgttatcttagataaagacctaggggaacaagggtttgacggtttcaagatgctacagactggtgttatttacttagttgataaattagaagatgaaagaaaatcagtagaaagtgaactactttcttttttgggtttagataatgcgaatacagaaaaaagagagcgacttttagttgatgaagttaacgctaataatgaatacattgaaactaacttagatatggaatataaaactaggttattagcttgtaaaatgataaatgaaaaattcggaactaacataaaagtaactaaggttgttgatagctttggaggtgattaaaatggcgaagtacacactagagttgaggacattggagaataatttatttgactttgattatccttactatgaagaagaggcaaaatgttactttgaggaaaaatttttaaatcattattactttcatgaaataggatttgaaactatagcaaggtttaaacaacaattaagagcataccttttaagagttatgccatattatcagcaactttatgaaatagagttaagatgtaaagatattgactttatgttaaataaagatttgaaagaaacttttattagggaactagaagagaatgaaataaataatttattaacttcacaaaatgaaactggtaacacttctacaaatattaataataatgaaagcaataactcaaataataatacaaaagaaagttcacttgctgatggtgtatcgagttcaaaaattgctgacggatatttaacaaattcaagtcaatctacagatgtttatgatagtaatagtagtacaataagcaaaacagataataagagtaatttaaatataaataatactggtgataaaaacaacaaacaaaaagaaaaaacagaattaatttcacagggtaatattggggtaacttcatcagcagaattaaaagagaaatggcaaagttgtctaaataacatagatgaacaaattatattagacgctagaagtttatttatgtatgtttattaggtggtgaaataatgattataattgatagaaataaaataaaagttaatcaatttgatacttgtattttagatttaaatgtgtgtggatataaattacaagaaggtgataaaatagaatttatatttaattctataaaagaagaaaaagaatttaatgatactgatttaatattcccaactgatgaaaaaggtacttttaattattctgtaacaatattacaaaaaggaattattagaactgaaattattaatagtatatgtgaggtgatataatgagtaagataacgggaaaagttaattataaagaagtgttaaagggtgacactggttatacgtttgaaccacatgttacagttgacggtattttatattggagtaataatggtgatttaccaaatcctttaccagtaaatataaaaggtaaagatggcaaagacggcgagaatggaagagatggtagagatggtataaatggtaaagatggtaaagacggtgagaatggaagagatggtagagatggtataaatggtaaagatggaataggaaatggtgtcatatcaagtgaaataattgaaaatggttatatactccaatattctgataacataaaagaatatataataaattctaaaatagatatagaaaaaaattttgttgatataacttttccatttaattttaaaaatgataaatacaatgttgtattaactaaaaaacaaaacaataggagtggtgatatatattattataatttaacaaacaataaggtaagagtttttagtagtaatagctcaaccagtagtgaacaatatttttcaatgcgtgttgttggggaggttaatatataatgaataacataaataaattaaaccctattgaccttaattgtagcatctttacagtttatgattatacaggactatctatgcaagaaattttatatcaattctttagtaaaataaatgaattaattgactcacaaaatcaagtaatagatttaacaaaatggttagtaggtcaaggtttaaaagaagaagtcgctaaacaacttaatcaatggttaattgatggtactttatccacaataataaatgaaacagtattcagagatttaaataataaaattaactctaatatagaaaatattaaaaacaataaaaaagaaatagataaactagtaacagtttggaataatcttaaatgttttaatgttgctgattttggagcaatcggcgatggagaatctaacaacaataaagcctttgaggatgctataaattcatgtgttgaaaatagtatacgtggtttattaattcctagtggaaaattttatattacagaaggtattaacacaaaaggagtcaaaattataggtatgggagacccctatattccatttttagaatgggaatatacaagaccaaactcaaaattagatgaatataaaaaatatttaaataaatgtcgtggtagtataataacatcagataaaaatattaatatatttactaatggtttatatgctgaaaatataggtatttttggtaatagaagagcattatctcaaaatggtataggtcaaatggatggtgggtttggtaactggattgaaatagaaaaagttaaaatccatggatgtggtaataatggtattaacgctgaatatggtttaataacccctatcataaaccaatgttggatgtatcaaaatggtaataatggaataagaattggtaaaaataaaggaacttacacaggagaaacaaacgccttaataatagaaaaatgctttattaatagaaatgaaagccacggtatttacttagatgttaaaggtagagcatatactattaaaaataatgacttagagcaaaatggtgaaatgagtgacccattgagaaataataaaggtactgattataatgatatagtttatggttgttatatgaaggttgagggtgaaggtggatttactagtggttcaatagattttagtaataactattcagaggaaactttaggtttattatacttggaaagtcccgataataaaatatgtcaaggtgtaaagtttgaatctaatatgtggcgaccttataatcaagaattatattcaaatggtttactattaaaaggttggattgaaggtgttaaaattggtaataataatatgtacggtagacataaagtaagagttatcaatgcaaatacttatggtatagaaactgacacagatataacaaacccagtatataaaaataaaaatatatctatcgaaaaacattatgactataatggtacaatggttgactttagaagtactgggagagttgaaaaatattctattgaaaatatgatacaatcagcatcttatgacggtagcacatacaccaatgtatatttaaataaacctagcgtaccatatgagttgcaagaagacggacaaggtaataaattaataggaacaacgtttttaacatcagacggtacaagtattggtttagttgttgacttctcatggactcatggattatttaaaataagaaaggacagaacatcattaataaaaactggtaacgctaagtttatggagaatggtgggtttacaacaataaacggtggtaatacaccagtaaaaatctatgttgaccatgacaatgaaatagtaattttataaaagaaaagagggtttatttatcctcttttattctttcttttatatcttcataaaaatcaatttgtttttctagttgatttatattatcataagtgtcactataattttcaaaaaacattttttcgtatgctattcttgtattaagttcttgtattcttgtatttaataaatcatttaatacagtttctaaaccttccttatctttacactcataatttatgaagttatcaataaatttttgtgtgtagcgtgaccttcttagtaatgtataaatcatatctaatttgatttttctgtcatagtcctctaataaaatacatcttttagaatctatttttattaaatttaataatgttttaaatttcatttttatttaacctccttacaccatgatgctataacgtaataagtacctattttaccttcaaaattatcaattaaattttgtacttttctaccatttaatttgtttgcccataacttattcattttccaatcatggttgttatttctgtttttaacctttttagccacaaaaacgtactttttaccttcttgaaattttttcattttcaatcactccttaatataatatacagcttaaaaacattacaaataataagcttgatattatgaaaattgaacttgttaaaaatgtattgttatataaaaacataaatgatataccaactaatattcccatgataatgactaatattaattgaattaattttttaatcaatattatcacctctttacataatataaataacctagaacaaataaattaaatagtaaccagtaacatattatttttaacactttaacacctctcttaattgatactttaattatagcaaattatgttaagctattcaatgctttttatgaaatatatttgtaaacaatttgttaactagaatggcaataaaaagagccatgataggctctattttatattaaatcatcatataatattataaacatttgtgatttattatcataatcaacttttttaactgcatactctttatcatgtaattctttaagatgtatattaacagtctcctcactttcaacacaacttaaatgcatagttggtactatttttatttgcctcatttataatcacctcttaaattatattgaatcgttataaatttcgaatacttctatattattcacttttgcaaatattttatacttctttttaggcaaccattttagccattttggattgtttaaattttcatataatccaaccctttcaattaatttattatcatcatagaataccattatgaattgtttttccattttaaatcacctctatagttttatacactcattatgttgttttttatatacatccaaataaacctcatttttatcaccgttatatgttatctcgtaatataacccatcttgtatattagtgcttaataacgccttgttgttttgtaatgttttacaagaccatacaacataaatgtcttttgtaaaaactgtccatcctttacgttctaatagataatcctgtatagatttaatacaaatatctataaactttttattatccataatatcacctctcttaattgatactttaattatagcaaattatgttaagctattcaatgctttttatgaaatatatttgtaaacaatttgttaacagatagtgtcgctttctctactggt

SEQ ID NO: 7 - DNA, 19339 bp, Unknown
  Clostridium phage vB_CpeP_ACP10, complete genome
catattccccagttattaccagtgtagaaagcagcactctatgttaacaaattgtttacaaatatatcttataaaatcattgaatatcttaacataatttgctataattaaagtatcaattaagagaggtgatattatgtttagaaagcatgaattattaagcaccaaactagattcaaaacaaggacgcaaatagaaaatgaaattaaaaggatgcaagataagggtaagtttcctgttagaattatctatgattgtttatttaatcaattttctgtttggtatatagagtaggtgatattatgagtgatgacagttttataacgtttatatgcataaatatgttaggtgttctaatagaacttataatattttatttattaggtttaatgtgagaggtgatatagtgttaaaaataatatgttactggttactatttaatttatttgttctatgttatttatattatgtaaagaggtgataacattgattaaaaaactaattcaattaatgctaataattatcatggggatattagttggaatatcatttatgtttttatataacaatacatttttaacaagttcaattttcattatatcaagcttattatttgtaatgtttttaagctgtatattatattaagaggtgattaaaaatgaaattcaaaacattattaaatttaataaaaatagattctaaaagatgtattttattagaagactatgacagaaaaatcaaattagatatgatttatacattactaagaaggtcacgctacacacaaaaatttattgataattttataaattatgagtgtaaagataaagaaggtttagaaactgtattaaatgatttattaaatacaagaatacaagaacttaatacaagaatagcatacgagaaaatgttttttgaaaattatagtgacacttatgataatataaatcaactagaaaaacaaatcgatttttatgaagatataaaagaaaggttaaaaggtaaaaagaatgattaaactttataaagaatttattgaaattaaggatatgataataatatctaagaatttaagtgatagtaaatttatagatttttctaaagacttcacatattgtaacacttatattacaaataatgattttattacattaaaagataaaaagtttttagagagggtaaaaataaaatttaaacttatattacaaataataaaattataaaagagggcaaataaaccctcttttctttttataaaattactatttcattgtcatggtcaatatagatttttacaggtgtattaccaccatttattgttgtaaatccaccattctccataaacttagcgttaccagtttttattaatgatgttctgtcttttcttattttaaataatccatgagtccaagagaagtcaacaactaaaccaatacttgttccgtctgatgttaaaaacgttgtaccaattaatttattgccttgtccgtcttcttgtaattcatacggaacgctaggtttatttaaatatacgttggtgtatgtgctaccatcataagatgctgattgtatcatattttcaatagaatatttttcaactcttcctgtacttctaaagtcaaccattgtaccgttatagtcataatgtttttcaatagatatatttttgtttttatatactgggtttgttatatctgtgtcagtttctataccataagtatttgcattgataactcttactttatgtctaccatacatattattgttaccaatttttatgccttcaatccaaccttttagtagtaacccatttgaatataattcttgattataaggtctccacatattagattcaaattttacaccttgacatattttattatcgggactttctaagtataataaacctaaggtttcctctgaatagttgttactaaaatctattgaaccactagtgaatccgccttcaccctcaaccttcatataacaaccataaactatatcattataatcagtacctttattatttctcaatgggtcgcttatttctccattttgctctaagtcattatttttaatagtatatgctctacctttaacatctaagtaaataccgtgactttcatttctattaataaagcatttttctattattaaagcgtttgtttctcctgtataagttcctttgtttttttcaattcttataccattgttaccattttgatacaaccaacattgatttattataggtgttattaacccgtattcagcattaataccacaattaccacaaccatgtattttaactttttctatttcaatccagttaccaaaacccccgtccatttgacctacaccattttgagataatgctcttctattaccaaaaatacctatattttcagcatataatccattagtaaatatattaatatttttatctgatgttattatactacctctacacttatttaaatattttttatattcgtctaattttgagtttggtcttgtatattcccattctaaaaatggaatataggggtcacccattcctataatttttacaccctttgtgttaataccttctgtaatataaaattttccacttggaattaataaaccacgtatactattttcaacacatgaatttatagcattttcaaaggctttattgttgttggtttcaccatcgccaattgctccaaaatcaacaacattaaaacatttaaggttattccaaactgttactagtttatctatttcttttttgttgtttttaatatcttctatattagagttaattttattatttaaatccctaaatactgtttcatttattatgttatctaaagtaccgtcaattaaccattgattaagctgtttagcaacctcttcctttaatccttgccctactaaccattttgttaaatctataatttgattttgtgactcaattaattcatttattttactaaagaattggcataaaatttcttgcatagataaccctgtataatcgtaaactgtaaagatgctacaatttaaatctataggttttaatttatttatcatatctctcacctctataatataaattcactttgtataatttgtcctttgccgtttaatgaaggtatagtttcgtcattaattaaataaaaatcaatattttctcttgaggtgaaaatatctattacttttagttttaatttagcatatattttagaatttgctttataataaatgttataaattactttttcaccttctactttatatgtcatttgaaaacattcgtttttatttaaatatttgtttttgaaatacgatgaaaaaaacattccacttttattttcattgtctactgaaccgatagcaacatgtatttttaaaacaccactatctccacatagaacttcatcatcaactaaactgtaatttaatataattgtacatttagcatttttattttgtgttttaattgttgctatttttttaaaacattcttcatttgatttaacttcctcaattataattgtatcatttacaacggttttattgcttatacttttagtattatttagtaatattgcttgttttaagttatttataaataaattatttataaaactaaatatatcatcatcagatgattttaatataaaacttgaatttttatcataaccatatttattaataaattcatctttatttaatatggtaaattcttcatcattttttaataccgactctttatatccaattattaaattattatcaacacattcatattgagggtttactctaaaaaaataaattggatattcgtctatttcaaatatattatttttaatttttatattaattttttcattgtgtaggtttgtttcataaaaaagtatgtgacaatttacccaataattaggtcttgtttttgataaaacaccttttgcgttttttatacacatgttatttgtaaacatacagtttcttatacttgaattatcattacctttaacccatatctcaaaactttgcccacattcttctattttattattttttatttttatattttcataattgtattctttattataatcattactcccttgaattgttgtcgcggcatcataaatatttttaaatttacaatttgtaacttcaatgttagagccatatgtccatatttctacgccattaccaaatcttttgccatctaaatccaattttgaaccacctatattttcaaattcacagccatttattgttatatttttacagctaaatgatagagcgttaccaccattattcataaattttatattgttaattgacatattgtcatatatatccatcatggtctgtctagttacaaaactaatattattgatatcattctttttacattttatatataaagtgttgttatttgaaaaaaaatcaaatttgttttttaattcattaatactcatttttttattagcatacataacaccattcactttgataaaaccaatattatctattgtgttagtttcaacataaccgttaaaattactaatatcatttaatgggattgaaataatgtcgtctatttcatctataatttcattgtttactaaatttattaaaaaatcaaatattggtatatctcctgcaccatatgaattaacattacacccaatatcagtttttaatttatgattgtatatactattacactcaattaaaattgtatctcctttttttattacagaattaattttttttattgttttaaaggggtaatttatagaaattccattgttattatcattacctttgttacttaaataatatatcattaaaactgacaccaccctttaaattattttttaatatattattttcaatgtactcgattgtatctaaccttttatttaaatttaaatatttacctctagcttgaactatttcactatttgttacactttcatttccatttgttaaatttgctattgctttatcataatcatttataactttatcaatctttaatctatctgtttcttttaaatcaccttcttcacctttttcacctttttcaccttgtttaccttttatatttacaggtaaaggatttggtaaatcaccattattactccaatacaaaacaccgtcaactgtaacatgaggttcaaacgtataaccagtatcaccctttaacacttctttataattaacttttcctgttattttactcaattatatcacctcacatatactattaataatttcagttctaataattcccttttgtaatattgttacagaataattaaatgaacctttttcatcagttggaaatattaaatcagtatcattaaattctttttcttcttttatagaattaaatataaattctattttatcaccttcttgtaatttatacccacacacatttaaatctaaaatacaagtatcaaattgattaacttttattttatttctatcaattattatcattatttcaccacctaataaacatacataaataaacttctagcacctaatataatctgttcatctatgttatttaagcaactttgccatttttcttttagttcagctgatgaagttacaccaatattaccctgtgaaattaattctgttttttctttttgtttgttgtttttatcaccagtattatttatatttaaattactcttattatctgttttacttattgtactactattaccatcataagcctctattgattgagttgaatttgtcaaatatccgtcagcaatttttgaacttgatacaccatcggcaagtgaactttcttttgtattattatttgagttattgctttcattattattaatatttgtagaagtgttgccagtttcattttgtgaagttaataaattatttatttcattctcttctaattccctaatgaaagtttcttttaaatctttatttaacataaagtcaatgtctttacaccttaactctatttcataaagttgttgataatatggcataactctcaaaaagtatgctcttaattgttgtttaaaccttgctatagtttcaaatcctatttcatgaaagtaataatgatttataaatttttcctcaaagtaacattttgcctcttcttcatagtaaggataatcaaagtcaaataaattattctctaatgtccttaattctagtgtgtacttcgccatcttcaccacccccaaaactatcaacaactttggacacttttatattagtaccaaatttttcatttatcattttacaagctaataacctagttttatattccatatccaaatttgtttcaatatattcgttattagcgttaacttcatcaactaaaagtctttctcttttttctgtattagcattatctaatcctaaaaatgaaagtagttcactttctactgattttctttcatcttctaatttatcaactaaatataaaacacctgtttgtaacagtttaaatccatcaaaaccctgttcggctaattctttgtctaagataacaacttcctccccattgtcaacatcattcacaatattttttactgacaataaattgttatttgtagctgtaacaaagaaaggtttcatttgttgctttaaatttctttttattactgtttctatatcgtccattttttgagcataatgggaaataaaatgcgatgtcggtaggcataaatcattcgctcttatttttataccatcttttccatcttctaagatattttgatatttgccatttcttgaaactatgcttaatttgatattatcaccatagatatttaaatcacccaaaccgtgacacggtaaacacattaaacccaatttatcatctttaaagaaaaaacattgcccattttcaaataaaaagtcctcaatatgtctactttctaatccagtcggtaagttctcccacttaaacctattacaagccataagtttatagtgtaaaaatagtcttgtttgattatcgcatattacattttcatttagtaaaatattaggattttgagatattatagattttcttttccccatctaatcaccacctttttaaaaggggaaatttattccccttgtttttgattattttttaaactgtcaatatctgattgtatttttgttaatgttatgttaagttctgaaattgcatttgatagtttgctattttgattaaacattatcatacacgcaactataggaaacccaacgctatttattatatttattatttcttgcattaattacactcctccacaaaacaataccatcttttagcgatttcttgttgacagttttcagcatcttctttgctgttaaaatattgtacaacaactctataaagttcattttctttgatagttgggtctatagcatttgcaattagtaaagccattttttcgtatggtgtaggtgaccaaacttcgtttatgtcatgagggttatcgcaaaacatggtttcaaatattatgttaggcgcttttacgtttttcatttcatataaattaacttcttttacacccctatttactaaacctaaactagcaaagtttttacatagccttgatgcaatttcttttatagaatttcttgccccaactgtaacaagtgcctctgttccttgagcttgtccgttaaagctattcatgtgtaatgaaataaatatatcaacatttgaattattagcttttctcacaccttccgaaagttcactattttgtgtacttgcatcactaccacaataaactacctcatgaccatgtttttctaatatttctatgacttctttacataaaactcgcatacatgattgttcatcacgtaaacctatagcacccttacaattcggactatgaccgtctctaatacctattttcattttaaacctccctgttatccatagaataattaccaatttcgactccgtcattatcaatatgccatattgtagtaccattgtcaaaaatacctttgaggatatttaagtaattattaggaattttattagtttttatattaattcctattgtttttatatagttataataataccttgaattaatattaattttcatcattttattttgcttataaccaaactgtgcaaaataatcccctattttttcataatatctttctgttaatccatatctataaagtcttaactcttgaccaccatttcgtaaaccatagattatatttgaaccttgactaagtaaagtgttaggtgttgacctcatgtcgttcgctgttgctaaagctgaactgatagctgattccctagttaattgactctgcatattagtatgttttttatccatgtatgaaccaacaccaccaaatacactatctattacaccgccaacattaccagtaaacgcactagcaacaccaccaacaacactatttgctatccctaaattcatgttatttttcgctattttatcatttaaaattgtttggtttacagtattttgaattgattgtgctgtctgattttttgaagttgcgacccaatttgcatatgctgaactagtgcaaggcaattcgagtgcatcatttgataccaaagcctccgtcattccgtctgtatcacccttataagtttgtacaaatataccatacgaacaacggtctgaaacagataattttataccaactgaacagttgggaattgaacaaaattgaggctttaaaattaatgggtcatttatgccatcacttagcattaaataagtgtacggataattgtataatttactctcatttctccaatttcttttaccaccaatactcttttttactttgtaagtttcaaacccaccaacaaactttgagcctttcaataattttttaattctgaaaacgtgtggatttgcttttatttcaggtctaatttctgaaatattaccaaatcttttattgtcatatgggatttcttctagttctaaatcagcaatatcaagaaatggcgtgtaaacaacactaaaaacattgtcaccacttgcccctataatagctgtattttcaccagccacggtagagtcaatataataactatatacagcatatggtaacccatacatttttgattcccacgtatcataatctttattagttattgtcatactttgtcacccaccttatctgtcctgttgttgggattaaccagtttgaccctgcatcattatcaacatattttccatcttttttaccactaggcaaccaaccgttaccatctgtataaccaactatttgaccattttgaccatactttaaaatttgtctttgaggataaacgccactttcaaataatgctccctctctctgccttctatttggatttgcttttaaattttttattgctgttgcaatatttttcgtatcttttggattagctaaaaacatttgccacgcatttaatccacttaaaccacccattccatagttaaatgctattgatacccaaacatcaaattgatatataggaatatcacttaaatttatattagcttttaacatagcatttttaactggtattccaaactcattatttaataaatcataagtaacttgactagctttttcttctgatactgggaatggttctaattgtctataatatttagtttggtaattttctgttacaccatacccaccagttttaaaagattctccattaaaatatgctccatattctgcaaaaccttcttcctgttttacaaataaaagtccattcgctgatattattccatttgctatatcaccacttgctgaacctgtcccatcagaaccaccaccaacgtttgtttcaacttttcccaaaggtactgtagaagtgacaacaacaccacgccccatatctgctattttttcatattctagcatgatattttcaccgtaagaaatatcttcagtttcatattcattcgttggattatcaccgttccatctattaacgtgacatctatcaacaaaactttctaaaatttcaaagtcaaattgataagtttggaatacatcaactgttattactaatagtgttgagttttctgttttataaacaaagtcgtctatgaaataataaatgtttttcatgttagtatcataagccgaaatataattaatattattctccataaaccaaccgtaaggcttgtctaccactaagcttacacgtgaaggctctacctttatgttatattgacccacccacactactttagaagaaaaataattattttgattacttatacttttaaagttaaaaaggttctgatatgaggggtcaatataaggtaaagaataaaaatatacgtttttattcacttgttagaattgcaaaatttgaaaaagtatttactggtagactataattttcatagtaataataatttgttgttaaactattagggtcgtcaattctatctaatccattgtaattcaacattataggcattaatcttttatccataattaaagctataggattgatcgtttcatttgataccaaattagtaatgttatttggtaaatcactaattgacttaatattatattctcctcttaaaagtatgctatctgtacttactaattttgcgtaaatttcaggtttcacaaatattattatgtcactttttaagcattgtgtattaaatttgctaggattatatttatcacttggcaaagtgaattcttctacaatttttacaatactctcaattaagtttgaacttgcaacttctttatgtggagcttgtttaaaattaaggttttctccgttagaatctaatcccttagcaaattcctttaaagctttttctatttctttgtatttatctttactcatagaagtttttaaattagataacattttagatattaaatcatatagtccaaactcttttaaaaatgctgactttaatctttcaacactaagactcattttaacttttttgtcaaatctactatttgcaactaacccgtttaatcttggtaaagaagagttgaaaacttctttttcatcaccttctagtaatgattgaacagttgagtcctctaagaacaactcctcaattgaataaccaaatggcatttcaccataaaaaagttcattgaaagggtttgaaaaatgttgttctatatataacattttccctattttattacatatagtttcgataaaactattcatatcaatatttattaaattcaaccttaaagcctcttcataagaaggaaaagagttgcccaaatccttgggcatggttgaaattaatttatcgtaaatttccttattagttaacttttccattttaaatcacctcattatgatgctgatttagtgtatattatagcatttaaaaatggcactataccacataaaccttgcttgtgtaagaagtagtttgtagatagattatttggattgttaaaatatcttgtttcaaatacaaaatctttaaatcttagaaggtctttgtcaactaacataccaatacaagtttcaccctcatgtgtatttgtaccactctcaccactttttatttgtgttggtagttcatccattagtataattctttgttgaacctctacctttgaaagattgaaaacatctcctaaaaaatcaacatctaactctgcctcaacttcaggagttgttataaatacacagtcatttaaagatgaaaattgcattaccttagcactattatatttatcactttcaaactgtaatcttttattcatagcctttaatgatttacaaagatttctaacaagatttgcatcatcatttaatttaataacttgagaatcttgaatatattgggtttttgtaggtgaactactcattccaacatcttgattagttaaacccttgcaatatttaaaaaacatatctttcatatatttatattcatcacgataagcacctcttaagttagcctctaataatttattaactaagtttgctaatccatattcatttctgaacgctgttcttaactgaatatctgatattgttattttgtatttatcagcaaaatttcttgaaagataatttgtatataccttaggcactaaggtagctactatgtcttttacttcatcaccactattaaaatgttctttaaacccaactttttctgatgcctctacatacatatcctctatagtataaccgtattcttccattccaccatggaatatttttaaaggatttttccaaacttttgaatgtatttgagtcataaatattttatttataagggaacttataaactcattttttacaacttggtaattgaataaagcatctgaaacagctctactatcagataaatctacttgacttaaaacctttttagtttcttctgttgcaccttctaaaaaagcctccatgatttctttgtttgccttaacagccattttatcactcccattcttttaatagcttgtccatttttgactcactatcttctttaatttcaatattattttcttcctttggtttgttagtaactttaagaaataattccatgttacttttttgaagtttttcaatttcttttgaattggtttccttttcagaatttaatgtactgattacattattctgattttctatagtttcctctaattcaacaattcttaaaagggcatcatcaagagtcatttctttactcatttttaaaacctcctcacaaaatattatacccccttacttcactatacaacgcccacggtcataggtggaaaaatgttaggcacgttctaccgtgtgacttcctaatcatttacgttatatagctaagtaagggggtatactttacattatgctaattgaaatattattctctttttagtttcagttactttaaactctattcctaaagaagttaaaagcgtatcaaacattacagcctcatgttctttagattttgaaaactctttttcttcttggtttataaaagaaactactctattaagttgtaatctcttttcattaactttttctaaagctaattcctttctaatttctaattctgttaactctctattttcttccatttttattcacctctatataaactttagtaagggtggggggttaccccaaccctttaaattatttatttctttcaaatattttttcatccccagtaattaagaagttctctaagtctttatttgttacttcataatatactgattcatttttcttgattaaatctaaaccttcaaatactttcttatctgatgctcctaaatactcaaattcatatgatgtattagtatcacccttagcccctaatcttgttacctcataagtatggttaataaggtcgggtcttttttcagctaagaaaattaatttttctattaaagtcttacctagttgccaaatttgtacactcttagtagtatgattataaacattaacagcaaccttttctgacatcttgaatccttcaccacatagagggcaagtatctttgattacttttaagtcattgtctaattcagccacacacttgacatttacttctctaccatctgctaattttactggatgaactaagtctaattctacttcatcaatgttattgattaagaaagttataacagccttttctctgttatcctttaatctaaaatataccccacttgaaacgttgtttcctaaattgaaaattgataattttttcatgttaaaatctctcctttaattttaaatatttttgttgttttggtttgtttttccttatgagttaattatacattacatttatatattatgtcaacacttttttgcattattttttaaaaaataatccaatttgattgaagtaaaattttgtttcagcattattaaagaaaacagcaccaaccttattattaaaattgattctatttataagccctttattacataaagatatatgtttattttcgtctgtctgataatctaaatctaaaaagtattcgtcacattttcccttattgtgattatcaacccagtaataaaacattgtatcactttgacatttccatattttaattaccttgtccacatctacacgaaccttaaaaactggttctaacttatcagtatcccataattcttttttactacaaacgttaaatctatcattgctaatattttcattatctatattataacgtgatgaaccactcattacagttaatttaccaaccttagaatttaaggcgtactctttaaactccttaggtgtttcaagtgttttaattgccataagctcatttttgacaactctgttcggttttgtactagggtataactttaaagtatcaaaaatggggttgtaataacttgtattatttccaacgcatattacttctattctatcctcaaatctatcgactgttacaattatagattctaatttcttatattcatcttttataattttctcaccaggttcgggcataacttcttcataaataatataataaacgttagggaatgatgaacctttagctaaactatttccgtttagccctgtaaatagacaaaatggttcaccatttataaatcccatatcaccctttaactcatactcaacacttggaaattccttttgtaatacatctgaaaagtatgtcgttgccatactttcagcttggttcttatatctccttaatatcaacgcttgtttaccatattctaaaaactcctctattattaatcttttcgttgagtatgattttccgacaccccttgcacctataacacaatgtattaaatggtttggatattcttctataaactcttttatttcaaaaaacttacccatgttacctccttaaacttaaatcttttattatatattgaacttctactaaatcaagacccccgtcacattttatttgtgctaatttaccctttacttctttacctaatttaaactcttcaaaaccttcatcgcaaagtatcttttgagcatcttttggaacaccagcacatttacaagttattttaccgtcattagttctaagcatatactttttagcccctatggttttaaactctttaaagtttttctcattatccatagctcctaagttgttagggtgaacttctacacccattttctttaaaccttcaattactttttccttaggtatagaacaataaaagctgtcagtatcgctgtataaaaagttttcaactccacaaccttctattatcattgattgtagatatactctagcatatgctgtaacaaaactggcaaatggcttataaaagggttcactttcataagtttctatatattgtccacttgctaaaatttgatttcttcttttttcattttttaactttttaaccctttctaagtttttctctttaccccctctatcgaatgtaaatattttatctttttttactaaatagtctattgtacgtgtagttcttgttccaaacttgccataaaaagaatttaggatagtttttatcgctgaatattgggcatctaatttctcgcccttatctattctatgctttatttctgattttaaattataaaagtgatttacaaagtttgaaaaaatacctttttcagccttatatactaagctagtaccaaactttaaaccattaaatttaatattatctttttctttaaataaaaaaccatcaacctcaacattttcataatgtccaaagtcataatattttgttaaatgttcaagttctacacttgtaatactcatattataattacataaattttcacaaccattacgataaatttcaataactctatcatctattatattagtatgaataaaagaattaccattcataagaaccatattcaatttatctctataaagtaaaggtaaatcagtatcagaaaagttctttgctcctaatctaatcatttgtaattcataatctttacacttaggtttaaaataatcaaaacctatattataaattgctacttcttcaccctcaactggtttaacataacctttgtattcaattggcataccataaggtaataaagcactttcaacttgaaatggatagcttgagtttatatcaaagcttgacccattgcatttcacaactttattaactaatttagggttgtaccaagtccaacctccttcataacttttattctccatttccctagccattttgcttaaaagcatctgtctttcctcaatctcaaattgatttataaaagcttttttcttgtttctatcttcaaaatatgttatctctaaagctgatttaaatgatattgaggaacttgttttacaatttgaacttatgtaaatctcttcatcattatattcaaactttaaaagtgagttgtaaaaataatcatttataacttcttttagtattctgatatcattataaagataccttctttcaatatctgttaaaatatgattatcttctcttattttatcataatcaaactcttctttcaatttataaaacatttcatctaagttattacaaaacttataagctttatctaaagaacatgggactatttttaaagtatcccaaaacttcacctttacactaaatgtattaacatgaccgtctttgtcagaaagttctaaagtgttaggtaagtttacttctaacccataataaacattgtcagtttgtactatagaaaaggtcattggtttaaaattctcaatagttctactaattcctaattcgttagtctgtaaggaaggattaacataagtaaaaccattatcagtcaaccaatattgcatataactaatttcataagcaacgttatgaacagcaacacttaaagttattactctcttatttttattcttctttatattttcttttttagcttgttcaaatcctatcatttgaaaggtataaaaagcctcgtctaaatcacacccaaatattacgttattcgtatttgtagcacctaatccaaacgcccaaacttttcttttcttgtctttttgtggataatctttatttaacattttttcatcaacgtaacaagtttcaatgtcaaatattaaatctctactatctattttactatattcagttacttctaaactatgttcgttgacatatgttaaatgtttattcattaattccacttccttatatttttattgtttcaatcccgttttaaatgtcgctagtaagtcctctaatatataacccatttcaacatcatgcaccccgtgagtgtaataacctttttcaaccatattttcttcgtagataaactcaaagtatttatctatattttcgtaaaaatcagatactttagatacagcatcatttgatgacataataaatttattaacttcatttacaaagttgtctattttgtcctttggtaaattcctttgaaatgctccttttaatattcccttaccgctaccatcaccaaaaaataaatcggtaactttattaactaattcctttctgatgttttcttccgcatcttctttaccaacaaactttaataaccctaaatgtgtatcacttataattttttgtttactctcatgtgttaaatctaatttgtttaaattttcgactatctttttgcgttcttttgttttctctctctcttttcttttttcttctttttcttttcttttctgttcattcttaacaaacctgtcagcatttcttttatagtctgtatctcttgccatatcaactattatgttaagtgtagacttatcataacctaataatttaatagctttcttttgaatatccttgtccattaaatagatataagcaccattttttacagctttgttaaattgtttttcaaagttctttttctcaatttgctttttcttactttttaatgttattctttttcttgccatgttttctcaccttcctaacgtttttacgtggtatgatatctataacatcactctctaaaaaatacttatcacctagaatatcaattacatatatgtcatcaccaatattataatttttagaatttaattcataataaaaaggttcaactactacagcgtgaaaagtttctaattccatgttatgtttattcctcactatccaccacaccccttatcttattagctaggtcataaagtttctcaagttcatcaatactcttagcttgtaccattctagtatctaaacataaatcaatagtgcttaattcatctagtgaaagctttacaatatattctttcaaatttgacacctctcttttattttggtataatttccccttaacataattctagcattttatacctatgatttcaaggtttttattgatttagttgtaaacaatttgttaactagaatgacaataaaaaagagccatgataggctcttttttatattaaatcatcatataatattataaacatttgtgatttattatcataatcaacttttttaactgcatactctttatcatgtaattctttaagatgtatattaacagtctcctcactttcaacacaacttaaatacatagttggtactatttttatttgtctcattttactcaccacctgtttaattccattctcttgatactgatatgattgtatatttttcttctaaatccattctagcataattcttagcttcttttatattatcagctgaaaccatagtttcgcataaactatcttttgtgttaaatttctctctgtaaattaccttgtattgtttcattttaaccactccttgtttggtttgttttgtttgttccttatgactatattatagcaaattatgttaagctattcaatgctttttatgaaatatatttgtaaacaatttgttaactagaatggcaataaaaagagccatgataggctctatttttataattttatacattgattaaattgttttttataagcatctaaatataactcattcttatccccattatacgttatttcataatacatgccatctgaaatagtagtgcttaataatgctttattattttgtaatgttttacaagaccatacgacataaacatcatcatcaaatggtgtggttttatatgttttagatatgtgtttcatgattaaatctttgcataaattgataaatttttcattatccataatatcacctctcttaattgatactttaattatagcaaattatgttaagctattcaatgctttttattgatttaattgtaaacaatttgttaacagatagtgctgttttatctactggtaataactggggaatatg

SEQ ID NO: 8 - DNA, 18171 bp, Unknown
  Clostridium phage vB_CpeP_ACP11, complete genome
gacatacacgtgttcccccaaattaatatttgtgttgacattaatcaattaattaatgtgttgacaattatttcattatatgttactataaatataacaaatcaattacacattcgtgttaaatcctaaacctatttcttaggtctgtttattaaacataatgagggggtaaaatgaaaacattcgaggtaacatatattatgtataatcgtaaatacaaagcaacattaaaaggaaaagacaagaaggaagtattaaggtttttcgcaatgtatatgttaggtaagttagtatcaattgaagaggtgataaaatgaacgactatgtcaaagacgagttatggaaattagaagtcactaataacatactaaaggattatgtagtacaatatgttgataacgaattaacaaggtgtttgttaatacaaaacttaaatttacaaggtagggtaataaacaatatacaagtggaggtcttaaagcatgagtaagaacaagaaaattgacagagagactaaacgttggattggacattgtctttccaggaatggttgttataaagacgaaatgtgtccatatatagaccaatgcaaacactacttatctaaacaatattttaaggggttttatccgccttcaatgttaggacatatagaattaaaggagatgataagatatgaaagagataataatagtaaataaggttaacggtgaaataaaccgtgtagggttgtatgtggttgataccaaccgtcgtatgaaatgggttaagtggttgcgtaagaaagattacagagaatatgcgttaatcaacaaagttgaaattttggaacaagaaatttaaaaaagttattgtctgttatggacatagatgttataataaagatgtcgaaggggaacaacactttgacaagtaaaatacaaaaagcatatagtggattaaaggaggtgaactcctccgtccacgtcttgtacactatcgcacatatcaacgacaaattttaataacaaggaggaaacgtaaaaatgacaaacggtttagtagtaattgacgagaaaatcaaggaactttctgctttaaagagaatagttaaaagaggggaagttacttacagaggagacaacagagaggaagacgcaagtaagctagaacaagcgttcaacatgtacggcatagcttataacagatatgaccacaaaacttctatagtaatagaaatcgaatagtaaacaaggttggcgacataccttaaatgtcgcacaataaattatcgtgatgaaggagagtaattaaaatgaatataacaagtgtaagagtaggaatattaccaaacccaaaaaattctatcgtaggaatggcaacaatcgaaattgacagatgtttagtattatctagcatgaaaattgttgaagggaaaaacggattatttgtgtccatgccttctatgaaaggaaaagataaggaaggtaaagacggctacagagacgtgtactacttcatggacaagtcaaaaattgataaacttagcgaggtaataatcgcagagtatattaagaaaagtggcgagagcatggagccagtaggagacggagatattccattctaatatagaaaggggcattatgccccttgcaatatgtaaacaatggtttatatattggagggggtatggtaaacaacaacgcacacacagaccgccaggggtggaacccgtctgtgagtggttcatgggttggcaccctccgttgttcggtctataccctttctaccattgacggtatttaacgtcacattaattcgtgttaggaggtaactaattatgacagatgaagagttacaagcattacaacaaactgtacttgatttacaggaaaaacaaaaaaactatgacttagaaaaacaagcatgggaaacagaaaaaactcaattaacacaaaaggttgaaacattacaaacaaccgcagatacttacaaagacatgaacgctaagctatcattaaacttagctacacaaatgacatcacctagcgttgtgagtgttcctggaaacacatcagaggaaacaaagccagttgacaacaccccttcactagacgatataataaaagatttataggaggataagaaatggcattagtaagaaacaaagaatttatacaagccatgttaaaagaccctaagacatctcaaaccgttaaaacggcgttaaaagatgttagcctagacgacatggagcttgtatttaataccttaacaggtatggacgacgtgagaaacgcctggatatctgatttagtaaacagatgtgtctcaactaggtttttccaaaaagtttatgaaaacccattaaaaatgttacatcatggtatgttagggtttggggacagtattcaacaaatatttgtgaaaatgggacaaagaaaagggttctacacaaattttgatgatagtaacggttcacccgaaaaagacctaattgggaagagagttccagacgttgaagttgacgtaatcaaacaaaactttgcacacagatataaagtgtcagtatcaatcgaagaactaagaaaggctttcatgaatgaaggtggtttacagtctatggcaagtggtttaataaactctaacatagatagcgcagaaacagacgagttcgaggacatgaaaggacttttaattagaagtgaagacgaaacagagtcaatcacagacccaactaacaaagaaggtcacaaatacgaaaaaggtgttgtatatcaaatcttagagggcgacttaaaaaataaggcggttagaagactaggtaaacaatacacacctcaacaaatttgtgaaacagttagagaggttgcgggtactatgagatttaagtcagataagtacaatttagcgcaagttaaaactttctctcgtaaagaggaattggtgtttgtaacaacccctcaaatatctgctaaaatagacgttcaagttttagcgcaagccttcaacgtatcaagtgcagatgtcaacatcagaacgattgagattgacgagttaccaactgtagatggtgaaactgttttaggtattgtaatggacaagtggttaatacaagcctttgacattataaacatggcagaacagttcaaaaacggagcgggtctatatgttaactatttcttacacaaacaaggaattatggcattatgtaaattcgctcaatgttgtttaataactgacggtactggagacatatagaagaagaaggggcaacccttctttttattttataaatgaggtgatattaatgttagtacaatgtttattaataaattgtccgttagaaatgggatacgagcatgtatttgattttgcgtcaagggagaaccaaatgtatttttttgaacataaattaaatgctagtagaataacggcggaggtcaatacaaaggttgacggttttttagaaaaaatgaatgtgaaatatccgtactctagtgtacgatatcacgattatataattgtaatagaaccaggaacagaaaaaccctatttcttttttattacaagaatgaaaatgttaactaaagatgtaacggaactgtctttaaaattagatgtatttcaaacatatcaatttgaatttgactttaaaacttctttcgttgacagatgtcatgttagaaggtgggagaaattcgacgataatatcccaacacctaataatttagacgaaggtttagagtatggagcaacagaggtgcaaggaatagaaactattaagaaaatgtctcctacttacattttagcaacaacaaccccaatcggtaaattatctgccaacacaggtggagggggaggaacaggtacaagcggttgtggtgacgtgtcaaagggaataccttctaataaatattacagatatttaaaaggaattgaaggactaaatcaatatccagtagacattggcgacggtgttacaacattcggttatggggttaccaaagaaaatgagccgacttattttgctaagttaggagcaccaccagtaagcgagaaaaaagcaagtgaagttttattcgagttgatacccgcaaaatacggcaacttagttgctagtcaaatgaaaaaagacgggttagatttaactaaggttgacattaataaattcgacgctttcgtagacctttgctataatacagggtattataattctaaattatacaaaatgtggttagcgaacgagagcatggacaaaataaagtctgagtggttagtatatgcaataatgccaggctccatatttgaggaaggattaagaagaagacgtaaagaagaatggttgatgttctccgaaggaacatattcaacttcaccaatcacaatacatggagatagaggacaatcaatcggaacagttgaaggcgacggttacatgatagaatgtgaaaatgttacagacaagtattttactgtgaacaatgaaggaggtcaaaactggattatgcccgccgaaggtaccgtgagcgcaacatatcctgtttatcctagcggttcgccacatagtggggtggacgtttcatgtcccgaaggcacaccagtacgagcaagtaaagacggcgtgtgtataaagcgtcgtgagattacaacatcttatggaaaatatttatttatagaacacgcagaaaatttaatctctatctatgctcacaatagtaaacttttaatcaatgagggcgaccacgtaaaagcgggggatattatcgcttatagtggtaacacaggaaacagcacaggagcacacagtcattttgaaattagagtaaatggggtcgcaataaatcccgcaccgaacttaaaagtaggtgacgtaataaaacataaaggggtggaataaaaatgcaagacaggtggaacacaaaaataaattattttgataacgaattttcagagggtgtctatacaggaatgtatttctatttaattgatgatagtaacaaagattgtttagacataatgggaaactgtaatgcaatacattcgttatggttctcccctattcttgaaagacaagacttgcaattgattgacgttgactatgatgtagataggtttggtaaaatagtggggatatctattgacaccaatccaaaactagacagaattttaactgtaaataattgtgttaaacagttgggttcttttaaatgctatgagcgtgttagaaccgagagtaaatataggaactggcaaaatgaaagtagattgtacaattatccttattcatacgccatgatatgtgatcatattaacccaccgtttgaagttcaatatcaccttgtaatgggaagtacaatgaatgtttgggcgaaggcttttatatctgacagaggtatgtactcaatatttttaagagggtataaaggagaccaacacggaactatggaggcgtccattaactccgcccctttagatatgccagtctcttcaagtgcttacagtcagtacagttctagccaaaaagcgtccgcaatgtttaacatgcaaacttctattgacaacgcaagagcaagcggttttcaaagtgttgcaaacgggattatgggtattaatcctatgaacccattatctgcctttggaagtgggatgaatataggaatgacaatgtatagaacccaaatgcaaattgaacaagctatcggtcaaaagtctgctatggaaaaagatttattaaacacaccacgtacaatggtcaactcatcatctgatattgggttctcactattgaactcaaataacaccatgcaacttataagatatcgtatcacacacgaatacctggagcgtttaggtgactatttcgcaatgtatggatataaacaatcaaaaatgatggttgtaaattataggaacaggtattattataattatataaagacaataggagccaacatcacacctaaaaactggacaggcataccaaaggaacatttaagagaactgaaagaaattttcaataacggtgttacagtttggcatatggacagagagggggtcgagttcctaaattacgattatgataattacgaaatagactaacaaaatgtttcacgtgaaacgttaaacaaatgtttcacgtgaaacattaatttgtaagaaagtgaggtaataaaaattgaaagaggtaacagacgtaatcgcaaccgttgggtttcccatagtagcgctaggatttattatgtattatggaggaaagtattttataagttttgtcaatcgcataatggacgaaaacaaacaaagagaagaaaacatgtatagatttatggatagcatgagtgataaaatggagagagtaagcgccaacatggaaaatttaggcgacatgattaaaaatcatatggaaaaggagcacgcagaaaatgagtaaaagaaaaagttactggaaccagggggacaaaattttattaaaaaatgatgtctttatggttgatagatattatgattactatagtaacatgggacttaaccgttttaggtggaaaaacctaccaccaggcatggaaagtcgacacatagaacaagcactatttaatgaaggacaagcggtattttttaagaatacagaccctaacgagccatacgggtttttatgtttaccgtgtgcgccctcaaatggacagaacatttacggcgaccctgttcactttaacggcataggtgttaacaagtattttacaaatttatcacccttgaacgccgttagaattttagataatgacaacggacttgcaccagttcgacacatagcatattacacatatctaatgtcacaaatagaaatgaccataaacatgaacttagaccaacaaaagtttcccattataattggagcaacacaaaagaataaattatcaatggaaaacttatatgaaaagtattctagtttcgagcctaatatattagtagatgaaaaacttgcacaagcacttcaagaaggtaaagggttcgacgccttaaacacacaggcaccttatttgcttgacaaactggcagactttaagaaaacctgtgagaatgaacttttaacatttttaggtatcaataataccaacaacgataagagggaaagattacttaccgacgaagtaaatgccaacaattcacaaataactttcgtgttagaaatggcatataaaaataggctagacgcatgtaagagaataaatgaaatgtttggtttaaatttagaggttgagaaagttgtcaatttactagaagttgacatgaaaggagatgtaaaaaatgaggggattaatggagagtagtaacaccattgaattaaggtacttagtcaacgacccaaattttgacatatttgattttccatatgacttttacgacgacaacctaagaactgttttcgaggagttatttattcaaaaatactatttttatgaaataggttgcgaggttccacaaaggtggaaacatatgctaatggcacacttaaacgaggtaatgccatactatcgtaaactttatgaaatagaacttgcacagaaggggattaacttcctattaaataaggatttaagggaagagttttcgagaaccttaacagaggacgaaaaacaaaaagaaaatatatcctctacaaataatagttccaataatacaactggaaacagttcaaacaggttcaaagaaagttcacttgataatggaatagccgacttatcagatgataggataacaactgtcaatgacaacatggttaattctacaatggcaaccatattaaaagacaatggattaacagacagagcaaacaataaacttaacaacttaatagaaaaaactgtcctagtttctcaaggtaatatcggtacaacttcatctgcggaacttgttcaaaaatggagggatagtgtaatcaacttaatgcaaatgctacttgaaagttgccgtgacctgtttatgttagtcttttaaaaataaaaatgtttcacgtgaaacatatacatgttagagcctttgggttcgtaccctctccgagggtacgcctaaaggcttttaccaaatgggtggggggttggcacaatttaaatgtaacaaggaggaaaacaaatgtcacattttgaccaaaactataagattgtaaagaaagaagaagaggaactgtattttaaggtgtacgcaatgtatgttgacaatagaattattgaaaatgctacttgcgtgttattggacaaagacagtataattaaggaagttaaaccaattgacattgacgaagacggtgtgttctttatgctaggtaaagacattccgaaaggtgaatatatttattatatcgtcggacatgttgaaggggttgacaaaattttaatggataggagggaggtcgtaataatatgataccaacctataaaagttttagaataggagccgttgaaactggagccagggggaaacctggaaaaagtgcttacgaattatggttggaaaatggtaacaaaggaactatagaagactttttaaattcgttaagaggtcgagacggtaaacagggaagacctgggagacaaggagaaaaaggagaaaagggcgaccctgggagaatagacaatgtaagattttttatttcagaagacggacatctacacgtagaaaatttagagtaaaggagaggatataatgtcatctttagatttaggattagtaagaggagaaagtgctttcgagacatggaaaaacaacctggtaatagtggtaaaaatgaaagtcaatttttagaaagcttaaaaggagcaaaaggagacccaggacaacagggggcaaaaggtgagaaaggagacccaggggcaccaggggcaaaaggtgaacaaggtatacaaggtttgccaggtgcaaaaggtgagaaaggagacccaggagcaacaggggcgcaaggggcaccaggtaaaaacggtcaaacacctagaattactatgagaatagatgaaaggggtcaccttttagccgacattacttacgaataaaaaggaggttaaacaatgatagatttagggaaggttaagggtgaggacgccgttgttaactctaaccataacgggttgttaggtacggttacgctaaatgataaaacatgtcttcaatggggttattttacaataacaaaaggcaatacctgggagcgtgttaatataaatgttccgtacaaagacgacaattataacatacaagttacagagtattacgtggacgggtacaaacctgtttacattggtaatatagataaagcgggattttccgtttcaactgacggtgacatagacgcaccaggtggaaaacaaaatatttattggttctgtataggggtggtagaataatgaaaataatacaatcaaatatccattttaatggtaataaagcgggaggtaataacccaaaggaaattatcgtgcatcattcggagcatagtaccgctaacgtgtacgatatcgacaggtggcataaagacaagggttggtgtggaattggatatcactattttatagataaacaaggtaacatctacacaggaagacccgaagactggacgggagcacattgtatagaccataacacaaaatcaatcggaatatgcttacaaggtagattacagaccgaaaaagtaacagacccacagtataaagccttattatggttaatacaggacataaaaaataggagaggtaacatgccagtttatgggcataaagaattaaactcaaccgattgtcctggaaacctggatttagacaagttaagaagagatttaaacaacgaagttgttgacactaacgacgacgaatacagagaaaatgcaacagttgttaacgttagttcatatttaaatgttagaagtaaaccgtcagatgaaatgattggtaaactatttccaaatgagagactacaagttaattgggttgatagtgattatttagggtggtattacgtaacatatagagttaacggaaccaacaaattaaagaatggttatgtgtctgcaaaatacattaaaaaagactaaattgtttcacgtgaaacgttaattaaatgtttcacgtgaaacattattttaaaggagcgtgaaacaatgattaactttaatcaacttgaaacattaaacaaatgtcttatagactggaatgttaactattctgtctatccatataatggttacacaattaacgaagtcttatgtcagtttttcgacgcaattaataagggaataataacaattaatgaatatactaaattgataagtgcaatcatgaactggattaaagatgaaggattaaaagaagaggtcgaaaatgcacttgacaaaatgatagaggacggaactttcgacgacattataaatgaaaaacttttaggcgacatcatggaaagactagacgagatagaaaacattaataaggaacagaatagggcaatagaaaaaaatactaatgccatagcagaaattgacaaacttgtcaagtcttatggtttcgttactgtcaatatgtttggagcaaaaggagacggagagacagacgacacaaaggctttcaatgacgccataaattattgcaaagaaagaaacattacaagcttgtttattccatatggaaattacttgacaactggtttaataaatgctacaggtgttaacttagttggaatgtataaacctgttatccctttcttagaatgggaatacacaagaccaacatcacaacttgaccatttcgagaaatattttaagatgtgtaaaggttctgttattggttcaaaggataatacaatctttgtaggtggtttagactgtaagaatataggactttttggcaatagaagaaaagaaaatcaaagtggatatttacaactagaaggtggttacggaaaaggtgtaaatatgaccgattgttttgttagtggttttggtggtgtaggtgttaaagccgactatggattaattagttcacaattcattaacacaactataacacaatgtggtaaagagggtttatatataggtaagataaaaggtacttacacaggtgaaacaaacttcttgacaatagagcgttgtactatctacagaaacgaaagtcacggaatattagcagatgtcatgggacgtggtttcaacataatattaaatgactttgagtttaacggtgaaccgtccgaccctcaaagacctaagccaacatctgttgacgacatgatttttggttgtaaattactggtgagtggtaatggtggattttcaagtggagcaataaagtttgaaggaaactacacagaggaaactttaggtatgttatacattaagaatactggtggtacttgccagggcgtcgacattaattgtaatatggcatatccttacaataccacagaattagaaaacattggtatttatttagacggttggatagatagaattaacatatcaaacaataatatgtatttcgctaaaaaattagttttaaatggaaaccaaatccagtcaataagaacagacatagaaccaatacatggtggagatactactggaagtgtggcatggcaagtcactacagaaaacggatataacaaaatcgtgatgaacagtttatatgaagaagaactatttagcaaaaatggtttcataactagagaaggttaccacgaagacggtgttacacattacccattcgataaatcaaacactcatataaatggtgtggaaattgattacgacgacgcacgtaacccaatggtcggtaaaatcttaatactagacggaaactatgtcggttatgttcattcagtttcatggacacaaggcgttatcaacatacgtggtaatagagttatctcacgtactagcggtaatgttaagtttgttgattgtggagggttcatcactagagacggaacaggaacaaaaagaaaaatggttatcgactgggacggtacagtagttggaatataaaaagaagagggtgtaaaccctcttttttgttttataaatatttactaacttcttcaacaaacatctttttagttgactggttcatgtaataaacatcaccatttaagaaacattttttaatgtatttaatattccaatggtttctatctgccttgatgttcctccaacctgtgtaattattgttagataatatggtatagttccacctgttttcttttaagtatttgtctcctaaccatacgcccgtgtcagttgctccctctgaccatgctccgataatcctttcgccaattctaaatgctcccctgtaatagttgaaacctaacggcttgtttggaacaataaagtcgttggtgtcttcaagcgtattgttatcaaatgcgtaacttgaatatgttgaaccctttaacaacctagcaaacttggttttctttttcatttccctgtattcttgtgtgtctgttatttcaacacataacaacacgtcaccctcgtctttgattgttataaacttatctgtcttattaggttcaatttcaaagaactcgaaaaacggatttacataagaaacggcgttagcacaaatatacacatgagggtcacgtgttctgaaaactgtttctactaaatcattaagtaaaatcatttcattctttagataatcattcttacctgtttttgtgattatatattcatcaaaaaatataaagtcaacgtcgggaaatgatgaagactttttctttgatgatgttgacaacgctataaaataacaaaaacattgaccgtcgataaatccttgatacccttttacttctatgttaatccctgggtaatctttcgctatatcattaaacacggtgtcctttacctcgtcaatttccgtttgagttcttcttacataaattgactgtttaccatgttttaaaaatagatttatcattttcttttttccctggtatgttttaccgttacctctctcacctaaaacaaaactataaacggcattgtaactcattaatttatccatgctatagtaaacatttttattcacttcatttctagcaaaactactcattatattatacctcctatattgtttcacgtgaaacattttgtttaacgtttcacgtgaaacatttattaaaacattattttctttattgtaaatagtgtgtccaataacaggcaaccgcctattaccttcttacgttgttttttaccttcaacatttttccctagatagaactcgtcaaacccttgtcctattattatctttctagcgtcggaaggaagaccacaacattttaagtctgttttatcctctttacagtcatgatacatgtattttttctgtcctagaaccttgaatttatcaaatacatgctccacgtcccatttacctaatattgtcttgtcaatggtttcgcctattgcgttcatatcctcaattaaagagttcacctctctgttacagtaaatactatctgtgtcgcaatacaagaaattttcgacaccaacggcgtatattattgcgttccataattgtaaacgtccgtatgcggttacgaaacttgcgtatggacgataaaattctttaccctcatactctgttacagaaccagtaaatgtcaataacccgtttttgtccatgattaaatctttttcttctttgttttgtttagtcccaaattttccgtacgcaccgtttaggaacaatttcgcctggtttgtcaagggtttatttcctagttttttgttctcaactttcatcttagtaaaatggtcaacataaggtttaaattttcctttctctgccttgtaatataaaatcgaacctattttaagacctgtaaattctaaattgtcaacctcgaaacaatcatattcatctttcttaaacacaccgaaattaaagtgctttatccaaaactcatattctacacttgttaacacaacgttatagtttgtagttaactttgtgtctttaagttctttaaacacaggaatagccttaccgtcttttatgttagtacaaaaatattcttgtccactaacggctccagtaataggagacaatgccttagaattaaccgctcctatcttaaaaatatccaaagcgtattcttcgtgtttaggttctacaaagtcaaagcctacctctattaaataaacctcatttttctctgttttaggtttacgcccccatgtcttacgaactggtttcccgtacggaaaaaccttgtatgccatctgtgacggataactagagtttatgtctagggaacaacctatcttatttattgttttacccaccgccttatgattggcatgagtgtaaccccctgtgtaactctccatttctattctttttcttaaaaactcaaatttagttttcttgtctagttcaaaataagctatatatccttcctctgtcttttcctctccaaatgtcattttctttagaacattaaaagctatagaactagcggtgcgcataccagttaaaggaagttcacccccacataaaccgtcgatataaaattgttctataacttctcttaacataaagatgtcgttatattgatatcttaattcaagtgttgtttgtttatgtgttggacttctccatgtatcatagtcgtaatcctcacccattttataaaacatagggtcgacgtcatgaacgtaattatggaactgactttctgcacatgtgattattttgtaactatcaaaaaaatcaagacataaacctatttcagttatagacccgtctttattctcaacctcaaaaaacttgtccatgtaaacattacaaccatatactatattattattttgcactatatggaatgttttaggttcctcaacatctgtaaatgtttgatagggtgctcccttactaaatacagtttttaacaatcctttgtcataattaaaaccgttaaaaactaaactatattttagaaactcaacgtcccaacccaggttatgaaccgctataggaaacttaatgtatttacgtttgggaaaacctttcttagttttggttatttttatagtatgtttattatcatgataccagtcgttaaaaatgttttgacatacctcccaaaactggtttaaattttgaccatatatcatattatggtttcttgttacacctaacccccaaccatacacacgagcacctgtattatcattttcatagtttgtacaactttcagtatcgaatattagacattctattttatcaaaattagccttcttatattcatcataattaaaactatgctccttaacatattgtgtatgtttgtccatttataacaacccctatcttaataattctttatacttattcaacaactttcttgatgtgtcctggaagaaggacaatggcgccttagcgtgttttgtgtcatattcgggaggtattatgtcaccgtttaacgtttgttcctcgtatttttgtggaattattttttcatacaaatatgtcattaattctgacatattgcctaaagcattatcgtcctcaatcattaatttttcgatttcttgtagattttcttctacaaagtcctcctcggcgtgattaacgaaaaacgccccgttcgccctgttaataccttttttgtctgtgtactccatttcatcagttaatgtctccatgaaatgttttgccacttttttgtgaacatgtgtgctcccttttctaacgtctcttattgtgatacctatttcgtcctttctagcaaaatacttgcctgtctccttaccgtaaacgtcatgaagacctttctttatttccttttctatttgcttgtctcttttttccttataagcctgtagttcattttgtattttaatgcgttctttttctctacttctttgttgacgtacctttgtcatgtaacggtcataactctgtttactctttgcaacatcatagatgttaccacttctattcttaggtaattcccagttgttctctttaaattcacgtaatacctttgttatttgtttttctctgtatttaaacttattcatgttaaccacctcatatatattatagcatagtaattttttcatgtaaacctaaaaagagggtaaaaccctctttttatctattttttaatccattttattgtagtttcatctatgacacctttttcctgttttaacacaggactatatttaccgtcacacctcattatatcgtaccattctgcaaacctttgaaagtcaagccatgacgcctgtattttaaaacctctcttcttacaatatgtgtattgtagtcttctccagtagttacccgcggggtgtgtactaatatgatatttcccttgactattataaaatttttgttccatatgtttactcttatacttgtccaacttacaaagagatttaaatattttttcctcccagtcgtaacctggcacacgtccctcgaaactgttctttaaatagtcaatacacatttctctataaggacaaacaacgtcgtccttagagatacaatacttattcgttttgcaatgtctttgcatttccttaaccttattcctcaattccttcattttccttttcctccctctcgataagtctctcaatatattgtttagcttttttcaagtcttcaatgccgttcttgtacttgtatctagttatatacttaattatgttgccttctaagaagtccattctccaactgtccgtatagtcccaaacctctatgccgttgttataatgtccagggtgttgcaccttgtcatgtgcaaattcacgtattgttaatataggttcaaaatactctttcttaaaccaatcacattttacctctgtgttacctttagcaatgtattgtattaaatacatatcgtcgctttcgtccttagtctcaaatgtcatgttttgtcttatagaccatgttatgtctttcaaacttctaactaacattatttctcctccttattatatatacaatacatgtatattaacataaataaacaacctgttgcgtatcctaatataaaacctaatgccattttactcgttctccttttcaacttctcttataatttgtgctatttctaaaattggtatgttacaaggttcaacgtataaatagtaaccacatacacgtctacacttacaaaacttacattcttcaactcttagacactcgtcttttaagtctgttgctagttatatactttgtaacctgttcacggcttgtccctccttatgataaacagtagagcaatagtatgactattgctacaactgttaaaaataaattgattttgaagtatgtccaaatatcgtcccacatgttactcacctaatacctttccacaacctggaaagtctgtgttatagtaacagtaatgtatcatgttctcgaaatgttctgcggtgtcttctattatatattgattgggcacactcactttactaccgtacacgttgccgtcaatccacttctttaagaatatacttgttacaccgtactcgtctataaatgaaaccatagtatcatcaatcatatattctactgacatgttgtgtcctttccatttagtagccaatacctttgccacttccttaatactgttcttaatgccttgattattcattatctatcacctctatatttagtatatcatgttgacacgtatgtgtcaacacttttataaattcccaagacattcaatgtaactgaatgtcatatcctctagttcgtctgcgtctatatcatagaatttaacaaccgcataaccttcctcattatttatgcttgttattgtacttgatacatggtagttgctatataatgcgtctgtaacctcttgagctttagcaagtgaatactctttataacctactattttaactaatacgaagtctttaccttctgaccatactttgtaaccttgttcttgttccttctcttcttcatataaaaactttaatgtaatgcttaattgtgttctatagtcaacaccaaactctttaactaaacctcttgttatgtgatgtgcttttttcattaataatttttatccatgatactctctcctctcaacttcctttaccttatgtattaagtataacatgccaacacgattgtgtcaaccctttttgcaaaatttaattaa

SEQ ID NO: 9 - DNA, 17692 bp, Unknown
  Clostridium phage vB_CpeP_ACP22, complete genome
catattccccagttattaccagtagataaagcagcattctctgttaacaaattgtttacaattaaatttcaaaaactattgaatagcttaacataatttgctataatatagacataaggaacaaacaaaacaaatcaaataaggagtgattaaaatgataagtatttatggagaaataagaagtttaataaaacaagggaaatcaaacaaagaaataatggataaattatttgtaacacaatcacaagtacagtatcaaagaagaaaaataaaaaaggaagctatagcaaattggaatttataagaggctttacgcctcttttttattgtcattctagttaacaaattgtttacaactaaatcaataaaaaccttgaaattataggtataaaatgctagaattatgttaaggggaattataccaaaataaaagagaggtgtcaaatttgaaagaatatattgtaaagctttcactagatgaattatgtactattgatttatgcttagatactagaatggtacaagctaagagtattgatgaacttgagaaactttttgacctagctaacaagataaggggtgtggtagatggtgagaggtaaacataacatgggattagaaacttttcacgctgtggtagttgaacctttttactatgaattaaattctaaaaattataatattggtgatgatatatttgtaattgatattctaggtgataagtattttttagagagtgatgttatagacatcataccacgtaaaaacgttaggagagtgagaaaacatggcaagaaaaagaataacattaaaaagtaagaaaaagcaaattgagaaaaagaactttgaaaagcaatttaacaaagctgtaaaaaatggagcttatgtctatttaatggacaaggatatccaaaagaaagctattaaattattaggttatgacaaatctacacttaacataatagttgatatggcaagggatacaaactataaaagaaatgctgataggtttattaagaataaacagaaaagaaaagaaaaagaagaaaaaagaaaagagagggagaaaacaaaagaacgtaaaaagatagtcgaaaatttaaacaaattagacttaacacatgaaagtaaacagaagattataagtgacacacatttaggattattaaagtttgttgggaaagaagatgcagaggaaaacatcagaaaggaattagttaataaagttactgatttattttttggtgatggtaatggtaagggaatattaaaaggagcatttcaaaggaacttaccaaaggataaaatagacaactttgtaaatgaagttaataaatttattatgtcatcaaatgatgctgtatcaaaagtatctgatttttatgaaaatatagataaatactttgagtttatttatgaagaaaatatggttgaaaaaggttattacactcatggggtgcatgatgttgaaatgggttatatattagaggacttattagcgacgtttaaaacgggattgaaacaataaaaatataaggaagtggaattaatgaataaacatttaacatatgtcaacgaacatagtttagaagtaactgaatataacaaaatagatagtagagatttaatatttgacattgaaacttgttacgttgatgaaaaaatgttaaataaagattatccacaaaaagacaagaaaagaaaagtttgggcgtttggattaggtgctacaaatacgaataacgtaatattcggttgtgatttagacgaggctttttatacctttcaaatgataggatttgaacaagctaaaaaagaaaatataaagaagaataaaaataagagagtaataactttaagtgttgctgttcacaacgttgcttatgaaattagctatatgcaatattggttaactgataatggattcacttatgttaatcccagcttacagactaacgaattaggaattagtagaactattgagaactttaaaccaatgactttttctatagtacagactgataatgtttattatgggttagaagtaaatttacctaacactttagaacttactgacaaagacggtcatgttaatacttttagcgtaaaggtgaagttttgggatactttaaaaatagttccatgttctttagataaagcttataagttttgtaataacttagatgaaatgttttataaattgaaagaagagtttgattatgataaaataagagaagataatcatattttaacagatattgaaagaaggtatctgtataatgatatcagaatactaaaagaagttataaatgattatttttacaactcacttttaaaatttgaatatgaaggagaagagatttacataagttcaaattgtaaaacaagttcctcaatatcgtttaaatcagctttagagataacatattttgatgaacgtaacaagaaaaaggcttttataaatgagttcgaaattgaagaaagacagatgcttttaagcaaaatggctagggaaatggagaacaaaagttatgaaggtggttggacttggtacaaccctaaaatagttaataaagttgtaaaatgtaatggttcaagctttgatataaattcaagctatccatttcaagttgaaagtgctttattgccttatgggatgccaaccgaatacaaaggatatgttaaaccagttgaggatgaagaagtagcaatttataatataggttttgattattttaaacctaagagtaaagagtatgaattacaaatgattagattaggagcaaagaacttttctgatactgatttacctttactttatagagataaattaaaaatggttcttatgaatggtaactcttttatacatactaatataatagatggtagagttattgaaatttatcgtaatggttgtgaaaatttatgtaattataatatgagtattacaagtgtagaacttgaacatttaacaaaatattatgattttggttattatgaaaatgttgaggttgatggttttttatttaaagaaaaagataagattaaatttaatggtttaaagtttggtactagcttaatatataaggctgaaaaaggtattttttcaaactttgtaaatcacttttataatttaaaatcagagataaagcatagaatagataagggtgaaaaattagacgctcaatattcagcgataaaaactatcctcaattctttttatggcaagtttggaacaagaaccacacgtacaattgattatttagtaaaaaaagataaaatatttacattcgatagagggggtaaagagaaaaatttagaaagggttaaaaagttaaaaaatgaaaaaagaagaaaccaaattttagcaagtggacaatatattgaaacttatgaaagtgaacccttttataaaccctttgctagttttgtgacagcatacgctagagtatatcttcaatcaatgataatagaaggttgtggcgttgaaaactttttatacagcgatactgacagcttttactgttctatacctaaagaaaaagtaattgagggattaaagaaaatgggtgttgaagttcaccctaacaatttaggagctatggataatgagaaaaactttaaagagtttaaaaccataggggctaaaaagtatatgcttagaactaatgacggtaaaataacttgtaaatgtgcaggtgttccaaaagacgcacaaaagatattatgtaatgaaggttttgaagagtttaagctaggtaaagaagtaaagggtaaattagcacaaataaaatgtgatggtggacttgacttagtagaagttcaatatataataaaagatttaagtttaaggaggtaacatgggtaagttttttgaaataaaagagtttatagaagaatacccaaaccatttaatacattgcgttataggagcaaggggtgttggaaaatcatactcaacgaaaagattaataatagaagagtttttagaatatggtaaacaagcgttgatattaaggagatacaaaaatcaagctgaaagtatggcaacaacatactttacagatgtattgcaaaaggaatttccaactgttgaatatgagttaaagggcgatatgggatttataaatggtgaaccattttgtctattcacaggactaaatggtaatagcttagctaagggttcatcatttcccaacgtttattatattatatatgaagaagttatgcccgagcctggggagaaaattataaaagatgaatataagaaattagaatctataattgtaacagttgatagatttgaggatagaatagaagtgatatgtgttgggaataatacaagttattacaaccccatttttgatactttaaagttatatccttcaaccaaaccgaacagagttgtaaaaaatgagcttatggcaattaaaacacttgaaacccctacagagtttaaagaatacgctttaaattctaaggttggtaaattaactgtaatgagtgggtcgtcacgttataatatagacaatgaaaatattagcaatgatagatttaacgtttgtagtaaaaaagaattatgggatactgataaactagagccagtttttaaggttcgtgtagatgttgacaaggtaattaaaatatggaagtgtcaaagcgatacaatgttttattattgggttgataatcataataaaggtaaatgtgacgaatactttttagatttagattatcaaacagacgaaaataagcatatatctttatgtaataaagggcttataaatagaatcaattttaataataaggttggtgctgttttctttaataatgctgaaacaaaattttacttcaatcaaattggattattttttaaaaaataatgcaaaaaagtgttgacataatatataaatgtaatgtataattaactcataaggaaaaaacaaaccaaaacaacaaaaatatttaaaattaaaggagagattttaacatgaaaaaattatcaattttcaatttaggaaacaacgtttcaagtggggtatattttagattaaaggataacagagaaaaagctgttataactttcttaatcaataatattgatgaagtagaattagacttagttcatccagtaaaattagcagatggtagagaagtaaatgttaagtgtgtggctgaattagacaatgacttaaaagtaattaaagatacttgccctctatgtggagaaggtttcaagatgtcagaaaaggttgctgttaatgtttataatcatactactaagagcgtacaaatttggcaactaggtaagactttaatagaaaaattaattttcttagctgaaaaaaggcccgaccttattaaccacacttatgaggtaacaagattaggggctaagggtgacactaatacatcatatgaatttgagtatttaggtgcatcagataagaaagtatttgaaggtttagagttaatcaagaaaaacgaaacagtatattatgaagtatcagacaaagacttagaaaacttcttaattactggagatgaaaaaatatttgaaagaaataaataatttaaaggtgggttaattcccacctactaaaattaatatagaggtgaataaaaatggaagaaaatagagagttaacagaattagaaattagaaaggagttagctttagaaaaagttaatgaaaagagattacaacttaatagagtagtttcttttataaatcaagaagaaaaagagtttccaaaatctaaagaacatgaggctgtaatgtttgatacgcttttaacttctttaggaatagagtttaaagtaacagaaactaaaaagagaataatatttcaattagcataatgtaaagtatacccccttacttagctatataacgtaaatgattaggaagtcacacggtagaacgtgcctaacatttttccacctatgaccgtgggcgttgtatagtgaagtaagggggtataatattttgtaaggaggttttaaaatgagtaaagaaatgactcttgatgatgcccttttaagaattgttgaattagaggaaactgtagaaaatcagaataatgtaatcagcacattaaattctgaaaaagaaactaattcaaaagaaattgaaaaacttcaaaaaagtaacatggaattatttcttaaagttactaacaaaccaaaagaagaaaataatattgaaattaaagaagatagtgagtcaaaaatggacaagctattaaaagaatgggagtgataaaatggctgttaaggcaaacaaagaaatcatggaggcttttttagaaggagcaaccgaagaaactaaaaaagttttgagtcaagtagatttatctgatagtaaagctgtttcggatgctttattcaattatcaagttgtaaaaaatgagtttataagttcccttataaataaaatatttatgactcaaatacattcaaaagtttggaaaaatcctttaaaaatatttcatggtggaatggaagaatacggttatactgtagaagatatgtatgtagaggcatcagaaaaagttgggtttaaagaacattttaacggtggtgatgaagtaaaggacatagtatcaaccttagttcctaaagtatatactaattatctttcaagaaatttcgctgataaatataaaataacaatatcagatattcagttaagaacagcgttcagaaatgaatatggattagcaaacttagttaataaattattagaggctaacttaagaggggcttatcgtgatgaatataaatatatgaaagatatgttttttaaatattgtaagggtttaactaatcaagatgttggtatgagtacatcacctacaaagactcaatatattcaagactctcaagttattaaattaaatgatgatgcaaatcttgttagaaatctttgtaaatcattaaaggctatgaataaaagattacagtttgaaagtgataaatataatagtgctaaggtaatgcaattttcatctttaaatgactgtgtatttataacaactcctgaagttgaggcagagttagatgttgattttttaggagatgttttcaatctttcaaaggtagaggttcaacaaagaattatactaatggacgaactaccaacacaaataaaaagcggtgagagtggtacaaatacacatgagggtgaaacttgtattggaatgttagttgataaagaccttttaagatttaaagattttgtatttgaaacaagatattttaataatccaaataacttatctactaactactttttacacaaacaaggattatgtggtatagtaccatttttaaatgctataatatacactaaatcagcatcataatgaggtgatttaaaatggaaaagttaactaataaggaaatttacgataaattaatttcaaccatgcccaaggatttgggcaactcttttccttcttatgaagaggctttaaggttgaatttaataaatattgatatgaacagttttattgaaagtatatgcaataaaatagggaaaatgttatatatagaacaacatttttcaaaccctttcaatgaacttttttatggtgaaatgccattcggttattcaattgaggagttgttcttagaggactcaactgttcaatctttattagaaggtgatgaaaatgaagttttcaactcttctttaccaagactaaacggtttagttgcaaatagtagatttgataaaaaagttaaaatgactcttagtgttgaaagattaaagtcagcatttttaaaagagtttggattatatgatttaatatctaaaatgttatccaatttaaaaacttctatgagtaaagataaatacaaagaaatagaaaaagctttaaaggaatttgctaagggtttagactccaacggtgaaaatcttaattttaaacaagtcccatataaagaagttgcaagttcaaacttaattgagagcattgttaaaattgtagaagagtttacactaccaagtacaaaatacaaccctagcaaatttaatacgcaatgcttaaaaagtgacataataatatttgttaaacccgaaatttatgcaaagttagtaagtacagatagcatacttttaagaggtgaatataatattaggtcaattactgatttaccaactgatatcactaatttatctacaaatacaacaataaattcaattgctttaattatggataaaagattaatgcctataatgttgaattacaatggattagatagaattgacgaccctaatagtttaacaacaaattattattactatgaaaattatagtctaccagtaaatactttttctaattttgcaattctaacaagtgaataaaaacgtatatttttattctttaccttatgttgacccatcatatcaaaacctttttaactttaaaagtataagtaatcaaaataattatttttcttctaaagtggtatgggtgggtcagtataatataaaggtagagccttcccgtgtaagtttagtggtagacaaaccttacgggtggtttatggagaataatattaactatatttcagcttatgatactaacatgaaaaatatttattatttcatagatgattttgtttataaaacagaaaactcaacactattagtaatatctgttgatgtatttcaaacttatcaatttgactttgaaattttagaaagttttgttgatagatgtcacgttaatagatgggatggtgataatccaacaaatgaatatgaaactgaagatatttcttacggtgaaaatatcatgctagaatatgaaaaaatagctgatatggggcgaggtgttgttgttacttctacagtacctttagggaaagtcgaaacaaatgttgggggtggttctgatggtactggttcagcaaatggtgatatagcaaatggaataatatcagcaaatggacttttatttgtaaaacaagaagagggttttgcagaatatggagcatatttcaatggtgaatcttttaaaactggtggctatggtgtaacagaaaattaccaaactaaatattatagacaactagaaccattcccagtatcagaggaaaaagctagtcaagttacctatgatttattaaataatgaatttgggatacccgtcaaaaatgcaatgttaaaagctaatataaatttaaatgatataccaatttatcaatttgatgtttgggtatcaatagcatttaattatggtatgggtggattgagtaaattaaatgcgtggcaaatgtttttagctaatccaaaagacacaaaaaatatagcaacagcaatcaaaaatttacccgccaacccaaatagaagacaaagagagggtgcactatttgaaagtggtatttacccccaaagacaaattttaaagtatggacaaaatggtcaaatagttggatatacagatggtaacggttggttgcctagtggtaaaaaagacggcaaatatgttgataatgatgctgggacaaactggttaataccaacaactggacaaataagtgcatattacccaacttatccaagtggaaacccacacaatggtgttgatattgcaacaccaactggtacaccagtatatgcaagtaaagacggaactgttattaaaagaagagaattaaccactagctatgggaaatttttaattatacagcatggtgactcacaagttgtttacgctcataattcagagttaaaagtaaatgaaggtgacaccgtaaaacaaggacaattaattgcactaagtggaaatactggaaactcaagtggtgaccatttacattgggagattagaaacgaaaagggaacagttgttgcaaatggtgttaaaactgttaaccctatgccaaactataaggtaggtgaccaggtatgacaataactaataaagattatgatacatgggaatcaaaaatgtatggtttgccatatgctgtatatagctattatattgactctacagtagcgggtgaaaatacagctgttatcggtgcaagtggtgataatgtttttagtgttgtttacacaccatttttagatattgctgatttagaactagaagaaattccatatgataataaaaggtttggtaatatttctgaaattagacccgagattaaagcaaatcctcacgttttcagaatcaaaaaattattaaaaggttcaaaatttgttggcgagtttgaaacttacaaagtaaaaaagagtgtgggtggtaaaagaaactggagaaatgaaagtaaattatataattatccgtacacttatttaatgctatctgatggcataaatgacccattaattttaaagcctcaattttgttcaattcctaactgttcagttggtataaaattatctgtttcagaccgttgttcgtatggtatatttgtacaaacttataagggtgatacagacggaatgacagaggctttggtatcaaatgatgcacttgaattgccttgcacaagttcagcatattcaaattgggttgcaacttcaaaaaatcagacagcacaatcaattcaaaatactgtaaatcaaacaattttaaatgataaaatagcgaaaaataacatgaatttaggtatagcaaatagtgtggttggtggtgttgctagtgcgtttactggtaatgttggcggtgtaatagatagtttatttggtggtgttggttcatacatggataaaaaacatacgaatatgcagagtcaattaactagggaatcagctatcagttcagctttagcaacagcgaacgacatgaggtcaacacctaacactttacttagtcaaggttcaaatataatttatggtttacgtaatggtggacaagagttaagactttataggtatgggttaacagaaagatattatgaaaaaataggggattactttgcacagttcggatataaacaaaataaaatgatgaaaattaatattaattcaaggtattattataactatataaaaacaataggaattaatataaaaactaataaaattcctaataattacttaaatatcctaaaaggtatttttgacaatggtactacaatatggcatattgataatgaaggcgtcgaaattggtaattattctatggataacagggaggtttaaaaatgaaaataggtattagagacggacatagtccaaattgcaagggagcgattgctttaagagatgaacaggcgtgtatgagaattttatgtacagaagtaattgaggttctagaaaaacatggtcatgaagtagtttattgcggttctaatgctagtacagaaaatggtgagttaagtgagggaataaataaagctaattccaataacgttgatatatttatctcacttcatatggatgtttcaaaagaacataaagggaatggaacgtgttcatttgtagcaaaaggtgcaagaaaatcaataagagatattgcacaaagattagttgataactttgaaactttaggtttacaaaatagaggtgtaagggaaagtaattatagagaaatgagagaagttaatgcccctaacataatatttgaaactatgttttgtgataaccatcatgacataaacgaagtgtggtcacctacaccttacgaaaaaatggctttactaattgcaaatgctattgacccaactattaaagaaaatgaactttatagggttgttgttcaatattttaacagcaaagaagatgctgaaaactgtcaacaagaaatcgctaaaagatggtattgttttgtggaggagtgtaattaatgcaagaaataataaatataataaatagtgttgggtttcctatagttgcatgtatgataatgtttaatcaaaatagtaaactatcaaatgcaatttcagagcttaacttaacattaacaaagatacaatcagatattgacagcttaaaaaataatcaaaaacaaggggaataaatttccccttttaaaaggtggtgattatatgggaaaaagaaaatctataatatctcaaaaccctaacattttacttaatgaaaatacaatatgtgataatcaaacaaggttatttttacattataaacttatggcttgtaataggtttaaatgggaaaacttgccgactggtttagaaagtagacatattgaggattttttatttgaaaatggtcaatgttttttctttaaagatgataaattaggtttgatgtgtttaccatgtcatggtctaggcgatttaaatgtttatggggataatataaaattgagtatagtttcacgaaatggtaaatatcaaaaaatattagaagatggcaaagatggaataaaaataagagcgaatgatttatgcctaccaacatcaaattttatttcacattatgcccaaaaaatggatgatatagaaacagtaataaaaagaaatttaaaacaacaaatgaaacccttttttgttacagcaacaaataataatttattgtcagtaaaaaacattgtaaatgatgttgacaatgggaaagaggttgttatcttagataaagacttaggggaacaaggttttgacggttttaaaatgctacaaactggagttatttacttagttgataaattagaagacgaaagaaaatcagtagaaagtgaactactttcatttttgggtttagataatgcaaatacagaaaaaagagagcgacttttagttgatgaagttaacgctaataatgaatacattgaaactaacttagacatggaatataaaactaggttattagcttgtaaaatgataaatgaaaaattcggaactaatataaaagtaactaaagttgttgatagttttgggggtgatgaagatggcgaagtacacactagaattaaggaaattagagaataatttatttgactttgattatccttactatgaagaagaggcaaaatgttactttgaagaaaaattcttaaatcattattatttccatgaaataggatttgaaactatagcaaggtttaaacaacaactaagagcatattttttaagagttatgccatattatcaacaactttatgaaatagagttaagatgtaaagacattgactttatgttaaataaagacttaaaagaaacttttattagggaactagaagaaaatgaaataaataatttacaaacatcacaaaatgaagttggtaatacttctacaaatataaataataatgaaagtaataactcaattaataatacaaaagaaagttcattagcagatggtgtttcgagttcaaaaattgctgacggatatttaacaaattctagtcaatctacagatgcttatgatagtaatagtaatacagtaagtaaaacagataataaaagtaatttaaatataaataatactggtgataaaaacaacaaacagaaagaaaaaacagagttaatttcgcagggtaatattggggtcacttcatcagcagaattaaaagagaaatggcaaagttgcttaaataatatagatgaacagattatactaggagctagaagtttatttatgtatgtttattaggtggtgaaataatgattataattgacagaaataaaataaaggttaatcaatttgatacttgtattttggatttaaatgtatgtgggtataaattacaagaaggtgataaaatagaatttatatttaattctataaaagaagaaaaagaatttaatgatactgatttagtattcccaactgatgaaaaaggttctttcaattattctgtgtcaatattgcaaaaaggtgttgttagaacagaaattattaacagtatatgtgaggtgatataattgagtaaaataacaggaaaagttaattataaagaagtgctaaagggtgatactggttatacgtttgaaccccatgttacagttgacggtattttgtattggagtaataatggtgatttaccaaatccattacctgtaaatataaaaggaaaagatggtaaagacggtgagaatggaagagatggtagagatggtataaatggtaaagatggaaaagatggtataaatggtaaagacggtataaatggtaaggatggaataggaaatggtgtcatatcaagtgaaataattgaaaatggttatatacttcaatattctgataacataaaagaatatatagtaaatgctaaaatagatatagaaaaaaattttgttgatataactttcccatttaattttaaaaacgataaatataatgttgtattaactaaaaaacaaaacaatagaagtggtgatatatattattataatttaacaaacaataaggtaagagtttttagtagtaatagctcaaccagtagtgaacaatatttttcaatgcgtgttgttggggaggttagtatataatgaataacataaataaattaaaccctattgaccttaattgtagcatctttacagtttatgattatacaggtctatctatgcaagaaattttatgtcaattttttagtaaaataaatgaattgattgactcacaaaatcaagttatagatttaacaaaatggctggtaggacaaggattaaaagaagaagtagccaaacaacttaatcaatggttaattgatggtactttagctaacataataaatgaaacagtatttagggatttaaataataaaattaactctaatatagaaaatattaaaaataataaaaaagaaatagataaactagtaacagtttggaataatcttaaatgttttaatgtagctgattttggagcaattggagatggagaaaccaacaacaataaagtctttgatgatgctataaattcatgtgttgaaaatagtatacgtggattgttaattcctaatggaaaattttatattacagaaggcattaacacaaagggagtaaaaattataggaatgggtgacccctatattccatttttagaatgggaatatacaagaccaaactcaaaattagatgaatataaaaaatatttaaataaatgtcgtggtagtataataacatcagataaaaatattaatatatttactaatggtttatatgccgaaaatataggtatttttggtaatagaagagcattaacacagaatggtattggtcaaatggatggtggatttggtaactggattgaaatagaaaaagttaaaatacatgggtgtggtaattgtggaattaatgcagaatatggattaataacccctataataaatcaatgttggatgtatcaaaatggtaacaacggaataagaattggtaaaaacaaaggaacttacacaggcgaaacaaacgccttaataatagaaaaatgttttattaatagaaatgaaagtcacggtatttacttagatgtaaagggtagaggttatactattaagaataatgacttagaacaaaatggtgaaatgagcgacccattaagaaataacaagggtactgactacaatgatatagtatatggttgttatatgaaggtagaaggtgaaggtggattcactagtggttcaatagattttagtaacaactattcagaagaaactttaggattattataccttgaaagtcccgacaataaaatatgtcaaggtgtaaaatttgaatctaatatgtggcgaccttataatcaagaattatattcaaatggtttactactaaaaggttggattgaaggagttaaaattggtaataataatatgtacggtagacataaagtaagagttatcaatgcaaatacttatggtattgaaactgacacagatataacaaacccagtatataaaaacaaaaatatatctattgaaaaacattatgactataacggtacaatggttgactttagaagtacaggaagggttgaaaaatattctattgaaaatatgatacaatcagcatcttatgatggtagcacatacaccaatgtatatttaaataaacctagcgtaccatatgagttgcaagaagacggacaaggtaataaattaataggaacgacgtttttaacatcagatggtacaagtattggattggttgttgacttctcatggactcaaggtttatttaaaataagaaaagacagaacatcattaataaaaactggtaacgctaaatttatggagaatggtggttttacaacaataaatggtggtaatacaccagtaaaaatatatgttgaccatgacaacgaaatagtaattttataaaagaaaaagagggtttatttaccctcttttattctttcttttatatcctcataaaaatcgatttgtttttctagttgatttatattatcataagtgtcactataattttcaaaaaacattttttcgtatgctattcttgtattaagttcttgtattcttgtatttaataaatcatttaatacactttctaaaccttctttatctttacactcataatttataaaattatcaataaatttttgtgtatagcgtgaccttcttaataaagtataaatcatatctaatttgatttttcggtcgtagtcttctaatagtacacatcttttagattctatttttattaaatttaataatgttttgaatttcattttcttattcactcctattaagctcttttaaataatttattctctctatattcgttgatgaattaagtaataaatctattgtatcgtctttaagttctataggtaacatatggttagacttttcaaaaacaaaatcacaatcaccccaataaccatctaaattcttatcaaaaacacaccccttacatattggaaaaggataacctttctttttccccattaaatatctttctttacaaacttctttcatagttttgcacattttaatcactccttaatataatatacaacttaaaaatatcgttaaaagtaatataaatataatgaaaaacatagtattaattgatgaaccatacattaatgatataacaatgcctagtgataacccaattaatataccgcctaagcataataaaaatatttttgcaaacttataatatttattttctattattctaaaatattcactattaaatgaataataagtgtcattaccaatatttacaacaaaatataattcattattatcaatgaaagattttactaaattatattctttatccttaacgagtgtatgtttaccgttattatataaattaatttttgcttttacattcataatatcacctcaaatatctactcttattatttcacaattattaattatccatctattaaagaataatcgttcagcataaatactatataaatatacatctgttaacattattgagttgttattgttaaatattattgtcttatttgttggtcttataaatacatcttttaaaatccatatttcaatatgttctaaaagaccgtctagtttatattgttttaatacatcaatataattcatgttatcacctctttatataatataaataacctataacgaataaattaaataataaccagtaacatattattttaaacactatatcacctctcttaattgatactttaattataacaaattatgttaagctattcaatggttttataagatatatttgtaaacaatttgttaatagatagtgctgttttctctactggtaataactggggaatatg

SEQ ID NO: 10 - DNA, 51778 bp, Unknown
  Clostridium phage vB_CpeM_ACP45, complete genome
ttttatttttcgagattatagtgttaaaaataatatgttactgttactatttaatttattcgttctaggttatttatattatgaaggagatgttaactatggaactaacaagatcagaaaaacttaatgtaataaaaggaattgctcaaaaggctatggataataaaatctatccacaatttagaaaagaactagataaacaggtaaaatcctttggactaagcaacaaagttggggaaagtgaattaggagactacctttcttgctgtatggttgcctatgaagaggttatactaaaaatgaaaaaagccaattaatatgggtggccatttatacaatttatatttataaatattttatgtgttctaatagaactcataatattttatttattaggtttaatgtgagaatgaaaaatgactaaattagaaatgttaaatgagatagaaaaaataaataaggcggtttttgaaaagaacttccaaaaacaattcttggataaggtgatagaactactacaacaaatgggagtaactaaatattatgtatgtaaacatcataaaaaagctgagattgaaaaacatcttgaggcttgcatctgtgcaatatatgatataggaagaagggatgatctaggatggtaaaccccatcctttttcttcgaagtttttcgttgacagatccagagttctctcttataattaaatcataaattaactattggagaacaaaatgagagatatgatagaaaatcaattagctgaatatgaggtaaaggagttatacaatgaggccacaatgatcttaggaaaagcatttggtgtaagtgctgttgatctttatatagaatctgatgtggatgctaactactatggtctatgccatagatcaggtaataaatttactgggattacaattaaccttttccctttcaggtttgcacaggaaggagaaaaagcttatgataaaatcaaaaaatttggctgggatattaaggactgtgttcttgaaactatatgccatgaattggctcacttaacttattggtcacatagtcctttacataaaaaattaactagtctttattacaacaaagtgcaacaggctagaaaaggtgaaaacttagctgtagcacaaactagaagtgatatatacactttaaaagatttatgtgaattgatagaactagatccaagaaaggctagaagtatactaagaaagaataatattgagaaacctggtaaacaatgggagtgggagcaaggtatgcctaaagatatatatgatctattaatcaacttaaaggttaggggaagaaggtaaacttcttctctttttttcgttgacatatctcgagagtgctggtataattatattaacaaataacttaggaggtaaatcacatgatagatatatattatagtattcaagtagaagctgaaaacttctttggttgtcaaaaagaagatgggagctgggtagcaacaggagcatttagtatacctgttaatagcataacttttgaagaggctgataaattatccaatgaggccatagatggatggttaaaggagtatgaggttactaaggtaataagtaggagatcacatacacaggttaaattggattggtataatagttatatcaaggatgatccattaaagggtgatataattaatatggtgtaccctgatctttcagataaagctaaaatcaaatatggctttggtaagatatgggctagcactatgtcaaaggttgctaagaaaggtttacttaaaacttgccctagatgtggaggcactggccattactcaagaacctcagatggtaacactacttgctttaaatgtaatggctttaaatatgtgataccaactaagatctcaaagaaattttataaatctgtggagaaaacctttgacatctctggcaataactcttataataaacttgtaaacaaaaacaaagaggagatgttaaatatgaacttaactgcaatggaaataaagatattaaatgctatgagaaagaatgaatttgatgatggactagatgttgactgtgtatgggtattctcagtaatagaaaactcaggtatagaaggaactaaggctagaggtgtgatctcatcacttgttaaaaaaggtttagtatttgctgatggagaggtaataggttacacagaagaaggaagaaaggtctttgataatgctgatggagaagaatgtaactggggaggtccaaaattattaaaagaaatccctgacaaaccagaggaaatctcttataatgaaaatgtagataaaacaaataagaaagaagaggtaaatgatatgaaagatttaaatactatgaaagctgttgaaatcaaagaattagcaaaggaattaaaagttaaaaactggtggactatgaaaaaggctgatctaatagctgctatccaacaaatccaacaacctcaagaagaaaaagttgaagaggtacaacaaccagctgaacctgaagaaaaggctgaacaaactaaaattactcttgatctaacagatgttaaggaaccagagaaaaaggatgaaccaaaaactaaggaaggtaagttcacattaaaaatgatacttgaagaattaaatatgaacggtaaaaaggcaagaaggatcttaagaaacaaagaggttgtaaaaccaggaaagcaatgggaatgggataatgaagaagaatttaaaaaggttaaggacctactttctaaataaagtaggcccaacctctatcccctattgatttacaggaggatctgattttctcttatccattaactgcttccatacttgattaccaaaggaagctacagctgcacatattaaaccctgtataaagccattcaaaaaggtcttggctgtaaaacctaattctaattgaattgccatatataagattgataatgatatactgaatatcaaaagtattaatggtatagaccaatctggtatataaggcttagccttgataaacagacctaaacagtaacatgatacaatgattattaaaagctgtggatcaatgtaattgaatattaagtttactaaatttccttccatgataaataccccctaaactatctctacatattttgaacttacataaccttgcttcttaccgtggctagtattatattctatatgataccaaccattcttacttcccaatatagtaaccctctcattactaaatactgagcctattatattaccattcacatcatctcttacattaagagaagtgttaactttaactattccttgtttaacatcctcatagaatttctggctttgctcaccctcgtagcgaacgtatccatcagatggagtaccatcctttctataaaatgttattcttaaccagccattagttctatacattgggctagccttttgaccaggctgtagttcttcattatgatctttgatgttaacgtcttcagggttccaccacacataagttactccattgtcattttgatatttcatgtggtagtcaaaacttaatctgctgtaatgttttatgtcaatataagcatggtacttcttattctttccaggatatacaacttctatgaaatttctagaaggatatattccaaggacaaataaattatctaggctatcaataaatctaccagggactggcttacaatcctcatctagaacttctattgaaccttcccccactaaagtagcattagttcttgtctcattagactcatagaatcctttaggattattatctgctggaatgcttggagtactacctccactgatctccttattacaaatacccccagctattaactcaccaataagtttagggcctttagccttatagatatttacatcagtggttgcttcacagaagcatacttccactatgacagctggcatggtagtctttcttagctcatataatttagcattacatttaactcccctattctttaatcctgtacctttggcaacagcattaactattcttgtggcatatacttctgcttctccaccaatgccatatatccaagttcctgtacctaaagcaccttcatacatatcgtaagccttgtcaaagtgaatactaataaataagtcagctccccaggcctcagccttattaacaccatatgctaagtcagtgttaacatcacaatttccaggagtgacatctaggacactatggccttcatttcttaaagccagtattacagaatcttttacttttctatcctcagtagtttcatcaattaaaccataagcacctttagcttgaaagttatgtccacctcttactgctatcttcataataaacatcttaattccccttagatatgatattatagtattgtaatcaaattacaaacaccaaccaaaactttataattagttaatttcaaactatactaaatttgttattactgctgctaggcttataatttaacctttgttactaaataggtgatcttttatagtatctacatcttcttttatatcctcaactacgttgaatttatcaactagcttatctatcacttgatttaatttattctctctttctctagcctcttttctagtgtcatataatagccatacaaatagactacaaaagataccttggctaagaacttgagttagtagttctttttccatagattacaccctttcattaaagtcttcaccacatattcttttatagtcctctggtgtaatttctccttccggattagaagaacattttacggcctgatgtaatttgtccttacctattgctccaattctataagccatttcccagaaattcataatctctacctccttatttattcatcaatttaatttttatatcagctagttcttgtgttaatactttcattagggctttagtttgcattagctctaatttagtatcagcaagagcctgggcttgataaccaattaacttattaatatctaggggattaggatctatagcctcataggaagtaaataggtccttatcctttatgctatataagtctgatcttttcttatttatatcaaacttggtaggttctccttttatgtggtcaaataaacttacagtcatttgtataaccggaaggttatctaggtcaataacctcagtgttatccacaaaggctaaatgcccttgtgaattatgaggtatatggagacatataatatcttcgatattatttaatatctcactaccctctctgtagtatagtggctctggtctattaattaggtctaatattttagtaacttcttcatggtctttatctataaatggttttacatcttctgagtcttctgcctgatttatcagcatttctttttcatcatcatagaatataaggtctataaccttattagtttccacagaataattaaatcctactattctcatgttttatcaactcctttactcagtagctaagtaggttagtattatttgagttggtttaccagctaccatttgataacatccaccatttaggtatgagttagtccaatggtcaatagtacaataaccagaggtattattggaagcgtcattatagttatgtgatactagagatattggtaatacccaactccaagccttagggaaatgtatagtatagcttacacccccacctgagttagcatagcctgtcttaactatttgacgaactaaccttacacctccacccaagtggaataccctagtactgtctccattatcccaatagtctcctttagctacagatttattagctacctggaagtcttgaacattctcagtggctagtattctaccctcctgtttataccaaaatcttccgtcttctggaccaccataccagtcatttattctcatatattttctagcccataggttttcagctgatagagccttatccacatctagggatccatctccagcggttatcctcatatcaaaatctttattactgccttttaggtggaagtctataatatgccctacttccataaccccatcattaccgacattaggtaccccttgagttcctttactagagtcatatatacccatattagctagcttgttactgttggcagctgtatctcctttcttcaaataagcactatcatggttatggttagaagaggctgctccaatatcagatggagtaggtttaaatccttgatggtacacagaattgccatctacattaaattgtgttccccctgatgatacccacatatagatgcccttttttccagaaggttctaataccctaaaggcctttccatcagtttcagaaactagatccaaggaggtttctgctgcagttcctccctgttttaaaatccagcctctctcagtagcaaaccttattagcttatcggcttttctaagttctagagttccatttattactccatcaccttcagtagctacagcccctatagcctggggggttggtttattaacagaattatatacagctctccaaggactccagtcagcattattaattttatacctccagtagtgattacccctagtatcatgaaagtcttgccatatccagttattcacaccattgtaagagtcaccaacactaacgtatacatgaagtattccatatatgctactgccatcttgagcatatagaggaggtgaattaggtatagctgtactagaggataccagatgttgaccctcagttatagctataccagaattaaaatcagtaatagcctgtttaggtataggatagtacctcctatcatggttatgattagaaggggaagcccctatatcagaaggtgtaggtttgttttgtttactatatatttcagcccaattcgtccatccagtaccctccatgtttgtagctgttctagtaaaaactcctccaccatctgcaaagaacatttgagaagcccagtgtcctgtattccaggataaaacaatcaatctcccatctgctagagttggggcatttttttcaatagaatttctaactgtagaataaatacctccctctgtaatggtgtttagatctacaccttctagtcttttggctgttttatcccaagcccctatatctgctggggtaggcttattaccctcatcataaatcttcttccagctagaccatttatttgaatagcaggttctaatataagtagtatggttactatctgctctaactgaggtaaatctttgtaatacacaatcattattatacttaactacctccataattgctacaccattgggtacaggggcattgaccatattttgataaccccaatagaaaccaggttccttatgattattcaggtctgtatcagccatgggcatattaaccgcagctacacctaattcttggggtgttggcttattagcctcatcataaaccctataccacttactccaagaattatttactaatgtgtagtttctaatatatctggttatcttattggttgggtatagattaataaactcctgttggaatccccatcccccctcaccaatatttctaactataaaaactctattactcctatgggctaaggttccctctggggtatttttaaagtcaccagctcctgtatcaaaatagaagaaggcattcgcttgtttaaatgaattacaatctctatctataggagcactaacctttagaattacatcggttttagcagctttttggtttacttgattttgtaaactagtgtctttctcaaataataggtctaatctttttagcacatcctcaggggctaggttatagtcaggtaccacattacctctagctaataagatatttagtacctttaatgaggtacttgaggttgcaccctgtaccacaaattgtattttggtatcagtagctccagagggaatactaaatttatgagtttttaacttgtacccattactctgtggtgtactaccttgttttacaatactcttatattgttgacccccattagtgtaagccagtttaatttcatagctagtaaaactaccttcaatatcttctagaaaggctaatacgtaatcactatttggttctacaggcagagttttaatttcaagagtttgtggacctgttgacataccagaccatttaaaacctcgtctcttggttggagcatctgtaatagatgtgatattaccaggaccagtataagactcagagttgtaccactcaagcctgttaatggtaccaacccttaaagcatctagcctatcctcattagatttagacttcttttctacctcagtcattttctgtttatgttcggtagcaaagttctcaatgttttcattatccttattaaaatcagccatttttggcttatctgttaactcccacttatttaaacctagagtaggagttttaccagtacttggcattttgcatacctcctttattcagttatagcatattcttcccatttttcccaggtaaggtctagactgtcccacttgctccacatataatcatacctatcgaacatctcccaagtaatataaagtaatataatttgatatattaaatgagcaggcattctttccctgaatactctttctagagcctttaggttaggtggtacccctggtaggtcagtaaagctaattttgatcttactctcttcagctatgaactctacctttatctttccacccgtataggccttagtaagatttttgatagatgtactattaaactttttccaagcagatagaactacggtaataatagctcttttctcttggtcagttggtgggtaatcaaagtatttatataattcaagatcagtgaaaaatctttctagaccttccttaggagtggtcttgggatccacatagtcttgtaattcctgtcccttagtatatagcttctgaagttcagtagccataatagtgtttactaagttatcagcctcttttatattatcataaaattcaggaacatacaatagtaattctttttgaacctgttctagactatacataagttaccaccacctcagtaatggttggtatttgattagcaggaatagttaaagattgactactcccattaagtgtaatggttgtataatcttctactccctcaatatttaaaagcatagcccctattctatttatagataggctagtaattttaaatacttgactatttagaaattcttctagttctttcttgaagtttaattttatttgttcttctgtaactgaactcaatttcttaacctgagctcttacagtgatattaaaggtttgagctgactcaacagtaaaaacacatcctataggagctttaccctcacctaaacctttcttgcctggatccatgtattcttgaactttattaactagctcaggattagcaggcttattttcaactgtggttataatacccttagcagtatttattccattccaacagggaagaaccacagctttacctactccagctacacttttacaccagtcagttaactgttggacatttccatctcccgatggttgtatagtggctttagtagctctttctcttaactcatcatctgtctcttgatctctaccaataacaagtatatctcccatagtagcactcttaagttctgctatagcattgataggaattagatgtgtacctgttgatactttattaggtcctgagcccaaggtctcacattcaataatattaggctctatcacttgccagaaataatctcctccttggaatcttgaaccaacatcgggttcatagccaacaaacttagctgaccttctacatttagttggaattattctggatatccctacattggcagctctctcatctaagaacttacctgtagcagtacttagaaatactgagtctaatatttgattaacattaaccgcatagaactcagctagcttaacagcaaaaaccatagccatatcataggtgaaacttccagttctagtgtcaatatcatcaggggataggcctatcatctcttctaataattctttatgtgttcgggagaataagtcttcagaaaatcccattatatcaccccttccagttttgtatctccatatatggtatttatccatacttcaatatatacgttcttaccttgcacattatactttaggtcagttactttagtcactctatcatcttctaagagacattcagggatagccctattaatctccatttcaagaagttcaggagttactagagagtcattaatcaaagctctaatatcacaaccataagaagtatcttgataaatcatataggcatttttaggagttattaatcttttatataaagattgttttaaggcctcaagaccatctacactatcataaatcctattaagatttagatctagtttataggtcttggtattctccattgttttctcatcaactataagggtatctttaaagccattaggtagcattatatcaacctcctaagatccctaacataaaatatctttgtccatcatcatagggcataaggtaaatggtatcaccctcactaatagactcaatatgttttggaacatctataatatttccagatagtactaatttcctattgtgaagacactcacattttatagggctggtactaagtactttagctctataaatatttaggggggtagacatattactatatacttgttttacacaatctaataaattattagccattaaatatcgccctcctcagtacatttaagttttaagttcatggtgtagtcagtatcgaatttatgagtatcggactcaatgtagaaagccttattaatttttaaatgaggtatactaaccacaatggctttaccactaatacagtcaggtacacctataacagttatagagatatcagttttaggtctcttacccgtttctagctttattctagccttctcatttaattgagcttggtttagttcatcactgatagtatcgtataattgaaataccccatatttcttttggctgctatcatctttagcagaggcaaataaggtaatctcgggatcatctttacctttaccctgtttatatttacatactagctttacctggttgacaatactatcaaagtttctacttcttgaaaaagttccaatatttttaccatattcgaatttccacaattcattattggttcttcggtttataaaatcaaacttaccagtaatagagttatagtaaatataaaactgttccccagtttgttcataaacctcttttattaccttcttcataatatcatatggagtttgtttctcacacactaaactagagaaagtatatccagttttagctactactccaggggcaaccccaaaggctttacataatgactcaaatatagcttcaccagtttggttttcaaatacaaaactatctttattattagcccaataaatcatagggtcataagctttaactttaagtaaatgcttcttctcattatctgagatattcataataattccattaaatatattcttaccctcatggactagtactacctgatctccatcaaaaattggatgtatatcttgagcattatattggaatgatagggtacgaggggcagatgaagaagaccctgcccaactcttaccactggaggtgtttgtaatatcaaaggatttatctccttgtaccacaattaattgcatattataacctccttatataatcaccagaactccaaccttccttatagcctgaaccattatctagtttatactttacataatgccatccattttgagatcttaatactgtaaagtgttcattaggccatatcttacccttagtaggattattgatacctggtccagttcttacccatagccatgagtaaacattagtagtagtagctgcccaaggttgagtacctccagaagaactaccacctgtattaccactataggctagtttaggttttggaattgtgaaactaactgatttaacctcaggcttcttatactcagttagttttatttttacttgaagagtaccaatatctccacctttctcagtatatgagatattatcaatagtaactaataagttaatatcagtactagtaatagtaaatcttactggatctttacccatctcaataagttttgcataacttactcgagggtctgcaatatcagtatactcacatccatcgtagtatgagcttggtaactcaaaattaaaagaaaaagattttagcttagggtctcctataaaagagagttcccctaagccttcaatggaaactgtactattatctctactatcagagatatttatatcagtgggatttactggtaacctaaacctagtatctccctgtattaaccagaattgatatttactatgcaaggtccagttcacctccctcgaacatttcctgatttaacatttccacaataaatttgtatactctcattaattcactttctgagattgttccatcttcagcctgtactgtgaaattaaatgagttgttaatgttcataactttagatccatcatcacttcctgctccagattgtcttagtaaatcctgagttttatctgcaggtaatacttgagagcctctaggcatttttgagaatgtaggacccataactaattcgggaccagcttcaccaaccatggcagctttatcagtgaagaaatcagtaccttcagcaagcattggtatctgtggaatattgatacctttacctcctatgattggaacccagtctggtattttaatagtattaagagcaccaattaatccattgattaaactaattattccatttataggtcctttagcaagagctagcataccattgaatactccaccgaatatattcttaactccctcccaagctttagaccagttacctgtaaatactccggctataaatgcaattataccacttagggtttgcattaaagcttgaagaatatttgagatattttgtacggctccatttacagccgcagcaatcattggccaaacagcattaaatactgctcctaaagcataaagtataggagttagcatatccacaagtccttggaatatcataccaaaactttctatagcagattgtatatatggaactacactctcagcaaagcttaagaacattggtaatagtgtattattccaccaatcagctaatccctgtaagattggcattaagatgttacctattacttccacaacttgtgaaattgcaggagctatataagtagcaaaggcctcagctaaagaagtagcaattggcatgaaaacattagttatcaattccccaagagctgtaaaagtagcagagaacatagagcctaatacttctaatataggacctaacacttccaatacagtttgccatatttgagtaaatccctgaacataaaggactactaggtctatggctggaattaggttattcataaatatagaagctaaagttccaaacatatcaagaagtacttgacctataggagctaatactcccatgataggctcagctaaaccattaaaggcttcttgtatattattgttagcctctgataaataatctggtagacctactaagatctctccaaaattattatatatgtcttccccaagactccatatcttctcattaatttgatctgccacatcagcaggtaagaacttatataatatatcattggttagatctataaaaggttgcatagcatccccaaactcccaaccattcatgatgtaatatatagtctctcctaaaccttggaaagcagaaacaaccattgagaatataggtcctagtttttcaaagccttcttgtataaatgtgataccatttaggacagcattagctataacctcaacagcaggggctagggcatctcccacatctaacataactacagcagctttggattttattctctccatggttctattaaagcccttatccattttttcataggcagcttcagtggccccagcactattcttcatctcattaagagcattagtaaaggcctcagtaccttcaccagtcaaggctagtgcagctgaaccagcttctacagaaccaaagaggtcattgatacccagattacttttctgagcgtgtttttctaacatctgaagggcctcttggatattacctccacctttaatgaagtctttgaaacccttaccagctatttcccggaagactttatcagtcttggttcccgatttagatagctcatctatggcagctcttatcttagtggtagctacagatgttggaaccccctgagctgttaatgcagctatagaggcagatacttcttcaaaagataccccagcagctgaggctgaaggtagcacattgaatagggattgtgatagttgctcaaagttagtcttaccaagtcttactgtagtaaacataaggtctgaggccctttgaacatccatattcttagctccatatgagttaataacagtagttagtccatcaaccgcagtttctattgatgtgataccacctatagaagctttaccagctgtttctaagaaactaaaaacgttatcctctggaactgatgaggatatggcttgatagagagcaggtactactttatcaggcaatatccctatatcagaacttaacttttttacctggccggacatcttatcaaaggtctcttgagtagagttgggtaatagagtcataacctcatttaaaccttgctcaaaatccccaaaggctttaatactgtccttagtaaaatcccagacagccttagctgagaatactccagcagccaccccaccaattttggcaagagtacccatgaaggaattaccactatcagccatctggttaaatgcaccagagatcttatcttgggcagagaatatggcagtaaatgcagcttgacttgccattatatccctcctttatgattaagagcctttctttaacttatccctagcttttttatcttcttctaaagccagttcacatgaagctataagaaaggctttcctattgggagatagtttatcaaattcccagggtagtatattatgcttctggaataacacatgagcccaaaatgcactagtgtcggcctttattagttttttgcttctttgattagatcatccttattatcacctaatccatttatgtccataacctcactagttattctagtgaaatctcctggtaagcttagtacatccataagtaaatctactggatccacaaacttagtagactctaaccattttggatctttaaagtcagggaatactattgtttctagtatcatttccctagtagctcttctttggtctgttattgagttagttatacctttgtttctagtaattactgtatttctcttttggatatcctccatttgcttagtagtgattggctttaatatccaaggtataggttggcccgctaattcatgaccctcagggaaactaaatctagctaccccttcaacttcaatttcgggtgattgtttacccttatcattttccatgaaaaattccatactaaattttgacataataatctacctccacaatattcttaatcttttataaatatcttacttctgaagctgagaatgatatttcttcttgaatttcttctccctcagcatctagtccaattagtggtaaatctccttctaattgtactcctagtaattgtactctgtcctcaccataattcttaacatagtcagaatcgggatcatcacatacagcttgtatatccatcttagggaatatacctgtctttaagtattctttaatagcttctcttatcattggagtagctttgaattgagttatagttccagttatatcataacctataacttttgaggttaatcctttctgacccataactcttttcttagtcacctcaggagtaaatagagcttctaattttattaagctagttacttgcttacctgatacaaaagctctaccctctctagcagacattctattaacttcactcatgggctatacctccttaattattcttaacagttacaaagtatttcttgaaagttcttacaggttgtgtaagtatagtagcatacatagtctttcctttactagcttcttcatctaccactacatcagagtcagggtccacattctttaatgctccttgagcttgtagtgtagttaatagacttattagtctatcctgagctagttttataccatctttgtggttagggaaaacattagggactaagatctgagatcctagatctataacagcatctaatacagccacaactttattatctgtaaagctttcatcccattcttctgtaaaagtatggtgtgtatttatatcttcctcaactactacctttactctttcagaatttgagtcagtttgagttgagaatataaattgacctgtttttaatccatcaactatttgagagtgttttagtcttggattagcatctactgctccaggatatgctttataagtaaggtcttcagtatatccagctgcagctctagcaccagctacccatgcagttgcttgagaagctgttaacttagtgcctccatccagtacaaccccatttttaacattgatgatatatgggtaatcacaagtagcaaagtcagaagtaactgccacaatgttcttacccacctcatctctgaagtatttaagttttgataatagagcagtgtgtaattcactctcagtaaatgggaaggccatagttttaaagttgtataactcacagttatctaagaagttaataacatcagagttattcatctgagtagaagtaccagtactaaactcagtcttagcagtctcatctatagaaccacttccacttactgtaaaccattgagacttaactgttgatatttcagttactcctgtagcctcaaagattttagatccttcaagatataataatatatcctttcctccaagaggattggtcttaatagttagagataatttattacccctttctccggctttagtagctgtaaatgttagagaccctactacagctttggctttagttccacctgttaagttatatactaaaacagttgcagcttcttttaaagcctctttaactagtaatacagtagggtgagttatatcatacccaagttgatataatgcttctaggccaccttcattggttaccttgataacatctccagcattaccccacttaagttgtaatggtaagatcataactcctagattattaagcttaggctcattaagtccaccacttacaaatctagtatacataccaggtcttactttattttgtcctggaataaaagttcctcctgccatctattttacctccttctttagccagtcttttataatctgactagtttgttttattgttaactcttcatcaggtggaagggattgaactgcagcgttgaagacaaactcctcaacctggaaaagttgttgacaatgggctcttaattgaggtatggaaaatttttgtactttcactattttaccccctttgttatctcaaagttttcgaagtaagttggatctttataaagactaaccacaatatcatagtttatctctacctgagaatcttcttcagtttcaccttcaaatacttccacattttttattttgagatttttgttggtcttactaccatccctattaagtatgggaatttttctatttctcaagtatatagcattagctatattctgagcaatttcaagagagtctttgggttcaaataccttaactcttagggtattctcagttctataactacaaagagtatcattctgaggattaaataccttcttgaaatataatgagggatacttgaatttttcaggtattcgctcaaaatagagggtagtaattccagttagatcagatatgaatttagcaatactccctatatctgctgtcattaataactaccccctaaaacttcggctaaccaatcttctaattttctttggaaagcctgttcaaatattttttcatatatttttactgcattgtcccaataaggtttaccaggtactttcttttgtcttagtaccataccagtcttagctccgggttgatagataaacttatctcctgaccatatacctggaacgaatctaaatgattgtccttgtggattagtgtagtgaccatcgtttacccacttagcatattttaggttagtaccaacttctaaagttagtcctccatcactacttacccatataccattcttacctcccttttgaaaggagttaagtaataatctagtgtctactacttgtagtctaattatttcatcttgaaccacatcaaggaagtctatccctgcagcttcaaaccatactgctaattgctttttgaaatcactattagctgcatctcttagcttctttataaacctttctatttgtttagcatctatttctatgcctccagaatttgaagccataactatctaaaatccttagcctctataccaaccttaatttttagaccccttatatccctaggagctttagctatatatgtgataccagtctcaaggtccacaattttatcattttgtctaacatcagttccaatagcaaaatctactgagtactcctgggtaatcttaaacctaggttctttgtactcaagtttttctagagttcctgacttaaaatgacaaggtacctcagtcatgtctggctcatcgggataactaaagctaggggtatgggctaggccatatccgggatcttcaggatcttctaataggtggtatatattacatctatgatttaagagcttgttatatgaatttctaatcatattacagtacccttaatctcatagtagttgttctgatagatggactatcatcttctaagaaaggtttaatcagaggcataatatcaggctctataacagccttatcggttcttcttgtatatgagtagtcatcataggtctcggattgaatatcagccttgctaatgaccaaggattgtagtccataatactcagcatacagtatatgggctaaccttaattctttactattgggatttttctccacaatagtattatagtctttatttgaatttttaaaaattcctgagatcttggctttagctcttaatatatccatctcaatatttttggggtctcttttcttaatctcaggattacttgaaaagtcaattatttcatccggagtaatattcatatgagccacccctatctgtttcttttattcttgtttgtaaatcttctagcaggttcttctttactgtcatctgtagaaggtatttcttctccagtaaaatcttctagaggttcatcctcaacaggttcatctatgacaatcacatcatagttctgctttaaataatttactaggttatctttaacttcttctacatggtcttgagtaattctatgacccttaataaccagagtttgtaatttaccctttgctaatttgaccttagccataatatctctcctttctaatgaaaaagagcccacatttatagtgggccttcatgattaagcttttaccttgattatcttagcagcagcctttggatcttcaaatttaacatccatcttaagagttaaaactacataagtagatctttctctaggagctctttctaattcaattctgatatctcttgagatacctataactatatttttagggtgagttaagattatatctgatacttgagtagcagtatcttcataatcttgtagcatagctatacctttaacaggtacaccataagcagacttaacagtaccatttataaatgagtcatcaccattagcagttaatctttctcctactaagtctacccaatcagtttctaaaccattggagcagtagaatctccattcaccagggtttcttaaatacttagctggtacagctttcttagcttgttttaataggtctttacttaaagcagctttgttagcatctactacatgagcagttaattgttttctaaccccatctaattgttgtaagaaagtatcgctactatgtccagtatcaccatttacaattatttcctccatatcaagagcaactctctcagctaataattgcattacagtgtcttctattgattttccttctatatttgtttctatagtatcatcagttaatttaaccacagctataaactctttagcttgtaaagtaactgttccagtagttggagctgaatatttagattgtcctggatctgttccttcagctgcaggatttaatactctttgaccaaatcctattgactcaatctttttagagtctcctttcattggtacagttctagcctcttttaatattgttggctcatcaattatcattttaataaaagcgttagcttgttctgtgtttaatttacctccaccatttaatacggcagcgttaaatacagctttatttacttttgacataactttccctcctaatggttattattcttggcttccgccaaataatcccttaaatactgatttttgaacttcttgtccttcttctccatttttagcaatttgtttacttcctacatatggagccatggctttagctactgcctcagctattttttcttcctcagttttttcttcttgtttctggattaagccagcctctaacatagctttttccacagccttagcaactatatcttcagtagtagcttgttgaccttcattcttattaacttcaccctcaacaggtttagttccatctaatgcctttttgatagcttcatctatcattttttgtacttgagcttcattcatctcttcttcctcctccactgagtctaataattcgttaatagcactttgtgctgtttttaacttttccacattttttgaagatattttcttacctgccttattgactggtaactcaaaggatttagctacttcttctggagttcctaataatattccctccatagtttcagaaaattcttgaagagcttcctttatcttatctacatcactttcaaaagacctagtgtaatcccaactagaatatggatatagaatatcttctaaggtactaactgcattccagaagttatttctttttgcagtctctttatatttttcagagtatgcacctttctgtatatgctttggagttaaccccaaggcctttccaatcttatcaagtatagattgacctatagacttctcaatctcatcaatatctacatcttgtgtagaatatttaccagtaccacccatagaccatccagtaatttcgcctttctcaatcttgctccatagatcatcatcgacaatctcagctttagctaaccaagttccagctttaactactgtaccctctattgtggtatcttcctcagttacccatgattttacaatcttgacaccctcaagagtgttctcattgtgttgtaaatcactaccaaggccattttcattaaaccactcacaagcttttttgatctcttcctcagtcataaaattatcatggtagtcagcagtcatgggctcatatactacaccgataacaaaatgattatctccttcagactcagactttaagattggcttatcatatctaaatctattaggagctccaagttctttagccacaatccattgccatttattagctgctttatcaactaatgaaaggtaatcaattttagcatctgagatctcaatagctttcttaactgattttactggcatttactctccctcctttacaaattatattctgctatcacttgatctctgatagcttctttttcctctttagataatcctagtatatccttatcaattacaggcccatgagtacaatgacaattcacagtctcagaagctggtaagtttatatctctaggaaatctagcttcataaattcctactgtgaagtattcacctttattaatgacagtaccatccagatcgacatggtgttgccttgggtgaataccccttccccctgagtgtttccactgtattcctgttactgcggggcattgattaaaagcctcatccttagcatatgagtgggctcttaacatttcagtaatagcagtagctctagctctagttctactaaagccataggtctcagataatttaccaactacatcttctacagactctccatcttcaagagccttatccagaatattctgtagtttttcatgggaagttaatttcatcatttccccaagtttagaagaccactcatctataaaggtagtagtatatggagaaaagactttggttagagataagtccttatcaataccttgtaagtaagtatctcctaaattttctatagcttcttgataacaactcttaagagtttctgctaaagaagtatcgatattatctgtacctatttgagactccacatattccataaattcttcaatggtttcaaagtctccatcttttataagatgttttaattcttctgtaaggagatcagaaatagtgtcttctaattcagagatcttttctattgtataatcaatctcctcatagccagcttctttcaattctttcttaaggctatcatcttcattctttaagatcttatcaattttagcgatcaactcatcagtacccactattacttacctccttttaattgatagtttaataactgtttcttagcctctttaagtacagctatcacctcatcactcccattagagttttgagccttctcaattatacttgttaggttattactcattgaagaggcaactgagtaagtagggatatttgcccattcttcatcaataggagtatagtctttattgattgatgaatagtactcctcattaatctggttaggagtcatagctctagaggctatagcaaacattcctttctgataatcagagttctcaatattaggactcttaaagtaagcttccacatatttaaaaccatagccagatagaataacattatttagtatccatgttaggttttctctttcaggaacaaatacttgctcttcagtttttcttgtagcctctctggctgtagccacattgtaatcttgagagtaccctacatatatgtctggtagtaagaaagaagattgtatctttttcctagtattatccatgtagtcttggaataactcatccttctgtaatatttcagataggggcttgacctcaacagtaactggcttagagtcatcaagggcagtgtctctctctgcagcttcagtttctaatactaagaaaccatgctgggaattttcaccttttacactattcatgtactcagttaactcattccaagatttatcattaagagtaccaccttgaattagtaacattagtggagtatgtttaccattagtaaaatatctccagtttaataattcggcctttcttgaaccttgagcggctaacataggaccttcccatcttggtgtaccatagacaccataacctgttttaaaatgaagtatactgttagctcgatctttaaggggtatctctcccttttcataatactttccagtattcatatctagatctctaggatctccccattccttaaagaagattgattgattatttacagtctgcttaaaccttctgaacctttttcttctagtgtactgtttaccataccgggttacagtgtactcggtactctcatcatctagcttaagaattttgattgtcttagggtcttcaatctcttttatctcttttacgagggatggatcttttggatcttctacaatctctaaaaaagaatacccaagtccttctctccaatcaactatctcttcaaagacattcttggttggtttatcaaaagacagggtatcaattaattcttccagctgtttatattcagctttcatttctggagtaggttcatctaccttctcaatatacctaatacctattccgaatccagcaatattatttcggtaggcctttttgcattggggtattattgtagactcccttataatcacttctaatagttgggggtcatattcaggagatatctcatcaacttcaattccaagtacactggtttgttgattagatttatccactggatccaaagacttttggatagggtctccacatatacttctggcggatatctttttcaaagttactttacctcctttcttacattatatatcaaatttcttacattatatatcaaattataaaatccagatggcctcatatttcagggaggattacatcaactccacatatagtgtcaaatatggccatacacatgatttaaaaataactataatttgatacgtttattataacataattcacaaaaatttgtcaacgttttatttttacattattttccggaagattttctgtatatattccccatataaaattatataattattaatgtatactatattactatattaatttatgaaataaattatatagtatatagtatacttatattacatatatataaaggtggtaactctgatatgccttctatatcacattattactaatatagagcctagtcaatttcaaaactgatatctcacatatcaataggcatatcagttacttagatttttcttaatttagatccaacaggaaggcaagctaagaatagagagtctgctaagtctggagaatgtagtcctcttttcttcatttcatccttactctccagctttatttgacctttggagttgaatactttaaactttctggtagtcaattcccccacaagatctgtattatctggtagtcttaactcatcatcctccagtaattccttaactacagaggctatatatgtggttgtatcataataaaaggcataagcctgggaccctctctttaatggcataccaaatttacatggtactacctttaaccagtccagtttttcttcgaattttatctcttttaatctatcagttactccaccacctacaccagtgtcgtctatttttacaactacagctagtttagggaactcaccatgtagggatttagcctttcttagcacttctcctacagttttcatggtgtccttaccttggtaatagataggttcttccacatatccatcaattttggtcatgatagaggtcttatctgaaccaaatctagctacgtctacacccatatctatagtaactggctctgtaggtctggttatttcactgttgatacccctatatactgtggataattgtataaatatgtcatcttcttctagtggaaagtccccttttaccagacatctgtagacattagactcttccccatactttttccttagcattttatggttttctttatcagttctatcaacttttgtagcgtcaatatgaatacatttaaaattttccttaagactgtggtgagaatcaaagaagataccagtatttttaacagggttaccaatcaaaattatcttattttcctcatctgatatagtaccaagtaatgcttctagtatctcatcatctattccaggagcctcatcacatataattagcatgaactcactatgaagaccaagcatattctctttcctattactagttttacacatagcgaaccaagtcataggactacaattcctagtcatagtggttttatttactgtaaagtaatcttccacaatagttccttgtagccattttgatatttcaggccataatacagatgttaactgtgaaaatgtaggagctgtagcaattaccttacattcaggtctaaaggtcatataccaaattataagacaagcacaggccatagtcttacctaccccctgtcctgaccttacagaggttctcttattgtctcttacacttattaaaatatccttttgatgtggatctaaatgtactcctagtatatcttctgagaatccacaaggatcgtcatagtataaatctactaattcttcaaaggatatttcttctagggcttccttgttaatgtggaacttatcaactaaagcatctagctctaatttctcaatcattctatctaaattaaacaattatatacacctccttagtaaagtgctttatttagtcctgtaatacctttttacttagtagtattagattttcctttacctgcattctttttctttctatcagccattttagctctaagaacctcaagatcttttcctttaccagcattattctgattttcctgtatttgtaaaagtaatttaaccttttcaagttcaactttcatcacatcagtacttaagccgaagtatacctcaagtctttttagtgcctgaagtctatcttccattttaactgataccccatctttactttgtttaatctcattgattaaagtaccatctacagccttactagaccttaatagtactagatcatccttgaattgacacacatcagttatatctgcaaaggctatcttcatgtactgccttattacatccataccatcgcatagtaattcttcatctctatcggctctgatagcctttagagcttcctgtattttcttcttttttaatagtttagatccatgtgcatgagaattattcacaggatagcctgctctagcagctgctgtagttgcattaaagcacttcatgtaatgaaatacaaacaactcttctttctcatcaagatcatacttctcagccaaggtcttaatagtcttttttgttttgctactcaatgccacaatatcaccttcttcttctaaaaatgttatgccctgctctttaaactctcttagacactgcctaacaaaggtctgtgataccttagcaaggttccttaattgtttaggatgtgtaaagggatcagccttcagggcattatatatcaattttttggctttcttcaaatgttcaggagtacagtactctggttcagtgtatgacttatagctcataatacaatatcacccccttaaaatccagattttactacgtgaaaaatggttggcacactaacattaatgctagtaataccaaccaaatggcaaaattaactatggatttataccactattataccacatctggataatgatgtcaacgttttataattttacctcaccctgtagtattttttcatacatttcttcatctatagttccctccatcaataggttcttagctgcaatttgtatttcttctggggaatatactggattacctttagtatcaagttccgtaccagtatatgagtctaccttgcccctgttcataccatatccaactgatttatgaacttctagtttttcattatggtcttcttgccaatgttttaattcttcctgatattccttattctcttctttagtttgtttacattcaataggtctattatctttcatcttttgctttagggctgctgtattaggtttacagttcttagatcctccacctgaacctctattcttatcttcctcataagctttattaatctcaaccccaattattagatccatcttctctatcaagtaaagacattcttttactattggattgttaggttgatttttagaaatgatgtcttgacactctttctttaattgatgtggattattacattggctagggcatttttctcctggtccatatggaattgtatatgctaagcagttacctgctggatcccttctataacatcttctcttacatattattttactcattgttgttatcctccttaaatgtaataaatattgctcttgatactctggaaatgttacatccttgaaatgcttctcttactttatcatgtactagatctactcctactaaaggttctatcacaactatattattagatcctagtacaggaaatacctcacattcagtatctagtccatctataaattctttaacttcttccaatcctttaaatggaactggcttaatccttgggctttcttcccacaataatttttcttctaaggctttttccccaagtagtctttcatactcatcaatgttactttcataaggatttttaatttcttcaatacctgcagaggccatagatcttatggcctttaattgactatcactaaagaattttcctttactcatattgttctacctccacatcattacaaactaatttagctattcctccacttaatctgtatttctcaagatataccactttcttaattcctgcattgattattaagttagcacagctctcacatggagataaggttacatacatagtagttccttcagttgagatcccatgtttagcacagtaacttattaagccagcttctgcatggattacaatatcacatccaggacctaaacaagttttacaatgttctactcctgaaggtactccattatatgaagtagcaactatcctgttatctctaactaatacacaaccaactgcttttcttaggcaagtagatcttcctgtcataagtttagcaacttccatgtaaagtgtttctctgcttattctgttgttattattttccatagtttatcacctcaattttattatataatattccctatcatatgtcaagagaaactttagaagggtatatcaccataatccagatcagtacattgggctggctttagttttacttttttaggctctgggttttttttgactttatgatactttggagctctggctaaatcctttttcaactttctgactctcttcatatcccactcatgtatttcattgtctctaaaatattcattcaatactcttactgcggatctctttgaaggtgtctttatagagcagttattaggagcaagacagcatcctgtctctttatctgatatagtataacatactgtattataaggggctcctacaggcctatgaatgaataaggtcctgcccatataatcaaactcacataatacttccacatatcttttatactgttgtccttggtcattcttacataatacataagtatattctttcatgttaattctcctttctatgttaaaagggagatcatctctccccttaatatctttttattcttctgttgttctcatcaacctcaattacttcataccttctaaccttatcagatataccttccccctgtaatactttaggaatatatcctatggcctgtcttttgttatcaacttcaaaatatctggtcaagatcctttttgatgttacaatcactaatcttactctcatactcattacctccatgtggcctgaactacagctccaggtctatgcttagcttcaatcttagcttcttcctcagtatcaaaatagattatatcttccttgtgttcatccttaacagatacccagtgtactcctaaatatttattatgtgctaagacactaacatcagggttccaatccacatcaactgtaacttgtaaaactatatatcttcttttcatatcagttctccttcttgtatttatttgttgattttctttttcgttgacaatatccacagtatatggtaatataaatattgattcgtataatatcttccgagctgagcataatatagactataaaaacaaaatcaaggagttgtaataaatgaaagataataatttaataccattaagagatattgccaaagaggttggaataaatgaaaggaaagccagaaagctattaagatcacatgagaataaagcaaatcacaattattattggatgtttacaccaagagagaaaagacatattgtatcagttctaaagagactgcaataggttttctaaaaataagggagggattaaccctcctttttataattttttgagttctggaaaaatccttaattagttcaagtttcgtaaagttctcttgatcttttccattctcttcttaacttggatagcttcttcttcgcaaggagcatggccctctaggaattggtgccatcttctttcaagttctctgtattcagattgaagttccatgaactcaagtgaatgattgtccatactatcacctcctcaacactattataagtgatactgtcatattctgtcaaataaaaaagagggaatgttattctccccatcttaatatctttattgttgctccatcaactcccttgccatccacatcatggacatacttatcaagtctcatggttgagtcctgtataattcttttgtcttctctgtcaaaaaccttaatgacatcagggttacccctgtcaatgatgtaagcaatctcttgagttccatctgcatatgttacttgaagtaatgagtgtagaggaataaagtctggagctgcccagtcaccaaccttaagatctcctcctatactattcttggtctcatatcctgccacatctgtaggttcatctgatgagtaataagtaacttcaactttgaactccattcctttaaatgaatttatcatctcaattagattttgtttatcagttctcagtttatcattctgatattttaatccttctacattgtccatcagtatctcagtctgctcagttaattgtttattcgactcaatatactcttggacagcatttccccaatgattgtttaggctctgtatggttaacagtagccctcctatcacaattattgttagtaatgacataattcttttaatcattctcttttaactcctttatttttaatttgaatttctcaattccagatttatatttacccacaatatcaaaggtatcagttagtattgcataggtttcctcatcagaattaaatctgaagcttcctctcttgatgtctcctagcttattaaaggtcttaccctccttgtccagatatcccttatagtccatgtagcacagacaagcggtaataaactcctgtttagttctgaagtgatgagatatacccataccttctacccatttacttaatacaaataggctttcattatcctttggctttatcaattcagtccatacaaactcattagcttgctttagattagtaaactttttagccttgaacttcttactactctggatcttatccttatagtcatggaatacttcaggccaaaattcctgtaaatacatatataaagaaaactcatctaccttagctttaggatctttaaagttaattaatggatatccagacactgctcttatctggttctttagcatattattctcattttttagttgagaaacaatgaaagagagatgagtaacagctctggtaagctcatgctcatttaaagatctaccttctctattctggattagcttccttaattctccccaattatctatcatttatagcacctcctatgcttacattatacatatataagataaatatgtcaaagtccacataggcatatcagtatcattttttctgatatgggtagcatatcagcatagttacatctgatatcacatatagagtttacaatttttgtaatcaaatttgttggtatcactagcgtagagggatatcagaaataccccatattattacaatatatactatatataaataattatatattattatatatagtatatagccaatggctatagtccaggcattaactgatatctgatatgcctatatttttaccccatatcagtctgatatcccacatatcagttgaaactacaaaatcacaaaggtgttggtatcactggcgtagagggatatcagttatgccccctttatatatacatagtattatatatactatataataacccccgaacgttgacagttatacttacttctcttataatgacagtaaaggagggataaccatgaaaacattatttaatggaactgatgtaatattaattgaaactactaacttcttcattatgttacaagaagaaggtaagtatgtactgtctaaaacagtagataagaaaatgggagagcaaatatgtaaattacctaaatcagttgctcttgtatatactaatgaagaatacactaactctaatgtagaacctgaaccaagatctatatacttagatgacaatttcttatacgaagtgcttggtaatgggaagtatatcataagatacagtatataagagcccacaatcaattctaaggccttttagatatgtggtaatgtgattttacttataatgtgatagagacaccttacaggagctgctagaggcctttaaaggaggtatatcatgtatttatgtaatggtaaaatagtcaagaataaaaatgacttaaaagataagtttggtggtaaatcagtagtattcatcgatggggatgtagccacagtaatggactttagtgacagaaagccaaagataacaaaatatcttattagggaggtatcacatgataaacacatcattattgaagagactggaagggatagaggaaaagcataagcaaaatactgatgagataagaaagaacctaaactatgccagatatttgattgagaaacagtattgtcatttaacaaaagaacagaagataaaattgggacagttcttaggattgactgagaagaagttaaaagaaatacaagagataaataatcagttaataaatttgagagtggggctaagttatgaagtcgtatgtaataaccagaaaaaatagtggggtatctttagcagatacagacatgggacaagtattagctaagatactatgtaagcatggaggaacacttaccattgaggaaattgaaccagaacatattggtcaaactttttctattgtagaaggtaaaagaatatggtataatagaaaaggaggtgataatattggggagaagaagtgctatgttaattctcaagtatgactgcgaagaaactccagaaagaattgaattacgaaagaagtggttacagatttgtcttcattcaataatatattatagatatgacaacaatatctggacagaccaacaatgggatgagactgccagagaggtagttaaacttaagaatgagaatccaggtttagtaaacagtatgccatttaggaatgaccttaaagtctttgatggatccactggctttaacctttcatgtatgcaagatattaagatgctaaggtgggcccaaaacttacttgactttcatgttaaacgaggacttgatggaaagagtaagaaaaagaagaagaggaggaaaaagaaatgaacctacttgagcactatataaaaaaggtacacagtgtaagagagtaccatgagtttgataatgaaccttgggctaaaggtaaacaatatgtagaagtagacatggactatatttgttactctaacataccacaaagaactaaattggtatttactgttgatgagtgggagaagataaaatatagaggctactacatgggataggaggaatagttatgataagagaaatgggcatgagaatgctaataccagaaatacatgatataaaagatagggatatacaaaattttgtaagaaaggccctaaatgagatagttgatgagaagttctttataatacctgcaagtagtactgggaaatatcacccaacttatagtgcaggggtaggaggtttgctaagacatactaaagctgcttgttatataggtaaagcattatgcgaagctgagatgatatgtcaggaagataaggatctgatacaggctgctctgatactacatgatattaataaacctgctaaggagcatccatatctagttagagagacactatcaccactaaaggaagagtttccatcaacctatgaaaagataatagaactaattgagtctcatcatggtcaatggggagagtacccaatcaatacttatcaaaagagaatagtacacttggctgattatatcagttcacagaaaggattaatattctcatatgaccaaggaactgattattctgacctgttatgggaggagaagtaaatggtgctaggggtaataaccctagtatcaatatttttactaggagtgtttataggttttgaggtgaagaaatggaactaggttttgcgatactagctataatagtatcaatatttttagaaactaaatatataaaggagtagattattatgtcaagagcaagagctgcaagaagaagagctgaaagagagaacaaaaatcttaatgttcttaaagctgtggacatcatgttagccttatcatgctatacattaaagaaggaaggttatggtaagactagaatgactaggtttgtggaagggatgagtaaacacacagaggagattgaaaagggaacactcaattatgaaaccataatgggagagattaaggatttagtaccaccaggtatatttgatttagaggaggagtcaaaggagtgatagtcttgggatatttattatgggcatttagaatattaggcatacttgtaatagtatggtgtataatggaggcgatcagttgtgagcaataagttatattttagatatagtgctatgaactctggaaagactactcagttaatccaggtagcacataactatgaagaaagaggaatgaagccattagtagttaagccaggaattgatactaaaggtggaccatgtataattagtagaataggggtagcaaggaaggtagatgttcttctacctcctagagttaaattatcaaaaatactttctgactttgagtctaattattgggcagtagatattatattaattgatgaggtacaattcttatccagggaacaggttgatgatctgttaaatttatcattacattatcctatcatatgttatggattgagaactgactttaaaagggaaggatttgagggaagtaccagattattacaagtggcccataacattgaagaattgaagaacatttgtcaatgtggtaaaaaagccacattctcaatattgaaatatcaaggtaaatatactgaccagggtaaccagatacaaattgataaccagcccgaaagtgttgaatatgaagccgtttgtaatgagtgttataataaattaattttttctaaataactcgttgacaggctatacaggatatgatattatagtattgtaatcaaattacaaacaccaaccaaaactttataattagttaatttcaaactatactaaatttgttatttaaaaattaaaaatatggaggaatgattattatggcaaaaggaaaagcaaaatggttagaggaagcaaacaataaggagctaattgtaatcgtacaatctgctttaaattctggagatgagaaagaagttaaaaaggctttaaaagctattgtggaattagatgaaagaactcctgacacagatggacaaataactgtagaggaagcattagaatcattagaaatggctgtaggaactgtaaaggctgcataccaagcagaaatagatgctgtggaagatgaagatgtagtagatgctgacttcgaagaagttgatgaagacgaagcagaagaagatgaaaatgatttagaatcattatctaaaaaggaattagctgctatggctaaagacttaggtatcaaaggagctaagaaaaaagataaagatgagttaatcgctttaatcaaagaagctcaaggtgaagaagaagacgaagaggaagaagaagaggatgaaactcctgactactctgaaatgactaagaaagacttaattgctcttgctaaagaaagaggaataaaagttaataagaaaatgaaaccagctgagataattgaattattagaagctgatgatgaagaataattctcatcaaataataaggggagcaaacttgctcctcaaattttttacttattttagaaacagatgttagagggggatgtataaatggcaagaggtggaaattcaacttatactaaagcagtatattgttacaacaataatagggagtatagatccttcagatatgcagctaaacagttaggagtatcggctgttagtataatgagagtagttaacgggactaaaccacatatagggggattagtattcgctgagatagaggaaggcactagaaaattaaagccaagaggagaagtagttccatacttgaaagaattaggttgggataatatagacttataatagggggtataatatatggacataaagttcttgaaggatatattctctaaacaatgtgaaaatggagattacattatcctagcagctagaaaaggtaaagaatggaaagatgtaccaatcaaatataataagaataacattgacaaaaaactaaaggactttgaacaacagtataagggttatgatttatattggagcccaatgccttatagtaatcctcaaagaaggatagttaacttcatagagactaaatacttaatacaagatatagatgagcatactgacccattggggattaaacctaaaccaagttatctgtgggaaagttctcctggaaaatatcaggggctatgggaaatggataggtatatagaggctaatcaatatgacgaaataaatccagcattagctaaacatataggctgtgactcctgctttgatgttactcatgtatatagaataccaggaactattaactataaatacaagaataaaccaaaagtaaaaagacctatacacactaaggagatatataagcctaaggtgatcgctaaggctgttaaggctgtaagtaaatctaatgatagtgttaaggtcaatataggaggatctgaggcctcacaatctgaaagaaagatatatgctaaatataatataccaaagaaggttagagacttactggctttagatgatattacttctttagatagaagttctaccatatggtatattgagaataaattacatgaaataggattagagcctaatgagattatattattagttaagggctcagcttttaataaatataagggaagaaaagatgaagaaacaagattaagaaaagaattggataaaatcataggaggagaaatagaggctgatattgaaaaggctgaaagtactaaactgagaatagatagttatcaggatgttatgggtaataatggagccttccctggttggttagtacaaggtttctggggtagaagatctcatggaattgtggctggacaaccaaaggtatttaaatccacatttacacaggacttagctatatcagttgctagtggaagaccattcctaggtcaatatcctgttctagaacctggcccagtaattgtagttcaaaatgagaatgctgactggattatgagggatagaactgaaaagataattagccacagaggagtagttggtaatgtggatataaaaggtaagagaagacttaaagttaggtttgctccagatcttcctatcacttttattaatcaacagggatttatgttagatgaagaatcccatagaaaacagatagaagaattaattgatgagataaaacctgtactagtaatattcgacccattatatcttatgtttagtggagatctaaataatgcagctgatctaaatcctgtactacaatggtgtcttaaacttaagaatgagaagcatacaggagttatgttaatacaccactataataaaggtggaaatgccactcaaaccagaggtggtcaaaagatggctggttcattcatattacatggttgggtagaatcagcattatatttaaaaagacctgatgacttagaaggtgatgatgaggagattgaagtagatatagataaccttgataaacaaagccatttaccaagtaaaatcattatggatagagagttccgtcttgcaggacaattccctcaaattgaattgaacttatcaatgggagaatttggagatccatattatcatgtagaagtagcaatacctggaaaagaagtaattgtgaaacctgaggacaaagccaaagttatagaggctgtaaaatcaggagctcataccaaggaggagatagtgtcaatatcaggattaaattcacagaaggttaatctagttctagatactttaaaagatactattgtatattctccggataaaggttataatataagtaagaaactaaatattgggaggaaaaaggcatgataataaagtgtaagaggccagcaggtcataatcatgtaaaggcctttgtagtagtaattattgctagaggtaagaagaataaaatcacttatcattcaggattgaaatatttctatacctataaatcagctaaattatttatagacaaagttatggaagaatgtccctgggctgaattttattgggtggatgtccatgaagctcataagatccctgaaggcgaagagatacctaagaagaagatgtggtgtccatattgtcaaaccattcaagaatttaagagaagccaaggaggttacaagaattgccctatctgtggcataagtgaccaagacttttatgtaaaatgtatgaatataaagtatagggaggctaaggctaatgcgaagaagaaacttgaaggaagtagtactaattctagggatatcattgtcaatggggataatgctaactcagggggcaaaactaatagaaagtcatcaccaaagaaaaagtcaagaaaagttagtagaaaagcttcataatgctttagactataatgttattgagacttctccatctgggctagatggggaggatcttatcatcaaactaaaccttaatagtcctaaggaccttacccctaacaacttatctaagttagctaatgaggttatggtcaataacttaggagctcagaattatactatacttgtttatgatagtaatagaccagaaaatcttctggctgttatagatatacctaaaaattctaaaataacctttgacataataaattaattatggtaatataaaaatgtagaaggaggagaatttaatggtcaataaaaagtattacatcttgaaagaccttaatgatgacaaaccccaactatggaagggaagtttaactactgctactaagatagccggagcattaggagttagtgtgataaaactaaataggtatttagtaagaaagggatttgccagaaggtataatgaacatacacttgaaataatagacccaatccttgctaaagaacttggttattatgtaactccaagtaagtcccttataaatcctttatttgaatacaatgataaaggagtacaatatatactaaacctaatagtagaagatctaaagaatggaggagagctttaatgattatcattttaggaatggataactcaggtaagaccacaacagctaagaacctaactaaacacagaggaggagaatatatccaatcaatgggacctggttcttatgaagaacaaagagaatgggtattaactcaaattgtgagaaaaggtgaaagggaagctatacatgatagatttacttgctttgaagaaatggtgtatggacctatcatcagagatagtagtaatttcaaccttgacagtaaggagctaaagatcttaaaacatttatgtaagccaactattgtatatgctagaccacctagagaagtgatcttcaattttggtgatagagaacagatgccaggagtaattgaaagagctgaacatctactagctagatatgatgaactaatatggaagttattctgtgatggatggaatgtactagtatatgattatactacttccaatgtggagaagctatcccagttaatagatgactcaagtgctaaagaagctataaaccaatttataaatatatcaagataagaggagagattgttatgaatataaaccatgcagtagaagaaaaagtagaaggagatagattacaagctatctttaatagacaaaaggagttaatggagaaatatcatcatatagaagctagatcaggattatgccaaactgaagactgtccagttaatttaaatgataaaagaggtcaagctagacttaaagattttgcttggagaatgactgaagaagtaggagaagcattagatgcttataatcatgaagaccattaccaagaagaattgattgatggattacacttcttaactgaatttacaatcctagctggtaaggattataatactatagatgataatgctatattagttgatagcctagagaacttatatcataaggctactacatcaccagaatttcctgaaatgttagtagaagaagctgtaactgacctggtaagagaaatgggtatgtgctgtaactgccttaagaataaaccttggaaacaaacttcaatgctaacagatgtaaatgcttttaatcaaagattgttcaatgtatgggtatgttatattaaattattagctgtatctggcttagaggtagatgacatagtaaatatataccttaagaaatcacaggtaaataaatttagacaaagatctaattactaaaataaatacttaggagaaggtaatatgctaataagagactataatgattttgatgacttattcttaaatcttaatagggaaatgattaccaatccagaagaaactatgatgtatactcagaacatacagggctttcaggaggacttagttctctcctgtaagtcccataaatgtactttaaatctaggggactttggatataaagaaggtaagtggggacacctattaagatcatacattgattatccacaattaattgaatttagagaaaagcttaccaagataagtggtatgagctatacttattatttcaacaggaaaaaagctactaatggttcttgcttgatagctgcagtagtaactagaccaaaaagaaaaggaccttggaaacacttaaagattatgtatagagtatgtgaattacaaaagaaattcgcagctgatctggtattaataaacagattcattgaagaactacctcaagaagtatgtgagatagataatattacttttcatatgtctcaagcttatttgtcaggaatgtttataaatggatactttaattattttaaggtaccaagaaagagtattgctaatagcaaacatccttggcataaatccttgaatagtaactataataggttctttaaatctgaggaccagatacactcttataaagctctccagaagatgcagttattacattttggtctggagaaatttccaaagatagatatcaataaattatctattgacaagtactttaataagtagtataattaacttaacaaaattaaataaatggaggaattaaactatgagaatttatattaatgctcaagaagcatttgaagaagtaaaaagggatttatgtgaaatgggtattgaggtaagacctaaaactatgcaagataaagttatagaaggtaatcccgattatttcacaaaggagttacaaaactatagttacacaattctagaaagtaaacctgaagaagttccaggtgtatctcaaccttgggcagatgctgaatttagagaaaggatctacgatccacaaggtgtcatcaatcaatactctcttgaagaaagagaagaattatttggtatacatccacatcatactagggaggctttcataaatcctggtaaagcttatcaattaagacctgaggtatggaatgaatatctaagagatggaaaatttggatattcttacaatgaaagaatttggcaatacagacaaattgaggatatcatcaatagaattaaagaagatccaggctcaagacaattatggttatctctgtggaatccagctattgatccattcaatatagggggagtaactagagtaccatgttcattaggatataatttccaagtaagagaaggtaagctaaatatccattatgttatgagaagttctgattttgctactcactttgctaatgatgtttatcttgctatgaaactattacattgggtagctgaacaaactggctatgaaccaggaagtttctctcatacaattttctctcttcatgtttacaacaaagatattaagggggtattctaataaatgagttacatgagaaaagaaagatataaacattctgatcttaataataaggttaacaagaaggaaaccctaaagcaatttattaggaatactgaaaaggaatttgatttagagccagctaaccttggggacatgactaagaatgaacttaataactatataaattatatgaatgaactgtggagtaagtaatatgtataattatagtgatgataacagacctaagaataataacaatggctgtcttggttgtttaattataatcttagcagctataggattatgggtaataatttttgatattgccaatataatatatcatatgatattctaatttaaagggggctatcctaatgaaaaatataatttgtcctatgtgtaaaaactcagtaacattaaaaagaaaatatggagctatgttttggatcatgattttcttaactggaggcctatgggtagtaactatacctttcaaaaaacataaggtctgtccagtatgtaattcaatcataaaataaagataaggacctttaggggtcctttctatttaggaggtaataatttaaatgagatgtaacaactgtaacctatatactcactcagctccctcatgtatagaaggaacttgtgtaggaaagaaaaagaaaccaagaataatggttataaatagtttagctaatgatagggatgaggccaatagaatagctactcctgataaatcattacttgataaaatggaaggtctagacttttattataccaatgctattaagtgtagaactcctaagggtactaaaatcaaagtatcggagattaagaaatgccaagaacacttacttaaggaaattgaaaaatataaacctgaatatattatgatcttaggatctcaagccttaaagatgctaagtaatgaaggtataacctcaatatgtggagtacctaaaaagcatgagaaatatggctttaagtttattgccagctattcaccgggtgtagttgcatatgacccaactaaagcacaatttgtagatcaggcctttaataattttaaagccatggtaaaaggtaaagagcatgaattaccagagcttaacataaaacttattaccagtatgaaagagttaaaccaggctttcaaatatttaagagaggaaggttataatagagtatcatatgatatagaaactagaggcctagataggtttaataataacattaccctatttggttttggtaatactcaagtacagtatatactgcctttagaagtcaaatatagcccattaaggggtaagcccatagcacaaagaagattagctaaatccctgattaaaagacttaattctgagatgaaggaaagaatagcccagaatggtaagtttgatgataatttcttaaaggagaaatatggtattaagccaatcataacctttgatactttactagcctcacattgtttagatgagaatacccctaatggtcttaaggagaatgctttattacattgtaatgctaaagactgggatataaataagaaattaaagactggaaatgtggagactaaatcagactttgaggattatgttagatatctgggatatgatatatactatacatttgctctatataaaatatttaataagagacttaaaagggatgagagcttatataaattattccaccacttatacatcccagctagtaaagcttatgaggatgttcagttcaaaggtatatatgtaaatcaggagaaattcaaagaggttgaaaaatacttaagatcggaactagataagattgagactggacttaagaagtatactaatggtaaggatattaactggagctcacctaaacaggttggagaatttttatatgatatccttggtcttcctgtgattgaggttactgactcaggagcacctgctactggggaaagtgtattattaagactaagagataaacatccagcagtagaactactattacagcataggggagtccatatacaaatttctcactttatagatggttggttaaatagaatgcacaatcatagattatatccaaactttaaacttcatggaactgtaacaggaagaacctcaagtaataatcctaatctacagcaagttcctagagataagaaaatcaggagtttattaggaccatctcctggaagggtattcattgaagccgatctatcccaggccgaacttagaatagctgctatgatggccgatgaggataatatgaaatttatttaccagactggtggagatatacatgactccacatataatattatatctggggaagatatcaatgatgagaaagatccagcagttaagaaggagaaaaggaaaaaggctaaggctgtaaactttggtttcttatatggtatgcaatggaaaaaattcaaggactatggaagagacaactatggccttaaattaacagatgaggaagccaaaacatatagaaggaacttctttaataaatatcctaaactattaacatggcatgataaacaaagaaagattgttaaagccaatggtgaagtaagatctccaataggaagaattagaagattaccagatatatattcatctgatagatctaaagctgctgaagctgaaagacaatgtattaactcacctgttcaaggttttggttcagatatcactttattaggcctatgtgagattacaggctatgctaaatatgttaatcctgaatatgtactagataagtctaagtttgatgtattaggctcagtacatgactcaattttatttgaggtagataaagattatgtggaagaattagcttggaaagtaaaatcaatagttgaaaacaatatggtattaaagaaagtatttaaatttaccccaacagtcccaataatcatggatatatcagttggttattcttggggaggatgtgttgaactggattttaaaggtgattggaaaagccagataagaaaagtgttgacagatgaataattatctgataatataaatattgtaatgaaaaggaggtataaggatgctaaaaataagtaattccagaattaacaaattcttgtcttgtccttatgcccattatgttaaatactatgaaggtctggtacctaaaagaagtggagctgccttacaaaggggctctgctatccaccaggctatagaagactaccataatgggaagagttggaaaaaatctgtggataaattttccaaagagttttacaaaaatacatttaaagaagagatccttgaatttggagatattccaaaaatggtttattctttatgtgataattatttccactattatgatgaaaaagaagataatgtaacctatgtggaaaatgaacatcactttgaactaaaactatgtaagggtgtaaccctagaaggctatattgatagtgtcttagatgtggatggaaagatatgggctaaggaaactaaaacatataaaaagatgcctgatagaaacttcctgatcttcaatagacaatctgctatatatacctgggctcttctacatgaatacccaaaagtaagtggtactatatgggatataatattagctcagcaaccaggtagacctgaattaactcaaaaaggggtattatctcaaaagaggattaaatccacacctttagagttagaaaggggaataaaagaattaggattagatcctaaggattatgagtcttacattaattctgctagatgggaagacttctttgtaagacacccaataatattatcaaagaatatacttaacagtgtaatggatgatactattgagatagctaaacttattagagatgagggccacaaaagaaaagaaaagaaccttggaaaaggttgttctttctgtgaatataagtctttatgtcaagctgaacttttaaatcttgataaagaatttattattaaggctgactacaaacaaagggaggaaagtgacaatggcaaaaaagcaaaaatcaaaatcaaatagttttgaggacagattagtggatctatatgatatagatgagcccacaatactaacactttatggaagatctggttcaggtaaaactactatctcaggaacactacccaaaccaatattctttattgatgtaaaggacaaaggtactctatcagctagaaacaagcttagagttaaaagaggagatatacaagtatttagtctaaagagttttgatgacatatacgaggcttatgattacctatcagaaaacactgataaatttaaaacagtagttatagaccatttaactgctttacaagaattaggtaatgaaaaggtcaaagctgaagaaggtaaggaccagatgagccaaagaatgtttggaaatgtggctaattacatgaaagaggttataaacctttataaagaattaaatgaggaaggtatactaccatgttttatagtacaagataggttagaatctggtgatggtgaaggagaagaccaattaatgcctgaggtaggaccaggattaatgccatctgtatctaaatatttatgtgctgtatcaagagtaataggtcatacttatttatatgaacactcagaaaaagagggtatgaaggttaagaaagaaatccagtatagattaagactaggacctaacccttactatattactaaatttactagacctcaaggatctgaatgtccagcttatctagtacatgatttaaaatcaccaacaactatctgggaggatattgaaactattctggctggtgaatggaataataagccagctaaatctggtaaaaaaaccagtaagaaatctggtaaaaagaaaaaataaaaattttctaagaatacctttgacaaccaattaaaaatctgataatataaacttgtaaacaaagaggtacaatatcttgacaaatatttatctcataatgatattttaaaatttaaggaggacaataacatggctaaaaagactactaagagaggtaacaaaaacaaaggaggattaaagattgatctttcaaacgttgaaacttcagttactatcccagaaggaaattacattgtggaagtagaagatgtagaggttaaggtttctgaaaatagtggaagcaattatttatcatttacttttgtaatagcagaaggaaagatgaaaggacaaaagttataccacatttgctcacttcaaccacaagctttatttaacttaaaaggtgtgttagttgctttaggatttgatatccctgatgaggagttcgaattagatacagaagctctagttggtttacaatgtggagtagaagtatcacatgaaatatatgagggtaagaagaaatcaagaataactgattttataaaccttgacgaagctgactctgatgatgacgaagatgaggatgatgactcagatgacgaagaagatgatgaagacgatgagtctgaagttgatcttgaagaattagacaaggatgagctaaaagaattagctaaggctttaaaaatcccagctaagaaaatcaagaaggctaaaactgaagaagatctaattgatctaattgaggaagaagctgacgaagaagaaatagctgaacaatataatgacctattcggagactctgatgaagatgacgaagaagatgaggatgaagaagaggaagatgaagaagaaaatgactatgagtcaatgactttaaaagaacttaaagctgaagctaaggacagaggcttaaaggttaaaaaaggaatgtctaaggatgacatcatagaaatgctagaagaagatgatgaagaataaaatttaattattaaaatatgagggcctttaagctattatgcttgaaagagatgtggttaaatccataatgaatatgcttaaaaaagaatacccaggtttttggtttaaaactcatgggggaccctttcaaatagctggcttacctgatatactaggttgccacaaaggtaagtttattggtattgaagttaaacttcctggaaaagagaagaacctaactcaaaaacaaaaagacattataaataaaatagacattataaataaaataaatctagcaggaggaatagcttttatggctacctcagctgaatatacaaggaggagattacatgaaaaatttagaaagacaccaacaattcctaggagaactaggagatctgtatgaattaaagaataatctatacggggataatttccacaagacttatcttgaatatggaaaccctgttctatgtataagacttgaagataagctaggaagagctaaaagtttattactaggagatcaagatgacttcccttcatatgctgctcaaaaagaatctgtggttgatactttactagacttagctaactatgctattatggctgctatggaactaactagtgatgataactcagaaatacatgatcttagtaaagaagaatatgatgaagaagacatagatgataatgacgatgaggacttagatgatgatgacaatgtttacgacgaagaagaattagattttgatagtatgaataaagaatccttaaaacaatatctaaaagataatggggttaaattccatagtaaggcctcaagagatgaacttgtaaaactggctaaggaggtataaggaggaggctttaatgcctcttcttttttagtatgaagagaaaattatttaaacatcagaaagaagcattacaactatttttaaataaagaaaagtttgccctatttatggacatgggtacgggtaagaccttagtccctattgtggcccttgagaaacttgaaggtctagatactgtactaatattctcacctaaatctattgtatttaactgggagtctgagatccataaatttactaaacttaaagagtataaaatatttaaactacagggtaataaaaccaaggttatggaaacctatagagctataaaatcctactcaggattaaagattattattgccaattttgagaaggctaggttgatggataaataccttatgaacttaaagccacagtttattgttgttgacgaatcccataaggtaaagaatagaaatgcccagatatctaaggctctatataaaattgctactaaatgtaaatatagattgataatgacagggactcctactcctaatggttatgaagatttatttatgcaatataaaattatgaactcaaatattctaggagttaattggaaacagtttgaagatgactttataatcaaaggaggttatatgaactatgaaattgtggggtataagaatgaggaaatattaaagaaccttatgcaccagaattgctatatagtaagaatagaagattgtattgatttaccagaacaacttcctgatctgtatttaacctgtgaattaaataataaagccagaaaggcctataatgaccttagaaaggaaatgatagcacaactagatatagtacaggaaaacattcctaggaagcaattaaaggccttattaaggtctaatggtataccctatgagagtaatgagccatatgaggatttattcttaagggctaatatgtttatcaaccaattaacagctgatcttaccattacccaatatttgaggctacagcagatctcagggggatttattacaaacaatgtaggaaatagtattaatattgataaaggcaaattaggcttattacaggactatctagaaggatataaaaagcctgttgttgtaatatgtaattttcttgaggaaataaaacttatacatgatacttttaaaaagacccacagagtagaatgcttaacaggatctactaagaatagagctgagatcaataaggattttcaggagggcaagattgacatattaatactacaaataagttcaggtagtgttggtctaaacctcttcagggcctcaaggttaatattctatagttggaactataaatatgatgattatgtgcaagctattgccagaatcaaaaggaatggtcaaaaggagccttggcaaataatacacttaataactgagaatactattgatgagaaaatactaaaatcaatacagcttaaaagggatagagcagaaaaactgttgactattgataagtaatgtggtaatatagaattgaggtgataagtaatgagaaaagaacaacctagaataagtaatcatactgaaatagctaaagctttaattgaggctggtaaaactattaaagccctaaccaagattaaagtcaaggaggaaaagaaaaaacaaaagggacttgaaaggttaagactcctaaatgaggaaaagaagaaggagagagaattaatgaaagaatttaatcctaatgagcccaagaagccaagaaagaaaaaggacaaacctataaagaaggttgatctgaataaagttgaggccaaaggagattaccctactactcctggaagtaatgcacctactggaacagttgatttaaaatctttatgtgagtcattaggattagatccaagtaaagccagagccaagctaagaaagcaaggggtaaacaaaccctataagtggtcaggatctgaacttgaggatattaagaaaatgttaaaataggggaggtttaaaaacctcttctttttcgttgacaatatccacagtatatggtaatataaatattgtaatcaaacaaaaaggagagaactgaatatgaaagatcttgaaaaacttacaagggaagaattgattgaacttgtaaaggagttatctattgattggacaggagcctataatagaaactataagattgaccttgaggagtatgagattgtatatattgatcttaatgatctaaaagagttcaatgataaattaggtcatctagaaggagatgtgtatatacaatacactgtaaatcttattaaggcccatttacatcctgagaaggatatattgattagatggggtggtgatgagtttgttataatctgtgtaataccttgaatatataccctgagctatcatgtggctatggtaaaggtaaaactattgaagaagctatagtacaggccgactcaatgatgtataagaataaaaaatctaaaaagttaagatactcaaaggaggattagttattatgaggttattatggttaaaagagtatttggatatatgcaatatcttgggtattgagcccacaatggaatcttgtgctaggtggggaaagaataaaaaattgtcagggttttcattaaccccagtaggagcaactaaatagttgcttctttaagcgatccaattcctaactttcatgccaaaaagaaattcgggattttctttgacatagactccagagtctggtataataatcttgtaaacaaaattaattcaaagttgttaattatgaataacaaaaaatttcaagaaactttagaacaattaaaggaggaactagaaatacaaggggtagaatttaatgatgagaatgacttaatagctgctgttgaatacatcatagaatatgattaccacccagaacaatatgtggaagatactttatgtaactaccctgaaatgttcaaggatctataaaactaaggaggtgtaaaaacctcct

SEQ ID NO: 11 - DNA, 51409 bp, Unknown
  Clostridium phage vB_CpeM_SMS460, complete genome
aaatcataaattaactattggaggtaaacaaaatgagagatatgatagaaaatcaattagctgaatatgaagtaaaggagttatataatgaggccacaatgatcttaggaaaagcctttggtgtaagtgctgttgatctttatatagaatctgatgtggatgctaactactatggtctatgccatagatcaggtaacaagtttactggaatcacaataaaccttttccctttcaggtttgtacaggaaggagaaaaagcttatgacaaaatcaaaaaatttggttgggatattaaggactgtgttcttgaaactatatgccatgaattagctcacttaacttattggtcacatagtcctttacataaaaaattaactagtctttactataataaagtgcaacaggctagaaaaggtgaaaactcagctgtagcacaaactagaagtgatatatacactttaaaagatttatgtgaattgatagaactagatccaagaaaggctagaagtatattaagaaagaataacattgagaaaccaggtaaacagtgggagtgggagcaaggtatgcctaaagatatatatgatctattaatcaacttaaaggttaggggaagaaggtaaacttcttctcttttttcgttgacatatctcgagagtgctggtataattatattaacaaataacttaggaggtaaatcacatgatagatatatattatagtattcaagtagaagctgaaaacttctttggttgtcaaaaagaagatgggagctgggtagcaacaggagcatttagtatacctgttaatagcataacttttgaagaggctgataaattatccaatgaggccatagatggatggttaaaggagtatgaggttactaaggtaataagtaggagatcacatacacaggttaaattggattggtataatagttatatcaaggatgatccattaaagggtgatataattaatatggtgtaccctgatctttcagataaagctaaaatcaaatatggctttggtaagatatgggctagcactatgtcaaaggttgctaagaaaggtttacttaaaacttgccctagatgtggaggcactggccattactcaagaacctcagatggtaacactacttgctttaaatgtaatggctttaaatatgtgataccaactaagatctcaaagaaattttataaatctgtggagaaaacctttgacatctctggcaataactcttataataaacttgtaaacaaaaacaaagaggagatgttaaatatgaacttaactgcaatggaaataaagatattaaatgctatgagaaagaatgaatttgatgatggactagatgttgactgtgtatgggtattctcagtaatagaaaactcaggtatagaaggaactaaggctagaggtgtgatctcatcacttgttaaaaaaggtttagtatttgctgatggagaggtaataggttacacagaagaaggaagaaaggtctttgataatgctgatggagaagaatgtaactggggaggtccaaaattattaaaagaaatccctgacaaaccagaggaaatctcttataatgaaaatgtagataaaacaaataagaaagaagaggtaaatgatatgaaagatttaaatactatgaaagctgttgaaatcaaagaattagcaaaggaattaaaagttaaaaactggtggactatgaaaaaggctgatctaatagctgctatccaacaaatccaacaacctcaagaagaaaaagttgaagaggtacaacaaccagctgaacctgaagaaaaggctgaacaaactaaaattactcttgatctaacagatgttaaggaaccagagaaaaaggatgaaccaaaaactaaggaaggtaagttcacattaaaaatgatacttgaagaattaaatatgaacggtaaaaaggcaagaaggatcttaagaaacaaagaggttgtaaaaccaggaaagcaatgggaatgggataatgaagaagaatttaaaaaggttaaggacctactttctaaataaagtaggcccaacctctatcccctattgatttacaggaggatctgattttctcttatccattaactgcttccatacttgattaccaaaggaagctacagctgcacatattaaaccctgtataaagccattcaaaaaggtcttggctgtaaaacctaattctaattgaattgccatatataagattgataatgatatactgaatatcaaaagtattaatggtatagaccaatctggtatataaggcttagccttgataaacagacctaaacagtaacatgatacaatgattattaaaagctgtggatcaatgtaattgaatattaagtttactaaattttcttccatgataaatacctcctaaactatctctacatattttgaacttacataaccttgcttcttaccgtggctagtattatattctatatgataccaaccattcttacttcccaatatagtaactctctcattactaaatactgagcctattatattaccattcacatcatctcttacattaagagaagtgttaactttaactattccttgtttaacatcctcatagaatttctggctttgctcaccctcgtagcgaacgtatccatcagatggagtaccatcctttctataaaatgttattcttaaccagccattagttctatacattgggctagccttttgaccaggctgtagttcttcattatgatctttgatgttaacgtcttcagggttccaccacacataagttactccattgtcattttgatatttcatgtggtagtcaaaacttaatctgctgtaatgttttatgtcaatataagcatggtacttcttattctttccaggatatacaacttctatgaaatttctagaaggatatattccaaggacaaataaattatctaggctatcaataaatctaccagggactggcttacaatcctcatctagaacttctattgaaccttcccccactaaagtagcattagttcttgtctcattagactcatagaatcctttaggattattatctgctggaatgcttggagtactacctccactgatctccttattacaaatacctccagctattaactcaccaataagtttaggtcctttagccttatagatacttacatcagtggttgcttcacagaagcatacttccactatgacagctggcatggtagtctttcttagctcatataatttagcattacatttaactcccctattctttaatcctgtacctttggcaacagcattaactattcttgtggcatatacttctgcttctcccccagtgccatatatccaagttcctgtacctaaagcaccttcatacatatcgtaagccttgtcaaagtgaatactaataaataagtcagctccccaggcctcagccttattaaccccatatgctaagtcagtgttaacatcacaatttccaggagtgacatctagaacactatggccttcatttcttaaagccagtatcacagagtcttttacttttctatcctcagtagtttcatcaattaaaccataagcacctttagcttgaaagttatgtccacctcttactgctatcttcataataaacatctcctttaaatattatagtgttttattgggcctgagaagctcgctacagcattttaaccacctccttaatatattttcttatataaaacctaacaggccttaaatgacctgctaggcttataatttaacctttgttactaaataggtgatcttttatagtatctacatcttcctttatatcctcaactacgttgaatttatcaactagcttatctatcacttgatttaatttattctctctttctctagcctcttttctagtgtcatataatagccatacaaatagactacaaaagataccttggctaagaacttgagttagtagttctttttccatagattacaccctttcattaaagtcttcaccacatattcttttatagtcctctggtgtaatttctccttctggattagaagagcattttactgcctgatataatttgtccttacctattgctccaattctataagccatttcccagaaattcataatctctacctccttatttattcatcaatttaatttttatatcagctagttcttgtgttaatactttcattatggccttagtttgcattagctctaatttggtatcagcaagagcctgagcttgatagccaactactttattaatatctagaggattaggatctatagcctcatatgaagtaaataggtccttatctttgatactatatagatctgatctctttttactaatatcaaatttagctgggccattttttatgtggtcaaataaacttacagtcatttgtataacaggaagattgtctagatcaataacctcagtgttatccacaaaggctaaatgcccttgtgaattatgaggtatatggagacatataatatcttctatattatttaatatttcactaccctccctatagtatagaggttcaggtctattaattaggtctaatattttagtaacctcttcatggtctttatctataaatgatcttacatcttctgagtcttctgcctgatttatcagcatttctttttcatcttcatagaagataaggtctataaccttattagtttccacagaataattaaatcctactattctcatgttttatcaactcctttactcagtagctaaataggttagtattatttgagttggcttaccagctaccatttgataacatccaccattcaaatatgagttattccaatagtcaatagtacaataacctgaggtattattagaagcgtcattatagttatgtgatactagagatataggtaatacccaattccaagccttagggaaatgtatagtataacttacacctccacctgagttagcatagcctgtcttaactatttgacgaactaatcttagtcctcccccaagatgacatactctaacgttaccattgtcattatcccaatagtctcctttagcaatagacctatcatttacaacgaagtcagtaacattctcagtatataattttttgttctcttgtttgaaccacaatcttccaccttcagctccaccataccagtcatttattcttaggtattttctagcccataggttttcagctgatagagccttatcaacttctagagacccatctctagcagttattctcatatcaaaatctttcttactaccctttaggtggaaatctaatataggacctagttccataactccatcatgtgatataaaaggaatacctgcatagtcattactagctccagctaagtcccattcttttagtttattactgttagcagctgtagctgttttacctaaataagcactatcatggttatgtcccgaagaggctgctcctatctcagagggagtgggtttattactttgatgataaattaaactcccactataagctaatacaccgtcatcccttaactgtaaaaatttattagactttttattttggagatatacatctgttccccctactccacataataaagctctcatattatcatccctaagactagctaaggtagctactacatttacatttactaattgatccactgtaagtttgccttcaataaccccgtcacccttcttagctatagcccccacatctgctgcagttggtttgttatcttcatgatagatcttcttattgttcttattaacaatatctaaagagtctatctttacaggccttccagcataccctatatggactttattatctgcatctatatagatcacatagtcatcactgccttctttagttctacagctaaatccataattgtgggccccaataaatttctgacctggtctcatgattagatgacctgataattctccacccgtagtaggtatggcccctatatcagctggagtaggtttaaagttagttgagtattgttcagcccaagaagtccattcagtagcaagggcattagtgctacatctaacatataccttattagtaatatcagcatagaatacttgagttacccacttctggtcattgtcccaacctaatactaatagtcttccataggattgtggggcatgagttgccctatccacaatatataaccctggtgtagtgaatgtgttaagatcctgagttattctaagaggtatctgaggtatggcccctatatctgctggagtgggtttggcccctgagttataatcctttctccaagggaaccaatccttatcatttaccttatacctccaatatttattaccattagcgtcataatattcttgccatatacagttattactattattatgtttgtcctggtcacttacgtatataaccagtattccataccttacaccagttataggggcattaggtaactcacctgatattagatactgtccctctgtcaaagccagattaaaatcagttatggcgtccttaggaacggggtaagcctttaggtcagcaggggtaggcttattagcctcatcataaaccttataccacttactccaagaattatttactaatgtgtagtttctaatatacctagttattttattggttggatatagattaataaactcctgttggaatccccatcccccctcaccaatatttctaactataaaaactctattactcctatgggctaaggttccctctggggtatttttaaagtcaccagctcctgtatcaaaatagaagaaggcattcgcttgtttaaatgaattacaatctctatctataggagcactaacctttagaattacatcggttttggcagctttttggtttacttgattttgtaaactagtgtctttctcaaataataggtctaatctttttagtacatcctcaggggctaggttatagtcaggtactacattacctctagctaataagatatttaatacctttaatgatgtgcttgaggttgcaccctgtaccacaaattgtattttagtatcagtagctccagagggaatactaaatttatgagtttttaatttatacccattactctgtgatgtgctaccttgctttacaatgctcttatattgttggcctccactagtgtaagccagtttaatctcatagctagtaaaactaccttcaatatcctctaggaaggctaatacataatcactatttggttctataggcagagttttaatttcaagagtttgtgggcctgttgacatacctgaccatttaaaacctcgtctcttagttggagcatctgtaatagatgtgatattaccaggaccagtataagactcagagttgtaccactcaagcctattaatagtaccaacccttaaagcatctagcctatcctcattagacttagacttcttttctacctcagtcattttctgtttatgttcggcagcgaagttctcaatgttttcattgtccttgttaaaatcattcatttttggcttatctgttaactcccacttatttaaacctagagtaggagttttaccagtacttggcattttgcatacctcctttattcagttatagcatattcttcccatttttcccaggtaaggtctagactgtcccacttgctccacatataatcatatctatcgaacatctcccaagtaatataaagtaatataatttgatatattagatgggcaggcattctttctcggaatactctttctagagcctttaagttaggtggtaccccaggtaggtcagtaaagctaattttgatcttactctcttcagctatgaactctacctttacctttccacccgtataggccttagtaagatttttgatagatgtactattaaactttttccaagcagatagaactactgtaataatagctcttttctcttggtcagttggtggataatcaaaatatttatatagttcaagatcagtgaaaaatctttctagtccttccttgggagtggtcttgggatccacatagtcttgtaattcctgtcccttagtatatagcttctgaagttcagtagccatgatagtgtttactaagttatcagcctcttttatattatcataaaattcaggaacgtacaatagtaattctttttgaacctgttctagactatacataagttaccaccacctcagtaatggttggtatttgattagcaggaatagttaaagattgactactcccattaagtgtaatggttgtataatcttctactccctcaatatttaaaagcatagcccctattctatttatagataggctcgtaattttaaatacttgactatttagaaattcttctagttctttcttgaagtttaattttatttgttcttctgtaactgaactcaatttcttaacctgagctcttacagtgatattaaaggtttgagctgactcaacagtaaaaacacatcctataggagctttaccttcacctaaacctttcttgcctggatccatgtattcttgaactttattaactagttcaggattggcaggcttattctcaactgtggttataatacctttagcagtatttataccattccaacagggaagaaccacagctttacctactccagctacacttttacaccaatcagttaactgttggacatttccatctccagatggttgtatagtggctttagtagctctttctcttaactcatcatctgtctcttgatctctaccaataacaagtatatctcccatagtagcactcttaagttctgctatagcattgatagggattagatgtgtacctgttgatactttattgggtcctgaacctaaggtctcacattcaataatattaggctctatcacttgccagaaataatctcctccttggaatcttgaaccaacatcgggttcatagccaacaaatttagctgaccttctacacttagttggaattattctggatatccctacattggcagctctctcatctaagaacttacctgtagcagtacttagaaatactgagtctaatatttgattaacattaacagcatagaactcagctagcttaacagcaaagaccatggccatatcataggtgaaacttccagttctagtgtcaatatcatcaggggataggcctatcatctcttctaataattctttatgtgttcgggagaataaatcttcagaaaatcccattatatcaccccttccagttttgtatctccatatatagtatttatccatacttcaatatagacattcttaccttgcacattatactttagatcagttactttagtcaccctatcatcttctaagagacattcaggaatagccctattaatctccatctcaagaagttcaggagttactagagagtcattaatcaaagccctaatatcacaaccataagaagtatcttgataaatcatataggcatttttaggagttattaatcttttatataaagattgttttaaggcctcaagaccatccacattatcataaatcctattaagatttagatccagtttgtaggtcttggtattctccattgttttctcatcaactataagggtatctttaaagccattaggtagcattatatcaacctcctaagatccctaacataaaatatctttgtccatcatcatagggcataaggtaaacagtatcaccctcactaatagactcaatatgttttggaacatctataatatttccagatagtactaatttcctattgtgaagacactcacattttatagggctggtactaagtactttagctctataaatatttagaggggtagacatattactatatacttgttttacacaatctaataaattattagccattaaatatcaccctcctcagtacatttaagttttaagttcatggtgtagtcagtatcgaatttatgagtatcagactcaatgtagaaagccttattaatttttaaatgtggtatactaactacaatggctttaccactaatacagtcaggtacacctataacagttatagagatatccgttttaggtctcttacctgtttctagctttattctagccttctcatttaattgggcttggtttagttcatcactgatagtatcgtataattgaaatacaccatatttcttttggctgctatcatctttagcagaggcaaataaagtaatctcgggatcatctttacccttaccctgtttatatttacatactagctttacctggttgacaatactatcaaagtttctacttcttgaaaaagttccaatatttttaccatattcgaatttccacaattctttagtggttcttcggtttataaaatcaaacttacctgtaatagaattataataaatataaaactgttctccagtttgttcataaacctcttttattaccttcttcataatatcatagggagtttgtttctcacatactaaactagagaaagtatatccagttttagctactactccaggggcaaccccaaaggctttacataatgactcaaatatagcttcgccagtttgattttcaaatacaaaactatctttattattagcccaataaatcatagggtcataagctttaactttaagtaaatgctttttctcattatctgagatattcataataataccattaaagatattcttaccctcatggactagtactacctgatctccatcataaattggatgtatatcttgagcattatattggaatgatagggtacgaggggcagatgaagaagaccctgcccaactcttaccactggaggtatttgtaatatcaaaggatttatctccctgtaccacaattaattgcatattataacctccttatataatcaccagaactccaaccttctttatatcctgaaccattatctaatttatactttacataatgccatccattttgagaccttaatactgtaaagtgttcattaggccatatcctacccttagtaggattattgataccaggtccagttcttacccatagccatgagtaaacattagtagtagtagctgcccaaggttgagtacctccagaagaactaccacctgtattaccactataggctagtttaggttttggaattgtgaaactaactgatttaacctcaggcttcttatactcagttagttttatttttacttgaagagtaccaatatctccacctttctcagtatatgagatattatcaatagtaactaataagttaatatcagtactagtaatagtaaatcttactggatctttacccatctcaataagtttagcataacttactcgagggtctgcaatatcagtatactcacatccatcgtagtatgagcttggtaactcaaaattaaaagaaaaagattttagcttagggtctcctatgaaagagagttcccctaagccttcaatggaaactgtactgttatctctactatcagagatatttatatcagtgggatttactggtaacctaaacctagtatctccctgtattaaccagaattggtatttactatgcaaggtccagttcacctccctcgaacatttcctgatttaacatttccacaataaatttgtatactctcattaattcactttctgagattgttccatcttcagcctgtactgtgaaattaaatgagttgttaatgttcataactttagatccatcatcacttcctgctccagattgtcttagtaaatcctgagttttatctgcaggtaatacttgagagcctctaggcatttttgagaatgtaggacccataactaattcgggaccagcttcaccaaccatggcagctttatcagtgaagaaatcagtaccttcagcaagcattggtatctgtggaatattgatacctttacctcctatgattggaacccagtctggtattttaatagtattaagagcaccaattaatccattgattaaactaattattccatttataggtcctttagcaagagctagcataccattgaatactccaccgaatatattcttaactccctcccaagctttagaccagttacctgtaaatactccggctataaatgcaattataccacttagggtttgcattaaagcttgaagaatatttgagatattttgtacggctccatttacagccgcagcaatcattggccaaacagcattaaatactgctcctaaagcataaagtataggagttagcatatccacaagtccttggaatatcataccaaaactttctatagcagattgtatatatggaactacactctcagcaaagcttaagaacattggtaatagtgtattattccaccaatcagctaatccctgtaagattggcattaagatgttacctattacttccacaacttgtgaaattgcaggagctatataagtagcaaaggcctcagctaaagaagtagcaattggcatgaaaacattagttatcaattccccaagagctgtaaaagtagcagagaacatagagcctaatacttctaatataggacctaacacttccaatacagtttgccatatttgagtaaatccctgaacataaaggactactaggtctatggctggaattaggttattcataaatatagaagctaaagttccaaacatatcaagaagtacttgacctataggagctaatactcccatgataggctcagctaaaccattaaaggcttcttgtatattattgttagcctctgataaataatctggtagacctactaagatctctccaaaattattatatatgtcttccccaagactccatatcttctcattaatttgatctgccacatcagcaggtaagaacttatataatatatcattggttagatctataaaaggttgcatagcatccccaaactcccaaccattcatgatgtaatatatagtctctcctaaaccttggaaagcagaaacaaccattgagaatataggtcctagtttttcaaagccttcttgtataaatgtgataccatttaggacagcattagctataacctcaacagcaggggctagggcatctcccacatctaacataactacagcagctttggattttattctctccatggttctattaaagcccttatccattttttcataggcagcttcagtggccccagcactattcttcatctcattaagagcattagtaaaggcctcagtaccttcaccagtcaaggctagtgcagctgaaccagcttctacagaaccaaagaggtcattgatacccagattacttttctgagcgtgtttttctaacatctgaagggcctcttggatattacctccacctttaatgaagtctttgaaacccttaccagctatttcccggaagactttatcagtcttggttcccgatttagatagctcatctatggcagctcttatcttagtggtagctacagatgttggaaccccctgagctgttaatgcagctatagaggcagatacttcttcaaaagataccccagcagctgaggctgaaggtagcacattgaatagggattgtgatagttgctcaaagttagtcttaccaagtcttactgtagtaaacataaggtctgaggccctttgaacatccatattcttagctccatatgagttaataacagtagttagtccatcaaccgcagtttctattgatgtgataccacctatagaagctttaccagctgtttctaagaaactaaaaacgttatcctctggaactgatgaggatatggcttgatagagagcaggtactactttatcaggcaatatccctatatcagaacttaacttttttacctggccggacatcttatcaaaggtctcttgagtagagttgggtaatagagtcataacctcatttaaaccttgctcaaaatccccaaaggctttaatactgtccttagtaaaatcccagacagccttagctgagaatactccagcagccaccccaccaattttggcaagagtacccatgaaggaattaccactatcagccatctggttaaatgcaccagagatcttatcttgggcagagaatatggcagtaaatgcagcttgacttgccattatatccctcctttatgattaagagcctttctttaacttatccctagcttttttatcttcttctaaagccagttcacatgaagctataagaaaggctttcctattgggagatagtttatcaaattcccagggtagtatattatgcttctggaataacacatgagcccaaaatgcactagtgtcggcctttattagttttttgcttctttgattagatcatccttattatcgcctaatccatttatgtccataacctcactagttattctagtgaaatctcctggtaagcttagtacatccataagtaaatctactggatccacaaacttagtagactctaaccattttggatctttaaagtcagggaatactattgtttctagtatcatttccctagtagctcttctttggtctgttattgagttagttatacctttgtttctagtaattactgtatttctcttttggatatcctccatttgcttagtagtgattggctttaatatccaaggtataggttggcccgctaattcatgaccctcagggaaactaaatctagctaccccttcaacttcaatttcgggtgattgtttacccttatcattttccatgaaaaattccatactaaattttgacataataatctacctccacaatattcttaatcttttataaataccttacttctgaagctgagaatgatatttcttcttgaatttcttctccctcagcatctagtccaattagtggtaaatctccttctaattgtactcctagtaattgtactctgtcctcaccataattcttaacatagtcagaatctggatcatcacatacagcttgtatatccatcttagggaatataccagtctttaagtattctttaatagcttctcttatcattggagtagctttgaattgagttatagttccagttatatcataacctataacttttgaggttaatcctttctgacccataacccttttcttagtcacctcaggagtaaatagagcttctaattttattaagctagttacttgcttacctgatacaaaagctctaccctctctagcagacattctattaacttcactcatgggctatacctccttaattattcttaacagttacaaagtatttcttgaaagttcttacaggttgtgtaagtatagtagcatacatagtctttcctttactagcttcttcatctaccactacatcagagtcagggtccacattctttaatgctccttgagcttgtagtgtagttaatagacttattagtctatcctgagctagttttataccatctttgtggttagggaaaacattaggaactaagatctgagatcctagatctataacagcatctaatacagccacaactttattatctgtaaagctttcatcccattcttctgtaaaagtatggtgtgtatttatatcttcctcaactactacctttactctttcagagtttgagtcagtttgagttgagaatataaattgacctgtttttaatccatcaactatttgagagtgttttagtcttggattagcatctaccgccccaggatatgctttataagtaaggtcctcagtatatccagctgcagctctagcaccagctacccatgcagttgcttgagaagctgttaacttagtgcctccatctaatacaaccccattcttaacattgatgatatatgggtaatcacaagtagcaaagtcagaagtaactgccacaatgttcttaccaacctcatctctgaagtatttaagttttgataatagagcagtgtgtaactcactctcagtaaatgggaaggccatagttttaaagttgtataactcacagttatctaagaagttgataacatcagagttattcatctgagtagaagtaccagtactaaactcagtcttagcagtctcatctatagaaccacttccacttactgtaaaccactgagacttgactgttgatatttcagttactcctgtagcttcaaatattttagacccttcaagatataataatatatcctttcctccaagaggattagtcttaatggttagagataatttattacctctttctccagctttagtagctgtaaatgttagagaccctactacagctttggctttagttccacctgttaggttatatactaaaacagttgcagcttcttttaaagcctctttaactagtaacacagtagggtgagttatgtcatacccaagttgatataatgcttctaggccaccttcattggttaccttgataacatctccagcattaccccacttaagttgtaatggtaagatcataactcctagattattaagcttaggctcattaagtccaccacttacaaatctagtatacataccaggtcttactttattttgtcctggaataaaagttcctcctgccatctattttacctccttctttagccagtcttttataatctgactagtttgttttattgttaactcttcgtcaggtggaagggattgaactgcagcgttgaagacaaactcctcaacctggaaaagttgttgacaatgggctcttaattgaggtatggaaaatttttgtactttcactattttaccccctttgttatctcaaagttttcgaagtaagttggatctttataaagactaaccacaatatcatagttaatctctacctgagaatcttcttcagtttcaccctcaaatacttccacattctttattttgagatttttgttggtcttactaccatccctattaagtataggaatttttctatttctcaagtatatagcattagctatattctgagcaatttcaagagagtctttaggttcaaataccttaactcttagggtattctcagttctataactacaaagagtatcattctgaggattaaataccttcttgaaatataatgagggatacttgaatttttcaggtattcgctcaaaatagagggtagtaattccagttagatcagatatgaatttagcaatactccctatatctgctgtcattaataactaccccctaaaacttcggctaaccaatcttctaattttctttggaaagcctgttcaaatattttttcatatatctttactgcattgtcccaataaggtttacctggtactttcttttgtcttagtaccataccagtcttagctcctggttgataaataaatttatcccctgaccatatgccaggaacgaatctaaatgattgtccttgtggattagtgtagtgaccatcgtttacccacttagcatattttaggttagtaccaacttctaaagttagtcctccatcactacttacccatataccattcttacctcccttttgaaaggagttaagtaataatctagtatctactacttgtagtctaattatttcatcttgaaccacatcaaggaagtctatccctgcagcttcaaaccatactgctaattgctttttgaaatcactattagcggcatctcttagcttctttataaacctttctatttgtttagcatctatttctatgcccccagaatttgaagccataactacctaaaatccttagcctctataccaaccttaatttttagaccccttatatccctaggagctttagctatatatgtgataccagtctcaaggtccacaattttatcattttgtctaacatcagttccaatagcaaaatctactgagtactcctgggtaattttaaacctaggttctttgtactcaagtttttctagagttcctgacttaaaatgacagggtacctcagtcatgtctggctcatcaggataactaaagctaggggtatgggctaggccatatccgggatcttcaggatcttctaataggtggtatatattacatctatgatttaagagcttgttatatgaatttctaatcatattacagtacccttaatctcatagtagttgttctgatagatggactatcatcttctaagaaaggtttaatcagaggcataatatcaggctctataacagccttatcagttcttcttgtatatgagtagtcatcataggtctcggattgaatatcagccttgctaatgaccaaggattgtagtccataatactcagcatacagtatatgggctaaccttaattctttactattaggatttttctccccaatagtatcatagtctttatttgaatttttaaaaattcctgagatcttggctttagctcttagtatatccatctcaatatttttggggtctcttttcttaatctcaggattacttgaaaaatcaattatttcatccggagtaatattcatatgagccacccctatctgtttcttttattcttgtttgtaaatcttctagcaggttcttctttactgtcatctgtagaaggtatttcttccccagtaaaatcttctaggggttcatcctcaacaggttcatctatgacaatcacatcatagttctgctttaaataatttactagactatctttaacttcttctacatggtcttgagtaattctatgacccttaataaccagagtttgtaatttaccctttgctaatttgaccttagccataatatctctcctttctaatgaaaaagagcccacatttatagtgggccttcatgattaagcttttaccttgattatcttagcagcagctttaggatcttcaaacttaacatccatcttaagagttaaaactacataagtagctctttctctaggagctctttctaattcaattctgatatctcttgagatacctataactatattcttagggtgagttaagattatatctgatacttgagtagaagtatcttcataatcttgtagcatagctatacctttaacaggtacaccataagcagatttaacagagccatttataaatgagtcatcaccattagcagttaatctctctcctactaagtctacccaatcagtttctaaaccatttgagcagtagaatctccactcacttgggtttcttaaatacttagctggtacagctttcttagcttgttttaataggtctttacttaaagcacccttttgagcatctactacatgagcagttaattgttttctaaccccatctaattgttgtaagaagtcatcactacccccactagtatcaccatttacaattatttcttccatatcaagagcaactctttcagctaataattgcattacagtgtcttctattgattttccttctatatttgtttctatagtatcatcagttaacttaaccacagctataaattctttagcttgtaaagtaactgttccagtagttggagctgaatatttagactcccctggatcttttccttcagctgcaggatttaatactctttgaccaaatcctattgactcaatctttttagagtctcctttcattggtacagttctagcctcttttaatattgttggctcatcaattatcattttaataaaagcgttagcttgttctgtgtttaatttacccccaccatttaatactgtagagttaaatacggctttatttacttttgacataactttcccccctagttgttattattcttggtttccaccaaataatccattaaatactgatttttggacttcttgtccttcttctccatttttaacaatttgtttacttcctacatatggagccatggctttagctactgcctcagctattttttcttcctcagtcttttcttcttgtttctggattaagccagcctctaacatagctttttccacagccttagcaactatatcttcagtagtagcttgttgaccttcattcttattaacttcaccctcaacaggcttagttccatctaatgcctttttgatagcttcgtctatcattttttgtacttgagtttcattcatctcttcttccccctccactgagtctaataattcattaatagcactttgtgctgtttttaacttttccacattttttgaagatattttcttacctgccttattgactggtaactcaaaggatttagctacttcttcgggagttcctaataatattccctccatagtttcagaaaattcttgaagagcttcctttatcttatctacatcactttcaaaagatctagtgtagtcccaactagaatatggatatagaatatcttctaaggtactaactgcattccagaaattatttctttttgcagtctctttatatttttcagagtatgcacctttctgtatatgctttggagttaaccccaaggcctttccaattttatcaagtatagattgacctatagacttctcaatctcatcaatatctacatcttgtgtagaatacttaccagtaccacccatagaccatccagtaatttcgcctttctcaatcttgctccatagatcatcatcgacaatctcagctttagctaaccaagttccagctttaactactgtaccttctattgtggtatcttcctcagttacccatgattttacaatcttgacaccctcaagagtgttctcattatgttgtaaatcactaccaaggccattttcattaaaccactcacaagcttttttgatctcttcctcagtcataaaattatcatggtagtcagcagtcattggctcatatactacaccgataacaaaatgattatctccttcagactcagactttaagattggcttatcatatctaaatctattaggagctccaagttctttagccacaatccattgccatttattagctgctttatcaactaatgaaaggtaatcaattttagcatctgagatctcaatagctttcttaactgattttactggcatttactctccctcctttacaaattatattctgctatcacttgatctctgatagcttctttttcttctttagataatcctagtatatccttatcaattacaggcccatgagtacaatggcaattcacagtctcagaagctggtaagttgatatccctaggaaatctagcttcataaatccctacagtgaaatattcacctttattaatgacagtaccatccagatcgacatggtgttgccttgggtgaataccccttccccctgagtgtttccactgtattccagttactgcggggcattgattaaaagcctcatccttagcatatgagtgggctcttaacatttcagtaatagcagtagctctagctctagttctactaaagccataagtctcagataatttaccaactacatcttctacagactctccatcttcaagagccttatccagaatattctgtagcttttcatgggaagttaatttcatcatttcccctagcttagaagaccactcatctataaaggtagtagtatatggagaaaagaccttggttagggataagtccttatcaataccttgtaagtaagtatctcctaaattttctatagcttcttgataacaactcttaagagtttctgctaaagaagtatcgatattatctgtacctatttgagactccacatattccataaattcttcaatggtttcaaagtctccatcttttataagatgttttaattcttctgtaaggagatcagaaatagtgtcttctaattcagagatcttttctattgtataatcaatctcctcatagccagcttctttcaattctttcttaaggctatcatcttcattctttaagatcttatcaattttagcgatcaactcatcagtacccactattacttacctccttttaattgatagtttaataactgcttcttagcctctttaagtacagctatcacctcatcactcccattagagttttgagccttctcaattatacttgttaaattattactcatagaagaggcaactgagtaagtagggatatttgcccactcttcattaataggagtgtaatccttattgattgatgaataatactcctcattaatctgattaggagtcatagctctagaggctatagcaaacattcctttctgataatcagagttctcaatattaggactcttaaaataagcttccacatatttaaaaccatagccagatagaataacattatttagtatccatgttaggttttctctttcaggaacaaatacttgctcttcagtttttcttgtagcctctctggctgtagccacattgtaatcttgagagtaccctacatatatgtctggtagtaagaaagaagattgtatctttttcctagtattatccatgtagtcttggaataactcatccttctgtaatatttcagataggggcttgacctcaacagtaactggcttagagtcatcaagggcagtgtctttctctgcagcttcagtttctaatactaagaaaccatgctgggaattttcaccttttacactattcatgtactcagttaactcattccaagatttatcattaagagtaccaccttgaattagtaacattaatggagtatgtttaccattagtaaaatatctccagtttaataattcggcctttcttgaaccttgagcggctaacataggaccttcccatcttggtgtaccatagacaccataacctgttttaaaatgaagtatactgttagctcgatctttaagaggtatctctcccttttcataatactttccagtattcatatctagatccctaggatctccccattccttaaagaagattgattgattatttacagtctgcttaaatcttctgaacctttttctcctagtgtactgtttaccatacctggttacagtgtactctgtactctcatcatctagcttaagaattttaattgtcttagggtcttcaatttcttttatctcttttacgagagctgagtctttaggatcttctacgatctctaaaaaagaatacccaagtccttctctccaatcaactatctcttcaaagacattcttggttggtttatcaaaagacagggtatcaattaattcttccagctgcttatattcagctttcatttctggagtaggttcatctaccttctcaatatatctaatacctattccgaatccagcaatattatttcggtaggcctttttacattggggtattattgtagactcccttataatcacttctaatagttgggggtcatattcaggagatatctcatcaacttcaattccaagtacactggtttgttgattagatttatccactggatccaaagacttttggatagggtctccacatatacttctggcggatatctttttcaaagttactttacctcctttcttacattatatatcaaattataaaatccagatggcctcatatttcagggaggattacatcaactccacatatagtgtcaaatatggccatacacatgatttaaaaataactataatttgatacgtttattataacataattcacaaaaatttgtcaacgttttatttttacattattttccggaagattttctgtatatattccccatataaaattatataattattaatgtatactatattactatattaatttatgaaataaattatatagtatatagtatacttatattacatatatataaaggtggtaactctgatatgccttctatatcacattattactaatatagagcctagtcaatttcaaaactgatatctcacatatcagaataaaaattattggctttaataggcatatcagttactagatttttcttaatttagatccaacaggaaggcaagctaagaatagagagtctgctaagtctggagaatgtagtcctcttttcttcatttcatccttactctccagctttatttgacctttggagttgaatactttaaactttctggtagtcaattctcccacaaggtcagtattattcggtagccttaactcatcatcctccagtaattccttaactacagaggctatatatgtggttgtatcataataaaaggcataagcctgggaccctctctttagtggcataccaaacttacatggtactaccttcaaccagtccagtttttcttcaagttttatctcttttaatctatcagttactccaccacctacaccagtgtcgtctatttttacaactacagctagtttagggaactcaccatgtagagatttagcctttcttagtacttctcctacagttttcatagtgtccttaccttggtaatagataggttcttccacatatccatcaattttggtcatgattgaggtcttatctgaaccaaatctagctacgtctacacccatatctatagtaactggctctgtaggtctggttatttcactattaatacctctatatactgtggataattgtataaatatgtcatcttcttctagtggaaagtccccttttaccagacatctgtagacattggactcttccccatactttttccttaacattttatggttttctttatcagttctatcaacttttgtagcgtcaatatgaatacatttaaaattttccttaagactgtggtgagaatcaaagaagatacctgtatttttaacagggttaccaatcaaaattatcttattttcctcatccgatatagtaccaagtaatgcttctaggatctcatcatctattccaggagcctcatcacatataattagcatgaactcactatgaagaccaagcatattctctttcctattactagttttacacatagcgaaccaagtcataggactacaattcctagtcatagtggttttatttactgtaaagtaatcttccacaatagttccttgtagccattttgatatttcaggccataatacagatgttaactgtgaaaatgtaggagctgtagcaattaccttacattcaggtctaaaggtcatataccaaattataagacaagcacaggccatagtcttacctaccccctgtcctgaccttacagaggttctcttattgtctcttacacttattaaaatatctttttgatgtggatctaaatgtactcctagtatatcttctgagaatccacaaggatcgtcatagtataaatctactaattcttcaaaggatatttcttctagagcttccttgttgatgtggaacttatcaactaaagcatctagctctaatttctcaatcattctatctaaatcaaacaattatatacacctccttagtaaagtgctttatttagtcctgtaatacctttttacttagtagtattagattttcctttacctgcattctttttctttctatcagccattttagctctaagaacctcaagatcttttcctttaccagcattattctgattttcctgtatttgtaaaagtaatttaaccttttcaagttcaactttcatcacatcagtacttaagccgaagtatacctcaagtctttttagtgcctgaagtctatcttccattttaactgatactccatctttactttgtttaatctcattgattagagtaccatctacagccttactagacctcaataatactagatcatccttgaattggcacacatcagttatatctgcaaaggctattttcatgtactgccttattacatccataccatcgcatagtaattcttcatctctatcggctctgatagcctttagagcttcctgtattttcttcttttttaatagtttagatccatgtgcatgagaattattcacaggatagcctgctctagcagctgctgtagttgcattaaagcacttcatataatgaaatacaaacaactcttctttctcatcaagatcatacttctcagccagggtcttaatagtcttttttgttttgctactcaatgccacaatatcaccttcttcttctaaaaatgttatgccctgctctttaaattcccttagacactgcctaacaaaagtctgtgataccttagcaaggttccttaattgtttaggatgtgtaaagggatcagccttcagggcattatatatcaattttttggctttcttcaaatgttcaggagtacagtactctggttcagtgtatgacttatagctcataatacaatatcacccccttaaaatccagatattactacgtgaaaaatggttggcacactaacattaatgctagtaataccaaccaaatggcaaaattaactatggatttataccactattataccacatctggataatgatgtcaacgttttataattttacctcaccttgtagtattttttcatacatttcttcatctatagttccctccatcaataggttcttagctgcaatttgtatttcttctggagaatatactggattacctttagtatcaagttctgtaccagtatatgagtctaccttgctcctgttcataccatacccaactgatttatgaacttctagtttttcattatggtcttcttgccagtgttttaattcttcctgatattccttattttcttctttagtttgtttacattcaataggcctattatctttcatcttttgctttagagctgctgtattaggtttacagttcttagatcctccacctgaacctctattcttatcttcctcataggctttattaatctcaaccccaattatcagatccatcttctctatcaagtaaagacattcttttactattggattgttaggttgatttttagaaatgatgtcttgacactccttctttaattgatgtggattattacattggctagggcatttttctcctggtccatatggaattgtatatgctaagcagttacctgctggatcccttctataacatcttctcttacatattattttactcattgttgttatcctccttaaatgtaataaatattgctcttgatactctggaaatgttacatccttgaaatgcttctcttactttatcatgtactagatctactcctactaaaggttctatcacaactatattattagatcctagtacaggaaatacctcacattcagtatctagtccatctataaattctttaacttcttccaatcctttaactggaactggcttaatccttgggctttcttcccacaatgatttttctcttatatatttttcttctgataatctatcaaactcatcaatgttactttcataaggatttttaatatcttcaatacctgcagaggccatggatcttatggcctttaattgactatcactaaagaattttcctttactcatactgttctacctccacattattacaaattaatttagctattcctccgcttaatctgtatttctcaagatataccactttcttaattcctgcattgattattaagttagcacagctctcacatggagataaggttacatacatagtagttccttcagttgagatcccatgtttagcacagtaacttattaagccagcttctgcatggatcacaatatcacatccaggtcctaaacaagttttacaatgttctactcctgaaggtaccccattatatgaagtagcaactatcctgttatctctaactaatacacaaccaactgcttttcttaggcaagtagatcttcctgtcataagtttagcaacttccatgtaaagtgtttctctgcttattctgttgttattattttccatagtttatcacctcaattttattatatactattccctatcatatgtcaagagaaactttagaaaggtatatcaccataatccagatcaatacatttctatgttaaaagggagatcatctctccccccctaatatctttttattcttctgttgttctcatcaacctcaaccacttcataccttctaaccttatcaggtataccttcctcctgtaatactttaggaatatatcctatggcctgtcttttgttatcaacttcaaaatacctagtcaagatcctttttgatgttacaatcactaatcttactctcatattcattacctccatgtggcctgaactacagctccaggtctatgcttagcttcaattctagcttcttcctcagtatcaaaatagattatatcttccttgtgttcatccttaacagatacccagtgtactcctaaatatttattatgtgctaagacactaacatcagggttccaatccacatcaactgtaacttgtaaaactatatatcttcttttcatatcagttctccttcttgtatttatttgttgattttattataagagagcctcggaagtttgtcaaagattttctcgaagaaattcgtataatatcttccgagctgagcataatatagactataaaacaaaatcaaggagttgtaataaatgaaagataataatttaatacccttaagagatattgccaaagaggttggaataaatgaaaggaaagccagaaagctattaagatcacatgagaataaagcaaatcacaattattattggatgtttacaccaagggagaaaagacatattgtatcagttctaaagagactgcaataggttttctaaaaataagggagggattaaccctcctttttataattttttgagttctggaaaaatccttaattagttcaagtttcgtaaagttctcttgatcttttccattctcttcttaacttggatagcttcttcttcgcaaggagcatggccatctaggaattggtgccatcttctttcaagttctctgtattcagattgaagttccatgaactcaagtgaatgattgtccatactatcacctcctcaacactattataagtgatactgtcatattctgtcaaataaaaaagagagaatgttattctccccatcttaatatctttattgttgctccatcaactcccttgccatccacatcatggacatacttatcaagtctcattgttgagtcctgtataattcttttgtcttctctgtcaaaaaccttaatgacatcagggttacccctgtcaatgatgtaagcaatctcttgagttccatctgcatatgttacttgaagtaatgaatgtagaggaataaagtctggagctgcccaatcaccaatcttaagatctcctcctatactattcttggtctcatatcctgccacatctgtaggctcatctgatgagtaataagtaacttcaactttgaactccattcccttaaatgaatttatcatctcaattagattttgtttatcagttctcagtttatcattctgatattttaatccttctacattgtccatcagtatttcagtctgctcagttaattgtttattcgactcaatatattcttggacagcatttccccaatgattgtttaggctctgtatggttaatagtagccctcctatcacaattattgttagtaatgacataattcttttaatcattctcttttaactcctttatttttaatttgaatttctcaattccagatttatatttacccacaatatcaaaggtatcggttaatattgcataggtttcctcatcagaattaaatctaaagcttcctctcttgatgtctcctagcttattaaaggtcttaccctccttgtccagatatcctttatagtccatatagcacagacaagcagtaacaaactcttgcttattcctgaaattatgcttgatacccataccatctacccatttacttaatacaaataggctttcattagtcttctttactttactattctggattagatccttccacacataatcattagcctctttaaatgacttaaatctcttgcctttcagtatcttactactctggatcttggctttataatcctcaaatatacttggccagaagtcttttagatataaatacaaggaaatatcatctaatttagcatttggatcgttaatctggattacaggatgaccatccattgctctgatattattccttaattgggtattctctctcttcattttagatacttcaatatataatttaacgaatgcgtcagacaattcatgattggttaattccctttttaggggcttaccatctttactttctttagtagttacaatattaaagtagtcttgtaacctatcccagttattcttcattagatccacctcctatgcttacattatacatatattgtactgatatgtcaaagtccaggcattaactgatatctgatatgcctatatttttaccccatatcagtctgatatcccacatatcagttgaaactacaaaatcacaaaggtgttggtatcactggcgtagagggatatcagttatgccccctttatatatacatagtattatatatactatataataatatattattatatatagtatatatatattacatgtggtgatacaagggcaggctattgccttgcctccattatcattacggcaggcatatccatgcttccgagcttttgctctatatagaaaaacccagacggtcagcgatcgagctcttgattttgtttggttggaaagagagaggggaagagagggaaagagagagttcttctctggttggttccctagttagtagtaatcgttgacagttatacttacttctcttataatgacagtaaaggagggataaccatgaaaacactattcaatggaactgatgtaatattaattgaaactactaacttcttcattatgttacaagaagaaggtaagtatgtactatctaaaacagtagataagaaaatgggagagcaaatatgtaaattacctaaatcagttgctcttgtatatactaatgaggaatacactaactctaatgaaccaagatctatatacttagatgacaatttcttatacgaagtgcttggtaatgagaagtatatcataagatacagtatataagagcccacaatcaattctaaggcctctaaaaaataaggtaatgtattttgatgttctatactattagaattaattacaggagctgctagaggcctttaaaggatatataggaggtactactaatatgtataaattatacttatgttacaataccaatgaacaaaaacattattgtggtattactaagaataatattgtggatcgcaagatatcaaaaaagcttgattgtaacttcacactaataatattaaaaaatgataatattaaatatctggaaagtatgagggataagttgaagggtgactataattatcagaagaaaagggtttatagaaaaagcactgaacaatattataatactaaacaagaagcggctagagataatggtttaactatgttacaacttaaaaatttattgaaagatggaattgaatatacttggaggtaaatgatgtatttatgtaatggtaaaatagttaagaataaaaatgacttaaaagataagtttggtggtaaatcagtagtattcatcgatggggatgtagccacagtaatggactttagtgacagaaagccaaagataacaaaatatcttattagggaggtatcacatgataaacacatcattattaaagagactggaagggatagaggaaaagcataagcaaaatacagatgagataagaaagaacctaaactatgccagatatttgattgagaaacagtattgtcatttatctaaggagcaaaaaataaagctaggtcagtttatgggattgactgagaagaagctaaaagaaatacaagagataaatagtcaattaataaatttgagagtggggctaagttatgaagtcgtatgtaataaccagaaaaaatagtggggtatctttagccgatacagacatgggacaagtattagctaagatactatgtaagcatgggggaacacttacaatagaggaaattgaaccagaacatattggtcaaactttttctattgtagaaggtaaaagaatatggtataatagaaaaggaggtgataatattggggagaagaagtgctatgttaattctcaagtatgactgcgaagaaactccagaaagaattgaattacgaaagaagtggttacagatttgtcttcattcaataatatattatagatatgacaacaatatctggacagaccaacaatgggatgagactgccagagaggtagttaaacttaagaatgagaatccaggtttagtaaacagtatgccatttaggaatgaccttaaagtctttgatggatccactggctttaatctttcatgtatgcaagatattaagatgctaaggtgggcccaaaacttacttgactttcatgttaaacgaggacttgatggaaagaataagaaaaagaagaagaggaggaaaaagaaatgaacctacttgagcattatataaaaaaggtacacagtgtaagagagtaccatgagtttgataatgaaccttgggctaaaggtaaacaatatgtagaagtagacatggactatatttgttattctaatacaccacaaagaactaaattagtatttactgttgatgagtgggagaagataaaatatagaggctactacatgggataggaggaatagttatgataagagaaatgggcatgagaatgctaataccagaaatacatgatataaaagatagagatatacaaaattttgtaagaaaggccctaaatgaggtagttgatgagaagttctttataatacctgcaagtagtactggaaaatatcacccaacttatagtgcaggggtaggaggtttgttaagacatactaaagctgcttgttatataggtaaagcattatgcgaagctgagatgatatgtcaggaagataaggatctgatacaggctgctctgatattacatgatattaataaacctgctaaggagcatccatacctagttagagagactctattaccactaaaggaagagtttccatcaacctatgaaaaggtaatagaactaattgagtctcatcatggtcaatggggagagtacccaatcaatacttatcaaaagagaatagtacacttggctgattatatcagttcacaaaaaggattaatattctcatatgaccaaggaactgattattctgacctgttatgggaggagaagtaaatggtgctaggggtaataaccctagtatcaatatttttactaggagtgtttataggttttgaggtgaagaaatggaactaggttttgcgatactagctataatagtatcaatatttttagaaactaaatatataaaggagtagattattatgtcaagagcaagagctgcaagaagaagagctgaaagagagaacaaaaaccttaatgttcttaaagctgtggacatcatgttagccttatcatgctatacattaaagaaggaaggttatggtaagactagaatgactaggtttgtggaagagatgagcaaacacacagaggagattgaaaagggaacacttaactatgaaaccataatgggagagattaaggatttagtaccaccaggtatatttgatttaggggaggagtcaaaggagtgatagtcttgggatatttattatgggcatttagaatattaggcatacttgtaatagtatggtgtataatggaggcgatcagttgtgagcaataagttatattttaggtatagtgctatgaactctggaaagactactcagttaatccaggtagcacataactatgaagaaagaggaatgaagccattagtagttaagcccggaattgatactaaaggcggaccatgtataatcagtagaataggggtagcaagaaaggtggatgttcttctacctcctagggttaaactatcaaaaatactttctgattttgaaaagaattattgggcagtagatattatattaattgatgaggtacaattcttatccagagaacaggttgatgacctgttaatgttatcattacattatcctatcatatgttatggattgagaactgactttaaaagggaaggatttgaaggaagtacccgattattacaagtggcccataatattgaagaattaaagaacatttgtcaatgtggtaaaaaagccacattctcaatattgaaataccagggtaaatatactgaccagggtaaccagatacaaattgataaccagcccgaaagtgttgaatatgaagccgtttgtaatgaatgttataataaattaattttttctaaataactcgttgacaggctatatgggatatgatattatagtattgtaatcaaattacaaacaccaaccaaaactttataattagttaatttcaaactatactaaatttgttatttaaaaattaaaaatatggaggaatgattattatggcaaaaggaaaagcaaaatggttagaggaagcaaacaataaggagctaattgtaatcgtacaatctgctttaaattctggagatgagaaagaagttaaaaaggctttaaaagctattgtggaattagatgaaagaattcctgacacagatggacaaataactgtagaggaagcattagaatcattagaaatggctgtaggaactgtaaaggctgcataccaagcagaaatagatgctgtggaagatgaagatgtagtagatgctgacttcgaagaagttgatgaggacgaagcagaagaagaagaagatgaaaatgatttagaatcattatccaaaaaggaattagctgctatggctaaagacttaggtatcaaaggagctaagaaaaaagataaagatgagttaatcgctttaatcaaagaagctcaaggtgaagaagaagacgaagaggaagaagaagaggatgaaactcctgactactctgaaatgactaagaaagacttaattgctcttgctaaagaaagaggaataaaagttaataagaaaatgaaaccagctgagataattgaattattagaagctgatgatgaagaataattctcatcaaataataaggggagcaaacttgctcctcaaattttttacttattttagaaacagatgttagagggggatgtataaatggcaagaggtggaaattcaacttatactaaagcagtatattgttacaacaataatagggagtatagatccttcagatatgcagctaaacagttaggagtatcggctgttagtataatgagagtagttaacgggactaaaccacatatagggggattagtattcgctgagatagaggaaggcactagaaaattaaagccaagaggagaagtagtttcatacttgaaagaattaggttgggataatatagacttataatagggggtatagtatatggacataaagttcttgaaggatatattctctaaacaatgtgaaaatggagattacattatcctagcagctagaaaaggtaaagaatggaaagatgtaccaatcaaatataataagaataacattgacaaaaaactaaaggactttgaacaacagtataagggttatgatttatattggagcccaatgccttatagtaatcctcaaagaaggatagttaacttcatagagactaaatacttaatacaagatatagatgagcatactgacccattggggattaaacctaaaccaagttatctgtgggaaagttctcctggaaaatatcaggggctatgggaaatggataggtatatagaggctaatcaatatgacgaaataaatccagcattagctaaacatataggctgtgactcctgctttgatgttactcatgtatatagaataccaggaactattaactataaatacaagaataaaccaaaagtaaaaagacctatacacactaaggagatatataagcctaaggtgatcgctaaggctgttaaggctgtaagtaaatctaatgatagtgttaaggtcaatataggaggatctgaggcctcacaatctgaaagaaagatatatgctaaatataatataccaaagaaggttagagacttactggctttagatgatattacttctttagatagaagttctaccatatggtatattgagaataaattacatgaaataggattagagcctaatgagattatattattagttaagggctcagcttttaataaatataagggaagaaaagatgaagaaacaagattaagaaaagaattggataaaatcataggaggagaaatagaggctgatattgaaaaggctgaaagtactaaactgagaatagatagttatcaggatgttatgggtaataatggagccttccctggttggttagtacaaggtttctggggtagaagatctcatggaattgtggctggacaaccaaaggtatttaaatccacatttacacaggacttagctatatcagttgctagtggaagaccattcctaggtcaatatcctgttctagaacctggcccagtaattgtagttcaaaatgagaatgctgactggattatgagggatagaactgaaaagataattagccacagaggagtagttggtaatgtggatataaaaggtaagagaagacttaaagttaggtttgctccagatcttcctatcacttttattaatcaacagggatttatgttagatgaagaatcccatagaaaacagatagaagaattaattgatgagataaaacctgtactagtaatattcgacccattatatcttatgtttagtggagatctaaataatgcagctgatctaaatcctgtactacaatggtgtcttaaacttaagaatgagaagcatacaggagttatgttaatacaccactataataaaggtggaaatgccactcaaaccagaggtggtcaaaagatggctggttcattcatattacatggttgggtagaatcagcattatatttaaaaagacctgatgacttagaaggtgatgatgaggagattgaagtagatatagataaccttgataaacaaagccatttaccaagtaaaatcattatggatagagagttccgtcttgcaggacaattccctcaaattgaattgaacttatcaatgggagaatttggagatccatattatcatgtagaagtagcaatacctggaaaagaagtaattgtgaaacctgaggacaaagccaaagttatagaggctgtaaaatcaggagctcataccaaggaggagatagtgtcaatatcaggattaaattcacagaaggttaatctagttctagatactttaaaagatactattgtatattctccggataaaggttataatataagtaagaaactaaatattgggaggaaaaaggcatgataataaagtgtaagaggccagcaggccataatcatgtaaaggcctttgtagtagtaattattgctagaggtaagaagaataaaatcacttatcattcaggattgaaatatttctatacttataaatcagctaaattatttatagacaaagttatggaagaatgtccctgggctgaattttattgggtggatgtccatgaagctcataagatccctgaaggcgaagagatacctaagaagaagatgtggtgtccatattgtcaaactattcaagaatttaagagaagccaaggaggttacaagaattgccctatctgtggcataagtgaccaagacttttatgtaaaatgtatgaatataaagtatagggaggctaaggctaatgcgaagaagaaacttgagggaagtaatactaactctagggatatcattgtcaatggggataatgctaactcagggggcaaaactaatagaaagtcatcaccaaagaaaaagtcaagaaaagttagtagaaaagcttcataatgctttagactataatgttattgagacttctccatctggactagatggggaggatcttatcatcaaactaaaccttaatagtcctaaagaccttacccctaacaatttatctaagttagctaatgaggttatggtcaataacttaggagctcagaattatactatacttgtttatgatagtaatagaccagaaaatcttctggctgttatagatatacctaaaaattctaaaataacctttgacataataaattaattatggtaatataaaaatgtagaaggaggagaatttaatggtcaataaaaagtattacatcttgaaagaccttaatgatgacaaaccccaactatggaagggaagtttaaccactgctactaagatagcaggagcattaggagttagtgtgataaaactaaataggtatttagtaagaaagggatttgccagaaggtacaatgagcatacacttgaaataatagacccaatccttgctaaagaacttggttattatgtaactccaagtaagtcccttataaatcctttatttgaatataatgataaaggagtacaatatatactaaacttaatagtagaagatctaaagaatggaggagagctttaatgattatcattttaggaatggataactcaggtaagaccacaacagctaagaacctaactaaacacagaggaggagaatatatccaatcaatgggacctggttcttatgaagaacaaagagaatgggtattaactcaaattgtgagaaaaggtgaaagggaagctatacatgatagatttacttgctttgaagaaatggtgtatggacctatcatcagagatagtagtaatttcaaccttgacagtaaggagctaaagatcttaaaacatttatgtaagccaactattgtatatgctagaccacctagagaagtgatcttcaattttggtgatagagaacagatgccaggagtaattgaaagagctgaacatctactggctagatatgatgaactgatatggaagttattctgtgatggatggaatgtactggtatatgattatactacttccaatgtggagaagttatcccaattaatagatgactcaagtgctaaagaagctataaaccaatttataaatatatcaagataagaggagagattgttatgaatataaaccatgcagtagaagaaaaagtagaaggagatagattacaagctatctttaatagacaaaaggaattgatggagaaatatcatcatatagaagctagatcaggattatgccaaactgaagactgtccagttaatttaaatgataaaagaggtcaagctagacttaaagattttgcttggagaatgactgaagaagtaggagaagcattagatgcttataatcatgaagaccattaccaagaagaattgattgatggattacacttcttaactgaatttacaatcctagctggtaaggattataatactatagatgataatgctatattagttgatagcctagagaacttatatcataaggctactacatcaccagaatttcctgaaatgttagtagaagaagctgtaactgacctggtaagagaaatgggtatgtgctgtaactgccttaagaataaaccttggaaacaaacttcaatgctaacagatgtaaatgcttttaatcaaagattgttcaatgtatgggtatgttacattaaattattagctgtatctggcttagaggtagatgacatagtaaatatataccttaagaaatcacaggtaaataaattccgccaaagatctaattactaaaataaatacttaggagaaggtaatatgctaataagagactataatgattttgatgacttattcttaaatcttaatagggaaatgattaccaatccagaagaaactatgatgtatactcagaacatacagggctttcaggaggacttagttctctcctgtaagtcccataaatgtactttaaatctaggggactttggatataaagaaggtaagtggggacacctattaagatcatacattgattatccacaattaattgaatttagagaaaagcttaccaagataagtggtatgagctatacttattatttcaacaggaaaaaagctactaatggttcttgcttgatagctgcagtagtaactagaccaaaaagaaaaggaccttggaaacacttaaaaattatgtatagagtatgtgaattacaaaagaaattcgcagctgatctagtattaataaacaggttcattgaagaactacctcaagaagtatgtgagatagataatattacttttcatatgtctcaagcttatttgtcaggaatgtttataaatggatatttcaattattttaaggtaccaagaaagagtattgctaatagcaaacatccttggcataaatccttgaatagtaactataataggttctttaaatctgaggaccagatacactcttataaagctctccagaagatgcagttattacattttggtctggagaaatttccaaagatagatatcaataaattatctattgacaagtactttaataagtagtataattaacttaacaaaattaaataaatggaggaattaaactatgagaatttatattaatgctcaagaagcatttgaagaagtaaaaagagatttatgtgaaatggggattgaggtaagacccaaaactatgcaagataaagttatagaaggtaatccagattatttcacaaaggagttacaaaactatagttacacaattctagaaagtaaacctgaagaagttccaggtgtatctcaaccttgggcagatgctgaatttagagaaaggatctacgatccacaaggtgtcatcaatcaatactctcttgaagaaagagaagaattatttggtatacatccacatcatactagggaggctttcataaatcccggtaaagcttatcaattaagacctgaggtatggaatgaatatctaagagatggaaaatttggatattcttacaatgaaagaatttggcaatacagacaaattgaggatattatcaatagaattaaagaagatccaggctcaagacaattatggttatctctgtggaatccagctattgatccattcaatataggaggagtaactagagtaccatgttcattaggatataacttccaagtaagagaaggtaagctaaatatccattatgttatgagaagttctgattttgctactcactttgctaatgatgtttatcttgctatgaaactattacattgggtagctgaacaaactggctatgaaccaggaagtttctctcatacaattttctctcttcatgtttacaacaaagatattaagggggtattctaataaatgagttacatgagaaaagaaagatataaacattctgatcttaataataataaggttaacaagaaggaaacactaaagcaatttattaggaatactgaaaaggaatttgatttagaaccagctaaccttggggacatgactaggaatgaacttaataactatataaattatatgatgaactgtggagtaagtaatatgtataattatagtgatgataacagacctaagaacaataacaatggctgtcttggttgtttaattataatcttagcagctataggattatgggtaataatttttgatattgccaatataatataccatatgatattctaatttaaagggggctatcctaatgaaaaatataatttgtcctatgtgtaaaaactcagtaacattaaaaagaaaatatggagctatgttttggatcatgattttcttaactggaggcctatgggtagtaaccatacctttcaaaaaacataaggtctgtccagtatgtaactcaatcataaaataaagataagggcctttaggggtcctttctatttaggaggtaatattttaaatgagatgtaacaactgtaacctatatactcactcagctccctcatgtatagaaggaacttgtgtaggaaagaaaaagaaaccaagaataatggttataaatagtttagctaatgatagggatgaggccaatagaatagctactcctgataaatcattacttgataaaatggaaggtctagacttttattataccaatgctattaagtgtagaactcctaagggtactaaaatcaaagtatcggagattaagaaatgccaagaacacttacttaaggaaattgaaaaatataaacctgaatatattatgatcttaggatctcaagccttaaagatgctaagtaatgaaggtataacctcaatatgtggagtacctaaaaagcatgagaaatatggctttaagtttattgccagctattcaccgggtgtagttgcatatgacccaactaaagcacaatttgtagatcaggcctttaataattttaaagccatggtaaaaggtaaagagcatgaattaccagagcttaacataaaacttattaccagtatgaaagagttaaaccaggctttcaaatatttaagagaggaaggttataatagagtatcatatgatatagaaactagaggcctagataggtttaataataacattaccctatttggttttggtaatactcaagtacagtatatactgcctttagaagtcaaatatagcccattaaggggtaagcccatagcacaaagaagattagctaaatccctgattaaaagacttaattctgagatgaaggaaagaatagcccagaatggtaagtttgatgataatttcttaaaggagaaatatggtattaagccaatcataacctttgatactttactagcctcacattgtttagatgagaatacccctaatggtcttaaggagaatgctttattacattgtaatgctaaagactgggatataaataagaaattaaagactggaaatgtggagactaaatcagactttgaggattatgttagatatctgggatatgatatatactatacatttgctctatataaaatatttaataagagacttaaaagggatgagagcttatataaattattccaccacttatacatcccagctagtaaagcttatgaggatgttcagttcaaaggtatatatgtaaatcaggagaaattcaaagaggttgaaaaatacttaagatcggaactagataagattgagactggacttaagaagtatactaatggtaaggatattaactggagctcacctaaacaggttggagaatttttatatgatatccttggtcttcctgtgattgaggttactgactcaggagcacctgctactggggaaagtgtattattaagactaagagataaacatccagcagtagaactactattacagcataggggagtccatatacaaatttctcactttatagatggttggttaaatagaatgcacaatcatagattatatccaaactttaaacttcatggaactgtaacaggaagaacctcaagtaataatcctaatctacagcaagttcctagagataagaaaatcaggagtttattaggaccatctcctggaagggtattcattgaagccgatctatcccaggccgaacttagaatagctgctatgatggccgatgaggataatatgaaatttatttaccagactggtggagatatacatgactccacatataatattatatctggggaagatatcaatgatgagaaagatccagcagttaagaaggagaaaaggaaaaaggctaaggctgtaaactttggtttcttatatggtatgcaatggaaaaaattcaaggactatggaagagacaactatggccttaaattaacagatgaggaagccaaaacatatagaaggaacttctttaataaatatcctaaactattaacatggcatgataaacaaagaaagattgttaaagccaatggtgaagtaagatctccaataggaagaattagaagattaccagatatatattcatctgatagatctaaagctgctgaagctgaaagacaatgtattaactcacctgttcaaggttttggttcagatatcactttattaggcctatgtgagattacaggctatgctaaatatgttaatcctgaatatgtactagataagtctaagtttgatgtattaggctcagtacatgactcaattttatttgaggtagataaagattatgtggaagaattagcttggaaagtaaaatcaatagttgaaaacaatatggtattaaagaaagtatttaaatttaccccaacagtcccaataatcatggatatatcagttggttattcttggggaggatgtgttgaactggattttaaaggtgattggaaaagccagataagaaaagtgttgacagatgaataattatctgataatataaatattgtaatgaaaaggaggtataaggatgttaaaaataagtaattccagaattaacaaattcttgtcttgtccttatgcccattatgttaaatactatgaaggtctggtacctaaaagaagtggagctgccttacaaaggggctctgctatccaccaggctatagaagactaccataatgggaagagttggaaaaaatctgttgataaattttccaaagagttttacaaaaatacatttaaagaagagatccttgaatttggagatattccaaaaatggtttattctttatgtgataattatttccactattatgatgaaaaagaagataatgtaacctatgtggaaaatgaacatcacttcgaattaaaactatgtaagggtgtaaccctagaaggctatattgatagtgtcttagatgtggatggaaagatatgggctaaggaaactaaaacctataaaaagatgcctgatagaaacttcctgatcttcaatagacaatctgctatatatacctgggctcttctacatgaatacccaaaagtaagtggtactatatgggatataatattagctcagcaaccaggtagacctgaattaactcaaaaaggagtattatctcaaaagagaattaaatccacacctttagagttagaaaggggaataaaagaattaggattagatcctaaagattatgagtcttatattaattctgctagatgggaagacttctttgtaagacatccaataatattatcaaagaatatacttaacagtgtaatggatgatactattgagatagctaaacttattagagatgagggccacaaaagaaaggaaaagaaccttggaaaaggttgctctttctgtgaatataagtctttatgtcaagctgaacttttaaatcttgataaagaatttattattaaggctgactataaacaaagggaggaaagtgacaatggcaaaaaagcaaaaatcaaaatcaaatagttttgaggacagattagtggatctatatgatatagatgagcccacaatattaacactttatggaagatctggttcaggtaaaactactatctcaggaacactacccaaaccaatattctttattgatgtaaaggacaaaggtactctatcagctagaaacaagcttagagttaaaagaggagatatacaagtatttagtctaaagagttttgatgacatatacgaggcttatgattacctatcagaaaacactgataaatttaaaacagtagttatagaccatttaactgctttacaagaattaggtaatgaaaaggtcaaagctgaagaaggtaaagaccagatgagccaaagaatgtttggaaatgtggctaattatatgaaagaggttataaacctttataaagaattaaatgaggaaggtatactaccatgctttatagtacaagatagattagaatctggtgatggtgaaggagaagaccaattaatgcctgaggtaggaccaggattaatgccatctgtatctaaatacttatgtgctgtatcaagagtaataggtcatacttatttatatgaacactcagaaaaagagggtatgaaggttaagaaagaaatccagtatagattaagactaggacctaacccttattatattactaagtttactagacctcaaggatctgaatgtccagcttatctagtacatgatctaaaatcaccaacaactatctgggaggatattgaaactattctggctggtgaatggaataataagccagttaaatctggtaaaaaaaccagtaagaaatctggtaaaaagaaaaaataaaaattttctaaaaatacctttgacaactaattaaaaatctgataatataaacttgtaaacaaagaggtacaatatcttgacaaatatttatctcataatgatattttaaaatttaaggaggacaataacatggctaaaaagactactaagagaggtaacaaaaacaaaggaggattaaagattgatctttcaaacgttgaaacttcagttactatcccagaaggaaattacattgtggaagtagaagatgtagaggttaaggtttctgaaaatagtggaagcaattatttatcatttacttttgtaatagcagaaggaaagatgaaaggacaaaagttataccacatttgctcacttcaaccacaagctttatttaacttaaaaggtgtgttagttgctttaggatttgatatccctgatgaggagttcgaattagatacagaagctctagttggtttacaatgtggggtagaagtatcacatgaaatatatgaaggtaagaagaaatcaagaataactgattttataaaccttgacgaagctgactctgatgatgacgaagaagatgaggatgatgactcagatgacgaagaagatgaggatgaagacgatgagtctgaagttgatcttgaagaattagacaaggatgagctaaaagaattagctaaggctttaaaaatcccagctaagaaaatcaagaaggctaaaactgaagaagatctaattgatctaattgaggaagaagctgacgaagaagaaatagctgaacaatataatgacctattcggagactctgatgaagatgacgaagaagatgaggatgatgactcagatgacgaagaagaaaatgactatgagtcaatgactttaaaagaacttaaagctgaagctaaggacagaggcttaaaggttaaaaagggaatgtctaaggatgacatcatagaaatgctagaagaagatgatgaagaataaaatttaattattaaaatatgagggcctttaaggtcctcctttatttacccttggaggctactattatgcttgaaagagatgtggttaaatccataatgaatatgcttaaaaaagaatacccaggtttttggtttaaaactcatgggggaccctttcaaatagctggcttacctgatatactaggttgccacaaaggtaagtttattggtattgaagttaaacttcctggaaaagagaagaacctaactcaaaaacaaaaagacattataaataaaataaatctagcaggaggaatagcttttatggctacctcagctgaatatacaaggaggagattacatgaaaaatttagaaagacaccaacaattcctaggagaactaggagatctgtatgaattaaagaataatctatatggggataatttccacaagacttatcttgaatatggaaaccctgttctatgtataagacttgaagataagctaggaagagctaaaagtttattactaggagatcaagatgacttcccttcatatgctgctcaaaaagaatctgtggttgatactttactagacctagctaactatgctattatggctgctatggaactaactagtgatgataactcagaaatacatgatcttagtaaagaagaatatggtgaagaagacatagatgataatgacgatgaggacttagatgatgatgacaatgtttacgacgaagaagaattagattttgatagtatgaataaagaatccttaaaacaatatctaaaagataatggggttaaattccatagtaaggcctcaagagatgaacttgtaaaactggctaaggaggtataaggaggaggctttaatgcctcttcttttttagtatgaagagaaaattatttaaacatcagaaagaagcattacaactatttttaaataaagaaaagtttgccctatttatggacatgggtacgggtaagaccttagtccctattgtggcccttgagaaacttgaaggtctagatactgtactaatattctcacctaaatctattgtatttaactgggagtctgagatccataaatttactaaacttaaggaatataggatatttaaattacagggtaataagaccaaggttatggaaacctatagagctataaaatcctactcaggattaaagattattattgccaattttgagaaggctaggttgatggataaataccttatgaacttaaagccacagtttattgttgttgacgaatcccataaggtaaagaatagaaatgcccagatatctaaggctctatataaaattgctactaaatgtaaatatagattgataatgacagggactcctactcctaatggttatgaagatttatttatgcaatataaaatcatgaactcaaatattctaggagttaattggaaacagtttgaagatgactttataatcaaaggaggttatatgaactatgaaattgtggggtataagaatgaggaaatattaaagaaccttatgcaccagaattgctatatagtaagaatagaagattgtattgatttaccagaacaacttcctgatctgtatttaacctgtgaattaaacaataaagccagaaaggcctataatgaccttagaaaggaaatgatagcacaactagatatagtacaggaaaacattcctaggaagcaattaaaggccttattaaggtctaatggtataccctatgagagtaatgagccatatgaggatttattcttaagggctaatatgtttatcaaccaattaacagctgatcttactattacccaatatttgaggctacagcagatctcaggaggatttattacaaacaatgtaggaaatagtattaatatcgataaaggcaaactaggcttgttacaggactatctagaaggatataaaaagcctgttgttgtaatatgtaattttcttgaagaaataaaacttatacatgatacttttaaaaagacccacagagtagaatgcttaacaggatctactaagaatagagctgagatcaataaggattttcaggaaggcaagattgatatattaatactacaaataagttcaggtagtgttggtctaaacctcttcagggcctcaagattaatattctatagttggaactataagtatgatgattatgtgcaagctattgccagaatcaaaaggaatggtcaaaaggagccttggcaaataatacacttaataactgagaatactattgatgagaaaatattaaaatcaatacagcttaaaagggatagagcagaaaaactgttgactattgataagtaatgtggtaatatagaattgaggtgataagtaatgagaaaagaacaacctagaataagtaatcatactgaaatagctaaagctttaattgaggctggtaaaactattaaagctctaaccaagattaaagttaaggaggaaaagaaaaaacaaaaaggacttgaaaggttaagactcctaaatgaggaaaagaagaaggagagagaattaatgaaagaatttaatcctaatgagcccaagaagccaagaaagaaaaaggacaaacctataaagaaggttgatctgaataaagttgaggccaaaggagattaccctactactcctggaagtaatgcacctacgggaacagttgatttaaaatctttatgtgagtcattaggattagatccaagtaaagccagagccaagctaagaaagcaaggggtaaacaaaccctataagtggtcaggatctgaacttgaggatattaagaaaatgttaaaataaggggaggtttaaaaacctcttctttttcgttgacaatatccacagtatatggtaatataaatattgtaatcaaacaaaaaaggagagaactgaatatgaaagatcttgaaaaacttacaagggaagaattgattgaacttgtaaaggagttatctattgattggacaggagcctataatagaaactataagattgaccttgatgagtatgagattgtatatattgatcttaatgatctaaaagagttcaatgataaattaggtcatctagaaggagatgtgtatatacaatacactgtaaatcttattaaggcccatttacatcctgagaaggatatattgattagatggggtggtgatgagtttgtagttatcagtgatatagcttataatctgtgcaataccttgaacatataccctgagttatcatgtggctatggtaaaggtaaaactattgaagaagctatagtacaggccgactcaatgatgtataagaataaaaaatctaagaagttaagatactcaaaggaggattagttattatgaggttattatggttaaaagaatatttggatatatgcaatatcttgggtattgagcccacaatggaatcttgtgctaggtggggaaaggataaaaaattatcagggttttcattaaccccagtaggagcaactaaatagttgcttctttttaaagcgatccgatacctagcaactttcatgccaaaaagaaattcgggattttctttgacatagactccagagtctggtataataatcttgtaaacaaaattaattcaaaggagttgttaattatgaataacaaaaaatttcaagaaactttagaacaattaaaggaggaactagaaatacaaggggtagaatttaatgatgagaatgacttaatagctgctgttgaatacatcatagaatatgattaccacccagaacaatatgtggaagatactttatgtaactaccctgaaatgttcaaggatctataaaactaagggggtgtaaaaacctcctttttatttttctaaaaaatctggatttaatcgttgacaggatcgggactatctagtataataatattgtaatcaaaattaattgagggagatgttaactatggaactaacaagatcagaaaaacttaatgtaataaaaggaattgctcaaaaggctatggataataagatttatccacaatttagaaaagaacttgataaacaggtaaaatcccttggactaagcaacaaagttagggaaagtgaattaggagactacctttcttgctgtatggttgcctatgatgaggttatcctaaaaatgaaaaaagccaattaatatgggtggccaaaaggccatctttttttttttcgaaaaaatctcgagaatctcgttgacagatccagagttctctcttataatt